(19) United States
(12) Patent Application Publication
(10) Pub. No.: US 2025/0313832 A1
(43) Pub. Date: Oct. 9, 2025

(54) COMPOSITIONS FOR TREATING SYNGAP-1 RELATED NEURODEVELOPMENTAL DISORDERS

(71) Applicants: The Trustees of the University of Pennsylvania, Philadelphia, PA (US); The Children's Hospital of Philadelphia, Philadelphia, PA (US)

(72) Inventors: Benjamin Prosser, Swarthmore, PA (US); Beverly Davidson, Philadelphia, PA (US); Jennine Dawicki McKenna, Philadelphia, PA (US); Alejandro Jimenez Felix, Philadelphia, PA (US)

(73) Assignees: The Trustees of the University of Pennsylvania, Philadelphia, PA (US); The Children's Hospital of Philadelphia, Philadelphia, PA (US)

(21) Appl. No.: 18/865,068

(22) PCT Filed: May 12, 2023
(86) PCT No.: PCT/US2023/066948

§ 371 Date: Nov. 12, 2024

Related U.S. Application Data
(60) Provisional application No. 63/341,734, filed on May 13, 2022.

(51) Int. Cl. C12N 15/113

(52) U.S. Cl. CPC C12N 15/113; C12N 2310/11; C12N 2310/14

(57) ABSTRACT

A therapeutic composition is provided which comprises at least one agent which specifically interferes with PTBP2-binding in the SYNGAP1 gene region to prevent dysfunctional protein production caused by an alternative splicing event, which dysfunctional protein is associated with a disease or disorder. The agent can be an anti-sense oligonucleotide, an RNAi, or combinations thereof. The composition may further comprise a pharmaceutically acceptable aqueous diluent suitable for intrathecal injection. Also provided are methods of treating SYNGAP1-related neurodegenerative disorders.

### REFERENCE TO AN ELECTRONIC SEQUENCE LISTING

The electronic sequence listing filed herewith named “22-9943PCT.xml” with size of 221,337 bytes, created on date of May 10, 2023, and the contents of the electronic sequence listing (e.g., the sequences and text therein) are incorporated herein by reference in entirety.

### BACKGROUND OF THE INVENTION

SynGAP1 is a GTPase-activating protein (GAP) that is selectively expressed in brain and highly enriched in dendritic spines of excitatory neurons. SynGAP is a Ras- and Rap-GTPase activating protein that facilitates hydrolysis of small G protein-bound GTP (active) to GDP (Inactive), thus negatively regulates these small G proteins. [Carlisle et al, (2008) SynGAP regulates steady-state and activity-dependent phosphorylation of cofilin Journal of Neuroscience 28:13673-13683.] SynGAP1 is encoded by the SYNGAP1 gene and has at least 3 distinct transcriptional start sites and alternatively spliced to generate at least 4 distinct C-terminal isoforms (e.g., SYNGAP1.alpha.1 (α1), SYNGAP1.alpha.2 (α2), SYNGAP1.beta (β), and SYNGAP1.gamma (γ)., respectively). Human genetic studies have suggested that mutations in the human SYNGAP1 gene are linked to intellectual disability (ID), autism spectrum disorders (ASD), and other neurodevelopmental disorders (NDD), with high rates of epilepsy as well as schizophrenia. The ID-associated SYNGAP1 mutations cause MRD5-categorized ID.

The SYNGAP 1 gene have been linked to intellectual disability (ID), autism spectrum disorders (ASD), and other neurodevelopmental disorders (NDD), with high rates of comorbid epilepsy, seizures, and acquired microcephaly [Berryer et al., (2013), Mutations in SYNGAP1 cause intellectual disability, autism, and a specific form of epilepsy by inducing haploinsufficiency. Hum Mutat 34, 385-394; Carvill et al., (2013) Targeted resequencing in epileptic encephalopathies identifies de novo mutations in CHD2 and SYNGAP1 Nature Genetics 45:825-830; Cook, (2011) De novo autosomal dominant mutation in SYNGAP1 Autism Research 4:155-156; Hamdan et al., (2011) De novo SYNGAP1 mutations in nonsyndromic intellectual disability and autism Biological Psychiatry 69:898-901; Hamdan et al., (2009) Mutations in SYNGAP1 in autosomal nonsyndromic mental retardation The New England Journal of Medicine 360:599-605; Parker et al., (2015) De novo, heterozygous, loss-of-function mutations in SYNGAP1 cause a syndromic form of intellectual disability American Journal of Medical Genetics Part A 167:2231-2237; Rauch et al., (2012) Range of genetic mutations associated with severe non-syndromic sporadic intellectual disability: an exome sequencing study The Lancet 380:1674-16; Tan et al., (2016) Characterization of patients referred for non-specific intellectual disability testing: the importance of autosomal genes for diagnosis Clinical Genetics 89:478-483; UK-DDD-study, 2015; Vissers et al., (2010) A de novo paradigm for mental retardation Nature Genetics 42:1109-1112; Writzl and Knegt, (2013) 6p21.3 microdeletion involving the SYNGAP1 gene in a patient with intellectual disability, seizures, and severe speech impairment American Journal of Medical Genetics Part A 161:1682-1685).] The ID associated SynGAP1 mutations cause MRD5-categorized ID (OMIM #612621). Almost all reported cases of ID/ASD are de novo mutations within exons or splice sites. MRD5 is characterized by moderate to severe intellectual disability with delayed psychomotor development apparent in the first years of life. SYNGAP1 is the 4th most highly prevalent NDD-associated gene, and mutations in SYNGAP1 account for .about.0.7% of all NDD cases (UK-DDD-study, 2015). Some key pathophysiological symptoms of ID and ASD patients have been recapitulated in SYNGAP1 heterozygous (+/−) knockout mice [Clement et al., (2012) Pathogenic SYNGAP1 mutations impair cognitive development by disrupting maturation of dendritic spine synapses Cell 151:709-723)]. SYNGAP1 heterozygous mice exhibit epileptic circuit activity, altered synaptic transmission, and severe working memory deficits [(Clement et al., (2012) Pathogenic SYNGAP1 mutations impair cognitive development by disrupting maturation of dendritic spine synapses Cell 151:709-72; Guo et al., (2009) Reduced expression of the NMDA receptor-interacting protein SynGAP causes behavioral abnormalities that model symptoms of schizophrenia Neuropsychopharmacology 34:1659-1672)]. Some of SYNGAP 1 missense mutations in MRD5 also caused drastic SynGAP protein instability (Berryer et al., 2013). These data suggest that SYNGAP1 haploinsufficiency is likely pathogenic in ID/ASD-associated SYNGAP1 cases. Although SYNGAP1 haploinsufficiency likely affects all SynGAP1 isoforms equally, only the α1 isoform has been rigorously characterized in this context to date, and only few functional studies of non-α1 SynGAP1 isoforms are currently available in the context of neuronal functions and synaptic physiology [(Li et al., (2001) Characterization of a novel synGAP isoform, synGAP-β Journal of Biological Chemistry 276:21417-21424; McMahon et al., (2012) SynGAP isoforms exert opposing effects on synaptic strength Nature Communications 3:90)].

US Published Application No. 2021/0180062 describes a method for modulating syngap by using anti-sense oligonucleotides (ASOs) which target a sequence in Exon 11 or Exon 18 of the SynGap1 gene. K. Lim et al, “Antisense oligonucleotide modulation of non-productive alternative splicing upregulates gene expression”, Nature Communications, 11, Article number: 3501 (9 Jul. 2020), describes RNA sequencing data identifying non-produced splicing events in protein-coding genes, of which about 1246 are disease-associated.

Alternative splicing (AS) of precursor mRNA (pre-mRNA) is a crucial mechanism for post-transcriptional gene regulation that controls diverse cellular processes. There is a particularly high frequency of AS in the brain, where it is required for all aspects of nervous system development and function. Concurrently, aberrant AS is implicated in multiple neurological disorders [for reviews, see (Raj & Blencowe, 2015; Su et al., 2018; C. K. Vuong et al., 2016).

Therapeutic targeting of AS with antisense oligonucleotides (ASOs) that are capable of redirecting splicing has demonstrated clinical potential for the treatment of neurological disorders. ASOs are short, single-stranded nucleic acid analogs that take advantage of Watson-Crick base pairing to target RNA molecules. ASO binding can result in reduced gene expression or alterations in RNA processing depending on their chemistry [for reviews, see (Crooke et al., 2021; Khorkova & Wahlestedt, 2017). Since AS is controlled by specialized RNA-binding proteins (RBPs) that can promote or repress splicing events, steric-blocking ASOs that disrupt the interaction between these proteins and their target pre-mRNA can redirect AS to therapeutic benefit (Han et al., 2020; Lim et al., 2020). Perhaps the most prominent example of such therapeutic splice-switching is a treatment for spinal muscular atrophy (SMA), in which an ASO binds to the SMN2 pre-mRNA to disrupt a splice-silencing RBP, in turn promoting SMN2 exon 7 inclusion and augmented SMN protein expression (Finkel et al., 2017; Hua et al., 2008).

AS of neuronal genes is controlled by coordinated action of a host of RBPs. These include the Polypyrimidine tract binding proteins (PTBP1 and PTBP2), which are each essential for proper development and function of the nervous system. PTBP1 and 2 are structurally similar and bind overlapping RNA targets yet differ by their cell type expression patterns. PTBP1 is broadly expressed across cell types but largely absent from neurons, while PTBP2 is predominantly neuronal (also referred to as “nPTB”). PTBP2 is required for neuron development and survival, and functions primarily to suppress adult splicing patterns to control the temporal regulation of neuronal maturation (Li et al., 2014; Licatalosi et al., 2012; Weyn-Vanhentenryck et al., 2018). Analysis of differentially expressed transcripts upon PTBP2 ablation suggests preferential regulation of targets involved in pre- and post-synaptic assembly and synaptic transmission (Li et al., 2014).

There remains a need for treatment of SYNGAP-related neurodevelopmental disorders.

### SUMMARY OF THE INVENTION

Provided herein are compositions and methods useful for treating patients with SYNGAP-1 related neurodevelopment disorders.

In certain embodiments, a therapeutic composition comprises at least one agent which specifically interferes with PTBP2-binding in the SYNGAP1 gene region thereby preventing an alternative splicing event associated with a disease or disorder. The agent may be at least one an anti-sense oligonucleotide, an RNAi, siRNA, or combinations thereof. In certain embodiments, at least one agent is delivered via a viral vector selected from a recombinant parvovirus, a recombinant lentivirus, or non-viral vector. Additionally, or alternatively, at least one agent is delivered via non-viral vector. Examples of suitable non-viral vectors include, e.g., a lipid nanoparticle, a lipidoid, liposome, and/or polymers.

In certain embodiments, a therapeutic composition comprises at least one antisense oligonucleotide of 15 to 30 nucleotides in length, wherein the oligonucleotide comprises at least 15 consecutive nucleotides of a sequence comprising: (a) SSO_085: TCCAGGGAACATGCTGAG (SEQ ID NO: 1), a sequence at least 99% identical to SEQ ID NO: 1, a sequence having at least 95% complementarity to SEQ ID NO: 1, or a pharmaceutically acceptable salt thereof, or combinations thereof; (b) SSO_019: CACGTGGGAGAGAGATGG (SEQ ID NO: 2), a sequence at least 99% identical to SEQ ID NO: 2, or a pharmaceutically acceptable salt thereof, or combinations thereof; (c) SSO 061: CTTCCAGGGAACATGCTG (SEQ ID NO: 3), a sequence at least 99% identical to SEQ ID NO: 3, or a pharmaceutically acceptable salt thereof, or combinations thereof; (d) SSO_086: TTCCAGGGAACATGCTGA (SEQ ID NO: 3), a sequence at least 99% identical to SEQ ID NO: 4, or a pharmaceutically acceptable salt thereof, or combinations thereof; (e) a sequence comprising a sequence having at least 95% complementarity to SEQ ID NO: 1, 2, 3 or 4, or a sequence comprising at least 15 consecutive nucleotides of SEQ ID NO: 1, 2, 3, or 4, or a pharmaceutically acceptable salt thereof, or combinations thereof, or (f) combinations of (a), (b), (c), (d) or (e). In certain embodiments, the composition comprises (a) and/or (b). In certain embodiments, the at least one agent is at least one antisense oligonucleotide of 18 nucleotides in length.

In certain embodiments, a composition is provided which further comprises a pharmaceutically acceptable aqueous diluent suitable for intrathecal injection.

In certain embodiments, use of a composition for treating a patient having a SNGAP-1 related neurodegenerative disorder is provided. In certain embodiments, a composition as provided herein is administered to the patient intrathecally.

Other aspects and advantages of the present invention will be apparent from the following Detailed Description of the Invention.

### BRIEF DESCRIPTION OF THE FIGURES

FIGS. 1A to 1G illustrate differential gene expression upon PTBP2 depletion in iPS-neurons. FIG. 1A illustrates a differentiation protocol for generation of iPS-neurons. FIG. 1B is a Western blot of PTBP protein levels at different stages of neuronal maturation. FIG. 1C is a Western blot (top, bottom right quantification) and qPCR (bottom left) validation of PTBP2 depletion using “gapmer” ASO delivery to iPS-neurons. NegA, negative control gapmer. FIG. 1D provides Principal component analysis (PCA) of gene-level rlog-transformed normalized count data from RNA-seq iPS-neurons samples. FIG. 1E is a Volcano plot of differential gene expression comparing untreated and PTBP2 KD iPS-neurons. FIG. 1F is a Dotplot showing the top results from GO enrichment analysis (Biological Process, PTBP2 KD vs. untreated iPS-neurons). Gene ratio is number of differentially expressed genes (padj<0.05) relative to total genes in GO group. FIG. 1G is a Category netplot of the most significant GO terms and fold-changes of significant synapse-associated genes upon PTBP2 KD in iPS-neurons (padj<0.05, fold-change >=1.15). n=3 replicates for iPS-neurons RNA-seq data sets.

FIGS. 2A to 2C provide a differential splicing analysis following PTBP2 depletion in iPS-neurons. FIG. 2A (Top) shows Types of alternative splicing (AS) events, number of alternative events detected (number of nonoverlapping events in parentheses), and number of genes having at least one AS event for each type in PTBP2 KD vs. untreated iPS-neurons. Fractional inclusion level difference is inclusion level untreated-inclusion PTBP2 KD and refers to inclusion of the darkly shaded element. FIG. 2B (Bottom) is a Volcano plot for each alternative splicing type. Orphanet genes with the smallest FDRs are annotated. FIG. 2B is a Volcano plot of differential gene expression for the subset of Orphanet genes that are differentially spliced in PTBP2 KD vs. untreated iPS-neurons. FIG. 2C provides Representative AS events for DLG4 (top, encoding PSD-95) and GRIN1 (bottom) shown as sashimi plots (left; numbers indicate the number of reads spanning junctions±SD) with replicates overlaid; at right is the percent spliced in (PSI) for the AS event detected by rMATS. Statistical significance determined in rMATS by a likelihood-ratio test at a cutoff of 1% difference, **** p<0.01 and *** p<0.001. n=3 replicates for iPS-neurons RNA-seq data sets.

FIG. 3A to 3F illustrate PTBP2 CLIP-seq in iPS-neurons and human cortex. FIG. 3A provides representative PTBP2 eCLIP read density relative to size-matched input (replicates overlaid) on DLG4 (encoding PSD-95) showing called PTBP2 binding peaks (light arrows) at similar locations in human cortex (top) and iPSC-neurons (bottom) in the intronic region upstream of Exon 18, a known PTBP2 splicing target. FIG. 3B Top, fraction of PTBP2 CLIP-seq peaks by genomic feature for iPSC-neurons and human cortex. Bottom, fold-enrichment of each genomic feature with respect to size-matched input. FIG. 3C is a Motif enrichment analysis for PTBP2 CLIP-seq. Top-ranked motifs are shown for human cortex (p-val 1e-2761, 28.6% of targets) and iPSC-neurons (p-val 1e-677, 19.3% of targets). FIG. 3D is a Dotplot (clusterProfiler) showing the top 30 categories from GO enrichment analysis (Biological Process) of PTBP2 CLIP-seq peaks in human cortex. Gene ratio is number of genes with peak calls relative to total genes in GO group (count). Synapse-related terms are highlighted in blue. FIG. 3E shows a SynGO enrichment analysis of PTBP2 CLIP-seq peaks in human cortex relative to a background set of brain-expressed genes represented as a sunburst plot. (Top) Biological Process, 623 genes. (Bottom) Cellular Component, 755 genes. FIG. 3F shows RBP-Maps-mediated positional analysis of PTBP2 peak calls relative to AS events identified as differentially spliced by rMATS upon PTBP2 KD in iPSC-neurons. Red indicates a higher inclusion level of the darkly shaded element upon PTBP2 KD; hence it suggests that endogenous PTBP2 promotes exclusion of the darkly-shaded element (n=1217, 203, 245, 553, and 127 for SE, A5SS, A3SS, MXE, and RI events, respectively). Blue indicates the opposite (n=1806, 286, 342, 657, and 196 for SE, A5SS, A3SS, MXE, and RI events, respectively). n=3 biological replicates for PTBP2 CLIP-seq and size-matched input controls.

FIGS. 4A to 4F illustrate that PTBP2 binds and promotes differential splicing and nonsense-mediated decay of SYNGAP1. FIG. 4A is a Zoom-in for three regions of interest on SYNGAP1 showing (top) gene model of alternative splicing event (ENST00000418600 is the dominant isoform in brain), followed by (middle) human cortex CLIP-seq (peaks, PTBP2 eCLIP read coverage, size-matched input read coverage), then iPS-neurons CLIP-seq (as for cortex). The bottom two rows depict RNA-seq read coverage and sashimi plots for untreated control and PTBP2 KD. n=3 replicates (overlaid) for human cortex CLIP-seq and iPS-neurons RNA-seq and CLIP-seq data sets. FIG. 4B provides quantification of changes in AS at each of the 3 above regions of interest (aligned in column) upon PTBP2 KD by 1) rMATS (left, statistical significance determined in rMATS by a likelihood-ratio test at a cutoff of 1% difference) and RT-PCR splicing assays (right). Light shaded rectangles denote constitutive exons and red rectangles denote NMD-inducing AS events. FIG. 4C are results from RT-PCR splicing assays of each region of interest upon CHX treatment to inhibit NMD. FIG. 4D provides quantification (qPCR) of SYNGAP1 mRNA fold change upon CHX treatment and (FIG. 4E) PTBP2 KD. FIG. 4F shows SYNGAP1 protein expression by western blot upon PTBP2 KD. Statistical analyses performed using one-way ANOVA with Dunnett's multiple comparison tests. * p<0.05, ** p<0.01 and *** p<0.001.

FIGS. 5A to 5H show that disrupting PTBP binding in SYNGAP1 intron 10 site upregulates SYNGAP1. FIG. 5A is a Western blot from HEK293T cells transfected for 48 h with siRNA against PTBP1 or PTBP2. (siSC) is negative control, non-targeted siRNA. FIG. 5B Top and left panel: RT-PCR from HEK293T cells transfected for 48 h with siRNA against PTBP1 or PTBP2 alone or in combination. Right panel: qPCR showing SYNGAP1 mRNA levels. FIG. 5C: Top and left panel: RT-PCR from HEK293T cells transfected with 5 μM of PTBP decoy oligo for 48 h, including a non-targeting decoy (D_SCRM) as negative control. Right panel: qPCR showing SYNGAP1 mRNA levels. (FIG. 5D) Visualization of PTBP2 eCLIP-seq data from human cortex highlighting two highly enriched PTBP binding regions near SYNGAP1 exon 11: site 1 (intronic site) and site 2 (NMD-inducing Exon 11×). Zoom-ins for both sites are provided including information about the nucleotide content (CU rich regions in red) and the location of the ASO walks. Light arrows indicate called peaks. FIG. 5E shows a scheme depicting 1-nt resolution ASO walk on SYNGAP1 site 1. The target region spans 33 nt of intronic sequence. Black lines denote introns, white rectangles denote constitutive exons and red rectangle denotes non-productive alternative exon (exon 11×). FIG. 5F: RT-PCR from HEK293T cells transfected with 200 nM of ASO for 24 h, including a non-targeting ASO control (ET-SC) and no ASO control (Mock). FIG. 5G Top and left panel: RT-PCR from HEK293T cells transfected with increasing concentrations of lead ASO ET-019 and negative controls for 48 h. Right panel: qPCR showing SYNGAP1 mRNA levels. FIG. 5H is a Western blot from HEK293T cells transfected as in FIG. 5G. For statistical analysis in FIG. 5C, unpaired two-tailed T test was performed. All other statistical analyses were performed using one-way ANOVA with Dunnett's multiple comparison tests. *p<0.05, ** p<0.01 and *** p<0.001.

FIGS. 6A to 6F illustrate that disrupting PTBP binding in SYNGAP1 exon 11× upregulates SYNGAP1. FIG. 6A: Top, Scheme depicting the initial 5-nt resolution ASO walk. The target region spans 93 nt of non-productive 3′ss in SYNGAP1. Bottom, RT-PCR from HEK293T cells transfected with 100 nM of ASO for 24 h, including a positive control ASO targeting SYNGAP1 site 1 (ET-019), a non-targeting ASO control (ET-SC) and no ASO control (Mock). FIG. 6B: Top, Scheme depicting a combined 2-nt and 1-nt resolution ASO walk. The target region spans 38 nt of non-productive 3′ss in SYNGAP1. Bottom, RT-PCR from HEK293T cells transfected with 100 nM of ASO for 24 h, including the same controls as in FIG. 6A. FIG. 6C provides qPCR quantification of SYNGAP 1 transcript levels from samples in FIG. 6B. FIG. 6D shows non-productive and productive transcript levels calculated from densitometric analysis of RT-PCR products from FIG. 6B and represented as log 2FC values relative to Mock. Arrows indicate ASOs that increase SYNGAP1 productive transcript and mRNA levels. Black line indicates ASOs that lead to no-go decay. FIG. 6E: Top and left panel: RT-PCR from HEK293T cells transfected with increasing concentrations of STK-071, ET-085 and ET-SC for 48 h. Right panel: qPCR showing SYNGAP1 mRNA levels. FIG. 6F is a Western blot from HEK293T cells transfected as in FIG. 6G. Statistical analyses performed using one-way ANOVA with Dunnett's multiple comparison tests. * p<0.05, ** p<0.01 and *** p<0.001.

FIGS. 7A to 7E show the results of intracerebroventricular (ICV) injection of various ASOs in neonatal mice increases Syngap1 mRNA. FIG. 7A Experimental design for evaluation of Syngap1 ASOs in vivo. P2 mice were ICV-injected with PBS, a positive control ASO targeting the non-productive exon inclusion in Scn1a (STK-135, 10 μg) previously reported in (Lim et al 2020), Ms-ET-019 (10 μg) and Ms-ET-085 (4 and 40 μg). Mice were euthanized at P7, and brain tissues were harvested and analyzed for productive exon exclusion in Scn1a with STK-135, and productive exclusion of the alt. 3′ss in Syngap1 with Syngap1 ASOs. FIG. 7B shows the results of Scn1a RT-PCR assay from mouse brains injected with 10 μg of STK-135. The percentage of exon 21× exclusion (productive splicing) in Sen1a transcript was calculated based on densitometric analysis of RT-PCR products. FIG. 7C is from a qPCR showing Sen1a productive transcript levels. FIG. 7D is a Syngap1 RT-PCR assay. FIG. 7E is a Syngap1 qPCR. For statistical analyses in FIG. 7B and FIG. 7C, unpaired two-tailed T test was performed. All other statistical analyses were performed using one-way ANOVA with Dunnett's multiple comparison tests. * p<0.05, ** p<0.01 and *** p<0.001.

FIGS. 8A-8C provide characterization of iPS-Neurons. FIG. 8A provides identification of neuronal marker expression by immunofluorescence of iPS-neurons. iPS-neurons were immunostained with antibodies specific to MAP2, PSD-95, and Tuj-1, and counterstained with Hoechst-33342, indicative of neuronal phenotype. Bar scale=20 um.

FIG. 8B (Left) provides qPCR of iPS-neurons for transcripts of the excitatory cortical progenitor, TBR2, and cortical neuron markers TBR1, CTIP2, SATB2, REELIN, and NGN2 show expression of all subtypes, with the highest levels transcripts being TBR1, CTIP2, and NGN2. (Right) qPCR of iPS-Neurons defined neurons as excitatory subtypes with expression of excitatory markers, VGLUT1 and VGLUT2, and minimal expression of inhibitory markers, GAD67 and GAD65. qPCR data is represented as the average of technical replicates for 4-6 samples from 3-4 independent differentiations of the CHOP WT10 line. FIG. 8C demonstrates neurons loaded with a calcium indicator dye (Fluo-4 AM) and electrically stimulated with 20 Hz trains of depolarizing field stimuli lasting 10 s, with 20 seconds of rest between trains. The right hand panel shows the quantification of mean+/−SEM fluorescence intensity changes over time normalized to initial fluorescence levels (F/F0) using the 20 Hz stimulation protocol described above on 4 separate biological replicates. FIG. 8D provides qPCR from HEK293T cells transfected with 25 nM of PTBP2 gapmers for 24 h. A non-targeting gapmer (NegA) was included as negative control.

FIGS. 9A to 9C provide PTBP2 binding and alternative splicing of SYNGAP1. FIG. 9A (Top) is a gene model of SYNGAP1 followed by RNA-seq read coverage for human cortex and RNA-seq read coverage and sashimi plots for untreated control iPSC-neurons. (Insets) Zoom-ins for regions of interest. The human cortex RNA-seq represents 101 samples from GTEx Brain Front Cortex (BA9). n=3 replicates (overlaid) for iPSC-neurons. FIG. 9B is a Gene model of ENST00000418600, the dominant SYNGAP1 isoform in brain. FIG. 9C provides human cortex CLIP-seq (peaks, PTBP2 eCLIP read coverage, size-matched input read coverage), then iPSC-neurons CLIP-seq (as for cortex). n=3 replicates (overlaid) for PTBP2 CLIP-seq and size-matched input controls.

FIGS. 10A to 10E illustrate disrupting PTBP binding in SYNGAP1 site 1 improves SYNGAP 1 productive splicing. FIG. 10A provides RT-PCR from N2A cells transfected with either Ptbp1 gapmer or negative control (Ms-neg). FIG. 10B provides RT-PCR from SH-SY5Y cells electroporated with 20 μM of ET-019 or negative control ASO (ET-SC) for 24 h. (FIGS. 10C and 10D) RT-PCR from HEK293T cells transfected for 24 h with 100 nM of ASO targeting SYNGAP1 site 1, including a positive ASO control (ET-019), a non-targeting ASO control (ET-SC) and no ASO control (Mock). For statistical analyses, unpaired two-tailed T test was performed. * p<0.05 and *** p<0.001.

FIGS. 11A to 11E illustrate disrupting PTBP binding in SYNGAP1 exon 11× upregulates SYNGAP1. FIG. 11A is a qPCR showing SYNGAP1 mRNA levels from samples in FIG. 6B. (FIGS. 11B and 11C) Top and left panels: RT-PCR from HEK293T cells transfected with increasing concentrations of ET-061 (FIG. 11B) or ET-086 (FIG. 11C) for 48 h, including matching concentrations of the non-targeting ASO control (ET-SC) and no ASO control (Mock). Right panels: qPCR showing SYNGAP1 mRNA levels. (FIG. 11D is a Western blot from HEK293T cells transfected as in (C). (FIG. 11E) Top and left panel: RT-PCR from HEK293T cells transfected with 100 nM of ASO for 48 h, including matching concentrations of the non-targeting ASO control (ET-SC) and no ASO control (Mock). Right panel: qPCR showing SYNGAP1 mRNA levels. For statistical analyses, one-way ANOVA with Dunnett's multiple comparison tests (in FIGS. 11B, 11C and 11D) or Tukey's multiple comparisons test (in FIG. 11E) were performed. * p<0.05, ** p<0.01 and *** p<0.001.

FIGS. 12A to 12I show that disrupting PTBP2 binding in Site 1 and Site 2 upregulates SYNGAP1 and protein expression in SYNGAP1 haploinsufficient patient cell lines. FIG. 12A is a schematic of SYNGAP 1 mRNA showing the location of the heterozygous mutations present in the two independent SYNGAP1 patient iPSC lines. FIG. 12 B is a schematic depicting the generation of the SYNGAP1 R1240X patient-derived iPSC line and the corresponding isogenic control line in which the heterozygous mutation has been reverted using CRISPR/Cas9 technology. PBMCs, peripheral blood mononuclear cells.

FIG. 12C: Top and right panels: SYNGAP1 Western blot from corrected (isogenic control) and patient SYNGAP1 R1240X iPSC-neurons.

FIG. 12 D: Top and right panels: SYNGAP1 Western blot from WT and patient SYNGAP1 K1185X iPSC-neurons. (c-d) Left panel: qPCR showing SYNGAP1 mRNA levels.

FIG. 12E: Top and right panels: PTBP2 Western blot from K1185X NPCs treated for 3 d with PTBP2 gapmer. Left panel: PTBP2 mRNA fold change quantification (qPCR).

FIG. 12F: Top and left panels: SYNGAP1 RT-PCR from K1185X NPCs treated for 3 d with PTBP2 gapmer. Right panel: qPCR showing SYNGAP1 mRNA levels. FIG. 12G is a SYNGAP1 Western blot from samples in FIG. 12E.

FIGS. 12E and 12F show a-g, a non-targeting gapmer (NegA) was used as negative control. FIG. 12H) has top and left panels: RT-PCR from R1240X iPSC-neurons treated for 7 d with ET-019 at 10 μM. Right panel: qPCR showing SYNGAP1 mRNA levels

FIG. 12I has top and left panels: RT-PCR from K1185X iPSC-neurons treated for 7 d with Site 1 and Site 2 targeting ASOs at 10 μM. Right panel: qPCR showing SYNGAP1 mRNA levels. White color data points represent an independent experiment.

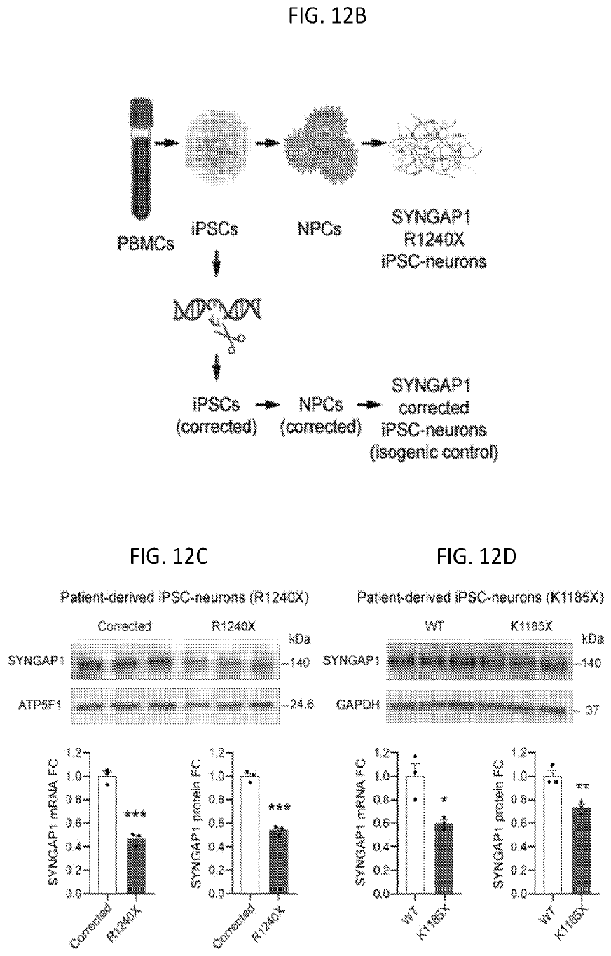

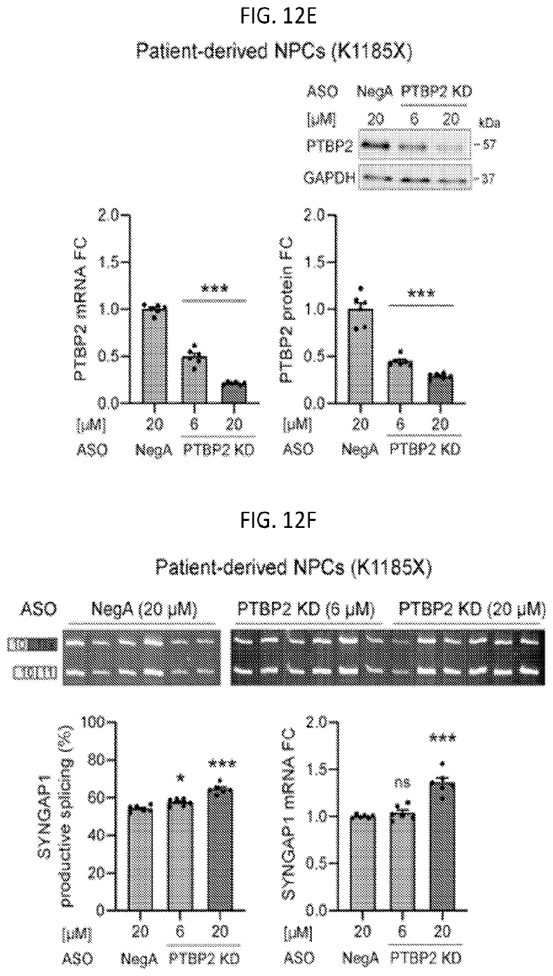

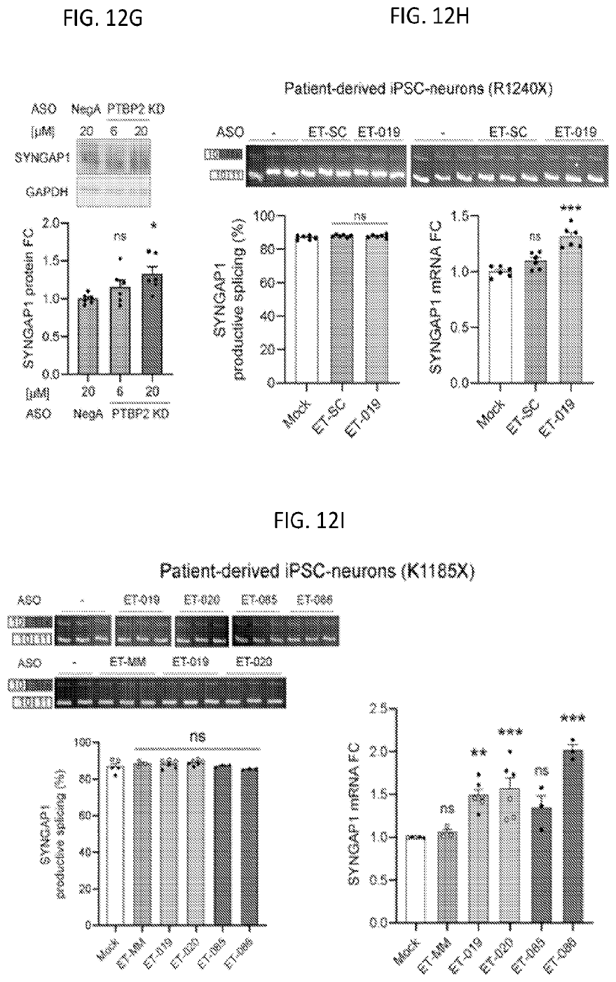

In FIG. 12H and FIG. 12I, non-targeting ET-SC or ET-MM ASOs were included as negative controls. Data are represented as mean values±SEM. All data points represent independent biological replicates. C-d (n=3). E-h (n=6). I (n=3 except n=5 for Mock and n=6 for ET-019 and ET-020). In c-d, Student's t-test.

In FIGS. 12E-FIG. 12G, one-way ANOVA with Dunnett's multiple comparison test vs. NegA-treated cells. In FIGS. 12H-FIG. 12I, one-way ANOVA with Dunnett's multiple comparison test vs. mock-treated cells. ns p>0.05, *p<0.05, ** p<0.01 and *** p<0.001.

### DETAILED DESCRIPTION OF THE INVENTION

The compositions provided herein are useful in therapies for treating genetic disorders associated with Polypyrimidine tract binding protein (PTBP) binding of a dysfunctional gene and causing alternative splicing thereof. The examples provided herein illustrate PTBP-binding of Synaptic GTPase Activating Protein (SYNGAP), and more particularly, PTBP2-binding of SYNGAP1 and that compositions provided herein which interference with this binding reduce alternative splicing events in SYNGAP1 and are useful therapeutically for treating a SYNGAP-associated disorder.

As used herein, a “SYNGAP-associated neurodevelopmental disorder” (or “NDD,” “neurodevelopmental disorder,” “neurodegenerative disease,” or “neurodegenerative disorder” as used herein) is a disease in which one or more isoforms of SYNGAP is aberrantly expressed. NDDs include, but are not limited to, an intellectual disability (ID), autism spectrum disorders (ASD), epilepsy, schizophrenia, or Pervasive Developmental Disorder--Not Otherwise Specified (PDD-NOS).

Without wishing to be bound by theory, the inventors believe that neither Polypyrimidine tract binding proteins (PTBP2) binding nor splicing has been assessed in human neurons or brain tissue. The inventors believe they are the first to identify PTBP2 binding sites in SYNGAP1 and other human genes as therapeutic targets for agents which interfere with PTPB2-SYNGAP1 binding and prevent alternative splicing events resulting in null and/or undesirable expression levels of SYNGAP1 protein and/or expression of undesirable alternative SYNGAP1 isoforms protein. Additionally, provided herein is the first comprehensive map of PTBP2-dependent alternative splicing in human neurons and cortical tissue. Other therapeutic targets are also identified for therapy of genetic disorders which are associated with PTPB2-mediated splicing events which result in dysfunctional protein. See, e.g., Table 3 (e.g., GRIN1, MVD, DNM1, CAMK2B, HNRNPA1, CTNND1), incorporated by reference in this section of the specification.

The SYNGAP 1 gene is located on chromosome 6 and is responsible for producing the SYNGAP protein. See, Human Gene SYNGAP1 (ENST00000646630.1) from GENCODE V39 [University of California, Santa Cruz, Genomics Institute Genome Brower, at: genome.ucsc.edu/cgi-bin/hgGene?hgg_gene=ENST00000646630.1&hgg_chrom=chr6&hgg_-start=33420064&hgg_end=33453689&hgg_type=knownGene&db=hg38 #rnaStructure].

See, also RefSEQ Gene (NCBI Reference Sequence): NM_006772.3. The sequence of the three common SYNGAP cDNA polynucleotide isoforms is provided in SEQ ID NO: 5 (NCBI 000006.12), 6 (NC_060930.1), or 7 (NT_167249.2). The amino acid sequence of isoform 1 is provided in SEQ ID NO: 8 and the amino acid sequence isoform 2 is provided in SEQ ID NO: 9.

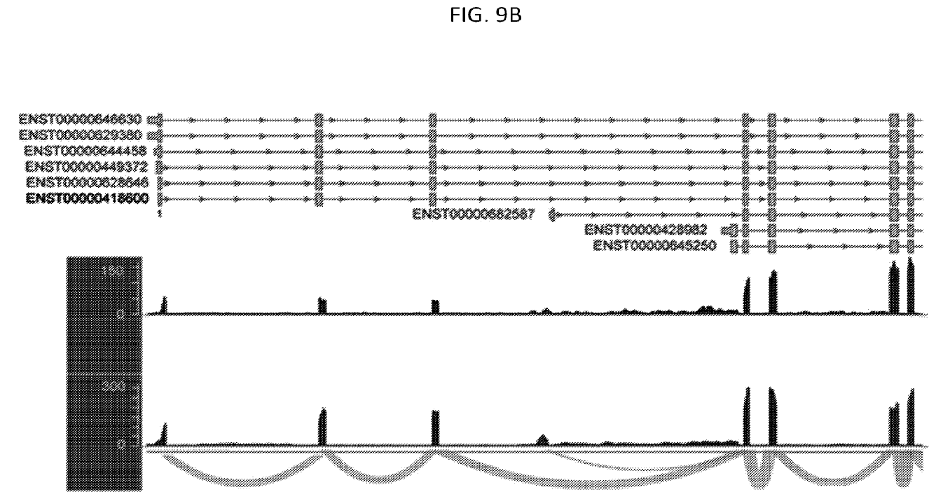

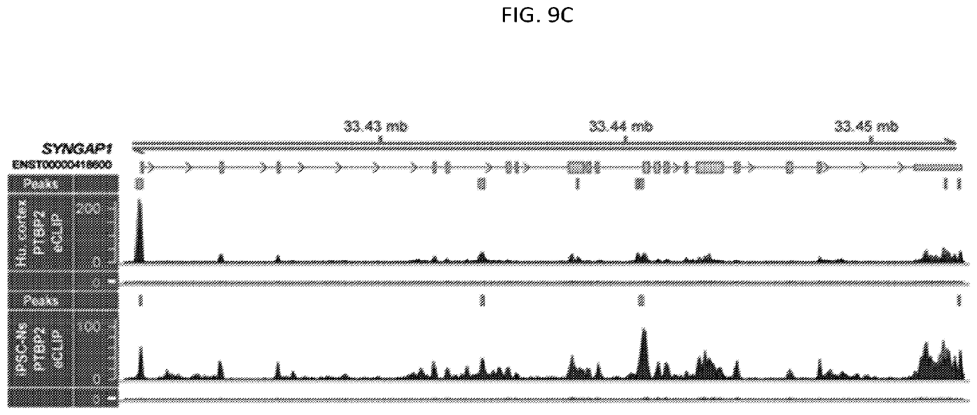

FIGS. 9A, 9B and 9C illustrate a gene model of ENST00000418600, the dominant SYNGAP1 isoform in brain, followed by human cortex (BA4) CLIP-seq (peaks, PTBP2 eCLIP read coverage, size-matched input read coverage), then iPS-neurons CLIP-seq (as for cortex). n=3 replicates (overlaid) for PTBP2 CLIP-seq and size-matched input controls. Examples of suitable therapeutic target sites for ASOs or RNAi directed to SYNGAP1 include those in Tables 1 and 2, which are identified by chromosome position, with reference to CRch38 genome build in the UCSC Genome Browser. Table 3 provides examples of suitable therapeutic target sites for other genetic disorders which are associated with PTPB2-mediated splicing events. See, e.g., Table 3 (e.g., GRIN1, MVD, DNM1, CAMK2B, HNRNPA1, CTNND1).

TABLE 1
PTBP2-binding sites on SYNGAP1

| chromosome | start | end | strand |
|---|---|---|---|
| chr6 | 33420061 | 33420111 | + |
| chr6 | 33420111 | 33420161 | + |
| chr6 | 33420161 | 33420211 | + |
| chr6 | 33420211 | 33420261 | + |
| chr6 | 33420261 | 33420311 | + |
| chr6 | 33434011 | 33434061 | + |
| chr6 | 33434061 | 33434111 | + |
| chr6 | 33434111 | 33434161 | + |
| chr6 | 33434161 | 33434211 | + |
| chr6 | 33434211 | 33434261 | + |
| chr6 | 33438011 | 33438061 | + |
| chr6 | 33440411 | 33440461 | + |
| chr6 | 33440461 | 33440511 | + |
| chr6 | 33440561 | 33440611 | + |
| chr6 | 33440611 | 33440661 | + |
| chr6 | 33440661 | 33440711 | + |
| chr6 | 33453011 | 33453061 | + |
| chr6 | 33453561 | 33453611 | + |

In certain embodiments, the DNA sense (positive (+)) strand or its complementary strand (−), or a transcript thereof (an RNA), may be targeted by an agent as provided here which interferes with PTBP2-binding to SYNGAP1 and/or interferes with alternative splicing of SYNGAP1. In certain embodiments, the agent is an ASO, RNAi, small interfering RNA (siRNA), microRNA (miRNA), or another interfering sequence which targets a region in SYNGAP1 to which PTBP2 binds. See, e.g., the chromosomal locations in the Table 1 and 2. In certain embodiments, the ASOs are designed as gapmer ASO's.

“Gapmer” means an ASO comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.” In certain embodiments, at least one antisense oligonucleotide in a composition of the invention a gapmer.

In certain embodiments, the ASOs and other therapeutic agents (e.g., RNAi) described herein are targeted to sequences which interfere the PTBP2 binding sites in the SYNGAP1 genome. In certain embodiments, an ASO is selected which has a sequence (5′ to 3′ of, at least 12 consecutive nucleotides of consecutive nucleotides of SEQ ID NO: 10-58, a sequence at least 99% identical to SEQ ID NO: 10-58, respectively, a sequence having at least 95% complementarity to SEQ ID NO: 10-58, or a pharmaceutically acceptable salt thereof. In certain embodiments, an ASO is selected which has a sequence (5′ to 3′ of, at least 14 consecutive nucleotides of SEQ ID NO: 10-58, a sequence at least 99% identical to SEQ ID NO: 10-58, respectively, a sequence having at least 95% complementarity to SEQ ID NO: 10-58, or a pharmaceutically acceptable salt thereof. In certain embodiments, an ASO is selected which has a sequence (5′ to 3′ of, at least 16 consecutive nucleotides of SEQ ID NO: 10-58, a sequence at least 99% identical to SEQ ID NO: 10-58, respectively, a sequence having at least 95% complementarity to SEQ ID NO: 10-58, respectively, or a pharmaceutically acceptable salt thereof. In certain embodiments, an ASO is selected which has a sequence (5′ to 3′ of, a nucleic acid sequence of 18 consecutive nucleotides of SEQ ID NO: 10-58, a sequence at least 99% identical to SEQ ID NO: 10-58, respectively, a sequence having at least 95% complementarity to SEQ ID NO:10-58, respectively, or a pharmaceutically acceptable, or a pharmaceutically acceptable salt thereof. In certain embodiments, combinations of two or more different ASOs targeted to one or more of the positions identified in the table below is provided. The ASOs in Table 2 below are targeted to the positive strand and are the reverse complement of the targeted sequence. In certain embodiments, an alternative agent may be targeted to the positive coding strand. Additionally or alternatively, another agent (e.g., an ASO or RNAi) may comprise a shorter sequence in this chromosomal position region, a longer sequence encompassing all or a portion of a sequence in the identified chromosomal region.

| | | | Position | position_ | | |
|---|---|---|---|---|---|---|
| ASO_ | | Target | (chr6)_ | (chr6) | | SEQ ID |
| ID | sequence(5′-3′) | strand | from | to | length | NO: |
| ET- | GGGAGAGAGATGGAGGG | + | 33440446 | 33440463 | 18 | 10 |
| 015 | G | | | | | |
| ET- | TGGGAGAGAGATGGAGG | + | 33440447 | 33440464 | 18 | 11 |
| 016 | G | | | | | |
| ET- | GTGGGAGAGAGATGGAG | + | 33440448 | 33440465 | 18 | 12 |
| 017 | G | | | | | |
| ET- | CGTGGGAGAGAGATGGA | + | 33440449 | 33440466 | 18 | 13 |
| 018 | G | | | | | |
| ET- | CACGTGGGAGAGAGATG | + | 33440451 | 33440468 | 18 | 2 |
| 019 | G | | | | | |
| ET- | ACACGTGGGAGAGAGAT | + | 33440452 | 33440469 | 18 | 14 |
| 020 | G | | | | | |
| ET- | GACACGTGGGAGAGAGA | + | 33440453 | 33440470 | 18 | 15 |
| 021 | T | | | | | |
| ET- | AGACACGTGGGAGAGAG | + | 33440454 | 33440471 | 18 | 16 |
| 022 | A | | | | | |
| ET- | AGAGACACGTGGGAGAG | + | 33440456 | 33440473 | 18 | 17 |
| 023 | A | | | | | |
| ET- | GAGAGACACGTGGGAGA | + | 33440457 | 33440474 | 18 | 18 |
| 024 | G | | | | | |
| ET- | GGAGAGACACGTGGGAG | + | 33440458 | 33440475 | 18 | 19 |
| 025 | A | | | | | |
| ET- | GGGAGAGACACGTGGGA | + | 33440459 | 33440476 | 18 | 20 |
| 026 | G | | | | | |
| ET- | GGAGAGAGATGGAGGGG | + | 33440445 | 33440462 | 18 | 21 |
| 027 | T | | | | | |
| ET- | GGGGAGAGACACGTGGG | + | 33440460 | 33440477 | 18 | 22 |
| 028 | A | | | | | |
| ET- | CACGTGGGAGAGAGATG | + | 33440450 | 33440468 | 19 | 23 |
| 029 | GA | | | | | |
| ET- | ACACGTGGGAGAGAGAT | + | 33440451 | 33440469 | 19 | 24 |
| 030 | GG | | | | | |
| ET- | GACACGTGGGAGAGAGA | + | 33440451 | 33440470 | 20 | 25 |
| 031 | TGG | | | | | |
| ET- | AGACACGTGGGAGAGAG | + | 33440451 | 33440471 | 21 | 26 |
| 032 | ATGG | | | | | |
| ET- | GAGACACGTGGGAGAGA | + | 33440451 | 33440472 | 22 | 27 |
| 033 | GATGG | | | | | |
| ET- | CTTCCAGGGAACATGCTG | + | 33440634 | 33440651 | 18 | 3 |
| 061 | | | | | | |
| ET- | CTCAGCTTCCAGGGAACA | + | 33440639 | 33440656 | 18 | 28 |
| 062 | | | | | | |
| ET- | AGACCCTCAGCTTCCAGG | + | 33440644 | 33440661 | 18 | 29 |
| 063 | | | | | | |
| ET- | CCCAGAGACCCTCAGCTT | + | 33440649 | 33440666 | 18 | 30 |
| 064 | | | | | | |
| ET- | TGAGCCCCAGAGACCCTC | + | 33440654 | 33440671 | 18 | 31 |
| 065 | | | | | | |
| ET- | GGGACTGAGCCCCAGAG | + | 33440659 | 33440676 | 18 | 32 |
| 066 | A | | | | | |
| ET- | AGACCGGGACTGAGCCCC | + | 33440664 | 33440681 | 18 | 33 |
| 067 | | | | | | |
| ET- | GAGAGAGACCGGGACTG | + | 33440669 | 33440686 | 18 | 34 |
| 068 | A | | | | | |
| ET- | AGAAAGAGAGAGACCGG | + | 33440674 | 33440691 | 18 | 35 |
| 069 | G | | | | | |
| ET- | GAGAGAGAAAGAGAGAG | + | 33440679 | 33440696 | 18 | 36 |
| 070 | A | | | | | |
| ET- | AGAGAGAGAGAGAAAGA | + | 33440684 | 33440701 | 18 | 37 |
| 071 | G | | | | | |
| ET- | CAGAGAGAGAGAGAGAG | + | 33440689 | 33440706 | 18 | 38 |
| 072 | A | | | | | |
| ET- | GGAGACAGAGAGAGAGA | + | 33440694 | 33440711 | 18 | 39 |
| 073 | G | | | | | |
| ET- | GTCGGGGAGACAGAGAG | + | 33440699 | 33440716 | 18 | 40 |
| 074 | A | | | | | |
| ET- | GAAGGGTCGGGGAGACA | + | 33440704 | 33440721 | 18 | 41 |
| 075 | G | | | | | |
| ET- | GGGGGGAAGGGTCGGGG | + | 33440709 | 33440726 | 18 | 42 |
| 076 | A | | | | | |
| ET- | AGGGGGGTGGTAGGAGG | + | 33440616 | 33440633 | 18 | 43 |
| 077 | T | | | | | |
| ET- | TGAGGGGGGTGGTAGGA | + | 33440618 | 33440635 | 18 | 44 |
| 078 | G | | | | | |
| ET- | GCTGAGGGGGGTGGTAG | + | 33440620 | 33440637 | 18 | 45 |
| 079 | G | | | | | |
| ET- | ATGCTGAGGGGGGTGGTA | + | 33440622 | 33440639 | 18 | 46 |
| 080 | | | | | | |
| ET- | ACATGCTGAGGGGGGTGG | + | 33440624 | 33440641 | 18 | 47 |
| 081 | | | | | | |
| ET- | GAACATGCTGAGGGGGGT | + | 33440626 | 33440643 | 18 | 48 |
| 082 | | | | | | |
| ET- | GGGAACATGCTGAGGGG | + | 33440628 | 33440645 | 18 | 49 |
| 083 | G | | | | | |
| ET- | CAGGGAACATGCTGAGG | + | 33440630 | 33440647 | 18 | 50 |
| 084 | G | | | | | |
| ET- | TCCAGGGAACATGCTGAG | + | 33440632 | 33440649 | 18 | 1 |
| 085 | | | | | | |
| ET- | TTCCAGGGAACATGCTGA | + | 33440633 | 33440650 | 18 | 4 |
| 086 | | | | | | |
| ET- | AGCTTCCAGGGAACATGC | + | 33440636 | 33440653 | 18 | 51 |
| 087 | | | | | | |
| ET- | TGGAGGGGTGTGGGAGA | + | 33440432 | 33440453 | 22 | 52 |
| X1 | GAGAC | | | | | |
| ET- | GATGGAGGGGTGTGGGA | + | 33440434 | 33440455 | 22 | 53 |
| X2 | GAGAG | | | | | |
| ET- | GAGATGGAGGGGTGTGG | + | 33440436 | 33440457 | 22 | 54 |
| X3 | GAGAG | | | | | |
| ET- | GAGAGATGGAGGGGTGT | + | 33440438 | 33440459 | 22 | 55 |
| X4 | GGGAG | | | | | |
| ET- | GAGAGAGATGGAGGGGT | + | 33440440 | 33440461 | 22 | 56 |
| X5 | GTGGG | | | | | |
| ET- | GGGAGAGAGATGGAGGG | + | 33440442 | 33440463 | 22 | 57 |
| X6 | GTGTG | | | | | |
| ET- | GTGGGAGAGAGATGGAG | + | 33440444 | 33440465 | 22 | 58 |
| X7 | GGGTG | | | | | |

In certain embodiments, the at least one agent comprises an ASO of 15 to 30 nucleotides in length comprising at least 15 consecutive nucleotides of a sequence comprising: SSO_085: TCCAGGGAACATGCTGAG (SEQ ID NO: 1), a sequence at least 99% identical to SEQ ID NO: 1, a sequence having at least 95% complementarity to SEQ ID NO: 1, or a pharmaceutically acceptable salt thereof, or combinations thereof.

In certain embodiments, the at least one agent comprises an ASO of 15 to 30 nucleotides in length comprising at least 15 consecutive nucleotides of a sequence comprising: SSO_019: CACGTGGGAGAGAGATGG (SEQ ID NO: 2), a sequence at least 99% identical to SEQ ID NO: 2, or a pharmaceutically acceptable salt thereof, or combinations thereof.

In certain embodiments, the at least one agent comprises an ASO of 15 to 30 nucleotides in length comprising at least 15 consecutive nucleotides of a sequence comprising: SSO_061: CTTCCAGGGAACATGCTG (SEQ ID NO: 3), a sequence at least 99% identical to SEQ ID NO: 3, or a pharmaceutically acceptable salt thereof, or combinations thereof.

In certain embodiments, the at least one agent comprises an ASO of 15 to 30 nucleotides in length comprising at least 15 consecutive nucleotides of a sequence comprising: SSO_086: a sequence at least 99% identical to SEQ ID NO: 4, or a pharmaceutically acceptable salt thereof, or combinations thereof.

In certain embodiments, the at least one agent comprises an ASO of 15 to 30 nucleotides in length comprising at least 15 consecutive nucleotides of a sequence comprising: SSO_061: a sequence comprising a sequence having at least 95% complementarity to SEQ ID NO: 1, 2, 3 or 4, or a sequence comprising at least 15 consecutive nucleotides of SEQ ID NO: 1, 2, 3, or 4, or a pharmaceutically acceptable salt thereof, or combinations thereof.

In certain embodiments, the at least one agent comprises an ASO of 15 to 30 nucleotides in length comprising at least 15 consecutive nucleotides of a sequence comprising a sequence having at least 95% complementarity to SEQ ID NO: 1, 2, 3 or 4, or a sequence comprising at least 15 consecutive nucleotides of SEQ ID NO: 1, 2, 3, or 4, or a pharmaceutically acceptable salt thereof, or combinations thereof.

In certain embodiments, a composition and/or a therapeutic regimen comprises an ASO of 15 to 30 nucleotides in length comprising at least 15 consecutive nucleotides of a sequence comprising: combinations of SSO_085, SSO_019, SSO_061, SSO_086, a sequence having at least 95% complementarity to SEQ ID NO: 1, 2, 3 or 4, or a sequence comprising at least 15 consecutive nucleotides of SEQ ID NO: 1, 2, 3, or 4, or a pharmaceutically acceptable salt thereof, or combinations thereof.

In certain embodiments, a composition comprises a combination of SSO-085 and SSO-019, and/or ASO having 100% complementarity to one of SEQ ID NO: 1 and/or an ASO having 100% complementarity to of SEQ ID NO: 2.

In certain embodiments, a composition comprises at least one ASO of 15 to 30 nucleotides in length which specifically target a sequence in the chromosomal location of Table 1 or Table 2. In certain embodiments, a composition comprises at least one agent (e.g., RNAi) targeted to a sequence in a chromosomal location of Table 1 or Table 2 for treatment of the symptoms of a SYNGAP1-related disorder. In certain embodiments, a composition comprises combinations of ASOs, combinations of one or more different ASOs with another agent having therapeutic effect, and/or combinations of the ASOs or another interfering agent as provided herein with gene replacement therapy and/or other therapies useful for treating a SYNGAP-1 related disorder symptom.

As described herein, an ASO or another moiety may be in the form of a pharmaceutically acceptable salt. The term “pharmaceutically acceptable salts” includes salts of the active compounds (agents, e.g., ASOs) that are prepared with relatively nontoxic acids or bases, depending on the particular substituents found on the compounds described herein. When compounds contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When the compounds contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolyl-sulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds contain both basic and acidic functionalities that allow the compounds to be converted into either base or acid addition salts. Other pharmaceutically acceptable carriers known to those of skill in the art are suitable. Salts tend to be more soluble in aqueous or other protonic solvents that are the corresponding free base forms. In other cases, the preparation may be a lyophilized powder that is combined with buffer prior to use.

Provided herein are additionally or alternatively, other agents which interfere with PTBP2-binding of the SYNGAP gene and prevent an alternative splicing event associated a disease or disorder. The agent may be any suitable genetic element or chemical moiety including, e.g., an anti-sense oligonucleotide (ASO), an RNAi, or combinations thereof. In certain embodiments, the agent is engineered to be delivered via a viral vector or another genetic element. Suitable viral vectors may include, e.g., selected from a recombinant parvovirus, a recombinant lentivirus, or non-viral vector. Additionally or alternatively, a non-viral vector may be selected which comprises one or more agent(s). In certain embodiments, the non-viral vector is a lipid nanoparticle, lipidoid, or liposome.

As used herein, an “antisense oligonucleotide” or “ASO” means an oligonucleotide having a nucleobase sequence that is complementary to a target nucleic acid or region or segment thereof. An antisense oligonucleotide is specifically hybridizable to a target nucleic acid or region or segment thereof, the hybridization of which results in RNase H mediated cleavage of the target nucleic acid.

“Contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

“Portion” refers to a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an oligomeric compound.

The term “complementary” is used to describe the relationship between nucleotide bases and/or polynucleotides that are capable of hybridizing to one another, e.g., the nucleotide sequence of such polynucleotides or one or more regions thereof matches the nucleotide sequence of another polynucleotide or one or more regions thereof when the two nucleotide sequences are aligned in opposing directions. Nucleobase matches or complementary nucleobases, as described herein, include the following pairs: adenine (A) with thymine (T), adenine (A) with uracil (U), cytosine (C) with guanine (G), and 5-methyl cytosine (^{m}C) with guanine (G). Complementary polynucleotides and/or nucleic acids need not have nucleobase complementarity at each nucleoside and may include one or more nucleobase mismatches. Accordingly, the present disclosure also includes isolated polynucleotides that are complementary to sequences as disclosed or used herein as well as those substantially similar nucleic acid sequences. The degree to which two polynucleotides have matching nucleobases can be expressed in terms of “percent complementarity” or “percent complementary.” In some embodiments, a polynucleotide has 70%, at least 70%, 75%, at least 75%, 80%, at least 80%, 85%, at least 85%, 90%, at least 90%, 95%, at least 95%, 97%, at least 97%, 98%, at least 98%, 99%, or at least 99% or 100% complementarity with another polynucleotide or a target nucleic acid provided herein. In embodiments wherein two polynucleotides or a polynucleotide and a target nucleic acid are “fully complementary” or “100% complementary,” such polynucleotides have nucleobase matches at each nucleoside without any nucleobase mismatches. Unless otherwise indicated, percent complementarity is the percent of the nucleobases of the shorter sequence that are complementary to the longer sequence.

An ASO or RNA agent may contain one or more mismatches to the target sequence. In one aspect, the sequence as described herein contains no more than 3 mismatches. If the sequence contains mismatches to a target sequence, in some aspects, the area of mismatch is not located in the center of the region of complementarity. If the oligonucleotide contains mismatches to the target sequence, in some aspects, the mismatch should be restricted to be within the last 5 nucleotides from either the 5′- or 3′-end of the region of complementarity.

“Specifically hybridizable” refers to a polynucleotide having a sufficient degree of complementarity between the polynucleotide and a target nucleic acid to induce a desired effect, while exhibiting minimal or no effects on non-target nucleic acids. In certain embodiments, specific hybridization occurs under physiological conditions.

“Specifically interfering” refers to an agent which blocks binding of a protein to its native target (e.g., PTBP binding to SYNGAP), while having minimal or no effect on non-target nucleic acids.

“Mismatch” or “non-complementary” means a nucleobase of a first polynucleotide that is not complementary to the corresponding nucleobase of a second polynucleotide or target nucleic acid when the first and second polynucleotides are aligned. For example, nucleobases including but not limited to a universal nucleobase, inosine, and hypoxanthine, are capable of hybridizing with at least one nucleobase but are still mismatched or non-complementary with respect to nucleobase to which it hybridized. As another example, a nucleobase of a first polynucleotide that is not capable of hybridizing to the corresponding nucleobase of a second polynucleotide or target nucleic acid when the first and second polynucleotides are aligned is a mismatch or non-complementary nucleobase.

In certain embodiments, the agent comprises is an antisense oligonucleotide having at least one modified internucleoside linkage, sugar moiety, or nucleobase.

In certain embodiments, one or more ASO is a chimeric oligonucleotide having a gap segment positioned between 5′ and 3′ wing segments. In certain embodiments, the gap segment of the chimeric oligonucleotide is comprised of 2′-deoxynucleotides and the wing segments are comprised of nucleotides having modified sugar moieties. In certain embodiments, the gap segment of the chimeric oligonucleotide consists of ten 2′-deoxynucleotides and each wing segment consists of five 2′-O-methoxyethyl-modified nucleotides.

In certain embodiments, one or more ASO comprises a modified sugar moiety is 2′-OMe or a bicyclic nucleic acid.

An oligonucleotide, or pharmaceutically acceptable salt thereof, can be chemically synthesized. An oligonucleotide, or pharmaceutically acceptable salt thereof, can be synthesized by standard methods known in the art as further discussed below, e.g., by use of an automated DNA synthesizer, such as are commercially available from, for example, Biosearch, Applied Biosystems, Inc.

An oligonucleotide, or pharmaceutically acceptable salt thereof, compound can be prepared using solution-phase or solid-phase organic synthesis or both. Organic synthesis offers the advantage that the oligonucleotide, or pharmaceutically acceptable salt thereof, comprising unnatural or alternative nucleotides can be easily prepared. A single-stranded oligonucleotide, or pharmaceutically acceptable salt thereof, can be prepared using solution-phase or solid-phase organic synthesis or both.

In some aspects, the oligonucleotide, or contiguous nucleotide region thereof, has a gapmer design or structure also referred herein merely as “gapmer.” In a gapmer structure the oligonucleotide comprises at least three distinct structural regions a 5′-flanking sequence (also known as a 5′-wing), a DNA core sequence (also known as a gap) and a 3′-flanking sequence (also known as a 3′-wing), in ‘5->3’ orientation. In this design, the 5′ and 3′ flanking sequences comprise at least one alternative nucleoside which is adjacent to a DNA core sequence, and can, in some aspects, comprise a contiguous stretch of 2 to 7 alternative nucleosides, or a contiguous stretch of alternative and DNA nucleosides (mixed flanking sequences comprising both alternative and DNA nucleosides).

The length of the 5′-flanking sequence region can be at least two nucleosides in length (e.g., at least at least 2, at least 3, at least 4, at least 5, at least 6, or more nucleosides in length). The length of the 3′-flanking sequence region can be at least two nucleosides in length (e.g., at least 2, at least 3, at least at least 4, at least 5, at least 6, or more nucleosides in length). The 5′ and 3′ flanking sequences can be symmetrical or asymmetrical with respect to the number of nucleosides they comprise. In some aspects, the DNA core sequence comprises about 10 nucleosides flanked by a 5′ and a 3′ flanking sequence each comprising about 5 nucleosides. In some aspects, the DNA core sequence comprises about 11 nucleosides flanked by a 5′ and a 3′ flanking sequence each comprising about 5 or about 6 nucleosides. In some aspects, the DNA core sequence comprises about 12 nucleosides flanked by a 5′ sequence comprising about 5 nucleosides, and a 3′ flanking sequence comprising about 6 nucleosides. In some aspects, the DNA core sequence comprises about 12 nucleosides flanked by a 5′ sequence comprising about 6 nucleosides, and a 3′ flanking sequence comprising about 5 nucleosides. In some aspects, the DNA core sequence comprises about 12 nucleosides flanked by a 5′ and a 3′ flanking sequence each comprising about 6 nucleosides.

“2′-deoxyfuranosyl sugar moiety” or “2′-deoxyfuranosyl sugar” means a furanosyl sugar moiety having two hydrogens at the 2′-position. 2′-deoxyfuranosyl sugar moieties may be unmodified or modified and may be substituted at positions other than the 2′-position or unsubstituted. A β-D-2′-deoxyribosyl sugar moiety in the context of an oligonucleotide is an unsubstituted, unmodified 2′-deoxyfuranosyl and is found in naturally occurring deoxyribonucleic acids (DNA).

“2′-deoxynucleoside” means a nucleoside comprising 2′-H(H) furanosyl sugar moiety, as found in naturally occurring deoxyribonucleic acids (DNA). In certain embodiments, a 2′-deoxynucleoside may comprise a modified nucleobase or may comprise an RNA nucleobase (uracil).

“2′-O-methoxyethyl” (also 2′-MOE) refers to a 2′-O(CH₂)₂—OCH₃) in the place of the 2′-OH group of a ribosyl ring. A 2′-O-methoxyethyl modified sugar is a modified sugar.

“2′-MOE nucleoside” (also 2′-O-methoxyethyl nucleoside) means a nucleoside comprising a 2′-MOE modified sugar moiety.

“2′-substituted nucleoside” or “2-modified nucleoside” means a nucleoside comprising a 2′-substituted or 2′-modified sugar moiety. As used herein, “2′-substituted” or “2-modified” in reference to a sugar moiety means a sugar moiety comprising at least one 2′-substituent group other than H or OH.

“5-methylcytosine” means a cytosine with a methyl group attached to the 5 position. A 5-methyl cytosine is a modified nucleobase.

“Overhanging nucleosides” refers to unpaired nucleotides at either or both ends of a duplex formed by hybridization of an antisense RNAi oligonucleotide and a sense RNAi oligonucleotide.

The 5′ and 3′ flanking sequences, flanking the 5′ and 3′ ends of an ASO core sequence (e.g., SEQ ID NO: 1, 2, 3 or 4), can comprise one or more affinity enhancing alternative nucleosides. In some aspects, the 5′ and/or 3′ flanking sequence comprises at least one 2′-O-methoxyethyl (MOE) nucleoside. In some aspects, the 5′ and/or 3′ flanking sequences, contain at least two MOE nucleosides. In some aspects, the 5′ flanking sequence comprises at least one, at least two, at least three, at least four, at least five, or at least six or more MOE nucleosides. In some aspects, the 5′ flanking sequence comprises at least one, at least two, at least three, at least four, at least five, or at least six or more MOE nucleosides. In some aspects, both the 5′ and 3′ flanking sequence comprise a MOE nucleoside. In some aspects, all the nucleosides in the flanking sequences are MOE nucleosides. In other aspects, the flanking sequence can comprise both MOE nucleosides and other nucleosides (mixed flanking sequence), such as DNA nucleosides and/or non-MOE alternative nucleosides, such as bicyclic nucleosides (BNAs) (e.g., LNA nucleosides (e.g., A-LNA, 5mC L-NA, G-LNA, T-LNA) or cET nucleosides), or other 2′ substituted nucleosides. In this case the DNA core sequence is defined as a contiguous sequence of at least 5 RNase H recruiting nucleosides (such as 5 to 16 DNA nucleosides or gapmers) flanked at the 5′ and 3′ end by an affinity enhancing alternative nucleoside, such as an MOE nucleoside.

In certain embodiments, the 5′ and/or 3′ flanking sequence comprises at least one BNA (e.g., at least one LNA nucleoside (e.g., A-LNA, 5mC L-NA, G-LNA, T-LNA) or cET nucleoside). In some embodiments, 5′ and/or 3′ flanking sequence comprises at least 2 bicyclic nucleosides. In some embodiments, the 5′ flanking sequence comprises at least one BNA. In some embodiments, both the 5′ and 3′ flanking sequence comprise a BNA. In some aspects, all the nucleosides in the flanking sequences are BNAs. In other aspects, the flanking sequence can comprise both BNAs and other nucleosides (mixed flanking sequences), such as DNA nucleosides and/or non-BNA alternative nucleosides, such as 2′ substituted nucleosides. In this case the DNA core sequence is defined as a contiguous sequence of at least five RNase H recruiting nucleosides (such as 5-16 DNA nucleosides) flanked at the 5′ and 3′ end by an affinity enhancing alternative nucleoside, such as a BNA, such as an LNA, such as beta-D-oxy-LNA.

The 5′ flank attached to the 5′ end of the DNA core sequence comprises, contains, or consists of at least one alternative sugar moiety (e.g., at least three, at least four, at least five, at least six, at least seven, or more alternative sugar moieties). In some aspects, the flanking sequence comprises or consists of from 1 to 7 alternative nucleobases, such as from 2 to 6 alternative nucleobases, such as from 2 to 5 alternative nucleobases, such as from 2 to 4 alternative nucleobases, such as from 1 to 3 alternative nucleobases, such as one, two, three or four alternative nucleobases. In some aspects, the flanking sequence comprises or consists of at least one alternative internucleoside linkage (e.g., at least three, at least four, at least five, at least six, at least seven, or more alternative internucleoside linkages).

The 3′ flank attached to the 3′ end of the DNA core sequence comprises, contains, or consists of at least one alternative sugar moiety (e.g., at least three, at least four, at least five, at least six, at least seven, or more alternative sugar moieties). In some aspects, the flanking sequence comprises or consists of from 1 to 7 alternative nucleobases, such as from 2 to 6 alternative nucleobases, such as from 2 to 5 alternative nucleobases, such as from 2 to 4 alternative nucleobases, such as from 1 to 3 alternative nucleobases, such as one, two, three, or four alternative nucleobases. In some aspects, the flanking sequence comprises or consists of at least one alternative internucleoside linkage (e.g., at least three, at least four, at least five, at least six, at least seven, or more alternative internucleoside linkages).

In an aspect, one or more or all of the alternative sugar moieties in the flanking sequence are 2′ alternative sugar moieties.

In a further aspect, one or more of the 2′ alternative sugar moieties in the wing regions are selected from 2′-O-alkyl-sugar moieties, 2′-O-methyl-sugar moieties, 2′-amino-sugar moieties, 2′-fluoro-sugar moieties, 2′-alkoxy-sugar moieties, MOE sugar moieties, LNA sugar moieties, arabino nucleic acid (ANA) sugar moieties, and 2′-fluoro-ANA sugar moieties.

In one aspect, all the alternative nucleosides in the flanking sequences are bicyclic nucleosides. In a further aspect, the bicyclic nucleosides in the flanking sequences are independently selected from the group consisting of oxy-LNA, thio-LNA, amino-LNA, cET, and/or ENA, in either the beta-D or alpha-L configurations or combinations thereof.

In some aspects, the one or more alternative internucleoside linkages in the flanking sequences are phosphorothioate internucleoside linkages. In some aspects, the phosphorothioate linkages are stereochemically pure phosphorothioate linkages. In some aspects, the phosphorothioate linkages are Sp phosphorothioate linkages. In other aspects, the phosphorothioate linkages are Rp phosphorothioate linkages. In some aspects, the alternative internucleoside linkages are 2′-alkoxy internucleoside linkages. In other aspects, the alternative internucleoside linkages are alkyl phosphate internucleoside linkages.

In certain embodiments, ASOs or RNA are chemically linked or encapsulated in one or more ligands, moieties, or conjugates that enhance the activity, cellular distribution, or cellular uptake of the oligonucleotide. Such moieties include but are not limited to lipid moieties such as a cholesterol moiety, an aliphatic chain, e.g., dodecandiol or undecyl residues, a polyamine or a polyethylene glycol chain, or the like.

The oligonucleotides used in the conjugates can be conveniently and routinely made through the well-known technique of solid-phase synthesis. Equipment for such synthesis is sold by several vendors including, for example, Applied Biosystems (Foster City, Calif.). Any other means for such synthesis known in the art can additionally or alternatively be employed. It is also known to use similar techniques to prepare other oligonucleotides, such as the phosphorothioates and alkylated derivatives.

In certain embodiments, compositions provided herein comprise polynucleotides (e.g., ASOs or RNA transcripts, e.g., siRNA, RNAi, or miRNA) linked or encapsulated in a lipid or lipid-like particle, polymer, or other non-viral delivery system. For example, sequences may be encapsulated in a lipid nanoparticle (LNP). As used herein, the phrase “lipid nanoparticle” refers to a transfer vehicle comprising one or more lipids (e.g., cationic lipids, non-cationic lipids, and PEG-modified lipids). Preferably, the lipid nanoparticles are formulated to deliver one or more ASOs and/or siRNA or RNAi to one or more target cells. Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides). Also contemplated is the use of polymers as transfer vehicles, whether alone or in combination with other transfer vehicles. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine. In one embodiment, the transfer vehicle is selected based upon its ability to facilitate the transfection of a target cell. Useful lipid nanoparticles for a cationic lipid to encapsulate and/or enhance the delivery of an siRNA (RNAi, or ASO) into the target cell. As used herein, the phrase “cationic lipid” refers to any of a number of lipid species that carry a net positive charge at a selected pH, such as physiological pH. The contemplated lipid nanoparticles may be prepared by including multi-component lipid mixtures of varying ratios employing one or more cationic lipids, non-cationic lipids and PEG-modified lipids. Several cationic lipids have been described in the literature, many of which are commercially available. See, e.g., WO2014/089486, US 2018/0353616A1, and U.S. Pat. No. 8,853,377B2, which are incorporated by reference. In certain embodiments, LNP formulation is performed using routine procedures comprising cholesterol, ionizable lipid, helper lipid, PEG-lipid and polymer forming a lipid bilayer around encapsulated mRNA (Kowalski et al., 2019, Mol. Ther. 27 (4): 710-728). In some embodiments, LNP comprises a cationic lipids (i.e. N-[1-(2,3-dioleoyloxy) propyl]-N,N,N-trimethylammonium chloride (DOTMA), or 1,2-dioleoyl-3-trimethylammonium-propane (DOTAP)) with helper lipid DOPE. In some embodiments, LNP comprises an ionizable lipid Dlin-MC3-DMA ionizable lipids, or diketopiperazine-based ionizable lipids (cKK-E12). In some embodiments, polymer comprises a polyethyleneimine (PEI), or a poly (β-amino) esters (PBAEs). See, e.g., WO2014/089486, US 2018/0353616A1, US2013/0037977A1, WO2015/074085A1, U.S. Pat. No. 9,670,152B2, and U.S. Pat. No. 8,853,377B2, which are incorporated by reference.

In certain embodiment, a non-viral vector is used for delivery of ASO (siRNA or RNAi) targeting the PTPB2-binding region of SYNGAP. In some embodiments, the ASO and/or RNAi is delivered at an amount greater than about 0.5 mg/kg (e.g., greater than about 1.0 mg/kg, 1.5 mg/kg, 2.0 mg/kg, 2.5 mg/kg, 3.0 mg/kg, 4.0 mg/kg, 5.0 mg/kg, 6.0 mg/kg, 7.0 mg/kg, 8.0 mg/kg, 9.0 mg/kg, or 10.0 mg/kg) body weight of nucleotide per dose. In some embodiments, the nucleotide is delivered at an amount ranging from about 0.1-100 mg/kg (e.g., about 0.1-90 mg/kg, 0.1-80 mg/kg, 0.1-70 mg/kg, 0.1-60 mg/kg, 0.1-50 mg/kg, 0.1-40 mg/kg, 0.1-30 mg/kg, 0.1-20 mg/kg, 0.1-10 mg/kg) body weight of nucleotide per dose. In some embodiments, the nucleotide acid is delivered at an amount of or greater than about 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, or 500 mg per dose.

In certain embodiments, ASOs and/or RNAi are encapsulated in a lipid nanoparticle (LNP). As used herein, the phrase “lipid nanoparticle” refers to a transfer vehicle comprising one or more lipids (e.g., cationic lipids, non-cationic lipids, and PEG-modified lipids). Preferably, the lipid nanoparticles are formulated to deliver one or more miRNA to one or more target cells (e.g., dorsal root ganglion, lower motor neurons and/or upper motor neurons, or the cell types identified above in the CNS). Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides). Also contemplated is the use of polymers as transfer vehicles, whether alone or in combination with other transfer vehicles. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine. In one embodiment, the transfer vehicle is selected based upon its ability to facilitate the transfection of a agent (e.g., ASO, miRNA, and/or siRNA) to a target cell. Useful lipid nanoparticles for and agent comprise a cationic lipid to encapsulate and/or enhance the delivery of the agent into the target cell that will act as a depot for protein production. As used herein, the phrase “cationic lipid” refers to any of a number of lipid species that carry a net positive charge at a selected pH, such as physiological pH. The contemplated lipid nanoparticles may be prepared by including multi-component lipid mixtures of varying ratios employing one or more cationic lipids, non-cationic lipids and PEG-modified lipids. Several cationic lipids have been described in the literature, many of which are commercially available. See, e.g., WO2014/089486, US 2018/0353616A1, and U.S. Pat. No. 8,853,377B2, which are incorporated by reference. In certain embodiments, LNP formulation is performed using routine procedures comprising cholesterol, ionizable lipid, helper lipid, PEG-lipid and polymer forming a lipid bilayer around encapsulated mRNA (Kowalski et al., 2019, Mol. Ther. 27 (4): 710-728). In some embodiments, LNP comprises a cationic lipids (i.e. N-[1-(2,3-dioleoyloxy) propyl]-N,N,N-trimethylammonium chloride (DOTMA), or 1,2-dioleoyl-3-trimethylammonium-propane (DOTAP)) with helper lipid DOPE. In some embodiments, LNP comprises an ionizable lipid Dlin-MC3-DMA ionizable lipids, or diketopiperazine-based ionizable lipids (cKK-E12). In some embodiments, polymer comprises a polyethyleneimine (PEI), or a poly(β-amino) esters (PBAEs). See, e.g., WO2014/089486, US 2018/0353616A1, US2013/0037977A1, WO2015/074085A1, U.S. Pat. No. 9,670,152B2, and U.S. Pat. No. 8,853,377B2, which are incorporated by reference.

As used herein, the terms “intrathecal delivery” or “intrathecal administration” refer to a route of administration via an injection into the spinal canal, more specifically into the subarachnoid space so that it reaches the cerebrospinal fluid (CSF). Intrathecal delivery may include lumbar puncture, intraventricular (including intracerebroventricular (ICV)), suboccipital/intracisternal, and/or C1-2 puncture. For example, material may be introduced for diffusion throughout the subarachnoid space by means of lumbar puncture. In another example, injection may be into the cisterna magna. In certain embodiments, the composition is administered using Ommaya reservoir.

As used herein, the terms “intracisternal delivery” or “intracisternal administration” refer to a route of administration directly into the cerebrospinal fluid of the cisterna magna cerebellomedularis, more specifically via a suboccipital puncture or by direct injection into the cisterna magna or via permanently positioned tube.

### Recombinant Viral Vectors

In some embodiments, one or more nucleic acid sequences provided herein may be delivered via a recombinant, replication-defective viral vector (e.g, a lentivirus, adenovirus, adeno-associated virus). In certain embodiments, a viral vector may further comprise one or more additional sequences (e.g., a replacement gene expressing functional SYNGAP1) for delivery to a subject. In certain embodiments, a viral vector is selected for its targeting specify to the brain, or subsets of cells therein, and/or other cells with in the central nervous system.

In certain embodiments, a composition is provided which comprises an aqueous liquid suitable for intrathecal injection and a stock of vector (e.g., rAAV having a AAV capsid which preferentially targets cells in the central nervous system and/or the dorsal root ganglia (e.g., CNS, including, e.g., nerve cells (such as, pyramidal, purkinje, granule, spindle, and interneuron cells) and glia cells (such as astrocytes, oligodendrocytes, microglia, and ependymal cells), wherein the vector having a nucleic sequence (e.g., of at least one an interfering agent, e.g., an miRNA target sequence) for delivery to the central nervous system (CNS). In certain embodiments, the composition comprising one or more vectors as described herein is formulated for sub-occipital injection into the cisterna magna (intra-cisterna magna). In certain embodiments, the composition is administered via a computed tomography-(CT-) rAAV injection. In certain embodiments, the composition is administered using Ommaya reservoir. In certain embodiments, the patient is administered a single dose of the composition. In certain embodiments, for administration to a human patient, the composition (e.g., vector, ASO, etc) is suitably suspended in an aqueous solution containing saline, a surfactant, and a physiologically compatible salt or mixture of salts. Suitably, the formulation is adjusted to a physiologically acceptable pH, e.g., in the range of pH 6 to 9, or pH 6.5 to 7.5, pH 7.0 to 7.7, or pH 7.2 to 7.8. As the pH of the cerebrospinal fluid is about 7.28 to about 7.32, or a pH of 7.2 to 7.4, for intrathecal delivery, a pH within this range may be desired. However, other pHs within the broadest ranges and these subranges may be selected for other route of delivery.

In certain embodiments, an agent (e.g., an ASO or RNAi) is expressed from a recombinant adeno-associated virus, which has an AAV capsid and a vector genome packaged in the AAV capsid.

In certain embodiments, a “vector genome” refers to the nucleic acid sequence packaged inside a parvovirus (e.g., rAAV) capsid which forms a viral particle. Such a nucleic acid sequence contains AAV inverted terminal repeat sequences (ITRs). In the examples herein, a vector genome contains, at a minimum, from 5′ to 3′, an AAV 5′ ITR, coding sequence(s), and an AAV 3′ ITR. ITRs from AAV2, a different source AAV than the capsid, or other than full-length ITRs may be selected. In certain embodiments, the ITRs are from the same AAV source as the AAV which provides the rep function during production or a transcomplementing AAV. Further, other ITRs, e.g., self-complementary (scAAV) ITRs, may be used. Further, the vector genome contains regulatory sequences which direct expression of the gene products. Suitable components of a vector genome are discussed in more detail herein.

In one embodiment, the rAAV is pseudotyped, i.e., the AAV capsid is from a different source AAV than that the AAV which provides the ITRs. In one embodiment, the ITRs of AAV serotype 2 are used. However, ITRs from other suitable sources may be selected. Optionally, the AAV may be a self-complementary AAV.

In certain embodiments, the AAV capsid is from clade F. See, e.g., [U.S. Pat. No. 7,906,111; WO 2018/160582; WO 2019/168961. See, e.g., AAVhu68, AAV9 or variants thereof, AAVhu31, AAVhu32, AAVhu68, AAV1, AAV2, AAV6, AAV6.2, or another AAV which targets the CNS, or subsets of cells therein. As used herein, an “AAV capsid” is a self-assembled AAV capsid composed of multiple AAV vp proteins. The AAV proteins are typically expressed as alternative splice variants encoded by a nucleic acid sequence which encodes the vp1 amino acid sequence. These splice variants result in proteins of different length. In certain embodiments, “AAV9 capsid” includes an AAV having an amino acid sequence which is 99% identical to AAS99264 or 99% identical thereto. See, also U.S. Pat. No. 7,906,111 and WO 2005/033321. As used herein “AAV9 variants” include those described in, e.g., WO2016/049230, U.S. Pat. No. 8,927,514, US 2015/0344911, and U.S. Pat. No. 8,734,809. See, also, AAV9 deamidation pattern and compositions as described, e.g., in WO 2019/168961. Optionally, AAV capsid may include, e.g., natural isolates (e.g., hu31 or hu32), or variants of AAV9 having amino acid substitutions, deletions or additions, e.g., including but not limited to amino acid substitutions selected from alternate residues “recruited” from the corresponding position in any other AAV capsid aligned with the AAV9 capsid; e.g., such as described in U.S. Pat. Nos. 9,102,949, 8,927,514, US2015/349911; and WO 2016/049230A1. However, in other embodiments, other variants of AAV9, or AAV9 capsids having at least about 95% identity to the above-referenced sequences may be selected. See, e.g., US Published Patent Application No. 2015/0079038. Methods of generating the capsid, coding sequences therefore, and methods for production of rAAV viral vectors have been described. See, e.g., Gao et al, Proc. Natl. Acad. Sci. U.S.A. 100 (10), 6081-6 (2003) and US 2013/0045186A1.

Another suitable Clade F capsid is the AAVhu68 capsid. See, e.g., WO 2018/160582, which is incorporated herein by reference in its entirety. Still other suitable AAV capsids may be selected. See, e.g., WO 2019/168961 and WO 2019/169004, published Sep. 6, 2019, which are incorporated by reference herein in their entirely. See also, e.g., WO 2020/223232 A1 (AAV rh.90), WO 2020/223231 A1 (AAV rh.91), and WO 2020/223236 A1 (AAV rh.92, AAV rh.93, AAV rh.91.93), which are incorporated herein by reference in its entirety. These documents also describe other AAV which may be selected for generating AAV and are incorporated by reference. In some embodiments, an AAV capsid (cap) for use in the viral vector can be generated by mutagenesis (i.e., by insertions, deletions, or substitutions) of one of the aforementioned AAV caps or its encoding nucleic acid. In some embodiments, the AAV capsid is chimeric, comprising domains from two or three or four or more of the aforementioned AAV capsid proteins. In some embodiments, the AAV capsid is a mosaic of vp1, vp2, and vp3 monomers from two or three different AAVs or recombinant AAVs. In some embodiments, an rAAV composition comprises more than one of the aforementioned caps.

Where the interfering agent and/or gene is to be expressed from an AAV, the expression cassettes described herein include an AAV 5′ inverted terminal repeat (ITR) and an AAV 3′ ITR. However, other configurations of these elements may be suitable. In one embodiment, the ITRs are from an AAV different than that supplying a capsid. In one embodiment, the ITR sequences from AAV2. However, ITRs from other AAV sources may be selected. A shortened version of the 5′ ITR, termed ΔITR, has been described in which the D-sequence and terminal resolution site (trs) are deleted. In certain embodiments, the vector genome includes a shortened AAV2 ITR of 130 base pairs, wherein the external A elements is deleted. Without wishing to be bound by theory, it is believed that the shortened ITR is reverts back to the wild-type length of 145 base pairs during vector DNA amplification using the internal (A′) element as a template. In other embodiments, full-length AAV 5′ and 3′ ITRs are used. Where the source of the ITRs is from AAV2 and the AAV capsid is from another AAV source, the resulting vector may be termed pseudotyped. However, other configurations of these elements may be suitable. Where a pseudotyped AAV is to be produced, the ITRs in the expression are selected from a source which differs from the AAV source of the capsid. For example, AAV2 ITRs may be selected for use with an AAV capsid having a particular efficiency for targeting CNS or tissues or cells within the CNS. In one embodiment, the ITR sequences from AAV2, or the deleted version thereof (ΔITR), are used for convenience and to accelerate regulatory approval. However, ITRs from other AAV sources may be selected.

Construction of vectors for expression of polynucleotides can be accomplished using conventional techniques. For generation of efficient expression vectors, it is necessary to have regulatory sequences that control the expression of the polynucleotide. These regulatory sequences include, e.g., at least one promoter, a poly A signal, and various other vector elements (e.g., one or more of each, an enhancer, an intron, a post-translational regulatory element) and are influenced by specific cellular factors that interact with these sequences, and are well known in the art.

As used herein, an “expression cassette” refers to a nucleic acid molecule which comprises a coding sequence, promoter, and may include other regulatory sequences therefor. In certain embodiments, a vector genome may contain two or more expression cassettes. In other embodiments, the term “transgene” may be used interchangeably with “expression cassette”.

As used herein, a “stock” of rAAV refers to a population of rAAV. Despite heterogeneity in their capsid proteins due to deamidation, rAAV in a stock are expected to share an identical vector genome. A stock can include rAAV having capsids with, for example, heterogeneous deamidation patterns characteristic of the selected AAV capsid proteins and a selected production system. The stock may be produced from a single production system or pooled from multiple runs of the production system. A variety of production systems, including but not limited to those described herein, may be selected.

In many instances, rAAV particles are referred to as “DNase resistant.” However, in addition to this endonuclease (DNase), other endo- and exo-nucleases may also be used in the purification steps described herein, to remove contaminating nucleic acids. Such nucleases may be selected to degrade single stranded DNA and/or double-stranded DNA, and RNA. Such steps may contain a single nuclease, or mixtures of nucleases directed to different targets, and may be endonucleases or exonucleases.

The term “nuclease-resistant” indicates that the AAV capsid has fully assembled around the expression cassette which is designed to deliver a transgene to a host cell and protects these packaged genomic sequences from degradation (digestion) during nuclease incubation steps designed to remove contaminating nucleic acids which may be present from the production process.

The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

Methods of preparing AAV-based vectors are known. See, e.g., US Published Patent Application No. 2007/0036760 (Feb. 15, 2007), which is incorporated by reference herein. The use of AAV capsids of AAV9 are particularly well suited for the compositions and methods described herein. The sequences of AAV9 and methods of generating vectors based on the AAV9 capsid are described in U.S. Pat. No. 7,906,111; US2015/0315612; WO 2012/112832; which are incorporated herein by reference. However, other AAV capsids may be selected or generated. For example, the sequences of AAV1, AAV5, and AAV6 are known as are methods of generating vectors. See, e.g., U.S. Pat. No. 7,282,199 B2, U.S. Pat. Nos. 7,790,449, and 8,318,480, which are incorporated herein by reference. The sequences of a number of such AAV are provided in the above-cited U.S. Pat. No. 7,282,199 B2, U.S. Pat. Nos. 7,790,449, 8,318,480, and 7,906,111, and/or are available from GenBank. The sequences of any of the AAV capsids can be readily generated synthetically or using a variety of molecular biology and genetic engineering techniques. Suitable production techniques are well known to those of skill in the art. See, e.g., Sambrook et al, Molecular Cloning: A Laboratory Manual, Cold Spring Harbor Press (Cold Spring Harbor, NY). Alternatively, oligonucleotides encoding peptides (e.g., CDRs) or the peptides themselves can generated synthetically, e.g., by the well-known solid phase peptide synthesis methods (Merrifield, (1962) J. Am. Chem. Soc., 85:2149; Stewart and Young, Solid Phase Peptide Synthesis (Freeman, San Francisco, 1969) pp. 27-62). These and other suitable production methods are within the knowledge of those of skill in the art and are not a limitation.

The recombinant adeno-associated virus (AAV) described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid; a functional rep gene; an expression cassette composed of, at a minimum, AAV inverted terminal repeats (ITRs) and a transgene; and sufficient helper functions to permit packaging of the expression cassette into the AAV capsid protein.

Any suitable method may be used to determine particle numbers or viral genome. For example, in certain embodiments, qPCR is used. Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25 (2): 115-25. doi: 10.1089/hgtb.2013.131. Epub 2014 Feb. 14.

Dosages of the vector will depend primarily on factors such as the condition being treated, the age, weight and health of the patient, and may thus vary among patients. For example, a therapeutically effective human dosage of viral vector is generally in the range of from about 25 to about 1000 microliters to about 100 mL of solution containing concentrations of from about 1×10⁹ to 1×10¹⁶ genomes virus vector (to treat an average subject of 70 kg in body weight) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10¹² GC per dose including all integers or fractional amounts within the range.

In certain embodiments, the dose is in the range of about 1×10⁹ GC/g brain mass to about 1×10¹² GC/g brain mass. In certain embodiments, the dose is in the range of about 1×10¹⁰ GC/g brain mass to about 3.33×10¹¹ GC/g brain mass. In certain embodiments, the dose is in the range of about 3.33×10¹¹ GC/g brain mass to about 1.1×10¹² GC/g brain mass. In certain embodiments, the dose is in the range of about 1.1×10¹² GC/g brain mass to about 3.33×10¹³ GC/g brain mass. In certain embodiments, the dose is lower than 3.33×10¹¹ GC/g brain mass. In certain embodiments, the dose is lower than 1.1×10¹² GC/g brain mass. In certain embodiments, the dose is lower than 3.33×10¹³ GC/g brain mass. In certain embodiments, the dose is about 1×10¹⁰ GC/g brain mass. In certain embodiments, the dose is about 2×10¹⁰ GC/g brain mass. In certain embodiments, the dose is about 2×10¹⁰ GC/g brain mass. In certain embodiments, the dose is about 3×10¹⁰ GC/g brain mass. In certain embodiments, the dose is about 4×10¹⁰ GC/g brain mass. In certain embodiments, the dose is about 5×10¹⁰ GC/g brain mass. In certain embodiments, the dose about 6×10¹⁰ GC/g brain mass. In certain embodiments, the dose is about 7×10¹⁰ GC/g brain mass. In certain embodiments, the dose about 8×10¹⁰ GC/g brain mass. In certain embodiments, the dose is about 9×10¹⁰ GC/g brain mass. In certain embodiments, the dose is about 1×10¹¹ GC/g brain mass. In certain embodiments, the dose is about 2×10¹¹ GC/g brain mass. In certain embodiments, the dose is about 3×10¹¹ GC/g brain mass. In certain embodiments, the dose is about 4×10¹¹ GC/g brain mass. In certain embodiments, the dose is administered to humans as a flat dose in the range of about 1.44×10¹³ to 4.33×10¹⁴ GC of the rAAV. In certain embodiments, the dose is administered to humans as a flat dose in the range of about 1.44×10¹³ to 2×10¹⁴ GC of the rAAV. In certain embodiments, the dose is administered to humans as a flat dose in the range of about 3×10¹³ to 1×10¹⁴ GC of the rAAV. In certain embodiments, the dose is administered to humans as a flat dose in the range of about 5×10¹³ to 1×10¹⁴ GC of the rAAV. In some embodiments, the compositions can be formulated in dosage units to contain an amount of AAV that is in the range of about 1×10¹³ to 8×10¹⁴ GC of the rAAV. In some embodiments, the compositions can be formulated in dosage units to contain an amount of rAAV that is in the range of about 1.44×10¹³ to 4.33×10¹⁴ GC of the rAAV. In some embodiments, the compositions can be formulated in dosage units to contain an amount of rAAV that is in the range of about 3×10¹³ to 1×10¹⁴ GC of the rAAV. In some embodiments, the compositions can be formulated in dosage units to contain an amount of rAAV that is in the range of about 5×10¹³ to 1×10¹⁴ GC of the rAAV.

In certain embodiments, the vector is administered to a subject in a single dose. In certain embodiments, vector may be delivered via multiple injections (for example 2 doses) is desired.

The composition for delivery may contain a buffered saline aqueous solution. In certain embodiments, the composition does not comprise sodium bicarbonate. Examples of suitable buffered saline aqueous solutions comprising one or more of sodium phosphate, sodium chloride, potassium chloride, calcium chloride, magnesium chloride and mixtures thereof, in water, such as a Harvard's buffer. The aqueous solution may further contain Kolliphor® P188, a poloxamer which is commercially available from BASF which was formerly sold under the trade name Lutrol® F68. The aqueous solution may have a pH of 7.2 or a pH of 7.4.

In another embodiment, the formulation may contain a buffered saline aqueous solution comprising 1 mM Sodium Phosphate (Na3PO4), 150 mM sodium chloride (NaCl), 3 mM potassium chloride (KCl), 1.4 mM calcium chloride (CaCl2), 0.8 mM magnesium chloride (MgCl2), and 0.001% Kolliphor® 188. See, e.g., harvardapparatus.com/harvard-apparatus-perfusion-fluid.html. In certain embodiments, Harvard's buffer is preferred.

In other embodiments, the formulation may contain one or more permeation enhancers. Examples of suitable permeation enhancers may include, e.g., mannitol, sodium glycocholate, sodium taurocholate, sodium deoxycholate, sodium salicylate, sodium caprylate, sodium caprate, sodium lauryl sulfate, polyoxyethylene-9-laurel ether, or EDTA.

In another embodiment, the composition includes a carrier, diluent, excipient and/or adjuvant. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The buffer/carrier should include a component that prevents the rAAV, from sticking to the infusion tubing but does not interfere with the rAAV binding activity in vivo.

Optionally, the compositions may contain, in addition to the vector and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host. Delivery vehicles such as liposomes, nanocapsules, microparticles, microspheres, lipid particles, vesicles, and the like, may be used for the introduction of the compositions of the present invention into suitable host cells. In particular, the rAAV vector delivered transgenes may be formulated for delivery either encapsulated in a lipid particle, a liposome, a vesicle, a nanosphere, or a nanoparticle or the like.

In one embodiment, a composition includes a final formulation suitable for delivery to a subject, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

The compositions and methods described herein are used to increase the production of a functional protein (e.g., SYNGAP1) by eliminating PTBP2-mediated alternative splicing events. See, e.g., Tables 1 and 3.

As used herein, the term “functional” refers to the amount of activity or function of a SYNGAP1 or SCNIA protein that is necessary to eliminate or reduce one or more symptoms of a treated condition, e.g., AD mental retardation 5 or Dravet syndrome. In embodiments, the methods are used to increase the production of a partially functional SYNGAP 1 or SCNIA protein. As used herein, the term “partially functional” refers to any amount of activity or function of the SYNGAP 1 protein that is less than the amount of activity or function that is necessary to eliminate or prevent any one or more symptoms of a disease or condition. In some embodiments, a partially functional protein or RNA will have at least 10%, at least 20%, at least 30%, at least 40%, at least 50%, at least 60%, at least 70%, at least 75%, at least 80%, at least 85%, at least 90%, or at least 95% less activity relative to the fully functional protein or RNA.

By the term “SYNGAP activity” or like term is meant those functions attributed to SYNGAP as discussed herein, e.g., PDZ domain and rasGTPase inhibition. Related activities can impact SYNGAP activity including synthesis of SYNGAP (transcription and translation), SYNGAP processing (e.g., protein maturation including modification such as glycosylation), protein stability in SYNGAP-expressing cells, and neuromodulation.

As used herein, the term “operably linked” refers to both expression control sequences that are contiguous with the gene of interest and expression control sequences that act in trans or at a distance to control the gene of interest.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene. Thus, with reference to the coding sequence, the promoter is heterologous.

Identity or similarity with respect to a sequence is defined herein as the percentage of amino acid residues in the candidate sequence that are identical (i.e., same residue) or similar (i.e., amino acid residue from the same group based on common side-chain properties, see below) with the peptide and polypeptide regions provided herein, after aligning the sequences and introducing gaps, if necessary, to achieve the maximum percent sequence identity. Percent (%) identity is a measure of the relationship between two polynucleotides or two polypeptides, as determined by comparing their nucleotide or amino acid sequences, respectively. In general, the two sequences to be compared are aligned to give a maximum correlation between the sequences. The alignment of the two sequences is examined and the number of positions giving an exact amino acid or nucleotide correspondence between the two sequences determined, divided by the total length of the alignment and multiplied by 100 to give a % identity figure. This % identity figure may be determined over the whole length of the sequences to be compared, which is particularly suitable for sequences of the same or very similar length and which are highly homologous, or over shorter defined lengths, which is more suitable for sequences of unequal length or which have a lower level of homology. There are a number of algorithms, and computer programs based thereon, which are available to be used the literature and/or publicly or commercially available for performing alignments and percent identity. The selection of the algorithm or program is not a limitation.

Examples of suitable alignment programs including, e.g., the software CLUSTALW under Unix and then be imported into the Bioedit program (Hall, T. A. 1999, BioEdit: a user-friendly biological sequence alignment editor and analysis program for Windows 95/98/NT. Nucl. Acids. Symp. Ser. 41:95-98); the Clustal Omega available from EMBL-EBI (Sievers, Fabian, et al. “Fast, scalable generation of high-quality protein multiple sequence alignments using Clustal Omega.” Molecular systems biology 7.1 (2011): 539 and Goujon, Mickael, et al. “A new bioinformatics analysis tools framework at EMBL-EBI.” Nucleic acids research 38.suppl 2 (2010): W695-W699); the Wisconsin Sequence Analysis Package, version 9.1 (Devereux J. et al., Nucleic Acids Res., 12:387-395, 1984, available from Genetics Computer Group, Madison, Wis., USA). The programs BESTFIT and GAP, may be used to determine the % identity between two polynucleotides and the % identity between two polypeptide sequences.

Other programs for determining identity and/or similarity between sequences include, e.g, the BLAST family of programs available from the National Center for Biotechnology Information (NCB), Bethesda, Md., USA and accessible through the home page of the NCBI at www.ncbi.nlm.nih.gov), the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used; and FASTA (Pearson W. R. and Lipman D. J., Proc. Natl. Acad. Sci. USA, 85:2444-8, 1988, available as part of the Wisconsin Sequence Analysis Package). SeqWeb Software (a web-based interface to the GCG Wisconsin Package

The term “a” or “an” refers to one or more. As such, the terms “a” (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of”′ language.

The term “about” encompasses a variation within and including±10%, unless otherwise specified.

Unless defined otherwise in this specification, technical and scientific terms used herein have the same meaning as commonly understood by one of ordinary skill in the art and by reference to published texts, which provide one skilled in the art with a general guide to many of the terms used in the present application.

### Example 1

Transcriptome wide AS are assessed after antisense silencing of PTBP2 in human induced-pluripotent stem-cell derived cortical excitatory neurons (iPS-neurons). This is combined with CLIP-seq analysis of PTBP2 binding in both iPS-neurons and human cortical tissue to determine direct targets of PTBP-dependent AS in human brain. We have identified several genes that are alternatively spliced and repressed by PTBP2, and which cause human disease when reduced in expression-indicating potential targets for therapeutic splice switching. We perform extensive follow up on one such gene, SYNGAP1, where variants lead to reduced expression (haploinsufficiency) and a phenotypically broad neurodevelopmental disorder that includes epilepsy, developmental delay, movement disorders and autism (Berryer et al., 2013; Clement et al., 2012; Hamdan et al., 2009). We utilize PTBP2 binding and splicing data to guide ASO disruption of PTBP2 binding to SYNGAP1, which effectively redirects splicing to increase gene and protein expression. Together, this work provides the first comprehensive map of PTBP2-dependent AS in human neurons and cortical tissue and identifies targets for ASO manipulation that may offer therapeutic potential.

### Results

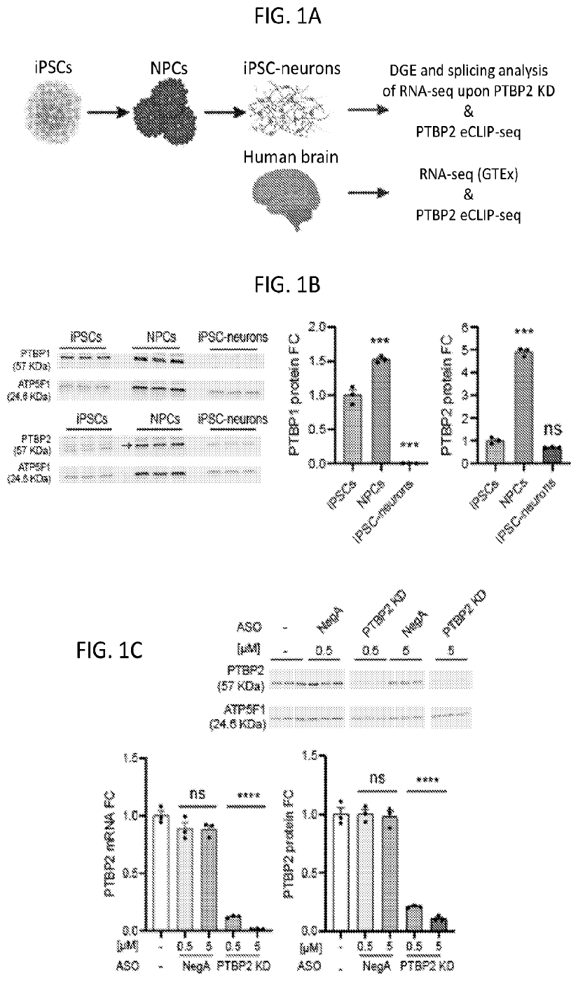

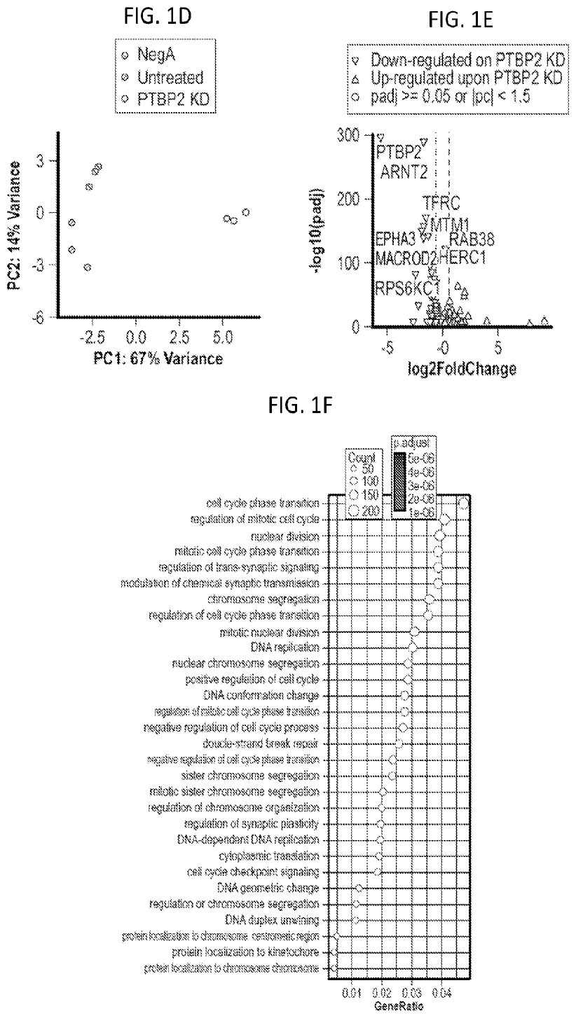

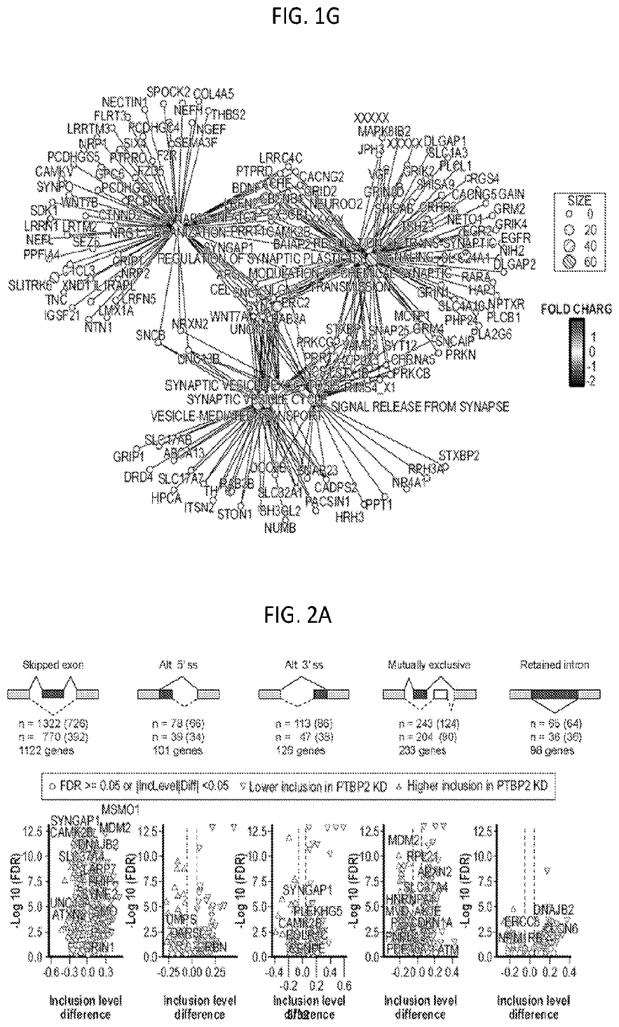

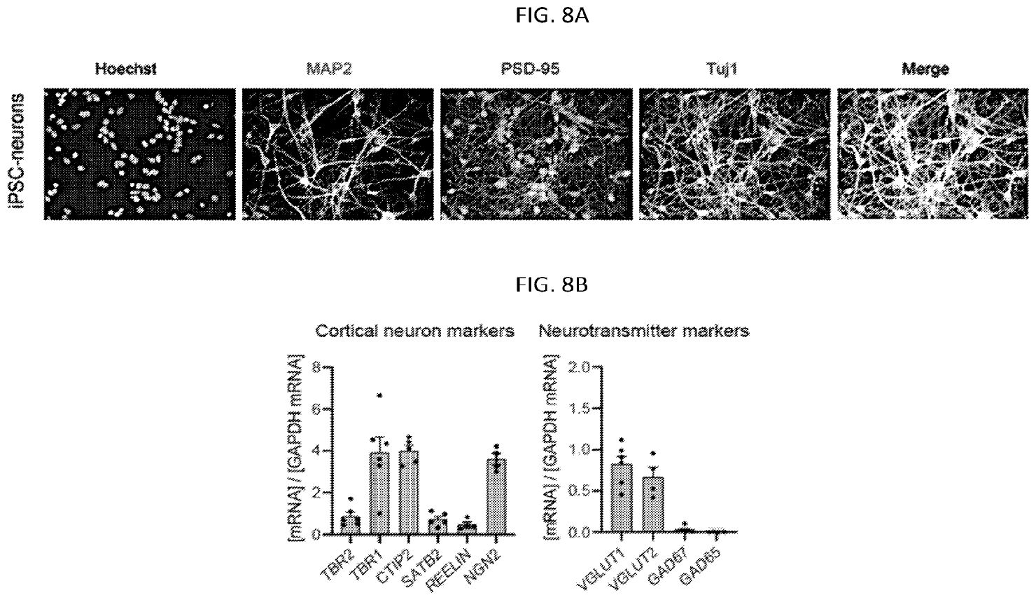

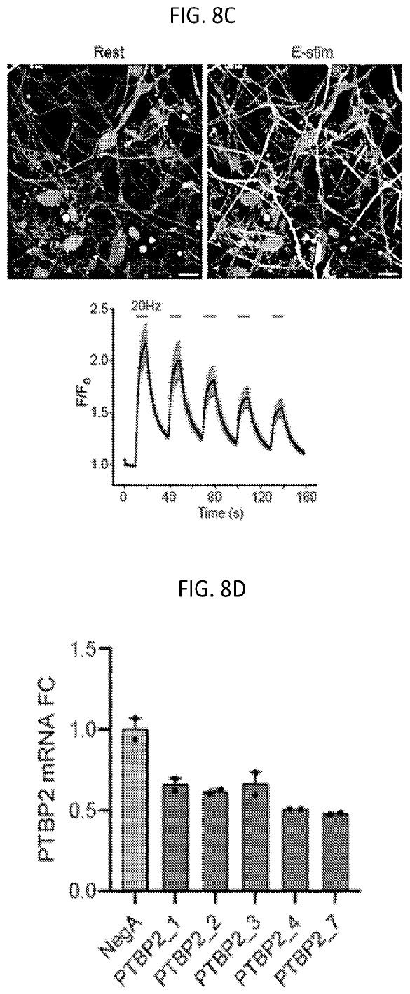

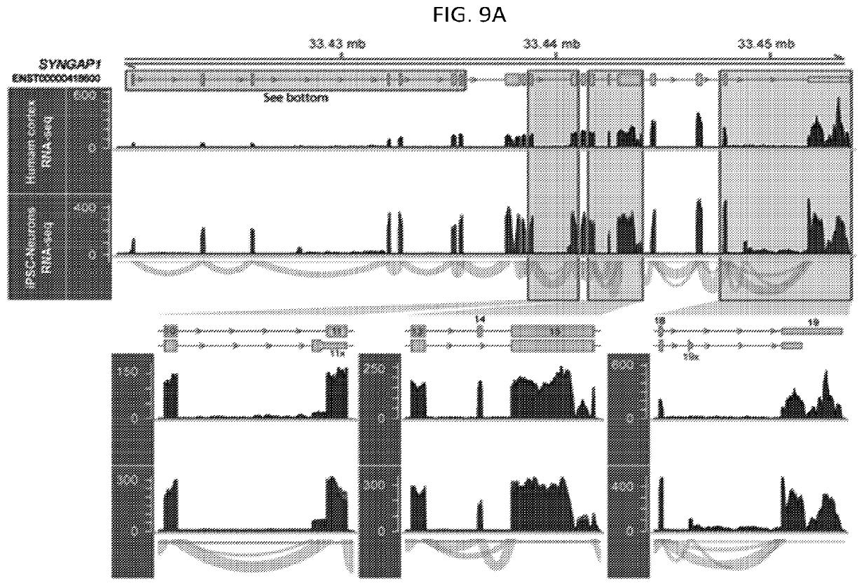

To identify targets of PTBP2-dependent splicing, we first depleted PTBP2 in iPSC-derived neurons (Materials and Methods; FIG. 1A; FIG. 8). Neuronal maturity was indicated by both spontaneous and electrically evoked calcium oscillations that demonstrate matured synaptic functionality (FIG. 8A-B). Consistent with previous literature on the developmental regulation of PTBP isoforms, we noted high levels of PTBP1 in iPSCs, increased expression of both PTBP1 and PTBP2 in NPCs, and expression restricted to PTBP2 in matured neurons (FIG. 1B). To deplete PTBP2 in neurons, we utilized locked-nucleic acid (LNA) modified ASOs (“gapmers”) that bind to complementary sequences and trigger RNase H1-dependent degradation of the target mRNA. We performed an initial screening with several gapmers targeting PTBP2 as well as a negative control gapmer in HEK293T cells and assessed PTBP 2 mRNA expression 24 h later (FIG. 8C). The most effective gapmer (PTBP2_7, hereafter referred to as PTBP2 KD) was examined for dose-dependent knockdown in iPS-neurons (day 40-50 of differentiation) via gymnotic delivery, and PTBP2 expression was assessed 7 days later. PTBP2 KD produced robust reduction of PTBP2 mRNA and protein expression, with PTBP2 protein levels reduced by >90% (FIG. 1C).

We next performed RNA sequencing on untreated, negative control, and PTBP2 KD samples. Principal component analysis indicated tight clustering between biological replicates and between control groups, with the majority of variance due to PTBP2 KD (FIG. 1D). Differential gene expression (DGE, PTBP2 KD vs. untreated) analysis identified 1421 upregulated and 1730 downregulated genes upon PTBP2 KD using an adjusted p-value of 0.05 and fold change of ≥1.15 (FIG. 1E). Gene ontology analysis of biological processes indicated prominent alterations in genes involved in cell cycle regulation, DNA repair, and synaptic transmission and plasticity (FIG. 1F). Within the synapse groups, 304 genes associated with synaptic transmission or plasticity were differentially regulated upon PTBP2 KD, including well-known targets of PTBP2 such as DLG4 that encodes the major scaffolding protein of excitatory synapses (PSD-95), as well as novel targets such as STXBP1 and SYNGAP1, which encode prominent regulators of pre- and post-synaptic function (FIG. 1G).

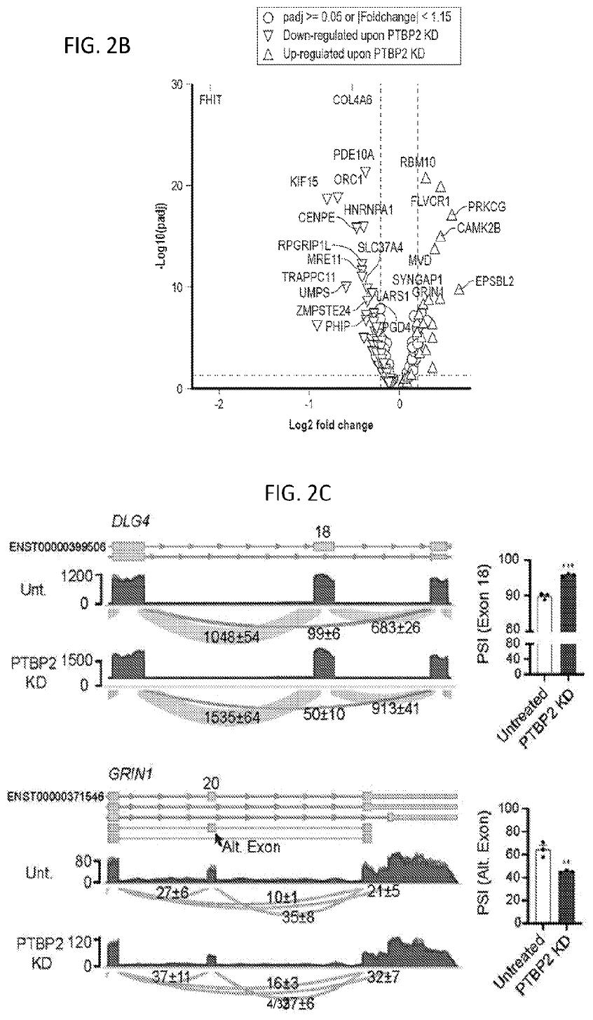

We evaluated transcriptome-wide differential AS upon PTBP2 KD using rMATS (Shen et al., 2014) (PTBP2 KD vs. untreated) (FIG. 2A). Using a false discovery rate (FDR)<0.05 and inclusion level difference of 5%, 1389 genes were differentially spliced upon PTBP2 depletion. rMATS identifies 5 distinct alternative splicing events: exon skipping, alternative 5′ and 3′ splice sites (SS), mutually exclusive splicing events, and retained introns. For each event type, >100 genes were differentially spliced upon PTBP2-depletion, with exon skipping the most predominantly regulated event (FIG. 2A). Both inclusion and exclusion of AS events were observed upon PTBP2 KD, although across all 5 types of events the depletion of PTBP2 was more likely to promote event exclusion. These findings are consistent with the endogenous presence of PTBP2 preferentially promoting the inclusion of AS events in human cortical neurons.

Given our interest in neurodevelopmental disorders, we cross-referenced this list of alternatively spliced transcripts regulated by PTBP2 with the Orphanet database of disease-causing genes. 342 genetic etiologies were differentially spliced upon PTBP2 depletion, including GRIN1, MVD, DNM1, CAMK2B, HNRNPA1, CTNND1 and others (FIG. 2A). The most significant 3′AS site regulation of an Orphanet gene was observed in SYNGAP 1 (FIG. 2A), the genetic cause of SYNGAP1-associated intellectual disability, which also exhibited a significant exon skipping event upon PTBP2 depletion (FIG. 2A). Intriguingly, AS of SYNGAP1 was recently demonstrated to promote nonsense-mediated decay (NMD) and restrict SYNGAP1 expression in HEK293 cells (Lim et al., 2020), but no mechanism for this AS nor its neuronal impacts were explored. Of these differentially spliced and disease-causing genes, 72 (including SYNGAP 1 and GRIN1) were also differentially expressed at the mRNA level upon PTBP2 KD (adjusted p-value<0.05 and fold change of ≥1.15), suggesting that PTBP2-dependent AS regulates the splicing and expression of several disease-causing genes in human neurons (FIG. 2B-C). FIG. 2C shows a closer examination of the AS of DLG4 and GRIN1. Consistent with previous literature, we found PTBP2 promotes the exclusion of DLG4 exon 18 (i.e. KD increases inclusion), which triggers NMD and restricts expression of PSD-95 (Linares et al., 2015). For GRIN1, rMATS identified significant PTBP2-dependent AS to an alternative exon that was not previously annotated, but which introduces a frame-shift from the canonical transcript.

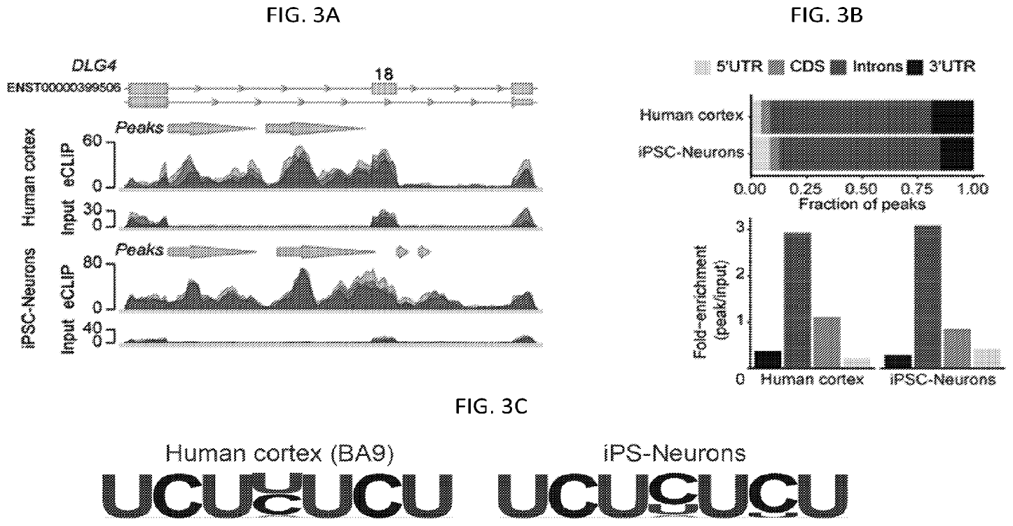

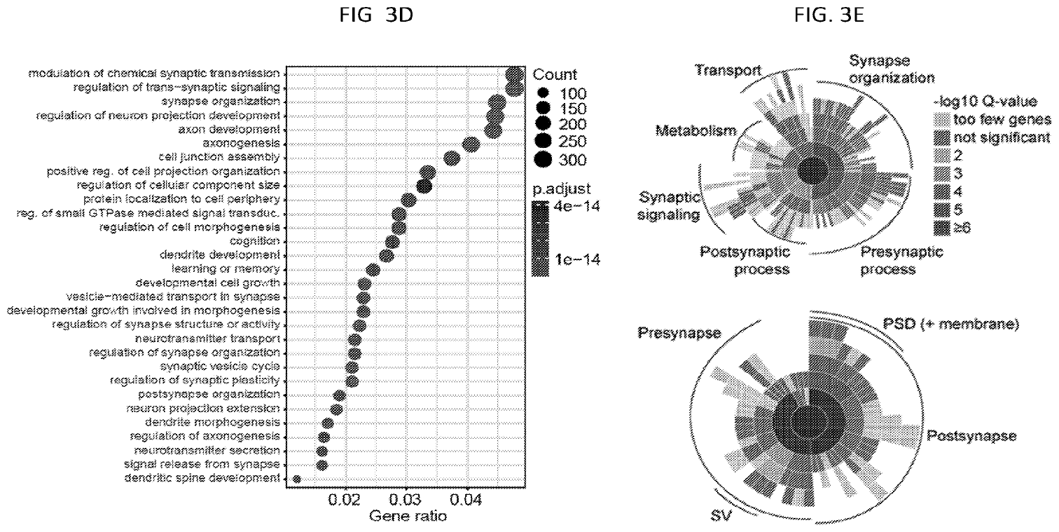

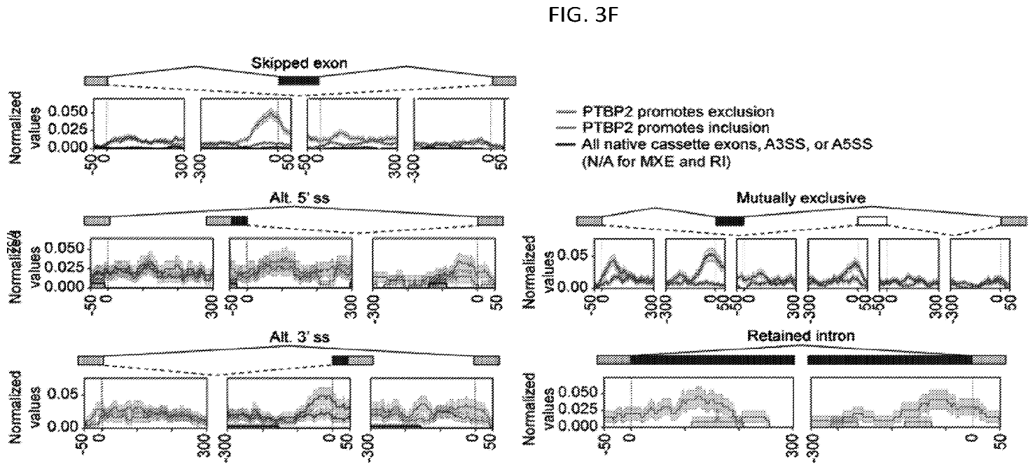

To increase confidence in PTBP2-dependent AS targets and to identify direct sites of PTBP2 binding, we next performed PTBP2 CLIP-seq in iPS-neurons and in human cortical brain tissue (Brodmann Area 4). Transcriptome wide CLIP-seq analysis and CLAM-mediated peak calling (Zhang & Xing, 2017) identified 49,875 and 57,162 high-confidence PTBP2 binding sites (bin size of 50 nucleotides) distributed across 8822 and 7869 genes in human cortex and iPS-neurons, respectively. This included overlapping peaks in iPS-neurons and human cortex just upstream of the AS exon 18 in DLG4 (FIG. 3A). PTBP2 binding sites were predominantly intronic (72%), yet when normalized to the number of nucleotides, PTBP2 binding was more evenly distributed across 5′UTRs, coding regions, and 3′UTRs (FIG. 3B). Motif analysis in both human brain and iPS-neurons indicated CUCUCU as the most enriched sequence observed in PTBP2 binding sites, consistent with previous identification of the preferred binding sequence for PTBP proteins (Xue et al., 2009) (FIG. 3C). Gene ontology analysis of genes with PTBP2 peak calls demonstrated an overrepresentation of synaptic terms (FIG. 3D), and SynGO ontology terms (Koopmans et al., 2019) were enriched in genes with PTBP2 peak calls relative to a background set of brain-expressed genes (FIG. 3E).

We also performed more detailed positional analysis using RBP-Maps (Yee et al., 2019) to interrogate the transcriptome-wide relationship between where PTBP2 binds and the AS events detected upon PTBP2 KD (FIG. 3D). This analysis indicates that PTBP2 binding within ˜100 nt upstream of a cassette exon promotes skipping (exclusion) of that exon, as is the case for DLG4 exon 18 (FIGS. 2B and 3A). A similar relationship was observed for PTBP2 upstream binding and the exclusion of the proximal exon in mutually exclusive AS events. While more variability is evident in the analysis of less frequently detected events (alternative 5′ and 3′ splice sites, retained introns), PTBP2 binding within ˜100 nt in the upstream intron was also associated with the exclusion of 3′ alternative splice sites, while PTBP2 binding proximal to a downstream exon promoted inclusion of alternative 5′ splicing upstream. Intriguingly, PTBP2 binding was also found to promote intron retention, regardless of whether this binding occurred at the 5′ or 3′ end of the retained intron (FIG. 3D).

Of the 342 Orphanet genes differentially spliced upon PTBP2 KD, 75 also showed PTBP2-binding proximal to an AS event (Table 3). 17 demonstrated direct PTBP2 binding, differential AS, and differential gene expression upon PTBP2 depletion, which prompts further evaluation of PTBP2-dependent splicing as a targetable therapeutic strategy for these genetic etiologies.

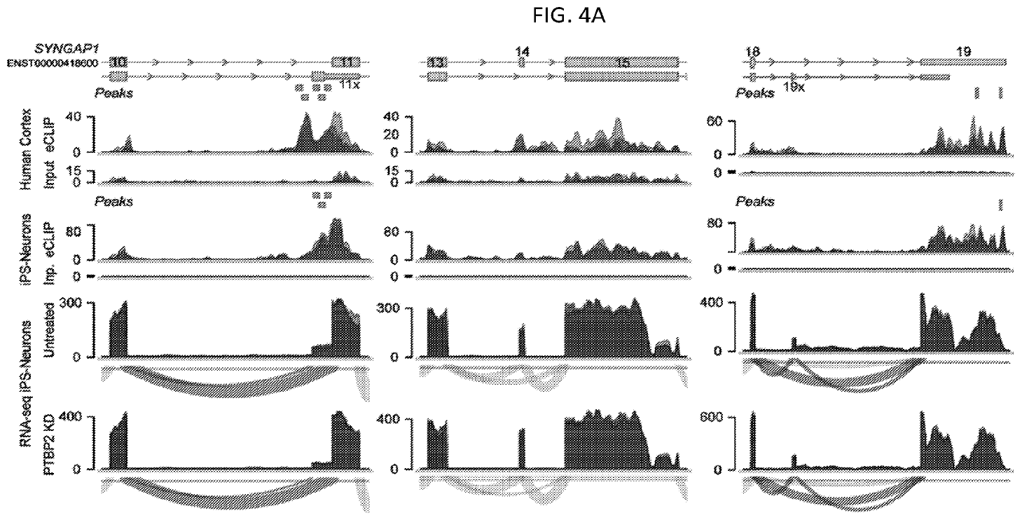

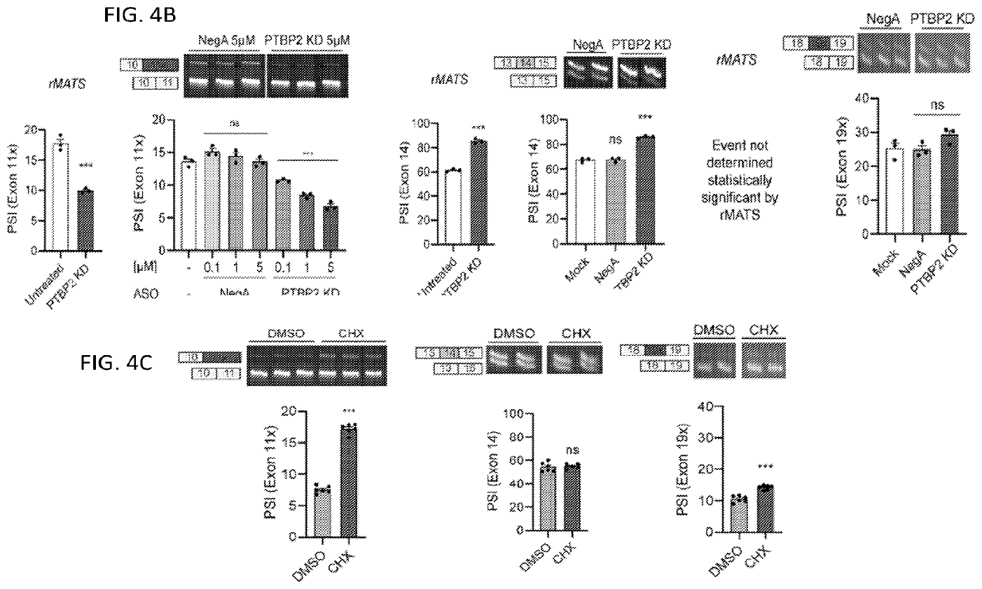

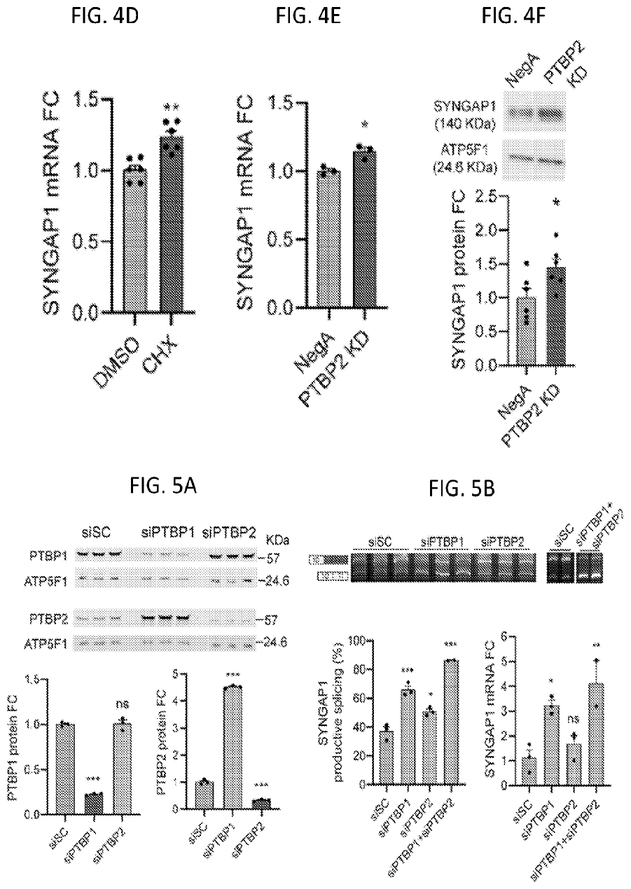

We performed such investigation into AS of SYNGAP1, where genetic variants drive a phenotypically diverse neurodevelopmental disorder through haploinsufficiency of gene expression. By comparing RNA sequencing data from adult human cortex with matured iPS-neurons, we identify several intriguing and well-conserved AS events on SYNGAP1 (FIG. 9). This includes the well-studied alternative transcriptional start sites (TSS) at the 5′ end (which leads to SYNGAP1 isoforms A and B N-terminal variants) and AS of the final 3′ exon, which creates SYNGAP1 C-terminal variants α1 and α2 (Gamache et al., 2020). Neither of these 5′ nor 3′ events were differentially regulated by PTBP2 KD. Also exhibited in both samples is the 3′AS event at Exon 11, a partial inclusion of exon 14, and inclusion of a predicted “poison” exon after Exon 18 (alternate exon 19, A.E. 19×) (FIG. 9). rMATS splicing analysis indicates that the presence of PTBP2 promoted inclusion of the 3′ AS of Exon11 (FIG. 4A-B, left), as well as exclusion of Exon 14 (FIG. 4A-B, middle), while not affecting the inclusion of A.E. 19× (FIG. 4A, right).

To confirm these findings, we designed RT-PCR splicing assays to assess PTBP2-dependent AS at each of these locations in human neurons. Consistent with rMATS analysis, PTBP2 KD led to a decrease in 3′AS of Exon 11, an increase in inclusion of Exon 14, and had no significant effect on A.E. 19× (FIG. 4B).

The 3′AS of Exon 11 and A.E. 19× are each predicted to lead to introduction of a premature termination codon that induces NMD, which has been demonstrated in non-neuronal cells for Exon 11 (Lim et al., 2020), while inclusion of Exon 14 does not alter the reading frame. We evaluated each potential site as a target for NMD in neurons by using the NMD inhibitor cycloheximide (CHX) and confirmed that NMD inhibition significantly increased 3′AS of Exon11 (FIG. 4C, left). CHX had a modest effect on A.E. 19 (FIG. 4C, right) while not affecting Exon 14 inclusion (FIG. 4C, middle). Consistent with relief from NMD, CHX treatment also increased total SYNGAP1 mRNA expression (FIG. 4D). Further, PTBP2 KD and exclusion of the NMD-linked AS event was concomitant with increased SYNGAP1 mRNA and a 44+/−14% increase in protein expression in human neurons (FIG. 4E-F).

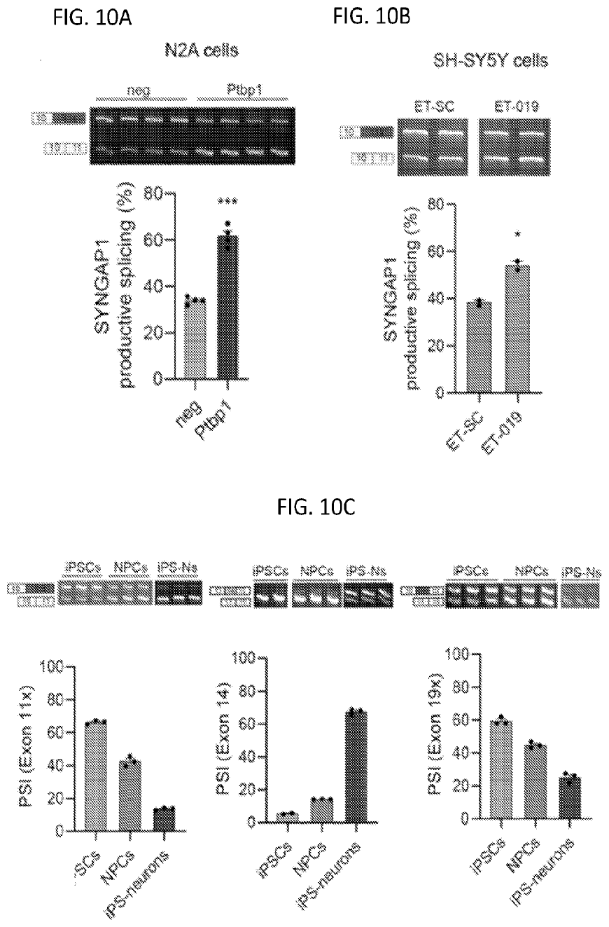

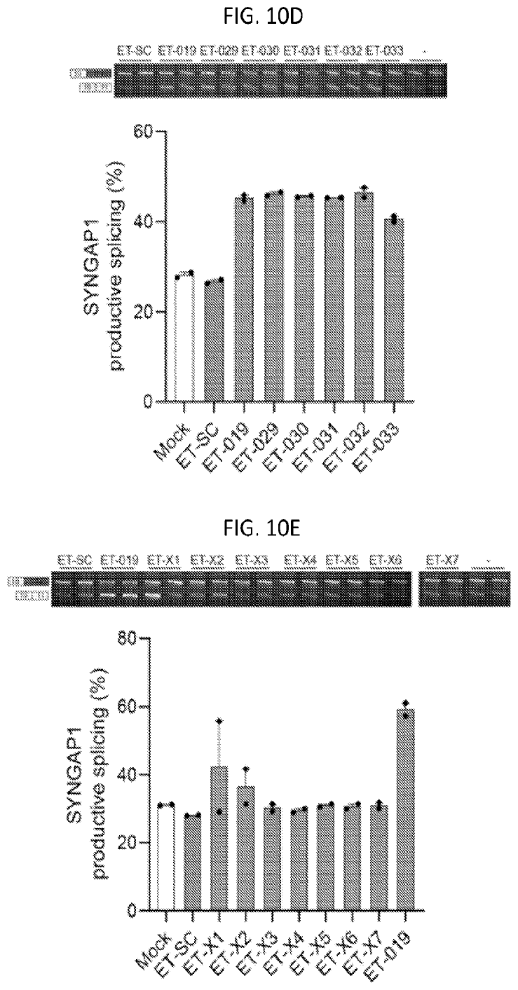

Taken together, the findings above indicate that PTBP2 binds directly to SYNGAP1 to promote inclusion of an alternative 3′ start site in Exon 11 that leads to NMD and repressed expression of SYNGAP1 in human neurons. As this AS event is readily detectable in adult human cortex (FIG. 10), disrupting PTBP2 binding to this region could be therapeutically beneficial in the context of SYNGAP1 haploinsufficiency. While targeting PTBP2 directly has limited therapeutic potential due to its numerous gene targets and pleiotropic actions (even on SYNGAP1), site-specific disruption using steric-blocking ASOs is an attractive option.

FIGS. 12A to 12I show that disrupting PTBP2 binding in Site 1 and Site 2 upregulates SYNGAP1 and protein expression in SYNGAP1 haploinsufficient patient cell lines. FIG. 12A is a schematic of SYNGAP 1 mRNA showing the location of the heterozygous mutations present in the two independent SYNGAP1 patient iPSC lines. FIG. 12 B is a schematic depicting the generation of the SYNGAP1 R1240X patient-derived iPSC line and the corresponding isogenic control line in which the heterozygous mutation has been reverted using CRISPR/Cas9 technology. PBMCs, peripheral blood mononuclear cells. FIG. 12C: Top and right panels: SYNGAP1 Western blot from corrected (isogenic control) and patient SYNGAP1 R1240X iPSC-neurons. FIG. 12 D: Top and right panels: SYNGAP1 Western blot from WT and patient SYNGAP1 K1185X iPSC-neurons. (c-d) Left panel: qPCR showing SYNGAP1 mRNA levels. FIG. 12E: Top and right panels: PTBP2 Western blot from K1185X NPCs treated for 3 d with PTBP2 gapmer. Left panel: PTBP2 mRNA fold change quantification (qPCR). FIG. 12F: Top and left panels: SYNGAP1 RT-PCR from K1185X NPCs treated for 3 d with PTBP2 gapmer. Right panel: qPCR showing SYNGAP1 mRNA levels. FIG. 12G is a SYNGAP1 Western blot from samples in FIG. 12E. FIGS. 12E and 12F show a-g, a non-targeting gapmer (NegA) was used as negative control. FIG. 12H) has top and left panels: RT-PCR from R1240X iPSC-neurons treated for 7 d with ET-019 at 10 μM. Right panel: qPCR showing SYNGAP1 mRNA levels FIG. 12I has top and left panels: RT-PCR from K1185X iPSC-neurons treated for 7 d with Site 1 and Site 2 targeting ASOs at 10 μM. Right panel: qPCR showing SYNGAP1 mRNA levels. White color data points represent an independent experiment. In FIG. 12H and FIG. 12I, non-targeting ET-SC or ET-MM ASOs were included as negative controls. Data are represented as mean values±SEM. All data points represent independent biological replicates. C-d (n=3). E-h (n=6). I (n=3 except n=5 for Mock and n=6 for ET-019 and ET-020). In c-d, Student's t-test. In FIGS. 12E-FIG. 12G, one-way ANOVA with Dunnett's multiple comparison test vs. NegA-treated cells. In FIGS. 12H-FIG. 12I, one-way ANOVA with Dunnett's multiple comparison test vs. mock-treated cells. ns p>0.05, *p<0.05, ** p<0.01 and *** p<0.001.

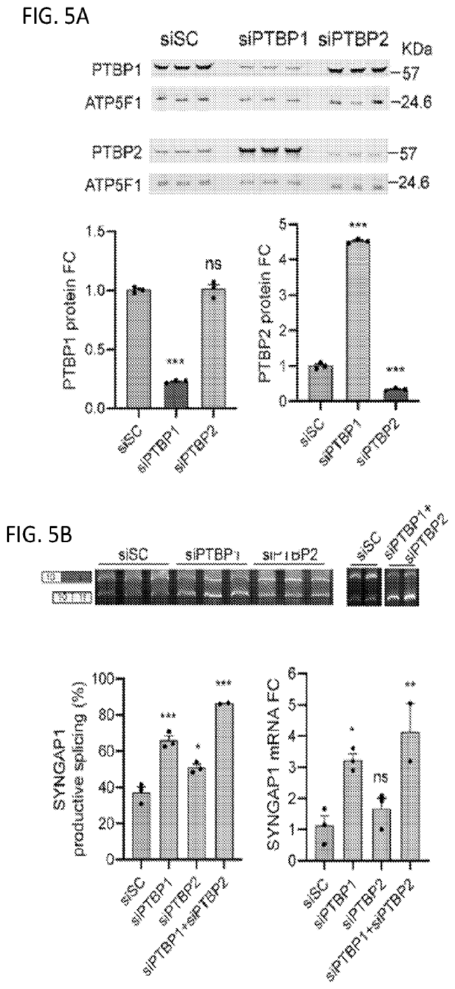

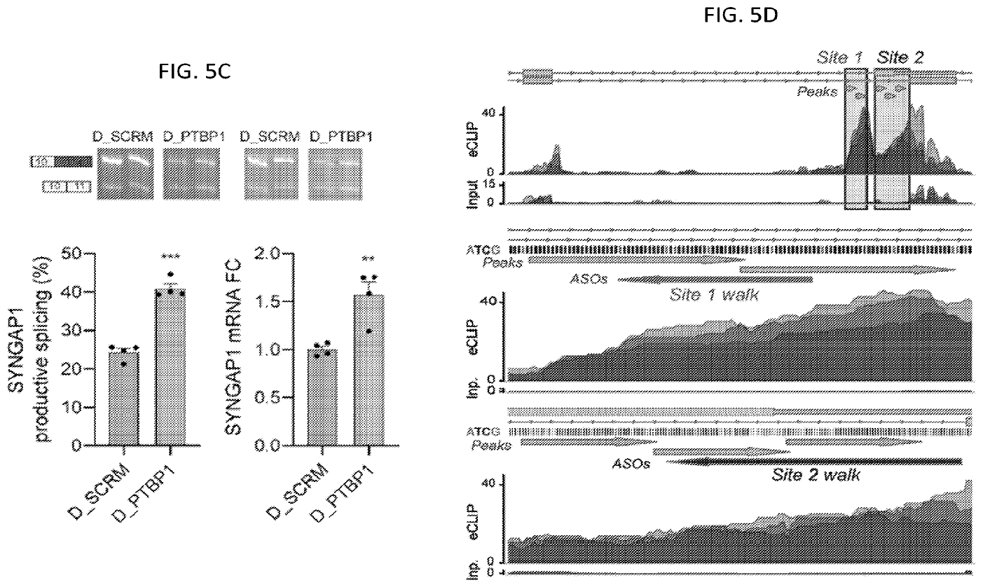

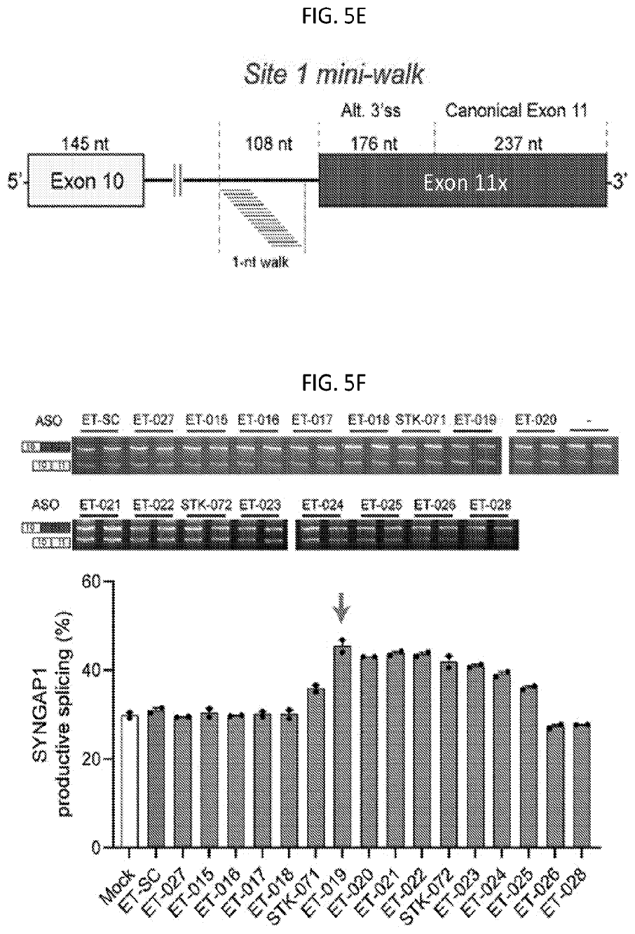

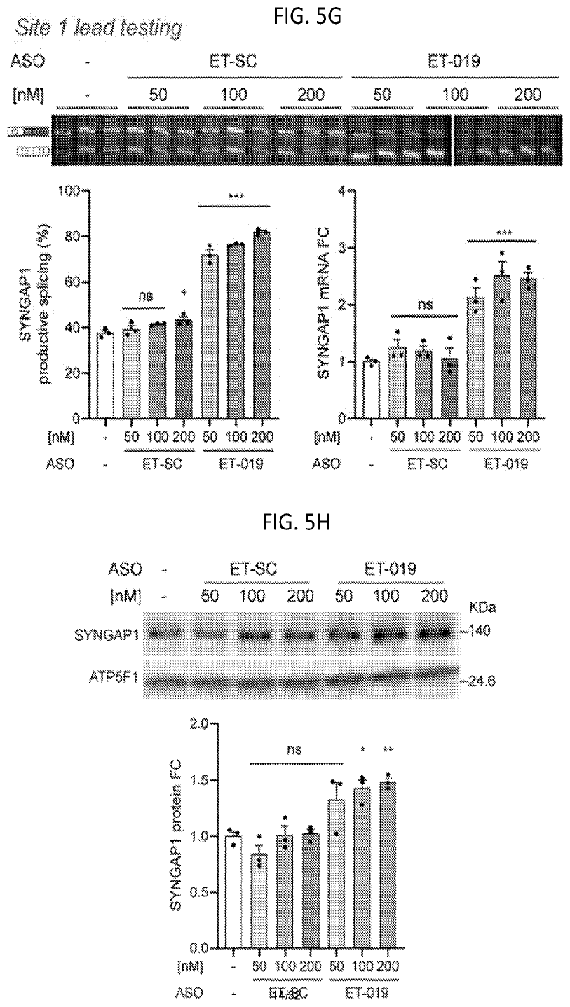

For high throughput screening of steric-blocking ASOs we utilized HEK293T cells, a cell type previously demonstrated to exhibit high levels of the 3′AS of SYNGAP1 Exon 11 (Lim et al., 2020). We first examined whether PTBP also regulates this AS event in HEK293T cells as it does in neurons. Unlike in mature neurons, PTBP1 is the primary isoform expressed in HEK293T cells, but these paralogs share a consensus binding sequence and often have overlapping targets (J. K. Vuong et al., 2016). We thus utilized siRNA to knock down both PTBP1 and PTBP2 in HEK293T cells and examined SYNGAP 1 splicing. We first confirmed robust depletion of each isoform at the protein level (FIG. 5A) and found that PTBP1 depletion led to increased expression of PTBP2, consistent with downregulation of PTBP2 by PTBP1 (Boutz et al., 2007).

HEK293T cells exhibited a high percentage of the NMD-linked AS of SYNGAP1 Exon 11 (FIG. 5B), consistent with this event being increasingly excluded during neuronal maturation. Depletion of PTBP1 and to a lesser extent PTBP2 each led to the exclusion of this AS event and a proportional increase in the percentage of non-NMD, “productive” splicing of SYNGAP 1, which was concomitant with increased SYNGAP1 mRNA expression (FIG. 5B). Dual depletion of both PTBP1 and PTBP2 lead to the greatest exclusion of the AS event and increase in SYNGAP1 mRNA (FIG. 5B).

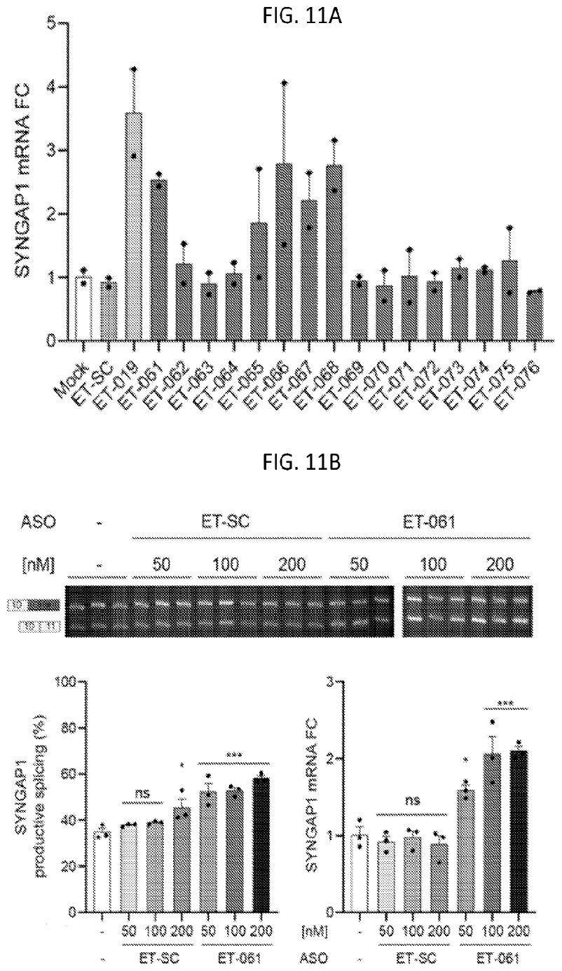

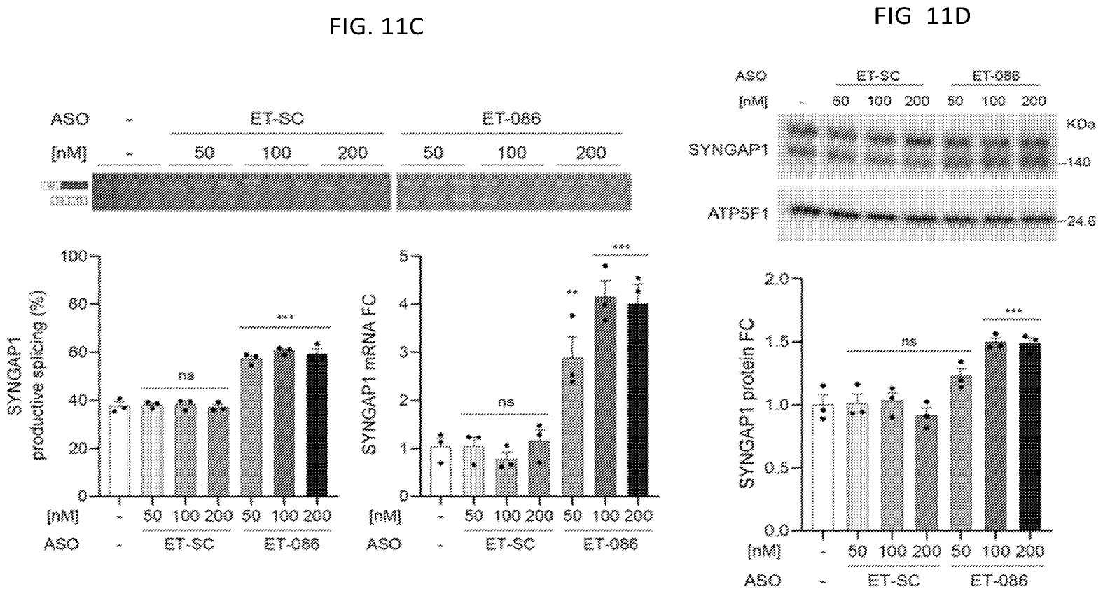

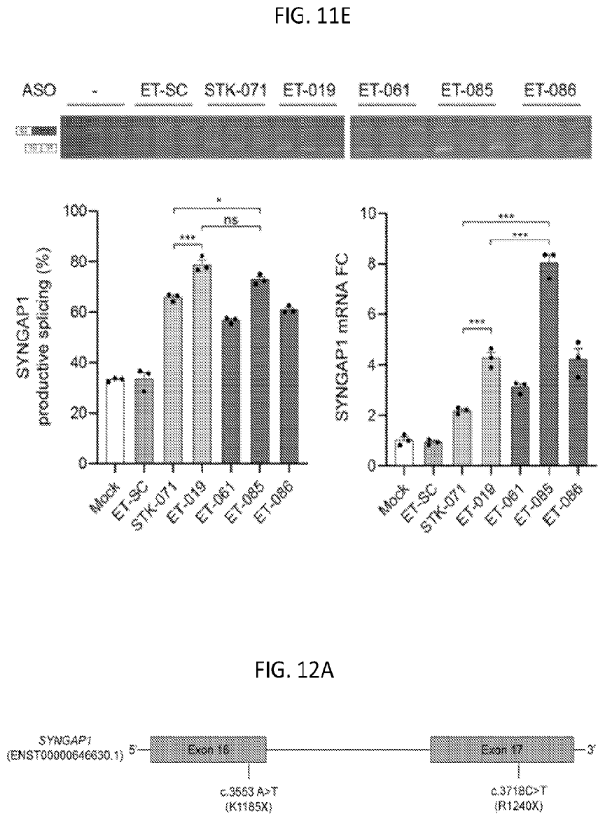

We next used an orthogonal approach to confirm whether PTBP is regulating this site using an established PTBP decoy assay (Denichenko et al., 2019). We transfected HEK293T cells with a high concentration (5 μM) of a decoy oligonucleotide containing 4 repeats of the PTBP consensus binding motif CUCUCU, which competes for PTBP occupancy of endogenous binding sites. Transfection of this decoy was sufficient to increase productive splicing of SYNGAP1 at Exon 11 and SYNGAP1 mRNA expression (FIG. 5C). Further, we also found high inclusion of this AS event in “immature” neuronal cells (mouse N2A, human SHSY5Y), and that PTBP1 depletion was sufficient to improve SYNGAP1 productive splicing (FIG. 11A-B). Together with the above, these findings provide strong support that 1) PTBP binding promotes inclusion of an alt. 3′ start site in SYNGAP1 Ex11 that leads to NMD and restricted expression across numerous cell types; 2) that both PTBP1 and PTBP2 can regulate this event; 3) the inclusion of this AS event decreases with neuronal maturity, concomitant with reduced levels of PTBP and increased SYNGAP 1 expression (FIG. 11C).

We next attempted to disrupt PTBP binding and improve productive SYNGAP1 splicing using steric blocking ASOs. PTBP CLIP-seq analysis from both iPS-neurons and human brain pinpointed two PTBP binding regions near SYNGAP1 Exon 11 to evaluate for therapeutic targeting (FIG. 5D). The first is a hot spot for PTBP enrichment and consensus binding sequences in Intron 10, approximately 100 nucleotides upstream from the alt. 3′ start site, hereafter referred to as “Site 1”. The second is a highly enriched PTBP binding region that lies directly in the alternatively spliced-in region (“Site 2”).

Informatively, a recent 5 nucleotide spaced ASO “walk” found that several ASOs directed toward Site 1 elicited improvement in SYNGAP1 productive splicing, although the mechanism for this was not interrogated (Lim et al., 2020). We overlayed these results with our PTBP-binding maps and found that the effective ASOs indeed overlapped with PTBP binding sites. We refined this interrogation by performing a 1-nucleotide resolution ASO “mini-walk” (FIG. 5E) around the most promising region (near the previously identified STK-071) and found that ASOs spanning position −102 to −85 (ET-019) and −101 to −84 (ET-020) showed the most improvement in SYNGAP1 productive splicing (FIG. 5F). We also confirmed the effectiveness of ET-019 in human neuronal SH-SY5Y cells (FIG. 10B). In additional screenings to attempt optimization, modified versions of ET-019 containing extra nucleotides either in the 5′ or the 3′ end of the ASO did not improve productive splicing compared to ET-019 (FIG. 10D), and longer ASOs (22-mer) targeting the adjacent upstream region of ET-019 were also not effective (FIG. 10E). In follow up lead testing, we found ET-019 causes a dose-dependent improvement in SYNGAP1 productive splicing, mRNA, and protein expression (FIG. 5G+H). From these experiments, we identify ET-019 and ET-020 as the most promising ASOs for SYNGAP1 upregulation targeting site 1.

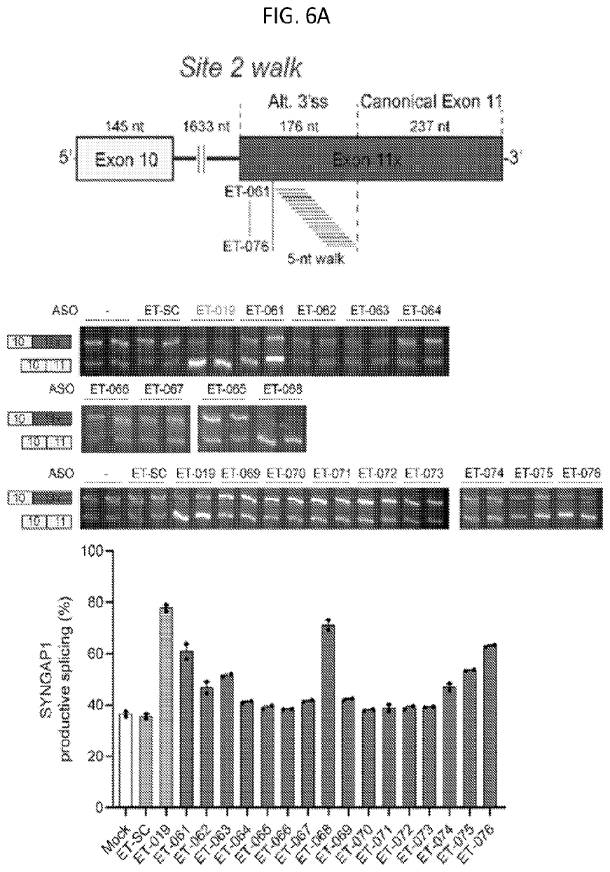

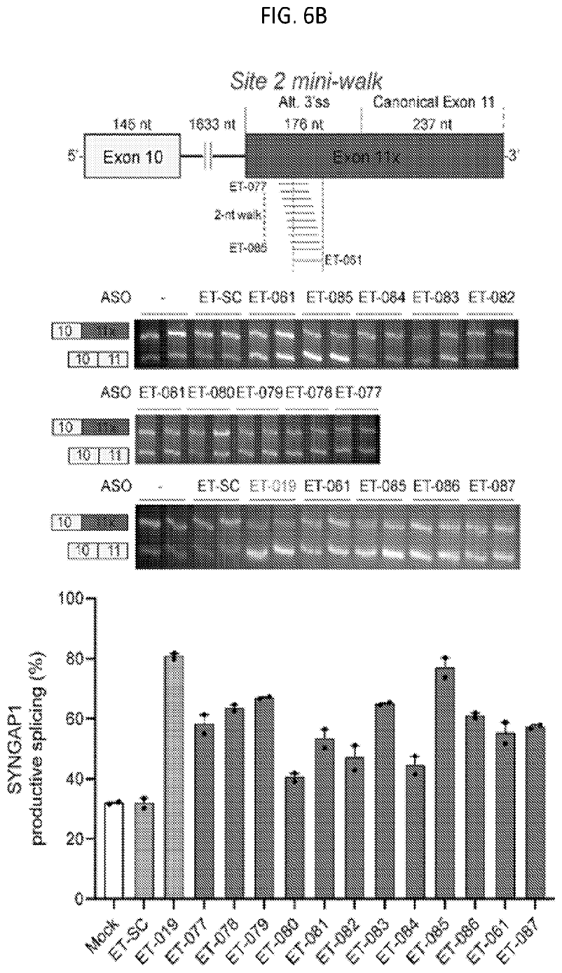

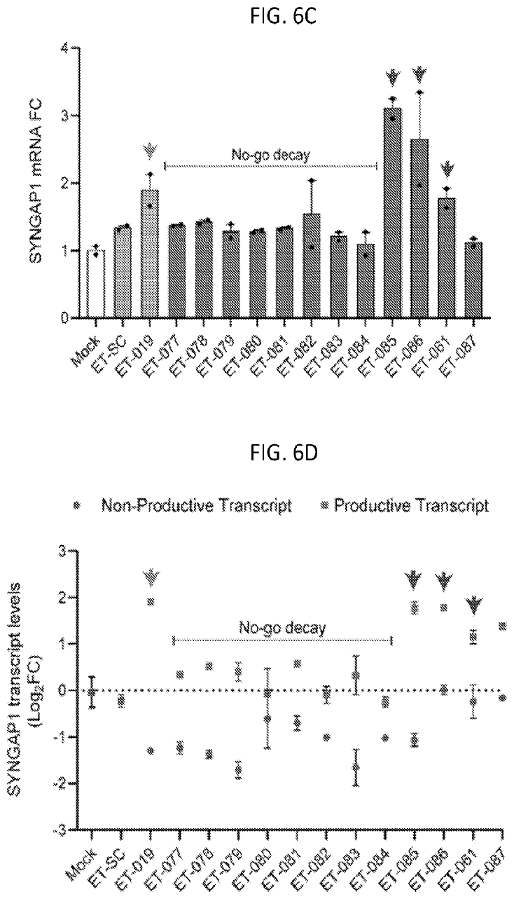

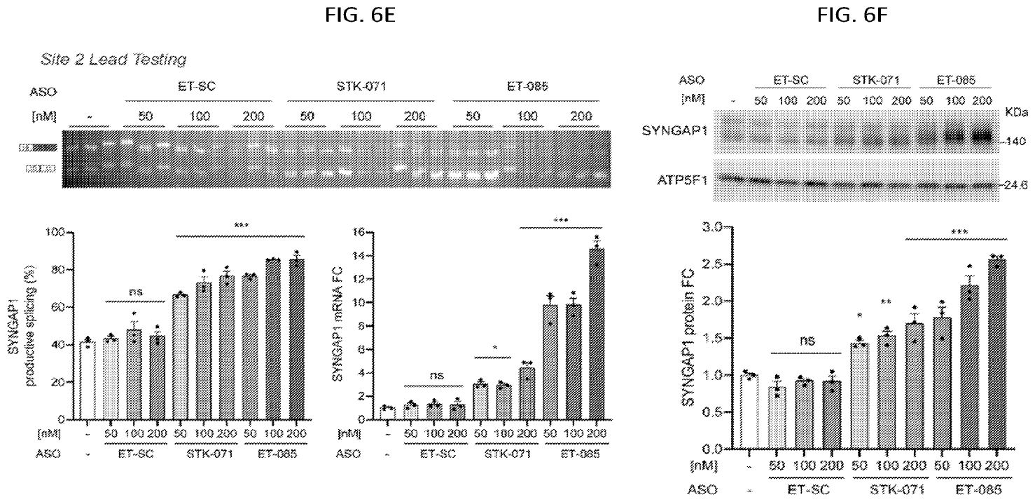

Our PTBP CLIP-seq data also led us to interrogate site 2, which lies within the exonic region introduced by the alternative 3′ start site, and which has not been previously explored. We first performed a 5-nt resolution ASO walk around this region and identified multiple candidates that could improve productive splicing of Exon 11 (FIG. 6A) and increase SYNGAP1 mRNA levels (FIG. 11A) We performed follow up on ET-061, which showed increased expression of the productive transcript concomitant with robust improvements in SYNGAP1 mRNA expression in a dose-dependent manner (FIG. 11B). As ET-061 was the most 5′ASO in the initial walk, we extended and refined this region upstream using an upstream mini-walk (FIG. 6B). We found that ASOs just upstream of ET-61 (ET-085 and ET-086) could robustly improve both productive splicing (FIG. 6B) and total SYNGAP1 mRNA levels (FIG. 6C). In follow-up studies, we confirmed that ET-086 led to a dose-dependent increase in SYNGAP1 productive splicing, mRNA, and protein expression (FIG. 11C-D).

Intriguingly, we also identified several candidates (e.g. ET-079) that could improve productive splicing (FIG. 6B) but not SYNGAP1 mRNA expression (FIG. 6C). Interestingly, ASOs that bound between +64 and +95 in the alternatively spliced-in region were found to reduce expression of the non-productive transcript, but without a concomitant increase in the productively spliced transcript, as would be expected if a splice-switching oligo promoted utilization of an alternative splice site (FIG. 6D). Instead, these results are consistent with these particular ASOs binding to the non-productive transcript and triggering “no-go decay”, a mechanism whereby steric blocking oligos bound to coding sequences can disrupt ribosomal translation and induce degradation of that particular transcript in an RNase H1-independent manner (Liang et al., 2019).

We noted that across (but not within) experiments the magnitude of effect of a given ASO may vary, which was anecdotally linked to differences in cell plating density. As this prevented quantitative comparisons across different studies, we next directly compared our top performing Site 1 and 2 oligos under identical conditions. We first evaluated the previous top performing ASO targeting site 1 (STK-071) with the newly identified ASO targeting site 2 (ET-085) and found that each offer a dose-dependent improvement in SYNGAP1 productive splicing, mRNA expression, and protein expression (FIG. 6E-F), but with ET-085 showing dramatic upregulation at the mRNA level (to >10-fold) coupled with a greater increase in protein expression (2.5-fold). This site 2 targeting ASO offers comparable levels of SYNGAP1 upregulation at 4-fold lower concentration, which may increase its therapeutic potential by limiting oligo dosage and off-target effects.

A similarly designed experiment comparing multiple site 1 and 2 targeting ASOs revealed ET-019 and ET-085 as the most robust ASOs in terms of improving productive splicing and SYNGAP1 mRNA levels, respectively (FIG. 11E). Of note, while improvements in productive splicing correlated with increased mRNA within both Site 1 and 2 targeting oligos, ASOs targeting Site 2 uniformly produced a larger increase in SYNGAP1 mRNA relative to the change in productive splicing. This suggests additional—and incompletely understood—gain by targeting this region.

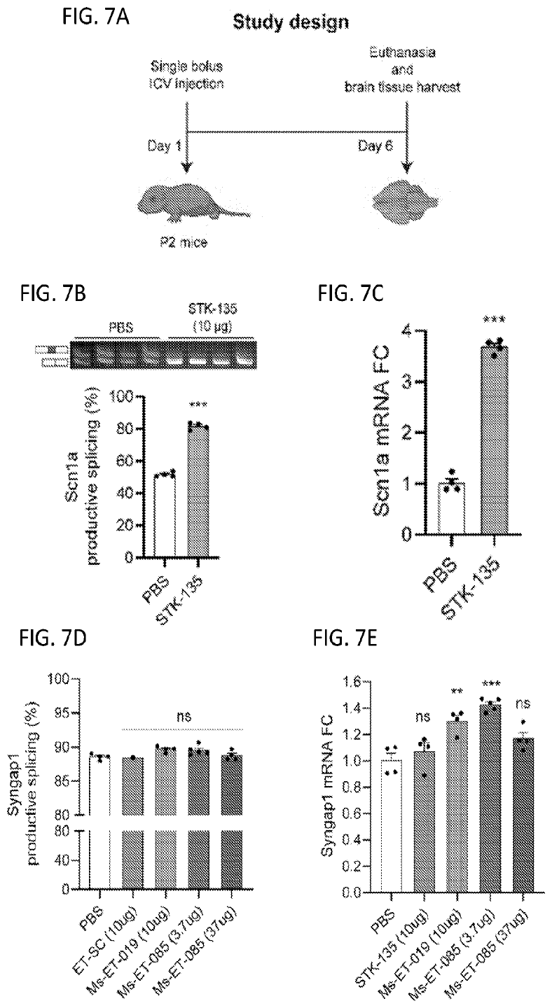

We next performed proof-of-concept studies to examine potential ASO-mediated SYNGAP1 upregulation in the post-natal brain in vivo. We performed intracerebroventricular injection (ICV) of our top performing site 1 (ET-019) and site 2 (ET-085) oligos into the mouse brain at post-natal day 2 and harvested the brain 5 days later (FIG. 7A). We included STK-135—a splice-switching oligo demonstrated to potently bind and manipulate SCNIA (Lim et al., 2020)—as a positive control to demonstrate target engagement of a neuronal gene in our hands, and as a negative control ASO de-targeted from SYNGAP1. Consistently, we found that STK-135 potently improved productive splicing and expression levels of SCNIA (FIG. 7B-C), with no effect on SYNGAP1 productive splicing or mRNA expression (FIG. 7D-E). In contrast, both ET-019 and ET-085 significantly increased SYNGAP1 mRNA expression despite modest effects on productive splicing. ET-085 produced a greater increase in SYNGAP1 mRNA at lower concentration than ET-019, consistent with our in vitro findings (FIG. 7D-E).

### Discussion:

Here we leverage multiple powerful approaches to produce the first combined binding and splicing maps of an RNA binding protein in human brain. We utilize these maps to guide steric blocking oligonucleotides that disrupt PTBP-dependent AS to modulate a gene of interest in a therapeutically tractable fashion. We hope this provides a rich database for further examination into PTBP-dependent AS and its targetability for neurological disorders.

Transcriptome-wide determination of direct, RBP-mediated AS requires the combination of splicing analysis upon RBP manipulation combined with mapping of RBP binding. We utilized rMATS to quantify AS events across the transcriptome upon depletion of PTBP2 and combined this with CLIP-seq and CLAM-mediated mapping of PTBP2 binding in human cortex and cultured human neurons to map PTBP2-driven AS. To our knowledge this is also the first mapping of PTBP2 binding in the adult brain, regardless of species. Importantly, a fraction of PTBP binding sites were shared between iPS-neurons and adult human cortex, supporting both the relative maturity of the iPS-neuronal platform, as well its suitability to reflect AS patterns likely relevant to adult neurons in situ. Further, it indicates the persistence of PTBP2-dependent AS for fine tuning neuronal gene programs into adulthood.

By examining the positional dependence of PTBP2 binding relevant to AS events, we identify novel features.

Our data also indicates a prominent regulation of synaptic gene programs by PTBP2. PTBP2-depletion drives differential expression of 304 genes associated with synaptic organization, function and plasticity, and we identified concomitant AS and differential gene regulation of numerous genetic causes of neurological disorders. We focus on SYNGAP1 as proof of concept for how combined splicing and binding maps can guide ASO-dependent manipulation of an AS event for potential therapeutic gain, for example by preventing NMD of a transcript that drives disease when haploinsufficient. Beyond SYNGAP 1, Table 3 lists other disease-causing genes that are both alternatively spliced and differentially expressed upon PTBP2 depletion, and which show direct PTBP binding near AS events.

For certain differentially spliced Orphanet genes, the identification of the precise AS event and its association with an NMD transcript were less clear than with SYNGAP1. For example, GRIN1 (associated with a spectrum of neurodevelopmental disorders) demonstrates PTBP binding and alternative splicing proximal to NMD associated events, as well as differential gene expression upon PTBP2 KD. However, the specific exon-exon junction identified as AS by rMATS is not annotated in commonly used genome browsers (UCSC Genome Browser on Human (GRCh38/hg38); the more precise identification (and thus targeting) of such events could benefit from long-read sequencing together with short-read sequencing, which was recently demonstrated to help detect many unannotated NMD events across the transcriptome (Karousis et al., 2021).

This study also emphasizes the importance for understanding the cell-type and developmental regulation of an AS event (as well as the location of RBP binding) for guiding and assessing splice switching therapeutic strategies. NMD events can be tightly developmentally regulated and expressed in a cell-specific fashion, necessitating the understanding of this process for precise targeting. As case in point, SYNGAP1 expression appears to be repressed in both non-neuronal and immature neuronal cells (FIG. 11) by predominant, PTBP-driven AS that induces NMD; the NMD exon becomes increasingly excluded upon neuronal maturation, concomitant with reduced PTBP expression and increased SYNGAP1 levels. However, this developmental regulation reduces the potential gain for targeting this AS event to upregulate SYNGAP1. This is evidenced by the >10-fold increase in SYNGAP1 mRNA via ASO disruption of PTBP binding (FIG. 6E) in HEK293T cells compared to far more modest effects of the same ASO delivered to the postnatal brain. As SYNGAP 1 is a strongly dosage-sensitive gene (with ˜50% expression likely sufficient to drive pathological phenotypes), modest upregulation may still hold therapeutic potential. Our pilot in vivo study motivates further, extensive screening of ASOs in vivo—ideally targeting a humanized SYNGAP1 gene—to identify oligos with maximal on-target upregulation of SYNGAP1 and minimal off-target toxicity.

### Cell Culture, Transfection and CHX Treatment

HEK293T cells were grown in DMEM (Corning, #10-013-CV) with 10% Fetal Bovine Serum (Corning, #35-010-CV) containing 50 μg/mL gentamicin (gibco #15750060) and 0.25 μg/mL amphotericin B (gibco #15290018). Cells (2.75×10⁵) were seeded in 12-well plates one day before transfection, which was performed with 50, 100 or 200 nM of ASO using Lipofectamine RNAiMax reagent (Invitrogen, #13778100) or Lipofectamine 2000 (Invitrogen, #11668027) according to manufacturer's instructions. For decoy experiments, HEK293T cells were transfected with 5 μM of the specified decoy. Total RNA was isolated from HEK293T cells 24 h or 48 h after transfection. Total protein was extracted 48 h post-transfection.

PTBP1 and PTBP2 knockdown experiments in HEK293T cells were performed with TriFECTa kit DsiRNA Duplex (IDT) using hs.Ri.PTBP1.13 (named siPTBP1) and hs.Ri.PTBP2.13 (named siPTBP2) predesigned siRNAs, respectively. Predesigned NC-1 siRNA (IDT, #51-01-14-04) was used as negative control (named siSc). siRNAs were transfected with Lipofectamine RNAiMax reagent using the concentrations recommended by the manufacturer and total RNA and protein was isolated 48 h post-transfection.

SH-SY5Y cells were grown in 1:1 Ham's F12: EMEM (Gibco #11765-047 and ATCC #30-2003, respectively) with 10% Fetal Bovine Serum (Corning, #35-010-CV) containing 50 μg/mL gentamicin (gibco #15750060) and 0.25 μg/mL amphotericin B (gibco #15290018). ASO (20 μM) was delivered into cells (1×10⁶) by electroporation in a Nucleofector device (Lonza) using the Amaxa Cell Line Nucleofector Kit V (Lonza #VCA-1003) and following the recommended instructions for SH-SY5Y nucleofection. Total RNA was isolated from SH-SY5Y 24 h after nucleofection.

For generation of mature iPS-neurons, the CHOP-WT10 cell line was maintained as an iPSC culture (Maguire et al., 2016), followed by transitioned to feeder-free cultures and maintained with mTeSR1 (StemCell Technologies) on hESC qualified matrigel (Corning). Feeder-free iPSC stocks were cryopreserved in 90% FBS/10% DMSO at a minimum of 2 passages after feeder free transition. Feeder-free stocks of CHOP-WT10 were passaged in mTeSR1 prior to the initiation of differentiation.

Prior to differentiation, plates were coated with growth factor reduced (GFR) Matrigel (Corning), where 1 mg of GFR Matrigel was added to 24 ml of DMEM/F12 (Invitrogen) and 2 ml of the mixture was added per 35 mm well and incubated at 37° C. for a minimum of 1 h (Ho et al., 2016). iPSCs were passaged with mTeSR1 onto the GFR Matrigel-coated plates at a density of approximately 50,000 cells/cm² well. When cultures were a minimum of 60% confluent, differentiation of iPSCs into neural progenitor cells (NPCs) was initiated (indicated as “Day 0”), as previously described in (Telezhkin et al., 2016) with modification. Briefly, cultures were treated with daily media changes containing SB431542 (10 μM; Tocris), LDN193189 (1 μM, Stemgent), IWR1 (1.5 μM; Tocris) and supplemented with B27 without vitamin A (Invitrogen) and passaged at days 4 and 8 of differentiation. Cells were passaged with accutase (Invitrogen) and replated on to GFR Matrigel-coated plates in the appropriate media, containing Y27632 (10 μM), at the approximately densities: 210,000/cm² (Day 4); 155,000/cm² (Day 8). After 8 days of daily treatment, forebrain neural ectoderm was confirmed by expression of FOXG1, SOX2, and PAX6 by flow cytometry (data not shown). From Day 8 to 14 of differentiation, NPCs were expanded in Invitrogen Neural Expansion Media, containing Neural Induction Supplement in 50% Advanced DMEM/F12 and 50% Neurobasal Medium (Invitrogen, as per manufacturer instructions). NPC identity was confirmed at Day 14 by expression of FORSE-1 (data not shown) and NPCs were cryopreserved in 90% FBS/10% DMSO for future use. NPCs continued directly or were thawed from cryopreserved NPCs for subsequent differentiation steps. NPCs were plated on to GFR Matrigel coated plates at 285,000/cm² into N2B27 (−): 2 parts DMEM/F12 (Invitrogen), 1 part Neurobasal Medium (Invitrogen) containing ⅓×N2 supplement (Invitrogen), ⅔×B27 without vitamin A (Invitrogen), 1× glutamine (Invitrogen), 50 μM beta-mercaptoethanol (Invitrogen) and 25-100 ng/ml of Activin A (Bio-Techne), followed by daily media changes of the same media with supplement. After 8-9 days of N2B27 (−)+Activin A, the majority of cells appeared as terminally differentiating neurons, and were therefore passaged with 0.5 mM EDTA, and replated at a density of approximately 155,000/cm² on to plates coated with poly-D-lysine (10 μM/mL, Sigma), followed by GFR matrigel, as above, into N2B27 (+): 2 parts DMEM/F12 (Invitrogen), 1 part Neurobasal Medium (Invitrogen) containing ⅓× N2 supplement (Invitrogen), ⅔× B27 with vitamin A (Invitrogen), 1× glutamine (Invitrogen), 50 μM beta-mercaptoethanol (Invitrogen), brain-derived neurotrophic factor (20 ng/ml) and glial-derived neurotrophic factor (20 ng/mL). Half-media changes were performed with the same media and supplements 3 times a week until final harvest. Neuronal identity was confirmed by immunofluorescence staining of MAP2 and Tuj1. qPCR analyses confirmed expression of TBR2, TBR1, CTIP2, SATB2, and VGLUT1 in the final neuron population (data not shown).

Experiments were performed when neurons matured to day 40-50 of differentiation, or the equivalent, in cases where neurons were cryopreserved at day 14.

To evaluate each potential site of SYNGAP1 as a target for NMD, we incubated iPS-neurons with 50 μM of cycloheximide (CHX) (Sigma-Aldrich, #01810) for 3h. ASO treatments in mature iPS-neurons were performed by gymnotic delivery of the ASO for 7 days.

ASO and decoy oligonucleotides were purchased from IDT. Gapmer ASOs were obtained from Qiagen. Sequences and chemistry information for all oligonucleotides can be found in Supplementary data.

### RNA-Seq Library Preparation and Sequencing

RNA was extracted from PTBP2 gapmer or control gapmer-treated iPSC-derived neurons with RNeasy Mini kit (Qiagen, #74104) with on-column DNAse treatment using Qiagen DNAse Set (Qiagen, #79254) at room temperature for 15 minutes. The RNA was further cleanup and concentrated with Quick-RNA Miniprep Kit (Zymo Research, #R1055). Purified RNA was analyzed on Agilent TapeStation systems, using D1000 Screen Tape & Reagents, and then quantified with a Qubit BR kit (Thermo Fisher, #Q10210). Poly(A)+RNA transcript was isolated from 1 μg purified RNA (RNA integrity number>9.0) with NEBNext poly(A) mRNA magnetic isolation module (New England Biolabs, #E7490]. RNA-seq libraries were prepared with NEBNext Ultra Directional RNA library preparation kit for Illumina (New England Biolabs, #E7420S) according to the manufacturer's instruction. The samples were sequenced using NovaSeq 6000 SP Reagent Kit v1.5 (200 cycles) with 150-bp paired-end reads at the Sequencing Core at Children's Hospital of Philadelphia.

### RNA-Seq Data Processing for Differential Gene Expression

### Genome Mapping

RNA-seq libraries were demultiplexed, and adapter sequences were removed with Cutadapt v1.18. Sequence quality was assessed by FastQC v0.11.2. Transcriptome indices were prepared for Salmon v1.5.2 using a decoy-aware transcriptome file (gencode.v38.transcripts.fa with GRCh38.primary_assembly.genome.fa genome as decoy). Transcripts were quantified from paired-end reads in mapping-based mode (selective alignment, --libType ISR--gcBias--validateMappings).

### Differential Gene Expression

Salmon transcript counts were aggregated to the gene level using the tximeta (Love et al., 2020) package in R. Differential expression analysis was performed using DESeq2 (Love et al., 2014) with Untreated as the reference condition. An adjusted pval cutoff of 0.05 was used for significance. Log2 fold change shrinkage was applied using the apeglm algorithm (Zhu et al., 2019). Ensembl gene annotations were added. Over-representation analysis was performed using the clusterProfiler package in R (Wu et al., 2021). All genes evaluated for differential expression were used as the background dataset for testing.

### RNA-Seq Data Processing for Alternative Splicing Analysis

### Genome Alignment

Demultiplexed, paired-end reads were aligned to the human genome (GRCh38.primary_assembly.genome.fa) with STAR v2.7.1a in two-pass mode (Veeneman et al., 2016). For first-pass alignment, GENCODE basic gene annotation (gencode.v38.basic.annotation.gtf) was used (--outSAMstrandField intronMotif--outFilterType BySJout--alignSJoverhangMin 8--alignSJDBoverhangMin 3--outFilterMismatchNoverReadLmax 0.04--alignIntronMin 20--alignIntronMax 1000000--alignMatesGapMax 1000000--scoreGenomicLengthLog2scale 0). The output splice junction files were concatenated and filtered (remove junctions on chrM, non-canonical junctions, junctions supported by multi-mappers or by too few reads) then used in on-the-fly second-pass alignment (--outSAMstrandField intronMotif--outSAMattributes NH HI AS NM MD--outFilterType BySJout--alignSJoverhangMin 8--alignSJDBoverhangMin 3--outFilterMismatchNoverReadLmax 0.04--alignIntronMin 20--alignIntronMax 1000000--alignMatesGapMax 1000000--scoreGenomicLengthLog2scale 0--quantMode TranscriptomeSAM GeneCounts). Alignments were filtered with samtools v0.1.19 to remove unmapped reads and reads mapping to 10 or more locations. Alignment quality was assessed with Qualimap v2.2.2-dev.

### Splicing Analysis

Differential splicing analysis was performed using rMATS v4.1.1 with bam files above as input (group 1 Untreated, group 2 PTBP2 KD). Prep and post steps were run (--gtf gencode.v38.primary_assembly.annotation.gtf-t paired--libType fr-firststrand--readLength 151--variable-read-length--novelSS--allow-clipping) followed by the statistical model (--cstat 0.01). rMATS output files were then loaded into R with the maser package (F. T. Veiga D, 2022). Downstream analysis was performed using the junction counts only file, and splicing events were filtered by coverage (average read counts of 20 or more).

### Annotation of Disease Relevance

A list of genes associated with rare diseases were downloaded from the Orphadata repository of Orphanet (http://www.orphadata.org).

### Visualization

Bigwig files were prepared using Yeo lab's makebigwigfiles [https://github.com/YeoLab/makebigwigfiles]. Visualization was performed in R using the Gviz package (Hahne and Ivanek, 2016).

Eclip for iPS-Neurons and Postmortem Human Brain

eCLIP was performed as described previously (van Nostrand et al., 2016; Spengler et al., 2016) except the adaptors sequence was from (He et al., 2021). For iPSC-derived neurons, 3×6-well plates were used for 3 eCLIP. The differentiated neurons seeded in 1×6-well plate (˜1×10⁶ cells/well) were irradiated with ultraviolet (UV) light at 400 mJ/cm² for once, and 200 mJ/cm² for once on iced water. For postmortem human brain BA4 region, the tissue from three different donors was first pulverized in liquid nitrogen, and the powder in a chilled 10-cm tissue culture plate on dry ice was UV-crosslinked at 400 mJ/cm² three times (Spengler et al., 2016). Crosslinked neurons or brain tissues were washed 1× with pre-chilled DBPS (Corning, #21-031-CV). The pellet was frozen in −80° C. or directly lysed in eCLIP lysis buffer containing proteinase inhibitor and RNAse inhibitor. Cell or tissue lysis was assisted with sonication by using a Bioruptor on the low setting for 5 min, cycling 30 s on and 30 s off. For each 1 mL lysate, 5 μl Turbo DNAse I (Thermo Fisher, #AM2239) and high dose RNAse I (Thermo Fisher, #AM2295; 1:5 diluted in cold DPBS) or low dose RNAse I (1:50 dilution for neurons, and 1:100 dilution for brain) were added. The microspin tubes were placed in a Thermomixer preheated to 37° C. for exactly 5 min, shaking at 1,200 rpm, and then put on ice to terminate the reaction. Cell lysales were spun for 20 minutes at full speed in a pre-cold centrifuge.

The cleared lysate was mixed with Dynabeads Protein G (Thermo Fisher, #10003D) pre-conjugated with 10 μg PTBP2 antibody (EMD Millipore, #ABE431), and incubated in a cold room overnight. Immunoprecipitated-RNA was dephosphorylated with FastAP enzyme (Thermo Scientific, #EF0652) and T4 PNK (New England BioLabs, #M0201L), and an adapter labeled with an IRDye®800CW fluorochrome (/5Phos/rNrNrNrN rArGrA rUrCrG rGrArA rGrArG rCrArC rArCrG rUrCrU rGrArA rArA/3IR800CWN/) was ligated to the 3′ end. Labeled RBP-RNA complexes were eluted using 1×LDS loading buffer (Thermo Fisher) and resolved on 4-12% Bis-Tris gels, transferred to nitrocellulose membrane, and imaged. RNA-protein complexed were visualized with Li-Cor Odyssey imaging system, and a region ˜75 kD above protein size (by comparing with high RNAse I sample) was cut from the membrane. RNA was isolated from the membrane via protease K/SDS treatment. After reverse transcription with a modified primer (5′-TTC AGA CGT GTG CTC TTC CG-3′. SEQ ID NO: 59), a 5′ adapter containing 8-nt UMI (/5phos/NNNNNNNN AGATCGGAAGAGCGTCGTGTAGGG/3ddC/, SEQ ID NO: 60) was ligated to cDNA, and all of the remaining steps were essentially performed according to the eCLIP procedure (van Nostrand et al., 2016). The uniquely barcoded eCLIP samples were prepared and analyzed on Agilent Tapestation D1000. The final libraries were pooled and sequenced using NovaSeq 6000 SP Reagent Kit v1.5 (200 cycles) with 150-bp paired-end reads at the Sequencing Core at Children's Hospital of Philadelphia.

eCLIP-Seq Data Processing

### Genome Alignment

eCLIP libraries were demultiplexed, and inline random 8-mers were removed from the start of read 1 and appended to the read name using a script modified from the Yeo lab's eclipdemux [https://github.com/YeoLab/eclipdemux]. Inline random 4-mers were removed from the start of read 2 using Trimmomatic v0.32. Adapter sequences were removed in two rounds with Cutadapt v1.18 using commands recommended by ENCODE's eCLIP-seq Processing Pipeline. Sequence quality was assessed by FastQC v0.11.2. Paired-end reads were aligned to the human genome (GRCh38.primary_assembly.genome.fa, gencode.v39.primary_assembly.annotation.gif) with STAR v2.7.1a (--alignEndsProtrude 15 ConcordantPair--outFilterMultimapNmax 100--outSAMattributes NH HI AS NM MD). Multimappers were further filtered prior to peak calling (see below). Next, rRNAs and tRNAs were removed in a two-step process. rRNA and tRNA tracks were downloaded from UCSC Table Browser (RepeatMasker's rmsk track and filtering for rRNA or tRNA). First, bedtools intersect (v2.15.0, −f 0.90) was used to identify all rRNA/tRNA reads. In the second step, qnames from round 1 were used to mask potentially multi-mapping rRNAs/tRNAs in the alignment file using Picard Tools v1.141 FilterSamReads.

### Peak Calling

PCR duplicate removal was performed using collapse_duplicates.py from the Xing lab (Zhang and Xing, 2017) with modifications. Peak calling was performed using CLAM v1.2 (Zhang and Xing, 2017). Briefly, CLAM preprocessor was used to separate multi-mapped and uniquely mapped reads. Multi-mapped reads (--max-multihits of 10) were re-aligned to the genome with a probability. CLAM peakcaller was used in multi-replicate mode (n=3 replicates) with size-matched input as background using default values for --qval-cutoff (0.05), --fold-change (2-inf), and -binsize (50). Finally, peak annotation was performed with CLAM peak_annotator.

### Peak Analyses

De novo motif finding was performed using Homer v4.11 (-len 5,6,7-size 100-S 10). Peak distribution by genomic feature was calculated using RSeQC v4.0.0 read_distribution.py.

### Splicing Regulatory Maps

Splicing regulatory maps were generated using RBPmaps (Yee et al., 2019) (--normalization_level 0, --sigtest zscore) with CLAM peaks and significant AS events identified by rMATS (pval<0.05, FDR<0.1, IncLevelDifference>=|0.05|). The rMATS output was first subsetted by overlaps to eliminate overlapping events using a script provided by RBPmaps (subset_rmats_junctioncountonly.py). Background controls provided by RBPmaps were used for SE (HepG2_native_cassette_exons_all), A5SS (HepG2-all-native-a5ss-events), and A3SS (HepG2-all-native-a3ss-events) events.

### Human Cortex RNA-Seq Data

The Genotype-Tissue Expression (GTEx) Project was supported by the Common Fund of the Office of the Director of the National Institutes of Health, and by NCI, NHGRI, NHLBI, NIDA, NIMH, and NINDS. The data used for the analyses described in this manuscript were obtained from: Brain Front Cortex (gtexCovBrainFrontalCortexBA9) table from the GTEx RNA-seq Coverage track on the UCSC genome browser on May 6, 2022.

Rna Extraction and cDNA Synthesis

Total RNA from cells was isolated using Quick-RNA Miniprep Kit (Zymo Research, #R1055) following manufacturer's instructions, including DNase treatment step. RNA was eluted in a final volume of 50 μL of RNase-free water.

Total RNA from brain tissue was extracted using TRIzol. Tissue section (right hemisphere) was mixed with 1 mL of TRIzol reagent (Invitrogen, #15596018) and a 5 mm stainless steel bead (Qiagen, #69989) in a RNase-free microcentrifuge tube. Brain tissue was then homogenized in a TissueLyser LT homogenizer (Qiagen, #85600) for 5 min at 50 Hz. The homogenate was centrifuge at 12,000×g for 5 min at 4° C. and then seated for an additional 5 min to precipitate insoluble debris. The supernatant was transferred to a new tube, mixed with 200 μL of chloroform (Acros Organics, #190764), shaken vigorously and centrifuged at 12,000×g for 15 min at 4° C. Aqueous phase was transferred to a new tube containing 500 μL of ice-cold isopropanol (Sigma-Aldrich, #190764) followed by incubation for 10 min on ice and centrifugation at 12,000×g for 10 min at 4° C. to precipitate RNA. Supernatant was discarded and 1 mL of ice-cold 75% ethanol (Decon laboratories, #2701) was added to wash the pellet followed by centrifugation at 7,500×g for 5 min at 4° C. Supernatant was again discarded and RNA pellet was air-dried for 20 min. RNA was resuspended in 200 μL of RNase-free water and allowed to reconstitute for 10 min at 56° C.

RNA concentration was determined by measuring OD₂₆₀ nm absorbance in a Synergy HTX reader (Biotek). cDNA synthesis was performed using the SuperScript IV First-Strand Synthesis System with ezDNase Enzyme (ThermoScientific, #18091300) using random hexamer primers according to manufacturer's instructions. The ezDNase treatment step was performed for all conditions.

### RT-PCR

PCR reaction was prepared in a 0.2-mL tube by mixing the following reagents: 5 μL cDNA template, 1×MyTaq reaction mix (BIO-LINE, #25042), forward and reverse primers (0.4 μM each) and nuclease-free water in a final volume of 25 μL.

For SYNGAP1 (exon 10-11), PCR analysis to assess both the productive and non-productive transcript was performed using forward primer 5′-AATTCATCCGTGCTCTGTATGA-3′ [SEQ ID NO: 61] and reverse primer 5′-AAGAGACTGGGCGACATAATC-3′ [SEQ ID NO: 62]. PCR cycling conditions were 15 s at 95° C. for denaturation, 15 s at 64° C. for annealing, and 30 s at 72° C. for extension for 26 cycles. The predicted molecular weights of the PCR products were 289 bp for the productive transcript (containing exon 10 and 11) and 465 bp for the non-productive transcript (containing exon 10, 11× and 11).

For SYNGAP (exon 14), PCR analysis to assess inclusion of exon 14 was performed using forward primer 5′-TCCTGAAGCTGGGTCCACTG-3′ [SEQ ID NO: 63] and reverse primer 5′-GGGTGGCTTTTCCTTGGTTG-3′ [SEQ ID NO: 64]. PCR cycling conditions were 15 s at 95° C. for denaturation, 15 s at 68° C. for annealing, and 30 s at 72° C. for extension for 26 cycles. The predicted molecular weights of the PCR products were 221 bp for exon exclusion (containing exon 13 and 15) and 263 bp for the exon inclusion (containing exon 13, 14 and 15).

For SYNGAP (exon 18-19), PCR analysis to assess both the productive and non-productive transcript was performed using forward primer 5′-AGGCAGAGAAGGATTCCCAGA-3′ [SEQ ID NO: 65] and reverse primer 5′-TCACACGCGGGTTTGTTGG-3′ [SEQ ID NO: 66]. PCR cycling conditions were 15 s at 95° C. for denaturation, 15 s at 64° C. for annealing, and 30 s at 72° C. for extension for 26 cycles. The predicted molecular weights of the PCR products were 174 bp for the productive transcript (containing exon 17, 18 and 19) and 254 bp for the non-productive transcript (containing exon 17, 18, 19× and 19).

For ms-Syngap1 (ex10-11), PCR analysis to assess both the productive and non-productive transcript was performed using forward primer 5′-AGACCCCATCAAGTGCACAG-3′ [SEQ ID NO: 67] and reverse primer 5′-GCCTGTCAGCAATGTCCTCT-3′ [SEQ ID NO: 68]. PCR cycling conditions were PCR cycling conditions were 15 s at 95° C. for denaturation, 15 s at 65° C. for annealing, and 45 s at 72° C. for extension for 25 cycles. The predicted molecular weights of the PCR products were 185 bp for the productive transcript (containing exon 10 and 11) and 356 bp for the non-productive transcript (containing exon 10, 11× and 11).

For ms-Scn1a, PCR analysis to assess both the productive and non-productive transcript was performed using forward primer 5′-CAGTTTAACAGCAAATGCCTTGGGTT-3′ [SEQ ID NO: 69] and reverse primer 5′-AAGTACAAATACATGTACAGGCTTTCCTCATACTTA-3′ [SEQ ID NO: 70] and cycling conditions from (Lim et al., 2020). The predicted molecular weights of the PCR products were 498 bp for the productive transcript (containing exon 21, 22, 23 and 24) and 562 bp for the non-productive transcript (containing exon 21, 21×, 22, 23 and 24).

PCR products were mixed with 1×GelRed prestain loading buffer (Biotium, #41010), separated on a 2% or 4% agarose gel (Seakem LE agarose, Lonza, #50004) by electrophoresis (120 V, 30 min) and imaged using an Alphalmager system (AlphaInnotech). Gels were quantified with Image Studio Lite software (LI-COR).

qPCR

Probe-based qPCR was prepared by mixing the following reagents: 1 μL of cDNA, 1× Prime Time Gene Expression Master Mix (IDT, #1055772), 1× primers/probe mix and nuclease-free water to a final volume of 10 μL. Three technical replicates were performed for each sample. qPCR was carried out on a QuantStudio 3 Real-Time PCR System (ThermoFisher) with the following cycling conditions: 95° C. for 3 min for 1 cycle, 95° C. for 5 s and 60° C. for 30 s for 40 cycles. ΔCt was calculated by subtracting the average Ct of the reference gene from the average Ct of the gene of interest for each sample. ΔΔCt values were obtained by subtracting the average ΔCt value of control samples from the ΔCt of the test samples, and then converted into 2-44Ct to obtain the fold change of gene expression. The following qPCR probes were used for mRNA expression analyses: SYNGAP1 exon 10-11 (Hs00405348_ml, ThermoFisher), RPL4 (Hs00973293_g1, ThermoFisher), ATP5F1 (Hs01076982_g1, ThermoFisher), GAPDH (Hs00266705_g1, ThermoFisher), PTBP1 (Hs.PT.58.25863276, IDT), PTBP2 (Hs.PT.58.20884110, IDT), ms-Syngap1 (Mm01306145_ml, ThermoFisher), ms-Scnla (Mm00450583_mH, ThermoFisher), ms-Atp5fl (Mm05814774_g1, ThermoFisher), SYNGAP1 exon 16-17 (Hs.PT.58.4622325, IDT).

qPCR For Neuronal Markers

iPS-Neurons were harvested between Day 40-50 of differentiation, or equivalent, in RNA lysis buffer (Zymo) and RNA was purified with the Quick-RNA Miniprep kit (Zymo R1054), as per manufacturer's instructions. Additional gDNA was subsequently removed with the TURBO DNase free kit (ThermoFisher), as per manufacturer's instructions. cDNA was generated with a mix containing 9 μl RNA, 1 ul N6 random hexamers (Invitrogen) that were held at 70C for 5 min, followed by 4C. 4 μl of 5× First-Strand Buffer, 1 μl 0.1M DTT, 1 ul 10 mM dNTPs mix, 1 μl RNaseOUT, 0.75 μl superscript III (each from Invitrogen), and 2.25 μl RNase/DNase-free water were subjected to five-stepwise incremental temperature increases from 25° C. to 70° C. The resulting reaction was kept at 4° C. until use and diluted 1:10 in RNase/DNase-free water.

qPCR was performed with 1 ul of a primer mix (2 μM each of forward and reverse primer), 2.5 μl SYBR green, and 1.5 μl of cDNA. Mastermixes of the primer mix+SYBR green were added to all applicable wells prior to the addition of cDNA, which was added individually in triplicate. A standard curve of gDNA (10, 1, 0.1 ng/ml) generated from H9 embryonic stem cells was also examined against each primer pair. All primer pairs were generated to be within exon such that they could be used to calculate a concentration of cDNA transcript in the given sample. Each transcript concentration was standardized against the concentration of GAPDH in the sample. Note that GAPDH primer sets additionally bind to 3 pseudo-genes, resulting in additional gDNA binding and thereby allowing for assessment of GAPDH in the same linear range as other transcripts.

qPCR samples were analyzed on a QuantStudio5 System (AppliedBiosystems) with a Passive reference of ROX and analyzed by QuantStudio5 software using the Relative Standard Curve setting.

Primer Sets:

| | | SEQ | | SEQ |
|---|---|---|---|---|
| Transcript | | ID | | ID |
| name | Forward | NO: | Reverse | NO: |
| GAPDH | TCATCCCTGAGCTG | 71 | GCCTGCTTCACCACCTT | 72 |
| | AACGGGAA | | CTTGATG | |
| TBR2 | ACTCAATCCCACTG | 73 | GCTTTGCCACAGGTCAC | 74 |
| (EOMES) | CCCACTACAA | | CCATTT | |
| TBR1 | TGGAGCACTGCCTT | 75 | CTCCAGGTTGTCAGTGG | 76 |
| | TCTCCTTCTATC | | TCGAGATAATG | |
| CTIP2 | AGTACTGCGGCAAG | 77 | GTTGCACAGCTCGCACT | 78 |
| (BCL11B) | GTGTTCAAGA | | TGTAAGG | |
| SATB2 | CCATCATGAGCCCT | 79 | CGGCTAACGGCAATCTG | 80 |
| | GGTCTTCTTTCT | | TTGGTTT | |
| REELIN | CCTCGTTATGCTGA | 81 | CTGGAGCTGTACACTGT | 82 |
| (RELN) | GACCTGGGATTT | | GGGAGTT | |
| NGN2 | CGAAACCGCATGCA | 83 | CTCGGTGAGTGCCCAGA | 84 |
| (NEUROG | CAACCTCAA | | TGTAGTT | |
| 2) | | | | |
| VGLUT1 | CCATCATGAGTGGT | 85 | GCGGTGGGTCGTGCTGT | 86 |
| (SLC17A7) | CTGGGCTTCT | | TATT | |
| VGLUT2 | AGACCCGGAGGAAA | 87 | AACCTCCATTGGCCTGT | 88 |
| (SLC17A6) | CAAGTGAAGA | | GTTGTG | |
| GAD67 | GGCTAAGAACGGTG | 89 | CAGCAACTGGTGTGGGT | 90 |
| (GAD1) | AGGAGCAAAC | | GATGAAA | |
| GAD65 | ACCAAATGCATGCC | 91 | TTTGCCCTCCACATCAG | 92 |
| (GAD2) | TCCTACCTCTT | | CCATAGT | |

### Immunofluorescence

iPS-Neurons were plated on to German-glass coverslips (Electron Microscopy services) for the terminal differentiation stage and then fixed at Day 46 of differentiation were fixed with 4% paraformaldehyde for 30 min at room temperature. Cells were permeabilized with PBS+0.3% Triton X-100 (Sigma) for 10 min. Cells were then blocked with Animal-Free Blocking solution (Cell Signaling Technology) for 1 h at room temperature, followed by primary antibody diluted in PBS+3% BSA (Sigma A1470) overnight at 4° C. Primary antibodies included: MAP2 (Sigma M1406, 1:500), Tuj 1 (Biolegend, 801201, 1:500), PSD-95 (Cell Signaling, 3450, 1:200). Cells were subsequently treated with secondary antibodies Goat anti-IgG1-Alexa488 (for MAP2), goat anti-IgG2A-Alexa647 (for Tuj1), Goat anti-rabbit Alexa568 (for PSD-95) (each 1:500, Invitrogen) diluted in PBS+3% BSA for 2 h at room temp, follow by post-fixation with 4% paraformaldehyde for 15 min at room temp. Cells were counterstained with Hoechst-33342 (Invitrogen, 1:2000) in PBS for 15 min at room temperature, and mounted with Fluoromount-G (Southern Biotech). Washes with PBS were performed between each step, and a final wash with water was performed prior to mounting.

Images were taken on a Leica DMi4000 inverted microscope, outfitted with a PL APO 40× objective and Leica DFC340 FX camera.

### Western Blot

Total protein extraction from cells was performed using 1.5× Laemmli buffer [15% glycerol (Amresco #M152), 3% SDS (Sigma #L5750), 3.75 mM EDTA (Bio-Rad #1610729) and 75 mM Tris, pH 7.5 (Invitrogen #15567027)] followed by 10 min incubation at 95° C.

Protein extracts were quantified with Pierce BCA protein assay kit (ThermoFisher, #23227) according to manufacturer's instructions, diluted to the same final concentration, mixed with 1× Orange O dye containing 10% β-mercaptoethanol (Sigma #M3148) and incubated 10 min at 100° C. before loading. Precast 4-15% TGX protein gels (Bio-Rad) were loaded with 10-30 μg of total protein lysate and run for 1 h at 110-135V. Transfer was performed with a Trans-Blot Turbo Transfer system (Bio-Rad) using the pre-determined high molecular weight transfer protocol (10 min, 2.5 A constant).

For SYNGAP1 blots, proteins were transferred to a 0.45 μm low fluorescence PVDF membrane (Bio-Rad, #1704275) and blocking was performed using 5% ECL prime blocking reagent (Cytiva, #RPN418) for at least 1h at room temperature. Incubation with primary antibodies (diluted in blocking buffer) was carried out overnight at 4° C. Rabbit anti-SYNGAP1 (Cell Signaling Technology #5539S, 1:1000 dilution) previously validated on (Lim et al., 2020) and mouse anti-ATP5F1 (Abcam #ab117991, 1:1000 dilution or Santa Cruz Biotechnology #sc-514419, 1:500 dilution) were used. Membrane was then rinsed with TBS-T 4 times for 5 min. Incubation with secondary antibodies (diluted in blocking buffer) was performed at room temperature for 1 h. Anti-rabbit-HRP (Cell Signaling Technology #7074S, 1:5000 dilution) and anti-mouse-HRP (Cell Signaling Technology #7076S, 1:5000 dilution) were used. Membrane was rinsed again with TBS-T 4 times for 5 min before imaging. Blots were developed using SuperSignal West Femto Maximum Sensitivity Substrate (ThermoScientific, #34095) and detection was carried out on a GBox imaging system (Syngene). Blot images were exported in 16-bit grayscale format for further analysis.

For PTBP1 and PTBP2 blots, proteins were transferred to a 0.2 μm nitrocellulose membrane (Bio-Rad, #1704271) and blocking was performed using Intercept (TBS) Blocking Buffer (LI-COR, #927-60001) for at least 1h at room temperature. Incubation with primary antibodies (diluted in blocking buffer containing 0.1% Tween-20) was carried out overnight at 4° C. Rabbit anti-PTBP1 (Cell Signaling Technology #72669, 1:1000 dilution), rabbit anti-PTBP2 (EMD Millipore #ABE431, 0.5 μg/mL), and mouse anti-ATP5F1 (as above) were used. Membrane was then rinsed with TBS-T 4 times for 5 min. Incubation with secondary antibodies (diluted in blocking buffer containing 0.1% Tween-20) was performed at room temperature for 1h. IRDye 680RD anti-rabbit (LI-COR #926-68073, 1:10,000 dilution) and IRDye 800CW anti-mouse (LI-COR #926-32212, 1:10,000 dilution) were used. Membrane was rinsed again with TBS-T 4 times for 5 min and imaged on Odyssey Imager (LI-COR) using a resolution of 169 μm.

Western blots quantifications were normalized to ATP5F1.

### Single Bolus ICV Injection in Neonate Mice

C57BL/6NCrl male and female mice were used in this study. All mice were maintained on a 12:12-h light: dark cycle and had ad libitum access to food and water throughout the experiments.

Lyophilized ASO was reconstituted in 1×PBS (Thermo Fisher, #10010023) and diluted to the desired concentration. For ICV injection in P2 mice, pups were immobilized by gently restraining them on a soft tissue padded surface with two fingers. A 10 uL Hamilton syringe was used for the injection. The coordinates of the injection were ˜1 mm lateral from the sagittal suture and −2 mm ventral. In total, 2 μL of ASO or PBS was injected slowly into one cerebral lateral ventricle. Injected mice were quickly returned to the nest and observed daily for survival and signs of stress. Animals were sacrificed at P7, and brain sectioned into R and L hemispheres. Cortex was separated from subcortical structures (striatum, thalamus+hippocampus all together), flash-frozen in liquid nitrogen and stored at −80° C.

### Densitometry Analysis

Blot images were imported into Image Studio Lite software (LI-COR) for quantification. Bands were selected using the rectangle tool and background was subtracted using a border width of 3 from top/bottom. The optical/fluorescence densitometry of the selected area was measured and exported for further analysis. Percentage of productive splicing (%) was calculated as the ratio between productive transcript levels and total transcript levels from RT-PCR assays.

### Statistical Analyses

Statistical significance for all experiments was defined as p<0.05 (or adjusted p value of <0.05 or FDR of <0.05 for sequencing data) and calculated using GraphPad Prism 9.3 software or R. Data plots were prepared using GraphPad Prism 9.3 software and R. Individual statistical tests applied to each data set are given in the respective figure legends or methods section.

Bar graphs represented as mean values±SEM

### REFERENCES

Berryer, M. H., Hamdan, F. F., Klitten, L. L., Møller, R. S., Carmant, L., Schwartzentruber, J., Patry, L., Dobrzeniecka, S., Rochefort, D., Neugnot-Cerioli, M., Lacaille, J. C., Niu, Z., Eng, C. M., Yang, Y., Palardy, S., Belhumeur, C., Rouleau, G. A., Tommerup, N., Immken, L., . . . di Cristo, G. (2013). Mutations in SYNGAP1 Cause Intellectual Disability, Autism, and a Specific Form of Epilepsy by Inducing Haploinsufficiency. Human Mutation, 34 (2), 385-394. https://doi.org/10.1002/HUMU.22248
Boutz, P. L., Stoilov, P., Li, Q., Lin, C. H., Chawla, G., Ostrow, K., Shiue, L., Ares, M., & Black, D. L. (2007). A post-transcriptional regulatory switch in polypyrimidine tract-binding proteins reprograms alternative splicing in developing neurons. Genes & Development, 21 (13), 1636-1652. https://doi.org/10.1101/GAD.1558107
Clement, J. P., Aceti, M., Creson, T. K., Ozkan, E. D., Shi, Y., Reish, N. J., Almonte, A. G., Miller, B. H., Wiltgen, B. J., Miller, C. A., Xu, X., & Rumbaugh, G. (2012). Pathogenic SYNGAP1 Mutations Impair Cognitive Development by Disrupting Maturation of Dendritic Spine Synapses. Cell, 151 (4), 709-723. https://doi.org/10.1016/J.CELL.2012.08.045
Crooke, S. T., Liang, X. H., Baker, B. F., & Crooke, R. M. (2021). Antisense technology: A review. Journal of Biological Chemistry, 296, 100416-100417. https://doi.org/10.1016/J.JBC.2021.100416
Denichenko, P., Mogilevsky, M., Cléry, A., Welte, T., Biran, J., Shimshon, O., Barnabas, G. D., Danan-Gotthold, M., Kumar, S., Yavin, E., Levanon, E. Y., Allain, F. H., Geiger, T., Levkowitz, G., & Karni, R. (2019). Specific inhibition of splicing factor activity by decoy RNA oligonucleotides. Nature Communications 2019 10:1, 10 (1), 1-15. https://doi.org/10.1038/s41467-019-09523-0
Finkel, R. S., Mercuri, E., Darras, B. T., Connolly, A. M., Kuntz, N. L., Kirschner, J., Chiriboga, C. A., Saito, K., Servais, L., Tizzano, E., Topaloglu, H., Tulinius, M., Montes, J., Glanzman, A. M., Bishop, K., Zhong, Z. J., Gheuens, S., Bennett, C. F., Schneider, E., . . . de Vivo, D. C. (2017). Nusinersen versus Sham Control in Infantile-Onset Spinal Muscular Atrophy. New England Journal of Medicine, 377 (18), 1723-1732. https://doi.org/10.1056/NEJMOA1702752/SUPPL_FILE/NEJMOA1702752_DISCLOSUR ES.PDF
Gamache, T. R., Araki, Y., & Huganir, R. L. (2020). Twenty Years of SynGAP Research: From Synapses to Cognition. The Journal of Neuroscience: The Official Journal of the Society for Neuroscience, 40 (8), 1596-1605. https://doi.org/10.1523/JNEUROSCI.0420-19.2020
Hamdan, F. F., Gauthier, J., Spiegelman, D., Noreau, A., Yang, Y., Pellerin, S., Dobrzeniecka, S., Côté, M., Perreau-Linck, E., Carmant, L., D'Anjou, G., Fombonne, É., Addington, A. M., Rapoport, J. L., Delisi, L. E., Krebs, M.-O., Mouaffak, F., Joober, R., Mottron, L., . . . . Michaud, J. L. (2009). Mutations in SYNGAP1 in Autosomal Nonsyndromic Mental Retardation. New England Journal of Medicine, 360 (6), 599-605. https://doi.org/10.1056/NEJMOA0805392/SUPPL_FILE/NEJM_HAMDAN_599SA1.PDF
Han, Z., Chen, C., Christiansen, A., Ji, S., Lin, Q., Anumonwo, C., Liu, C., Leiser, S. C., Meena, Aznarez, I., Liau, G., & Isom, L. L. (2020). Antisense oligonucleotides increase Sonla expression and reduce seizures and SUDEP incidence in a mouse model of Dravet syndrome. Science Translational Medicine, 12 (558), 6100. https://doi.org/10.1126/SCITRANSLMED.AAZ6100/SUPPL_FILE/AAZ6100_SM.PDF
Hua, Y., Vickers, T. A., Okunola, H. L., Bennett, C. F., & Krainer, A. R. (2008). Antisense Masking of an hnRNP A1/A2 Intronic Splicing Silencer Corrects SMN2 Splicing in Transgenic Mice. The American Journal of Human Genetics, 82 (4), 834-848. https://doi.org/10.1016/J.AJHG.2008.01.014
Karousis, E. D., Gypas, F., Zavolan, M., & Mühlemann, O. (2021). Nanopore sequencing reveals endogenous NMD-targeted isoforms in human cells. Genome Biology, 22 (1). https://doi.org/10.1186/S13059-021-02439-3
Khorkova, O., & Wahlestedt, C. (2017). Oligonucleotide therapies for disorders of the nervous system. Nature Biotechnology, 35 (3), 249-263. https://doi.org/10.1038/NBT.3784
Koopmans F, van Nierop P, Andres-Alonso M, Byrnes A, Cijsouw T, Coba M P, Cornelisse L N, Farrell R J, Goldschmidt H L, Howrigan D P, Hussain N K, Imig C, de Jong APH, Jung H, Kohansalnodehi M, Kramarz B, Lipstein N, Lovering R C, MacGillavry H, Mariano V, Mi H, Ninov M, Osumi-Sutherland D, Pielot R, Smalla K H, Tang H, Tashman K, Toonen RFG, Verpelli C, Reig-Viader R, Watanabe K, van Weering J, Achsel T, Ashrafi G, Asi N, Brown T C, De Camilli P, Feuermann M, Foulger R E, Gaudet P, Joglekar A, Kanellopoulos A, Malenka R, Nicoll R A, Pulido C, de Juan-Sanz J, Sheng M, Südhof T C, Tilgner H U, Bagni C, Bayés A, Biederer T, Brose N, Chua JJE, Dieterich D C, Gundelfinger E D, Hoogenraad C, Huganir R L, Jahn R, Kaeser P S, Kim E, Kreutz M R, McPherson P S, Neale B M, O'Connor V, Posthuma D, Ryan T A, Sala C, Feng G, Hyman S E, Thomas P D, Smit A B, Verhage M. SynGO: An Evidence-Based, Expert-Curated Knowledge Base for the Synapse. Neuron. 2019 Jul. 17; 103 (2): 217-234.e4. doi: 10.1016/j.neuron.2019.05.002. Epub 2019 Jun. 3. PMID: 31171447; PMCID: PMC6764089.
Li, Q., Zheng, S., Han, A., Lin, C. H., Stoilov, P., Fu, X. D., & Black, D. L. (2014). The splicing regulator PTBP2 controls a program of embryonic splicing required for neuronal maturation. ELife, 2014 (3). https://doi.org/10.7554/ELIFE.01201.001
Liang, X. H., Nichols, J. G., Hsu, C. W., Vickers, T. A., & Crooke, S. T. (2019). mRNA levels can be reduced by antisense oligonucleotides via no-go decay pathway. Nucleic Acids Research, 47 (13), 6900-6916. https://doi.org/10.1093/NAR/GKZ500
Licatalosi, D. D., Yano, M., Fak, J. J., Mele, A., Grabinski, S. E., Zhang, C., & Darnell, R. B. (2012). Ptbp2 represses adult-specific splicing to regulate the generation of neuronal precursors in the embryonic brain. Genes & Development, 26 (14), 1626-1642. https://doi.org/10.1101/GAD.191338.112
Lim, K. H., Han, Z., Jeon, H. Y., Kach, J., Jing, E., Weyn-Vanhentenryck, S., Downs, M., Corrionero, A., Oh, R., Scharner, J., Venkatesh, A., Ji, S., Liau, G., Ticho, B., Nash, H., & Aznarez, I. (2020). Antisense oligonucleotide modulation of non-productive alternative splicing upregulates gene expression. Nature Communications 2020 11:1, 11 (1), 1-13. https://doi.org/10.1038/s41467-020-17093-9
Linares, A. J., Lin, C. H., Damianov, A., Adams, K. L., Novitch, B. G., & Black, D. L. (2015). The splicing regulator PTBP1 controls the activity of the transcription factor Pbx1 during neuronal differentiation. ELife, 4 (December 2015). https://doi.org/10.7554/ELIFE.09268
Raj, B., & Blencowe, B. J. (2015). Alternative Splicing in the Mammalian Nervous System: Recent Insights into Mechanisms and Functional Roles. Neuron, 87 (1), 14-27. https://doi.org/10.1016/J.NEURON.2015.05.004
Shen, S., Park, J. W., Lu, Z. X., Lin, L., Henry, M. D., Wu, Y. N., Zhou, Q., & Xing, Y. (2014). rMATS: robust and flexible detection of differential alternative splicing from replicate RNA-Seq data. Proceedings of the National Academy of Sciences of the United States of America, 111 (51), E5593-E5601. https://doi.org/10.1073/PNAS.1419161111
Su, C. H., Dhananjaya, D., & Tarn, W. Y. (2018). Alternative splicing in neurogenesis and brain development. Frontiers in Molecular Biosciences, 5 (February), 12. https://doi.org/10.3389/FMOLB.2018.00012/BIBTEX
van Nostrand, E. L., Pratt, G. A., Yee, B. A., Wheeler, E. C., Blue, S. M., Mueller, J., Park, S. S., Garcia, K. E., Gelboin-Burkhart, C., Nguyen, T. B., Rabano, I., Stanton, R., Sundararaman, B., Wang, R., Fu, X. D., Graveley, B. R., & Yeo, G. W. (2020). Principles of RNA processing from analysis of enhanced CLIP maps for 150 RNA binding proteins. Genome Biology, 21 (1). https://doi.org/10.1186/S13059-020-01982-9
Vuong, C. K., Black, D. L., & Zheng, S. (2016). The neurogenetics of alternative splicing. Nature Reviews Neuroscience 2016 17:5, 17 (5), 265-281. https://doi.org/10.1038/nrn.2016.27
Vuong, J. K., Lin, C. H., Zhang, M., Chen, L., Black, D. L., & Zheng, S. (2016). PTBP 1 and PTBP2 Serve Both Specific and Redundant Functions in Neuronal Pre-mRNA Splicing. Cell Reports, 17 (10), 2766-2775. https://doi.org/10.1016/J.CELREP.2016.11.034
Weyn-Vanhentenryck, S. M., Feng, H., Ustianenko, D., Duffie, R., Yan, Q., Jacko, M., Martinez, J. C., Goodwin, M., Zhang, X., Hengst, U., Lomvardas, S., Swanson, M. S., & Zhang, C. (2018). Precise temporal regulation of alternative splicing during neural development. Nature Communications 2018 9:1, 9 (1), 1-17. https://doi.org/10.1038/s41467-018-04559-0
Xue, Y., Zhou, Y., Wu, T., Zhu, T., Ji, X., Kwon, Y. S., Zhang, C., Yeo, G., Black, D. L., Sun, H., Fu, X. D., & Zhang, Y. (2009). Genome-wide analysis of PTB-RNA interactions reveals a strategy used by the general splicing repressor to modulate exon inclusion or skipping. Molecular Cell, 36 (6), 996-1006. https://doi.org/10.1016/J.MOLCEL.2009.12.003
Yee, B. A., Pratt, G. A., Graveley, B. R., van Nostrand, E. L., & Yeo, G. W. (2019). RBP-Maps enables robust generation of splicing regulatory maps. RNA (New York, N.Y.), 25 (2), 193-204. https://doi.org/10.1261/RNA.069237.118
Zhang, Z., & Xing, Y. (2017). CLIP-seq analysis of multi-mapped reads discovers novel functional RNA regulatory sites in the human transcriptome. Nucleic Acids Research, 45 (16), 9260-9271. https://doi.org/10.1093/NAR/GKX646

Table 3 lists other disease-causing genes that are both alternatively spliced and differentially expressed upon PTBP2 depletion, and which show direct PTBP binding near alternative splicing (AS) events. The table shows 342 Orphanet genes differentially spliced upon PTBP KD, with 75 showing PTBP2-binding proximal to an alternative slicing event. 17 demonstrated direct PTBP2 binding, differential AS, and differential gene expression upon PTBP2 depletion, which prompts further evaluation of PTBP2-dependent splicing as a targetable therapeutic strategy for these genetic etiologies. In the following table, MXE refers to mutually exclusive exons; SE is a skipped exon; A5SS, alternative 5′ splice site; A3SS, alternative 3′ splice site; RI, retained intron.

TABLE 3

| | | | splice | peaks - | |
|---|---|---|---|---|---|
| geneSymbol | GeneID | DGE | event | brain | d |
| ABHD12 | ENSG00000100997.20 | | SE | 0 | 0 |
| ACADVL | ENSG00000072778.20 | | SE | 0 | 0 |
| ACADVL | ENSG00000072778.20 | | A3SS | 0 | 0 |
| ACTN4 | ENSG00000130402.13 | | SE | 8 | 2 |
| ACTN4 | ENSG00000130402.13 | | MXE | 10.72 | 2.72 |
| ACTN4 | ENSG00000130402.13 | | MXE | 0 | 0 |
| AGO2 | ENSG00000123908.12 | | SE | 0 | 0 |
| AGPS | ENSG00000018510.18 | | SE | 0 | 0 |
| AGRN | ENSG00000188157.15 | | SE | 0 | 0 |
| AGTPBP1 | ENSG00000135049.16 | | SE | 0 | 0 |
| AIFM1 | ENSG00000156709.15 | | SE | 0 | 0 |
| AIFM1 | ENSG00000156709.15 | | MXE | 0 | 0 |
| AKR1C2 | ENSG00000151632.17 | Up-regulated | SE | 0 | 0 |
| | | upon PTBP2 KD | | | |
| ANK3 | ENSG00000151150.22 | | SE | 0 | 0 |
| ANKRD26 | ENSG00000107890.17 | | SE | 0 | 0 |
| ANKS6 | ENSG00000165138.18 | | SE | 0 | 0 |
| AP1B1 | ENSG00000100280.17 | | SE | 0 | 0 |
| AP1B1 | ENSG00000100280.17 | | SE | 0 | 0 |
| AP1G1 | ENSG00000166747.13 | | SE | 0 | 0 |
| AP3D1 | ENSG00000065000.19 | | SE | 0 | 0 |
| APC | ENSG00000134982.17 | | SE | 0 | 0 |
| APP | ENSG00000142192.21 | | SE | 0 | 0 |
| ARF1 | ENSG00000143761.16 | | SE | 0 | 0 |
| ASAH1 | ENSG00000104763.20 | | SE | 0 | 0 |
| ASPH | ENSG00000198363.18 | | SE | 0 | 0 |
| ASXL1 | ENSG00000171456.20 | | SE | 0 | 0 |
| ATM | ENSG00000149311.20 | Down-regulated | MXE | 0 | 0 |
| | | upon PTBP2 KD | | | |
| ATP7A | ENSG00000165240.21 | | SE | 0 | 0 |
| ATXN2 | ENSG00000204842.18 | Down-regulated | SE | 0.44 | 0.44 |
| | | upon PTBP2 KD | | | |
| ATXN2 | ENSG00000204842.18 | Down-regulated | MXE | 0 | 0 |
| | | upon PTBP2 KD | | | |
| ATXN3 | ENSG00000066427.24 | | SE | 0 | 0 |
| ATXN7 | ENSG00000163635.20 | | SE | 0 | 0 |
| BCS1L | ENSG00000074582.15 | | RI | 0 | 0 |
| BDP1 | ENSG00000145734.19 | | SE | 0 | 0 |
| BIN1 | ENSG00000136717.15 | Up-regulated | SE | 6.82 | 0 |
| | | upon PTBP2 KD | | | |
| BIN1 | ENSG00000136717.15 | Up-regulated | MXE | 6.82 | 0 |
| | | upon PTBP2 KD | | | |
| BLOC1S6 | ENSG00000104164.12 | | SE | 0 | 0 |
| BMP1 | ENSG00000168487.20 | | SE | 0 | 0 |
| BPTF | ENSG00000171634.18 | | SE | 0 | 0 |
| BRCC3 | ENSG00000185515.15 | | SE | 0 | 0 |
| BRSK2 | ENSG00000174672.16 | | SE | 0 | 0 |
| BUD23 | ENSG00000071462.12 | | SE | 0 | 0 |
| BUD23 | ENSG00000071462.12 | | MXE | 0 | 0 |
| C2CD3 | ENSG00000168014.18 | | SE | 0 | 0 |
| CACNA1B | ENSG00000148408.13 | | SE | 0 | 0 |
| CACNA2D1 | ENSG00000153956.16 | | SE | 0 | 0 |
| CAD | ENSG00000084774.14 | | SE | 0 | 0 |
| CAD | ENSG00000084774.14 | | A3SS | 0 | 0 |
| CALM2 | ENSG00000143933.19 | | SE | 0 | 8.68 |
| CAMK2B | ENSG00000058404.20 | Up-regulated | SE | 1 | 0 |
| | | upon PTBP2 KD | | | |
| CAMK2B | ENSG00000058404.20 | Up-regulated | SE | 0 | 0 |
| | | upon PTBP2 KD | | | |
| CAMK2B | ENSG00000058404.20 | Up-regulated | A3SS | 0 | 0 |
| | | upon PTBP2 KD | | | |
| CAMTA1 | ENSG00000171735.19 | Down-regulated | SE | 0 | 0 |
| | | upon PTBP2 KD | | | |
| CARS1 | ENSG00000110619.18 | | SE | 0 | 0 |
| CAST | ENSG00000153113.24 | | SE | 0 | 0 |
| CCDC50 | ENSG00000152492.15 | | SE | 0 | 0 |
| CCM2 | ENSG00000136280.17 | | SE | 0 | 0 |
| CD46 | ENSG00000117335.20 | | MXE | 0 | 0 |
| CD55 | ENSG00000196352.16 | | SE | 0 | 0 |
| CDKN1A | ENSG00000124762.14 | Up-regulated | MXE | 0 | 2.92 |
| | | upon PTBP2 KD | | | |
| CELF2 | ENSG00000048740.18 | | SE | 0 | 0 |
| CENPE | ENSG00000138778.13 | Down-regulated | A3SS | 0 | 0 |
| | | upon PTBP2 KD | | | |
| CEP290 | ENSG00000198707.17 | | SE | 0 | 0 |
| CEP57 | ENSG00000166037.11 | | SE | 0 | 0 |
| CEP57 | ENSG00000166037.11 | | SE | 0 | 0 |
| CEP57 | ENSG00000166037.11 | | MXE | 0 | 0 |
| CEP63 | ENSG00000182923.20 | | SE | 0 | 0 |
| CEP83 | ENSG00000173588.15 | Down-regulated | SE | 0 | 0 |
| | | upon PTBP2 KD | | | |
| CHD2 | ENSG00000173575.22 | Up-regulated | SE | 0 | 0 |
| | | upon PTBP2 KD | | | |
| CHD8 | ENSG00000100888.15 | | RI | 0 | 1 |
| CHRNA7 | ENSG00000175344.19 | | SE | 0 | 0 |
| CHUK | ENSG00000213341.11 | Down-regulated | SE | 0 | 0 |
| | | upon PTBP2 KD | | | |
| CIBAR1 | ENSG00000188343.13 | Down-regulated | SE | 0 | 0 |
| | | upon PTBP2 KD | | | |
| CIZ1 | ENSG00000148337.21 | | RI | 0 | 0 |
| CLCN2 | ENSG00000114859.16 | | RI | 0 | 0 |
| CLCN6 | ENSG00000011021.23 | Up-regulated | RI | 0 | 0 |
| | | upon PTBP2 KD | | | |
| CLIP2 | ENSG00000106665.16 | | SE | 0 | 0 |
| CLIP2 | ENSG00000106665.16 | | MXE | 0 | 0 |
| CNOT1 | ENSG00000125107.19 | | RI | 0 | 0 |
| CNPY3 | ENSG00000137161.17 | | MXE | 0.18 | 0 |
| COL4A6 | ENSG00000197565.16 | Down-regulated | SE | 0 | 0 |
| | | upon PTBP2 KD | | | |
| COQ6 | ENSG00000119723.17 | | RI | 0 | 0 |
| COX4I1 | ENSG00000131143.10 | | SE | 0 | 5 |
| CPAMD8 | ENSG00000160111.15 | | SE | 0 | 0 |
| CPAMD8 | ENSG00000160111.15 | | A3SS | 0 | 0 |
| CPLANE1 | ENSG00000197603.16 | | RI | 0 | 0 |
| CRBN | ENSG00000113851.15 | Down-regulated | A5SS | 0 | 0 |
| | | upon PTBP2 KD | | | |
| CTNNB1 | ENSG00000168036.18 | | SE | 0 | 7.96 |
| CTNNB1 | ENSG00000168036.18 | | MXE | 0 | 9 |
| CTNND1 | ENSG00000198561.16 | | A5SS | 0 | 0.38 |
| CUL4B | ENSG00000158290.19 | | RI | 0 | 0 |
| DAB1 | ENSG00000173406.16 | | SE | 0 | 0 |
| DBT | ENSG00000137992.15 | | SE | 0 | 0 |
| DCAF17 | ENSG00000115827.14 | | SE | 0 | 0 |
| DCTN1 | ENSG00000204843.13 | | SE | 4 | 7 |
| DCTN1 | ENSG00000204843.13 | | MXE | 4 | 7 |
| DCX | ENSG00000077279.20 | | A5SS | 0 | 1 |
| DDB2 | ENSG00000134574.12 | | SE | 0 | 0 |
| DGAT1 | ENSG00000185000.12 | | SE | 0 | 0 |
| DGUOK | ENSG00000114956.20 | | SE | 0 | 0 |
| DLD | ENSG00000091140.14 | | SE | 0 | 0 |
| DLG3 | ENSG00000082458.12 | | SE | 0 | 1 |
| DLG3 | ENSG00000082458.12 | | SE | 0 | 1 |
| DLG3 | ENSG00000082458.12 | | MXE | 0 | 1 |
| DMPK | ENSG00000104936.20 | | A5SS | 0 | 0 |
| DNAJB2 | ENSG00000135924.17 | Up-regulated | SE | 5 | 1 |
| | | upon PTBP2 KD | | | |
| DNAJB2 | ENSG00000135924.17 | Up-regulated | RI | 3 | 1 |
| | | upon PTBP2 KD | | | |
| DNM1 | ENSG00000106976.21 | | SE | 0 | 0 |
| DNM1 | ENSG00000106976.21 | | MXE | 4 | 5.8 |
| DOCK7 | ENSG00000116641.18 | | A5SS | 0 | 0 |
| DOCK7 | ENSG00000116641.18 | | A3SS | 0 | 0 |
| DPF2 | ENSG00000133884.10 | | SE | 0 | 2 |
| DPH1 | ENSG00000108963.19 | | RI | 0 | 0 |
| DST | ENSG00000151914.21 | | SE | 4.02 | 2 |
| DST | ENSG00000151914.21 | | SE | 0 | 0 |
| DYM | ENSG00000141627.14 | Down-regulated | SE | 0 | 0 |
| | | upon PTBP2 KD | | | |
| EED | ENSG00000074266.22 | | SE | 0 | 0 |
| EIF4E | ENSG00000151247.13 | | SE | 0 | 0 |
| EIF4G1 | ENSG00000114867.22 | | SE | 0 | 12.54 |
| ELAC2 | ENSG00000006744.19 | | SE | 0 | 0 |
| ELAC2 | ENSG00000006744.19 | | MXE | 0 | 0 |
| ELMO2 | ENSG00000062598.18 | | RI | 0 | 0 |
| EMILIN1 | ENSG00000138080.14 | | SE | 0 | 0 |
| EML1 | ENSG00000066629.18 | Down-regulated | SE | 0 | 0 |
| | | upon PTBP2 KD | | | |
| EPB41 | ENSG00000159023.22 | Down-regulated | SE | 0 | 0 |
| | | upon PTBP2 KD | | | |
| EPB41L1 | ENSG00000088367.23 | | SE | 0 | 0 |
| EPB41L1 | ENSG00000088367.23 | | MXE | 0 | 0 |
| EPS8L2 | ENSG00000177106.16 | Up-regulated | SE | 0 | 0 |
| | | upon PTBP2 KD | | | |
| ERAL1 | ENSG00000132591.12 | | SE | 0 | 0 |
| ERBB2 | ENSG00000141736.14 | | RI | 0 | 0 |
| ERC1 | ENSG00000082805.20 | | SE | 0 | 0 |
| ERCC1 | ENSG00000012061.16 | Down-regulated | SE | 0 | 0 |
| | | upon PTBP2 KD | | | |
| ERCC8 | ENSG00000049167.16 | Down-regulated | RI | 0 | 0 |
| | | upon PTBP2 KD | | | |
| EWSR1 | ENSG00000182944.18 | | SE | 0 | 0 |
| EZH2 | ENSG00000106462.12 | | SE | 0 | 0 |
| EZH2 | ENSG00000106462.12 | | SE | 0 | 0 |
| EZH2 | ENSG00000106462.12 | | A3SS | 0 | 0 |
| FAM149B1 | ENSG00000138286.15 | | MXE | 0 | 0 |
| FANCA | ENSG00000187741.15 | Down-regulated | SE | 0 | 0 |
| | | upon PTBP2 KD | | | |
| FAR1 | ENSG00000197601.13 | | SE | 0 | 0 |
| FAS | ENSG00000026103.22 | | SE | 0 | 0 |
| FBXO7 | ENSG00000100225.18 | | SE | 0 | 0 |
| FBXW11 | ENSG00000072803.18 | | SE | 0 | 0 |
| FDPS | ENSG00000160752.15 | | A5SS | 0 | 3.54 |
| FDPS | ENSG00000160752.15 | | MXE | 0 | 6.1 |
| FDXR | ENSG00000161513.12 | | SE | 0 | 0 |
| FDXR | ENSG00000161513.12 | | SE | 0 | 0 |
| FDXR | ENSG00000161513.12 | | A5SS | 0 | 0 |
| FDXR | ENSG00000161513.12 | | A3SS | 0 | 0 |
| FDXR | ENSG00000161513.12 | | RI | 0 | 0 |
| FGD4 | ENSG00000139132.16 | Down-regulated | SE | 0 | 0.82 |
| | | upon PTBP2 KD | | | |
| FGD4 | ENSG00000139132.16 | Down-regulated | MXE | 0 | 3.82 |
| | | upon PTBP2 KD | | | |
| FGFR2 | ENSG00000066468.24 | | SE | 0 | 0 |
| FHIT | ENSG00000189283.10 | Down-regulated | SE | 0 | 0 |
| | | upon PTBP2 KD | | | |
| FIG4 | ENSG00000112367.12 | | A3SS | 0 | 0 |
| FKBP14 | ENSG00000106080.11 | | SE | 0 | 0 |
| FLII | ENSG00000177731.16 | | SE | 0 | 0 |
| FLII | ENSG00000177731.16 | | RI | 0 | 0 |
| FLNB | ENSG00000136068.16 | | SE | 0 | 0 |
| FLVCR1 | ENSG00000162769.13 | Up-regulated | SE | 0 | 0 |
| | | upon PTBP2 KD | | | |
| FLVCR1 | ENSG00000162769.13 | Up-regulated | SE | 0 | 0 |
| | | upon PTBP2 KD | | | |
| FMN2 | ENSG00000155816.21 | Up-regulated | SE | 0 | 0 |
| | | upon PTBP2 KD | | | |
| FSIP2 | ENSG00000188738.15 | Down-regulated | SE | 0 | 0 |
| | | upon PTBP2 KD | | | |
| GDAP1 | ENSG00000104381.14 | | SE | 0 | 1 |
| GFM1 | ENSG00000168827.15 | | SE | 0 | 0 |
| GMPPA | ENSG00000144591.20 | | A5SS | 0 | 0 |
| GNB1 | ENSG00000078369.18 | | MXE | 0 | 0 |
| GNE | ENSG00000159921.20 | | SE | 0 | 0 |
| GOSR2 | ENSG00000108433.17 | | MXE | 0 | 0 |
| GPD1L | ENSG00000152642.11 | | SE | 0 | 0 |
| GPHN | ENSG00000171723.16 | | SE | 1 | 1 |
| GRIN1 | ENSG00000176884.16 | Up-regulated | SE | 1 | 0 |
| | | upon PTBP2 KD | | | |
| GRIP1 | ENSG00000155974.13 | Down-regulated | SE | 0 | 0 |
| | | upon PTBP2 KD | | | |
| GTF2I | ENSG00000263001.7 | | SE | 0 | 0 |
| GTF2IRD1 | ENSG00000006704.11 | | SE | 0 | 0 |
| GTF2IRD1 | ENSG00000006704.11 | | SE | 0 | 1.04 |
| GUCY1A1 | ENSG00000164116.17 | | SE | 0 | 0 |
| GUCY1A1 | ENSG00000164116.17 | | A3SS | 0 | 0 |
| GUCY1A1 | ENSG00000164116.17 | | MXE | 0 | 0 |
| HDAC4 | ENSG00000068024.17 | | SE | 0 | 0 |
| HNRNPA1 | ENSG00000135486.19 | Down-regulated | MXE | 0 | 0 |
| | | upon PTBP2 KD | | | |
| HNRNPDL | ENSG00000152795.18 | | SE | 0 | 0 |
| HNRNPDL | ENSG00000152795.18 | | A3SS | 0 | 0 |
| HNRNPDL | ENSG00000152795.18 | | MXE | 0 | 0.96 |
| HNRNPU | ENSG00000153187.20 | | SE | 0 | 0 |
| HPS5 | ENSG00000110756.18 | | SE | 0 | 0 |
| HSPA9 | ENSG00000113013.16 | | RI | 0 | 8 |
| HTRA2 | ENSG00000115317.12 | | A5SS | 0 | 0 |
| IARS1 | ENSG00000196305.19 | | SE | 0 | 0 |
| IARS1 | ENSG00000196305.19 | | A5SS | 0 | 0 |
| IDH1 | ENSG00000138413.14 | | MXE | 0 | 0 |
| IDH3B | ENSG00000101365.21 | | SE | 4 | 5.26 |
| IDH3B | ENSG00000101365.21 | | A3SS | 4 | 5.26 |
| IDH3B | ENSG00000101365.21 | | MXE | 4 | 5.26 |
| IFNAR2 | ENSG00000159110.21 | | SE | 0 | 0 |
| IFT122 | ENSG00000163913.13 | | RI | 0 | 0 |
| IFT172 | ENSG00000138002.16 | | SE | 0 | 0 |
| IQSEC2 | ENSG00000124313.18 | Up-regulated | SE | 2 | 0 |
| | | upon PTBP2 KD | | | |
| IRF7 | ENSG00000185507.21 | | A3SS | 0 | 0 |
| KAT6A | ENSG00000083168.11 | | SE | 0 | 0 |
| KAT6B | ENSG00000156650.14 | | SE | 0 | 0.06 |
| KAT8 | ENSG00000103510.20 | | RI | 0 | 0 |
| KCNAB2 | ENSG00000069424.15 | | SE | 0 | 0 |
| KCTD17 | ENSG00000100379.17 | | SE | 0.28 | 0 |
| KCTD17 | ENSG00000100379.17 | | MXE | 0.28 | 0 |
| KCTD17 | ENSG00000100379.17 | | RI | 0.28 | 0 |
| KDM1A | ENSG00000004487.17 | | SE | 0 | 3.68 |
| KDM4B | ENSG00000127663.15 | | SE | 0 | 0 |
| KIAA0753 | ENSG00000198920.11 | | SE | 0 | 0 |
| KIF15 | ENSG00000163808.17 | Down-regulated | SE | 0 | 0 |
| | | upon PTBP2 KD | | | |
| KIF15 | ENSG00000163808.17 | Down-regulated | MXE | 0 | 0 |
| | | upon PTBP2 KD | | | |
| KIF1B | ENSG00000054523.19 | | A3SS | 3 | 2 |
| KIF1B | ENSG00000054523.19 | | MXE | 3 | 2 |
| KIF21A | ENSG00000139116.19 | | SE | 0 | 0 |
| KIF21A | ENSG00000139116.19 | | SE | 0 | 0 |
| KIF21A | ENSG00000139116.19 | | MXE | 0 | 0 |
| KMT2B | ENSG00000272333.7 | | A3SS | 0 | 0 |
| KMT2E | ENSG00000005483.21 | | SE | 0 | 0 |
| KMT2E | ENSG00000005483.21 | | SE | 0 | 0.3 |
| KRIT1 | ENSG00000001631.16 | Down-regulated | SE | 0 | 0 |
| | | upon PTBP2 KD | | | |
| LARP7 | ENSG00000174720.16 | Down-regulated | SE | 0 | 0 |
| | | upon PTBP2 KD | | | |
| LARS1 | ENSG00000133706.19 | | RI | 0 | 0 |
| LEMD2 | ENSG00000161904.12 | | SE | 1 | 4.44 |
| LMAN2L | ENSG00000114988.12 | | MXE | 0 | 0 |
| LTBP4 | ENSG00000090006.18 | | SE | 0 | 2 |
| LTBP4 | ENSG00000090006.18 | | A5SS | 0 | 2 |
| LTBP4 | ENSG00000090006.18 | | MXE | 0 | 2 |
| MACF1 | ENSG00000127603.31 | | SE | 0 | 0 |
| MACF1 | ENSG00000127603.31 | | A5SS | 0 | 0 |
| MACROH2A1 | ENSG00000113648.16 | | A5SS | 0 | 0 |
| MADD | ENSG00000110514.19 | Up-regulated | SE | 0 | 0 |
| | | upon PTBP2 KD | | | |
| MANBA | ENSG00000109323.11 | | SE | 0 | 0 |
| MAP2K1 | ENSG00000169032.10 | | SE | 0 | 0 |
| MAPK10 | ENSG00000109339.24 | | SE | 0 | 0 |
| MAPKBP1 | ENSG00000137802.14 | | SE | 2 | 0 |
| MAPKBP1 | ENSG00000137802.14 | | A5SS | 0 | 0 |
| MARCHF6 | ENSG00000145495.16 | | SE | 0 | 0 |
| MARS1 | ENSG00000166986.15 | | A3SS | 0 | 0 |
| MARS1 | ENSG00000166986.15 | | MXE | 0 | 0 |
| MASP1 | ENSG00000127241.18 | | SE | 0 | 2 |
| MBD5 | ENSG00000204406.14 | | A5SS | 0 | 0 |
| MDM2 | ENSG00000135679.26 | Up-regulated | SE | 0 | 0 |
| | | upon PTBP2 KD | | | |
| MDM2 | ENSG00000135679.26 | Up-regulated | SE | 0 | 0 |
| | | upon PTBP2 KD | | | |
| MDM2 | ENSG00000135679.26 | Up-regulated | SE | 0 | 2 |
| | | upon PTBP2 KD | | | |
| MDM2 | ENSG00000135679.26 | Up-regulated | MXE | 0 | 0 |
| | | upon PTBP2 KD | | | |
| MEF2A | ENSG00000068305.17 | Down-regulated | SE | 0 | 0 |
| | | upon PTBP2 KD | | | |
| MEG3 | ENSG00000214548.18 | Up-regulated | SE | 2 | 0 |
| | | upon PTBP2 KD | | | |
| METTL5 | ENSG00000138382.15 | | SE | 0 | 0 |
| METTL5 | ENSG00000138382.15 | | SE | 0 | 0 |
| METTL5 | ENSG00000138382.15 | | MXE | 0 | 0 |
| MFN2 | ENSG00000116688.18 | | SE | 0 | 1.06 |
| MLLT10 | ENSG00000078403.17 | Down-regulated | SE | 0 | 0 |
| | | upon PTBP2 KD | | | |
| MLX | ENSG00000108788.12 | | RI | 0 | 0 |
| MRE11 | ENSG00000020922.13 | Down-regulated | SE | 0 | 0 |
| | | upon PTBP2 KD | | | |
| MRPS7 | ENSG00000125445.11 | | RI | 0 | 0 |
| MSMO1 | ENSG00000052802.13 | Up-regulated | SE | 0 | 2 |
| | | upon PTBP2 KD | | | |
| MTMR14 | ENSG00000163719.20 | | SE | 0 | 0 |
| MTRR | ENSG00000124275.15 | | SE | 0 | 0 |
| MVD | ENSG00000167508.12 | Up-regulated | SE | 0 | 8.24 |
| | | upon PTBP2 KD | | | |
| MVD | ENSG00000167508.12 | Up-regulated | MXE | 0 | 8.24 |
| | | upon PTBP2 KD | | | |
| MYSM1 | ENSG00000162601.11 | | SE | 0 | 0 |
| NADK2 | ENSG00000152620.13 | | RI | 0 | 0 |
| NDUFA10 | ENSG00000130414.13 | | SE | 0 | 0 |
| NDUFS2 | ENSG00000158864.13 | | A3SS | 0 | 0 |
| NDUFV1 | ENSG00000167792.13 | | SE | 0 | 0 |
| NEPRO | ENSG00000163608.15 | | SE | 0 | 0 |
| NGLY1 | ENSG00000151092.18 | | SE | 0 | 0 |
| NGLY1 | ENSG00000151092.18 | | SE | 0 | 0 |
| NIPA2 | ENSG00000140157.16 | | SE | 0 | 0 |
| NIPBL | ENSG00000164190.19 | | SE | 0 | 0 |
| NIPBL | ENSG00000164190.19 | | MXE | 0 | 0 |
| NOTCH2 | ENSG00000134250.20 | | SE | 0 | 0 |
| NPM1 | ENSG00000181163.14 | Down-regulated | RI | 0 | 0 |
| | | upon PTBP2 KD | | | |
| NSUN2 | ENSG00000037474.15 | | SE | 0 | 0 |
| NT5C2 | ENSG00000076685.19 | | SE | 0 | 0 |
| NUBPL | ENSG00000151413.17 | | SE | 0 | 0 |
| NUMA1 | ENSG00000137497.18 | | RI | 0 | 0 |
| NUP160 | ENSG00000030066.13 | | SE | 0 | 0 |
| NUP62 | ENSG00000213024.12 | | SE | 0 | 5 |
| OBSL1 | ENSG00000124006.15 | | SE | 0 | 0 |
| ORC1 | ENSG00000085840.13 | Down-regulated | SE | 0 | 0 |
| | | upon PTBP2 KD | | | |
| ORC4 | ENSG00000115947.14 | | SE | 0 | 0 |
| PAK3 | ENSG00000077264.16 | | SE | 0 | 0 |
| PALLD | ENSG00000129116.19 | | SE | 0 | 1 |
| PALLD | ENSG00000129116.19 | | MXE | 0 | 1 |
| PBRM1 | ENSG00000163939.18 | | SE | 0 | 5.64 |
| PBX1 | ENSG00000185630.19 | | SE | 0 | 0 |
| PBX1 | ENSG00000185630.19 | | MXE | 0 | 0 |
| PC | ENSG00000173599.15 | Up-regulated | SE | 0 | 0 |
| | | upon PTBP2 KD | | | |
| PC | ENSG00000173599.15 | Up-regulated | MXE | 0 | 0 |
| | | upon PTBP2 KD | | | |
| PCLO | ENSG00000186472.20 | | SE | 0 | 0 |
| PCM1 | ENSG00000078674.18 | | SE | 1.3 | 1.3 |
| PCM1 | ENSG00000078674.18 | | SE | 0 | 0 |
| PCM1 | ENSG00000078674.18 | | A3SS | 0 | 0 |
| PDE10A | ENSG00000112541.18 | Down-regulated | MXE | 0 | 0 |
| | | upon PTBP2 KD | | | |
| PDE2A | ENSG00000186642.16 | | SE | 0 | 0 |
| PER3 | ENSG00000049246.15 | | SE | 0 | 0 |
| PEX1 | ENSG00000127980.16 | | A3SS | 0 | 0 |
| PGAP1 | ENSG00000197121.15 | | SE | 0 | 0.46 |
| PHF21A | ENSG00000135365.16 | | SE | 0 | 0 |
| PHIP | ENSG00000146247.14 | Down-regulated | SE | 0 | 0 |
| | | upon PTBP2 KD | | | |
| PICALM | ENSG00000073921.18 | | SE | 0 | 0 |
| PICALM | ENSG00000073921.18 | | MXE | 1 | 1.96 |
| PICK1 | ENSG00000100151.16 | | SE | 0 | 0 |
| PICK1 | ENSG00000100151.16 | | A3SS | 0 | 0 |
| PIK3C2A | ENSG00000011405.13 | | SE | 0 | 0 |
| PIKFYVE | ENSG00000115020.17 | | SE | 0 | 1 |
| PIKFYVE | ENSG00000115020.17 | | SE | 0 | 0 |
| PIKFYVE | ENSG00000115020.17 | | SE | 0 | 0 |
| PIP5K1C | ENSG00000186111.11 | | A3SS | 0 | 0 |
| PLD3 | ENSG00000105223.20 | | SE | 5.24 | 3.24 |
| PLEKHG5 | ENSG00000171680.23 | Up-regulated | A3SS | 0 | 0 |
| | | upon PTBP2 KD | | | |
| PMS1 | ENSG00000064933.18 | Down-regulated | SE | 0 | 0 |
| | | upon PTBP2 KD | | | |
| PNKP | ENSG00000039650.12 | | A5SS | 0 | 0 |
| POLR1C | ENSG00000171453.20 | Down-regulated | A3SS | 0 | 0 |
| | | upon PTBP2 KD | | | |
| POMGNT2 | ENSG00000144647.6 | | SE | 0 | 0 |
| POMT1 | ENSG00000130714.19 | | SE | 0 | 0 |
| PREPL | ENSG00000138078.16 | | SE | 0 | 0 |
| PRKCG | ENSG00000126583.12 | Up-regulated | SE | 8.48 | 1 |
| | | upon PTBP2 KD | | | |
| PRPF31 | ENSG00000105618.14 | | SE | 0 | 0 |
| PSAT1 | ENSG00000135069.14 | | SE | 0 | 2 |
| PSAT1 | ENSG00000135069.14 | | MXE | 0 | 2 |
| PTPRF | ENSG00000142949.17 | | SE | 4.26 | 3.26 |
| PTPRF | ENSG00000142949.17 | | SE | 0 | 0 |
| PTS | ENSG00000150787.8 | | SE | 0 | 0 |
| PXK | ENSG00000168297.16 | | SE | 1.42 | 0 |
| QARS1 | ENSG00000172053.18 | Down-regulated | A5SS | 0 | 0 |
| | | upon PTBP2 KD | | | |
| RAB18 | ENSG00000099246.18 | | SE | 0 | 1 |
| RAB3GAP1 | ENSG00000115839.18 | | SE | 0 | 0 |
| RAD51C | ENSG00000108384.15 | | SE | 0 | 0 |
| RAD51D | ENSG00000185379.21 | | SE | 0 | 0 |
| RANBP2 | ENSG00000153201.16 | | SE | 0 | 0 |
| RBM10 | ENSG00000182872.16 | Up-regulated | RI | 0 | 0 |
| | | upon PTBP2 KD | | | |
| RBPJ | ENSG00000168214.21 | | SE | 0 | 2 |
| RBSN | ENSG00000131381.12 | | SE | 0 | 0 |
| RMND1 | ENSG00000155906.20 | | SE | 0 | 0 |
| RMND1 | ENSG00000155906.20 | | MXE | 0 | 0 |
| RNF13 | ENSG00000082996.20 | | SE | 0 | 0 |
| RPGRIP1L | ENSG00000103494.16 | Down-regulated | SE | 0 | 0 |
| | | upon PTBP2 KD | | | |
| RPL21 | ENSG00000122026.11 | Down-regulated | MXE | 0 | 1 |
| | | upon PTBP2 KD | | | |
| SACS | ENSG00000151835.17 | Down-regulated | SE | 0 | 0 |
| | | upon PTBP2 KD | | | |
| SAMHD1 | ENSG00000101347.11 | | RI | 0 | 0 |
| SBF1 | ENSG00000100241.21 | | SE | 0 | 4 |
| SBF1 | ENSG00000100241.21 | | A3SS | 0 | 0 |
| SBF2 | ENSG00000133812.17 | | SE | 0 | 0 |
| SBF2 | ENSG00000133812.17 | | MXE | 0 | 0 |
| SGCE | ENSG00000127990.19 | | SE | 0 | 0 |
| SIRT6 | ENSG00000077463.15 | | SE | 0 | 0 |
| SIRT6 | ENSG00000077463.15 | | RI | 0 | 0 |
| SLC37A4 | ENSG00000137700.18 | Down-regulated | SE | 0 | 0 |
| | | upon PTBP2 KD | | | |
| SLC37A4 | ENSG00000137700.18 | Down-regulated | MXE | 0 | 0 |
| | | upon PTBP2 KD | | | |
| SLC39A13 | ENSG00000165915.14 | Down-regulated | SE | 0 | 0.02 |
| | | upon PTBP2 KD | | | |
| SMAD2 | ENSG00000175387.16 | | SE | 0 | 0 |
| SMARCC2 | ENSG00000139613.12 | | SE | 0 | 0 |
| SMARCC2 | ENSG00000139613.12 | | A3SS | 0 | 0 |
| SMARCE1 | ENSG00000073584.20 | | MXE | 0 | 0 |
| SMO | ENSG00000128602.11 | Down-regulated | SE | 0 | 0 |
| | | upon PTBP2 KD | | | |
| SNRPN | ENSG00000128739.23 | | SE | 0 | 0 |
| SNRPN | ENSG00000128739.23 | | MXE | 0 | 0 |
| SRC | ENSG00000197122.12 | | SE | 0 | 0 |
| SRCAP | ENSG00000080603.17 | | SE | 0 | 10.8 |
| SRGAP3 | ENSG00000196220.16 | | MXE | 3.22 | 4.22 |
| SSBP1 | ENSG00000106028.11 | | RI | 0 | 0 |
| SSBP1 | ENSG00000106028.11 | | RI | 0 | 0 |
| STAG2 | ENSG00000101972.19 | Down-regulated | SE | 0 | 0 |
| | | upon PTBP2 KD | | | |
| STT3B | ENSG00000163527.10 | | SE | 0 | 0 |
| SYNE1 | ENSG00000131018.25 | | SE | 0 | 0 |
| SYNE2 | ENSG00000054654.19 | Down-regulated | SE | 0 | 2.42 |
| | | upon PTBP2 KD | | | |
| SYNE2 | ENSG00000054654.19 | Down-regulated | SE | 0 | 0 |
| | | upon PTBP2 KD | | | |
| SYNGAP1 | ENSG00000197283.18 | Up-regulated | SE | 0 | 0 |
| | | upon PTBP2 KD | | | |
| SYNGAP1 | ENSG00000197283.18 | Up-regulated | A3SS | 4.66 | 3 |
| | | upon PTBP2 KD | | | |
| TBCD | ENSG00000141556.22 | | SE | 0 | 0 |
| TCF7L2 | ENSG00000148737.17 | | MXE | 0 | 1 |
| TGFBR1 | ENSG00000106799.13 | | SE | 0 | 1 |
| TIA1 | ENSG00000116001.17 | Down-regulated | SE | 0 | 0 |
| | | upon PTBP2 KD | | | |
| TK2 | ENSG00000166548.17 | | RI | 0 | 0 |
| TMCO1 | ENSG00000143183.18 | | SE | 0 | 0 |
| TMEM126A | ENSG00000171202.7 | | SE | 0 | 0 |
| TMEM126B | ENSG00000171204.13 | | SE | 0 | 0 |
| TMEM165 | ENSG00000134851.13 | | MXE | 0 | 0 |
| TMEM38B | ENSG00000095209.12 | | SE | 0 | 0 |
| TMTC3 | ENSG00000139324.12 | Down-regulated | SE | 0 | 0 |
| | | upon PTBP2 KD | | | |
| TNK2 | ENSG00000061938.21 | | SE | 5.02 | 0 |
| TNK2 | ENSG00000061938.21 | | RI | 0 | 0 |
| TPM1 | ENSG00000140416.23 | | MXE | 0 | 3 |
| TPM1 | ENSG00000140416.23 | | RI | 0 | 0 |
| TPM1 | ENSG00000140416.23 | | RI | 0 | 1 |
| TPM3 | ENSG00000143549.21 | | SE | 2 | 2 |
| TPM3 | ENSG00000143549.21 | | SE | 1 | 8.68 |
| TPM3 | ENSG00000143549.21 | | MXE | 1 | 8.68 |
| TPM4 | ENSG00000167460.17 | | SE | 0 | 0 |
| TPMT | ENSG00000137364.5 | | SE | 0 | 0 |
| TRAF3 | ENSG00000131323.16 | | SE | 0 | 0 |
| TRAPPC11 | ENSG00000168538.16 | Down-regulated | SE | 0 | 0 |
| | | upon PTBP2 KD | | | |
| TRAPPC4 | ENSG00000196655.12 | | SE | 0 | 0 |
| TRAPPC4 | ENSG00000196655.12 | | A5SS | 0 | 0 |
| TRIM2 | ENSG00000109654.16 | | SE | 0 | 2 |
| TRIM2 | ENSG00000109654.16 | | MXE | 0 | 2 |
| TRMU | ENSG00000100416.15 | | SE | 0 | 0 |
| TRPM7 | ENSG00000092439.16 | | SE | 0 | 0 |
| TSC2 | ENSG00000103197.18 | | SE | 0 | 1 |
| UBA5 | ENSG00000081307.15 | | SE | 0 | 5.04 |
| UBE3A | ENSG00000114062.22 | | SE | 0 | 2 |
| UBE3A | ENSG00000114062.22 | | A3SS | 0 | 0 |
| UBE3A | ENSG00000114062.22 | | MXE | 0 | 2 |
| UMPS | ENSG00000114491.14 | Down-regulated | SE | 0 | 0 |
| | | upon PTBP2 KD | | | |
| UMPS | ENSG00000114491.14 | Down-regulated | A5SS | 0 | 0 |
| | | upon PTBP2 KD | | | |
| UNC13A | ENSG00000130477.16 | Up-regulated | SE | 0 | 0 |
| | | upon PTBP2 KD | | | |
| UROS | ENSG00000188690.15 | | SE | 0 | 0 |
| USB1 | ENSG00000103005.12 | Up-regulated | SE | 0 | 0 |
| | | upon PTBP2 KD | | | |
| USP7 | ENSG00000187555.16 | | A3SS | 0 | 0 |
| VAPB | ENSG00000124164.16 | | SE | 0 | 0 |
| VPS13B | ENSG00000132549.20 | Down-regulated | SE | 0 | 0 |
| | | upon PTBP2 KD | | | |
| VPS4B | ENSG00000119541.10 | | SE | 0 | 0 |
| WASHC4 | ENSG00000136051.15 | | SE | 0 | 0 |
| WDR35 | ENSG00000118965.16 | | SE | 0 | 0 |
| WDR48 | ENSG00000114742.14 | | SE | 0 | 0 |
| WNK1 | ENSG00000060237.19 | | SE | 0 | 1 |
| WNK1 | ENSG00000060237.19 | | SE | 0 | 0 |
| WNK1 | ENSG00000060237.19 | | MXE | 0 | 1 |
| XPA | ENSG00000136936.11 | | SE | 0 | 0 |
| XPO1 | ENSG00000082898.19 | | SE | 1 | 2 |
| XPO1 | ENSG00000082898.19 | | SE | 0 | 0 |
| XPO1 | ENSG00000082898.19 | | A3SS | 1 | 2 |
| XPO1 | ENSG00000082898.19 | | MXE | 0 | 0 |
| XPO1 | ENSG00000082898.19 | | RI | 0 | 0 |
| YME1L1 | ENSG00000136758.19 | | SE | 0 | 0 |
| YY1AP1 | ENSG00000163374.20 | | MXE | 0 | 0 |
| ZC3H14 | ENSG00000100722.20 | | SE | 0 | 0 |
| ZC3H14 | ENSG00000100722.20 | | MXE | 0 | 0 |
| ZMPSTE24 | ENSG00000084073.10 | Down-regulated | SE | 0 | 0 |
| | | upon PTBP2 KD | | | |
| ZNF513 | ENSG00000163795.14 | | MXE | 0 | 0 |

All patents, patent applications, and publications, references to GenBank or another publicly available sequences database cited throughout the disclosure, and U.S. provisional patent application No. 63/341,734, filed May 13, 2022, are expressly incorporated herein by reference in its entirety. While this invention has been disclosed with reference to specific embodiments, it is apparent that other embodiments and variations of this invention are devised by others skilled in the art without departing from the true spirit and scope of the invention. The appended claims include such embodiments and equivalent variations.

1. A therapeutic composition comprising at least one agent which specifically interferes with PTBP2-binding in the SYNGAP1 gene region, thereby preventing an alternative splicing event which cause dysfunction protein production, which dysfunctional protein production associated with a SYNGAP1 disease or disorder.

2. The therapeutic composition of claim 1, wherein the agent is an anti-sense oligonucleotide, an RNAi, siRNA, or combinations thereof.

3. The therapeutic composition of claim 1, wherein the agent is delivered via a viral vector which is a recombinant parvovirus, a recombinant lentivirus, or non-viral vector.

4. The therapeutic composition of claim 1, wherein a non-viral vector comprises the at least one agent(s).

5. The therapeutic composition of claim 4, wherein the non-viral vector is a lipid nanoparticle, lipidoid, or liposome.

6. The therapeutic composition of claim 1, wherein the at least one agent comprises an antisense oligonucleotide 15 to 30 nucleotides in length comprising at least 15 consecutive nucleotides of a sequence comprising:
(a) SSO_085: TCCAGGGAACATGCTGAG (SEQ ID NO: 1), a sequence at least 99% identical to SEQ ID NO: 1, a sequence having at least 95% complementarity to SEQ ID NO: 1, or a pharmaceutically acceptable salt thereof, or combinations thereof;
(b) SSO_019: CACGTGGGAGAGAGATGG (SEQ ID NO: 2), a sequence at least 99% identical to SEQ ID NO: 2, or a pharmaceutically acceptable salt thereof, or combinations thereof;
(c) SSO_061: CTTCCAGGGAACATGCTG (SEQ ID NO: 3), a sequence at least 99% identical to SEQ ID NO: 3, or a pharmaceutically acceptable salt thereof, or combinations thereof;
(d) SSO_086: TTCCAGGGAACATGCTGA (SEQ ID NO: 3), a sequence at least 99% identical to SEQ ID NO: 4, or a pharmaceutically acceptable salt thereof, or combinations thereof;
(e) a sequence comprising a sequence having at least 95% complementarity to SEQ ID NO: 1, 2, 3 or 4, or a sequence comprising at least 15 consecutive nucleotides of SEQ ID NO: 1, 2, 3, or 4, or a pharmaceutically acceptable salt thereof, or combinations thereof, or
(f) combinations of (a), (b), (c), (d) or (e).

7. The therapeutic composition of claim 6, wherein the agent composition comprises (a) and/or (b).

8. The therapeutic composition of claim 1, wherein said composition comprises an antisense oligonucleotide having 100% complementarity to one of SEQ ID NO: 1 or an antisense oligonucleotide having 100% complementarity to one of SEQ ID NO: 2.

9. The therapeutic composition of claim 1, wherein said composition comprises an antisense oligonucleotide of SEQ ID NO: 1 and/or SEQ ID NO: 2. has at least 100% complementarity to SEQ ID NO: 2.

10. The therapeutic composition of any claim 1, wherein the agent is an antisense oligonucleotide having at least one modified internucleoside linkage, sugar moiety, or nucleobase.

11. The therapeutic composition of claim 1 wherein the agent is a chimeric oligonucleotide having a gap segment positioned between 5′ and 3′ wing segments.

12. The therapeutic composition of claim 11, wherein the gap segment of the chimeric oligonucleotide is comprised of 2′-deoxynucleotides and the wing segments are comprised of nucleotides having modified sugar moieties.

13. The therapeutic composition of claim 12, wherein the modified sugar moiety is 2′-OMe or a bicyclic nucleic acid.

14. The therapeutic composition of claim 11, wherein the gap segment of the chimeric oligonucleotide consists of ten 2′-deoxynucleotides and each wing segment consists of five 2′-O-methoxyethyl-modified nucleotides.

15. The therapeutic composition of claim 1, wherein the at least one agent is at least one antisense oligonucleotide of 18 nucleotides in length.

16. A method useful for treating a patient having dysfunctional SYGAP protein production associated with a SYNGAP1 disease or disorder comprising delivering a therapeutically effective amount of a composition according to claim 1.

17. (canceled)

18. The method of claim 16, wherein the composition further comprises a pharmaceutically acceptable aqueous diluent suitable for intrathecal injection.


SEQUENCE LISTING (92 sequences)

SEQ ID NO: 1 - DNA, 18 bp, synthetic construct
  misc_feature 1..18: note = SSO_085
  source 1..18: mol_type = other DNA
tccagggaacatgctgag

SEQ ID NO: 2 - DNA, 18 bp, synthetic construct
  misc_feature 1..18: note = SSO_019
  source 1..18: mol_type = other DNA
cacgtgggagagagatgg

SEQ ID NO: 3 - DNA, 18 bp, synthetic construct
  misc_feature 1..18: note = SSO_061
  source 1..18: mol_type = other DNA
cttccagggaacatgctg

SEQ ID NO: 4 - DNA, 18 bp, synthetic construct
  misc_feature 1..18: note = SSO_086
  source 1..18: mol_type = other DNA
ttccagggaacatgctga

SEQ ID NO: 5 - DNA, 35523 bp, Homo sapiens
  source 1..35523: mol_type = other DNA
ccaatcccgaccctatcatggcgcggtttgcccacctcctgatgaaggaggagccatgggagacttgaccaatcgactaccgcatctgcagacgtgcgcggaaatggcacgcccgtttccgtgtgaacccgaaactgccacttgctggctgaggagagccgctgcggcgttccactgtcacgtgagggggccggtcctcttcggcggcggcactcagtgagtgacgccaagtggccaatcacaggccagcctcgcaggccacgtgaggcgagagctggttccaggaaaggggggccagagaccccgcagagtcgatgcgtcaggctgtggagtggggaatcatccttaaaccccacaatgtgcctagtgacctcccctgcactgtttccgaggtcttaaggagtgggagggtctgagaaggacccttcaccccaaactcaggggttaagagagcgccccctgcccttttagttgggcagagatatgcctgggtttgggaatgaacaacggtgatctagaagcccgctgtccctgggtagcagggagtgattaggcaggcacgggcatggcaagacaatagctagctgaggaaagaagcattttaaaacagagcgagaccctgtggtagaggatggtgaaaatggggcctagccatcttaattgaaaacccaaagcttgagacagactactgaatcagagggaaggagaacgagggctgcaggatggggacagtggtgatggtccttctgtcccctcttccctgcctattccactccccaggattggtccacctctcagcctcgggtctcctttccctaacccatcccccaaggagccaggacctgccctacataacttccctggggcccaagcacatcctgtgacccaaaaatggagagaggggccaatgagaggcagagaggtgggaggaggtgcagagaaaaaagctcttgccactgcccccacccaatatttcccctcccttcaaggtctgaggtcattggtctggtggtggggatgcctcggagtttcccaggctgacgtcagggttatacctctcggaggttctagggcagggaagtggggaggggaggttccctgagatctctgtctttgagtttggagtttgtctctgagacagatttcggaggggtctttcagtatttgtgtggagatctttcttaggattgagtttccttttatgcttctggtttggggggtttctatgtttcagggtctgtggtcgcactctgaggtctttcgaagtcttcttcatgaattttgggtctctgagttgtccatctccggaccagctgaagtctctatctccccttaagtcaggagtgtctctgtctctaagttatcagtctctgagatctgagtctggggtctctttctaggtctgagttctctaagctctaattctacatctggctctttctgaatctgggcctcatctttttctgagtctaggtctcaggtcctggtccctgaaatcatcatctcagggtctggagtctaagatatctgtaggtatggagaaccctttttagactggattttcagattttgatattgagcttctctctctgggggatctggggtcgttttttcctcaaatcaggagtctcttttcctctagattttggccctgtgtctcaaattaccccacagtggggggtggtgaagtatctttctctgtgggtctagggtctctctgtctcagggtctggggtctgcatctttaggacctctgtctctctctggtgtgtttttgagtcaggggtctctctctctctcacaatctgggcctcccggagtaggggtgggggctgcagagtctctccctcctcctcctcctcctgctctcttcgctctcgctcgctcccccgccccccctctctctcggctgccgctgctgccgttggctcttattctcctcctcctcctcctctctcctcctctctgcttctctctgctcctctctcctcctctctcctcctcctcctcctccacctcctcctccttctccccctctttctccccctctttctctcttctttctcccccgtccccccgccccctccccccaggcctgatgagcaggtctcgagcctccatccatcgggggagcatccccgcgatgtcctatgcccccttcagaggtacgtggtggggggagggggagggccatatggggggcaacagggggaggggcagggggcgggggagagtcggggccgagggaagggagcggctgaaatggagggagaggggaggaccgggaaggcaggggtgggagcaaccgggaaggagggattggaggccagagggataggggtagataagggatggaggggccaggaaatgggccttgaaagggggctgaagggaaaatgatgaaggggaaagaaagcgccagaaaggggtgggaaggtagaaggattgggttggggaaaaagctgcagaaataatttgggggtgggagctgagtgatgaggaaaacatcaaggaaatagagggacagagtcttgggagaggggctaggaaggagaaatgaaaacataggggtgaaaagagaagccaagaagatgtggggggataatttggtgggggactcagagtatggtaaggggcggaaaaagagaggaggatggagggagagaaggggccagatggagagagagggaggcagtggggggtggggggatggagggtccagatggagggaagagagaagagagagggaacggtgggggaggggagaccagggcagggggagggccaaattatgtgtgcttgggggggtggcagtacaagtaagatgaaatgatgggagtaggggacaggttaggatcctgccccagactctgaccctcttaaaggctcttgaccagtaaaatgtaacctctggggtcttaatgctgtcttcacttcatcacttttacccccttcccccacccctccaatccttatttccattccccctccctcctctgctgtccatcacctatatctttccccccatatggtgtccgttccttatatgagtcccccttctgtccttcccctatatcagtcccctctctggtgttcattgcctctctcttatctcttttctatgtggtcgctcccttatatctctatcacttctttactagtccgtcttgaccccttctcatagcctccgcattaggtctgcctcctttgctctgacacatgtccagtcgagcccttcaggtgtattcccttgcccctgggcatttaccatcatttctgcctccttttctatctactcctattatttgtctcccagaccattccttgtcaccacaagtctgtgcctacccactttcccttaatacctcaactcttattatcatttctcttttcttgtctcctctacttattggggtgttggagatttggggtacaaagacacaggttttgatgattagaacaagggatccccatctcagcccttgagggatttctcatcctagtaaaaatttcccgtttctcagccatgttgctctgcctcttgcatctgcctcccagaatcattcttcaccaagtccagacagtatgtgtgtgtgtgtttctagagttgttcctaagttccctgctgccaggagaaagatgccctctgaagtctaacccgcctcccttggctgcagcctgaaccagccctttccccaaatgcttatgtgtatcaacagatttctcctctgggtataccaagtgtctaagtgcggtgggcatgttgcctgcaatgtctgtagtttcagaggctaggtattggtgatctctgtatctagagaacattagttagtctctgagatgggttgagatagagtttcaaagtatgtggtttctaaatggtggacattctgagggttcatcgctcactgtgatctggaggttgggtaaatttgagagacatatttatagacagaaaagggctgaatttgcaaaactctgctactcagtgcattctgttggaatgaagctcaaagaccagttctcctgcagtatactcatggttatcccataaaggtgacagagtttgatcttttttccatctaaaatctgataccttaagcctaatttctagctctttgctgtcgccaatgtgacatctctcttacttcaatagggcctgtgtggcattggggtttaaaggagaggcagtttgcagggagagcggcatggcaagaacaacagagctggggaaggaggaccctggtctcatctcagccataggcgacacttggtctactgttctctcttctcaaagttttgttcccatctccagctcttgtctttggaggcccctctgtgtcaccaggcctggatattccattctttccccctgacatctttcttcctcaagctcctcactgtgccttgagaaggctcctgacctatggagtggcttccatcctgtccactgagactaggtggggtgaggtggtgggggcagtgttgaatctctgggcactgaatgggagaagagataaatttggtctctacttgtccaaacaagggcatatttcccaccttctgccctggggcctccacagccagatgccgacaaaagccatcttctccttaccaccagaagaaccagaactttccctaaaggatgagcagggttgaactaggggaggagggcacagaggagggaagagttgggaggggaggccctctcatgtctctgactgtgaacaggtaagctccccgacttcttcttttctagatatttagccctcagaatctcatgatcccaatgttcctacacacacaccccatccttctttcccacagccatgtagctttcctcatactcttttatagaggggtggtgccaccttccaagtccttgtcttctgcagctatttggatgccttcctctacccagatatctgacaccaacttctctgtttttctcctgccaattttgcggcacctctaccttattttcctgaagtagatggagagctccttcatctttctctgcccccaaacgtgtaccttctctgtcatataattcccatccctgaacctctgcatttcttcccccaagtctccagagccttctgtgatgtgatcctctcctcttttcccctttgtataaatgcttgtcttgtgttcctttagagttgccaggtgtctcactttcaactgtgatctcctgaaggccggtagggtccccttctcactttgtggactctcttcttgccattttaggcctctgcttccaaatgcatagagcctcccttactgtttctgtgtgtctctgtcctccagatgtacggggaccctctatgcaccgaacccaatacgttcattccccgtatgatcgtcctggttggaaccctcggttctgcatcatctcggggaaccagctgctcatgctggatgaggatgaggtgagtgtggtgagaaggctgggaaaacgcatgtgggagaatggggagagtcctcattagtgaaggggagagagacacaaagaaggcagacaaagaagaaacatttgcaaatggcaggagggtggccattgagacttgggttggagtgtgacaagaggggtagtcattctctcatcttgggcagggagtctttgagggttatgagctgtgttggggaggagatgtggttctgaagatttcgggggtggggaggctcctgttttcacactgtccatattgtaggataggttagaatggggaaggggaactgaagggtttggagaaggggtgaagacatggaaggccctcccaaccaaagcaatcctcaggacccctctctgctttgcccactgagcaagactcccagtcctttctccatcagggggcagagtgcaagaagagaaattgcaggacgcagagaaaaggcaggggatagaggaggttttaggtagctggaggatactgggaaagaagactgaggtcagtggatcttggcatgatggggtgaagactgggagagtgattgagatcaaggtttagagagacactgagagggcttctgcaagggagtggttggacatcaggaaggaatataagggaggggaactgggagtatgtggagttatctaaaggagtacaagtggagtaggggcagggactagccagagtaaaggagatgaaacggacagaggcctcttatgggaggtggtggccaggtatggagatctgagagccagggaagtgtgggttctgatttgggagctgggtcagcaggagtgtccattggaggaaatagaggataaaaactggaatggtgccccacaagtgtgagtcagggaatgggggtgatttgggggacaagctttaaaaaatggtagggtagcctggaaaaccaaacgtggtgagagatggaccctgaaagttattgggaatggtgtgatgaggaagggttagaatttgagtcagggctaactagagtgtcaagggacctatgggggcaggtgagagtgtggagagatggctgggtagacagcttccaggccacagagtaaagtagaggtgttaaggagaggatatggtgactttgaaaggagaggtggcttggggagtctaagttttggacaccattgggagacatttgagtgactgtggtcaaacccctagatcaaggtttggagtctgtaggagaaatgggggaaactggaacccagagtggggactgttatagggatgaagagggaagacaatggattggggtaatgaaggaatgacagagatagggacagactgaagggaccagtggccagcaagttgaggggcaataggaatggccggtctggagcaggaagggcgtgtgtgtgggaggaggagtagatagaattgggaaaatgagcagcctgaacagacgcagggagttggaattggatgggtggtggaactggggctgggggatagtgaggggtctgttcctgtggctctttgtagaggtggaggagactgcaggggtggaggtgtatggaggtagggatggaggtggacagggggagctgggtgtgaagcttagggaaaggtgatggctgaggggaagaagggaactgagagatgctgggtcgggtgatggaaggagatgaaaggtgggggaaggtgaccaggccctctggcgggaggggaggttgggtcccagtctggccgcaagccgggccgtgggagggagacaagctgtggatcctgattataaatgcagcttctgctacccccgtgacaggctcggagggagagaagggggtaagggaggtgggaggttgggaggaaagcagggagaatggaggaactgaaggtcaagggaaaactggattcaggaagtagctaacttctgaggagttgcagaagagctgagtagggtttgggagaattctccggaaggtgtttcaagacaaatggagggagaaaagagggtgggacggcaaagaatgagagagagagactgggggagagaagggagagatccagtcaccaagtgtggaggggccggggcagaaaaagtgggcaggcttagggggaaaaggaggcccgcccttcctgggaggaggcggaggggggaagagggaggggaggggcggcgtgggccagggaggtctgacacacccccacctcccctagatacaccccctactgatccgggaccggaggagcgagtccagtcgcaacaaactgctgagacgcacagtctccgtgccggtggaggggcggccccacggcgagcatggtacggcggccgagcaggctctcatgacccggacaggcgcggggagagggctgaagatggaccgggctggcggggcgggggcgtcggggagcggcgggagctggccgggggcgggagaaggcggagacaaagtcttgcttcccgaggggagatggcgccctcctctggaaggtggagcgggacaggggctgtgggtgagaggcccctgtgggtcctcgctcggcttcgctccctttctcctctccagcagtgaagtttgactattttcctggtgcttgtaaaaatagtctccttagatctttagcttccagtccgtgtgttcctgcccctgcccctcccagcccctggtctgggatcttggaaagaatgacctagttcctgccctccccccagacctcttccccacccctatgctcctgattcttcttccggaccttcagtccttgaggtctccttttattaccccctgttttctcctctcatcagctgcttctattttctgcaattgtatcaccctttctctttcctccctgatttctcttattttctgtacccctcttgattaccccgtccctcccctctgctcccggcgaaagtccatcagactcccatctcatctcctttctgtctgtagagctatctgcgcggtctgtctctccaactctctccctctctccatctgtctcttcccctccctccccctttctgcccccccaaccccctctttagtcatttaaaaaaagatttttgtcttcctgagaagttcgcttaaaaggtttccatgggaacagtgaggggagatgaaggcagagaacagagatctgcagccaaggtgcagagagacgccctcagcccccggaaaccccctcgtcactgcccccttccacttcatttcccttcccggacctagcgatccaggttccccattcctccaacccccaggtgacccctttttcccaccagctccccctcccttcaggaattcaagctaccagtttctctcccttagagagccagtgacctgtccccatcctttggctccttccagccttctgataggtccaggccctactagccttgtgggccggtttggggaccttgggtgggtgggtgggtggtcagggatgtgtgtcattgtcatggaaaccccataccatcggggtgtgtgggtagcacatctctgtgaggcaaagtcaggtggtggtggagtgtatacagagaatgtgtgtgatgtctgtcacatctgtgtgtatatgtatatgtgtgtatgtcatgttcatgtctggggaagatgtgtgtgtttgtgtgcatgtgtgtacacctgcgtttccaggccagaatggtcacatgctcacagagcatgtttatcatgtgtcctgtggttggcagatctgcccacttattccccatctttctccttatcctgagtcctcagggtctaggggtctctggcgggggtctcccgtgtgttttgggtggggacaccgtgtgtgtttggagttgaggtggcatgtggaggttgctgtattggggttcagtaggggctgggaggggttccaggggcatatgtatttggggggagggcttagtgaaagttgatgggggtgaacgtttagggtcttcaggcagacatggaatgtgtaaagggcatggaggatgtgcagtggacataggggtcgttgggaccttggggacttggcaatgccaaggtgtttgctgaggctgtggactctccagcccgggagaggtcgccggacctttgaggggcattggaatcctgggctcctcctctgctgggtggagccgcgaaccctcgttctctccgcggttttgtgtgtgtgggggggtcctgctcaggaggggatggtgggtggcctggtttgttttgggggtccccttgcccccctcccccgtctctctcgcgctgtctccgggcgacagggctcgtgacagaggcggccacggcagcagcggtcgcctagcaacggcggcggggcccgggcaacggcggcagcggcgggagcggcggcggagggagccgttcccgcggccgtcaccgcgcggccgtcccggccgtcccgggccccgccgccgcccccaccgcgccgggcggcgcgagccgggccgtaccgccccctctccctccgcaccccgcccttcccccgcgcaggggcggggcccctcccccaccggggacacccctggcccccccggggggcggtgcgaggtaagggcggggccgggcggactaggagcgcgcgtagaggggggggcgccgcgcgcgtggggcgggggcgcgcgtgtgcgtgggcgcggggaggggggtggggaagccgcggtggcggcggcggcggcggcagctgctccctccgcatgttctcgctgcatcttccgagtggggggagttatggtaaccggagggggacagcggaggacgggagggtcggggaccggcctcgtgcggacagggtcttgagcacgcgggggcggccggtgtgtgtgggatcttggagcccctggtggaaggggcagcccgtgtgtccgcgtgtgtgttcgtgcccatgctcgtgtctcctgtgtgtcggtgccatgtgcacacgggggcccagttcctcgtgcacacatgtgcagccatccttgtgcaagtgtgtcctgtcttatcttgcctctgcctgtttcctgcctagggggctgttgggccagccacgggcttgtggggcagtccgcggccttgctggtccatgtcacctctgaccctgagcatgcatatccctggctggggctcacctcctgtcaggcttctgtgtgtgggaatgtgtggccctatgagcaggattttctgtgtccctggtggcaggtgtgtgtctttgcatgtgtgttccagcactggcctgagtatctgtgtcaatcccagcatgggtgtccagggcgtgtctggatgtgggtggtgtggctggggggagtaagtgtgtgaatgtgttccttggaattctactgtgtatgaaacacctttgggctgggcaggtgtaagccctgcttggtcagtaagtggggacagaaaagcttaaaaagggaagaaatggggtaaagaggaaaagtggggaggggataggagagaaggaatgagagaggcttgaagaagagagcagagagcagcaggccagggagaaagaatagctggagaatgggaatgtgggagaaagccaagccagacttagaagagagaacaggggataagagcttaagatggggaactggatagagatggtgggagaagaaggggtctgggagctggggaggcagtggggaggagagagtcaggccttctggtaaagtgatctctgaaagaagaaagggaagaagatgggagaaggcactggattcttgtaggccttgcaggatcaggcttggttttggatcagggaaggccactcaggcatacgggctggctcacagtgaggcctgtcatagcagggactgtcattagaggttcatatccccagcacagagctctgtctcccatttctctgctctgtgtccaaccctgttcttcgcccttctctggcaacctccctcctctggctccccttgagagctgccagctgggtgtggagaggaagaaaggccaaagggaaggttagggatttggagggcagggggagattctgtgggtgatggcaagattagagatgttggaggggcaggagtccaagcccttgctgtgtgtgtgtgagtgccattgtgtgtgtacacagggtgtgtacatgtgtgggtgtgtgcacaatgcctgtgttacgggtttgtgtaggtggctgttatgtgtgtgcatgcgtgtatacatcgtgcaggcatgggacatggagacccaggatcacagtttgtgcttttcaggtttgctgtgtgtgtgtgtacacgggtgtgtgttgttttctctgtacatgtcactgcatgcagcattaataacaggatgttctgtgtttctgtcactctgccccccctttcctctgtgtttctgctattcttttcctgcggcaacaaaagtgtttaataagcatatgtgtatgtggcctctgtgtacctgtgacttatgagtgagttcatggatcttagttcttggtaatatgtgtaaggttatattacttccaagaaccaagcaagccaattagcaaatttgttcttctgttaatgctttagttccaaagcggaattttaatgcatttaaagttagactttatgcctttattagatgagcttgcacatgaaaataatggctgagctcggttccctgaaatgagagtgaggcagccatacctgagaaatgcagaaaaaatgttactagggaagagatatcctgaaccattggatgcattgcttagtgtgtaccagaaggataattaacacatttgctcctggtttttctttactctggtggcaatctcggatgcctgtgttagagtgagagaagggctgggggaggaaagggggttgtcctgagacagagtggatgtggttgtacttttcacgtgaatgaaaggatgtttgtgcttctgagatatggggatacctttctgtggtcaggatcatggtgtactcaagcatgtatttcaggggttaaactggaattcccctgcatggggatatgtgtgtcttagggttataggtccatttctgtagtgtgctgtccacatgattgcccaagagacttgggccacaaagtccacaccagcagctctcatgtgggtcactgggttcaagtatgtactcccttgttgcagagctcacatatgtcaaagcatgtatcctgtggggtgtgtgggcctcagggtctcagaatgtggatccgtgctatgcccgtgctcacagttgtctgtatgtctcaaaagcacatagatccccagttatttttctgtattgtggttctcatatacacgtacctccatagctcagtatatgtttctgtactatacacctgttcctgagggagtgatagggttctcgtgtcatggggtccacatttttgtatgcaaacctcctaacacctgggttttacaggtaaagggaagctgaggactgattctagggcagtttgcaggcaatttgcaattctggaagcagacagtgaaaatataattgtggtcctcccttgttctgttctggaacctagagggttaacaggcagctcttccggtccacccccctccttccagcctgtagcttcttgttgccgtggagatggtggccccgccccgatgcagcacctgcccccccattggctgcagtggtgactgtggggctgaggggggagcccaggcctgggcagccattctggagctggagccggagcttggcatccccccacttacattctcttccagcccctcggcccctctaaattcccttgcagccgagcctcagcttcctttttcagcgcttgccatccctctaggctcttggcagactgagctgcatccgcattcacctcccctcctcctcctttccctatccttacttgggagtcccgagaacccccactgtccatccttcctgctccctgcccattcccctgcattctttgcttttcccctccccccaccaaccttctgagcccctgccactgcagtgcacgtggaaccggctccttctcccttccccccacctgccccttccttggattttctgtccccagcttctccccctgctactcttcacaggtctcttccaagacctccccctccagtccagggagggggtcatgggaggcagccctggccctccagacagacaggggttccaggaggtggggggcaggtggaagatgtgcccaccagggggaagaggaaagcctgcctatgggcaatgacctttaacccatctttccccccagctagccccttctggggtgggtgggcaccagggacctagcctccctggatctttggtgtccttcattactggggttttactgaccctctcagccatgctcccctgctgactgccagccccagtcatgggcctaaggcctcccaccccatccccctcagggggctcctgctcaggttccttgccccctccttcccgctgccagcctctccgccgtcgctgctcttcctgctgctttccggggggtaggtgaggccggagctgaggggcagagggaggtgggggcatgcactggggtcaggggtgggaacctgggttaacagcttcccttctggccccctccccaacttcccttctggccatttcctcccctccagaataccacttgggtcgctcgaggaggaagagtgtcccaggggggaagcagtacagcatggagggtgcccctgctgcgcccttccggccctcggtgagtggtgcctaccagatgtggctcagttgggccccctcccctccagccccaactggggccctaggagtctgagaaagagggcaggagggggagagagggagtgagagtgagaaaaagagtgtgtgtctgtgtgtgtctccccacctctttggtttcccccttcttggctctgcccccctgcttctgagaccagccctcccaccttctccaagctgtgtgtgtgtgtatatgtgtgtgtgtgtgtgcttgcactgtacccgtgggagcaaggaagacaaactggtattagggttcctatccccctttcctcgcctctggagacttcccttttccccatcccactttcatccaggggctctctaccagctgagggatgagtaggtagaactgaccctgccccaacccaccccatccccatttcccccccagcaaggcttcctgagccgacggctaaaaagctccatcaaacgaacgaagtcacaacccaaacttgaccggaccagcagctttcgccagatcctgcctcgcttccgaagtgctgaccatgaccggtacaggggctggagcatgtgggatgagattgatgtaatgtagggtctcctgtgtgagatgcagagggagggggttatctgtgtgcaaaggttgaaggattcaactcaagttggttgggggatgtcatggcacaggggacagaacagaaaagaactagaatagggatctgtgagcagcaggagaggggtagggtggcagagagaagacagacagacaggctggaaagggaatgaaggtgaagccaaggagggactcctcagggactcctcaggccaagaaggatgggctctagcccaggatcaaaggagctgtacaggaggagagtgaccctggaggaatgtttaaggaatgcagggaaggggttggtaggtgagtgagcaataggctgtaggtggaagggtgtcagggaaggtcaggaaatacaggggcagcaggttggagtggggctgggggtggctgaatgaatggatgatggctagggctcaaggacctcatcagtgagggaagagacagtatagagcatggcagagaaggggaggctgggacaggtgtgcagggtgacagaatgggaagcaacccatggactgaggcatgaagaagcagccagcggagaagtccagaaggcactgtccctgagaccaggctgaaggagacctccactgtttgcctttgttgcctgccatttggggttcctctctgggtttccccctcacccagtcactccccagggagaaccatgccctccctttcccccatgtctggccacccccaggattgggcaggtagggaggttgggataaagtgagtcacacctttccctgcccccctcccatgttgccagagctggatttggggccggcagggggtgagggcatggtattcctggccgcgggggcgggggggggggtccgggggccgggggagcgtcgcgctgacggcagccagagcctgcgatgacggggctgctataaataacttcttggaggctcccacacccaagctcccctcccgctttcccactgctctctactcttcatcccctgcccatctccataccgcttttgtattgctatcctacccctcattattccatgcccctagccccctttatcttctgccctcctgcagtgatttttttgcattccatcccctcttagccctcacctcggttctcccggccatctctccagttggccttcctcctcttctcctgtcctctgtcttgctgcacatacctttgtctccccctttcttcttcttgccctacctcctcttcttccctagtccgtgtattctgtcttttatcctctttgagctcttttctgcccacagctttctcctatttcttatgcttttccctcactctttcccctgcttctgctaaaacttgtcctcttatgctgtgttcattcattctttgaatcattaaatgtttatcaggcactagccgtgtgccaggcccaggctagacatatctcttctctgtgccttcacttctttacttccactttttcctttatactgaggctctggtttctggggttacctggaggtactacctagaagtgccccaggcccactttgttctctccttttttttttttcttttctgccatggtccatttctgggttgagatatttctagatgtccccagtcctcgcaatcccttaggtgtgagatggtgggagtttcttttttttcctttttttttttttaaatagaaatagggtctcactgtgttgcccagactggtcttgaactcctgggctcaagtgaccctcccacctcggcctttgaaatgttgggattacaggtgtgagccaccaggcccaggtggagcaggggagttccttaaaggattctgatttttctcacatccctcacgtccttcctgataggcagggtttctttctgtgtctgtttgggaagggtgttcagggggccttctctccaagtctccatcctggaacagactgatgatgcagggtacctatgtgtctaagaagagtaggggggccgggcgcggtggctcatgcctgtaatcccagcactttgggaggctgaatcacttgaggtcaggagtttgagaccagcctgaccaacagggtgaaaccccgtctcagctaaaaatacaaaaaaaaaaaagaaaaaaaaattagctgggtgtgctgaggcaggagagacgcttgagcccaggaggcagaagttgcagcaagccgagatcacaccactgtactccagcctgggcgacagagcaagactgtctcaaaaaaaaaaaaaaaaaaaaaaaaggaagagtgggaagccctgatcccttcctctcctgaacctcctgcctgccagggcccggctgatgcaaagctttaaggagtcacactctcatgagtccttgctgagtcctagcagtgcagctgaggcattggagctcaacttggatgaagattccattatcaagccagtgcacagctccatcctgggccaggagttctgttttgaggtactgggtctggtgggctggggagggccaaaggacaggggtgatggaaggtggggggcagagaggtctagagaaagtggcacaggtggggacatcaaggaaacagaaacttctgggactggaggaaagggtaggccagggaggaagagaaggtagcagagtctcctcccctctgtagccctttccctcaactccacactcctttctaggtaacaacttcatcaggaacaaaatgctttgcctgtcggtctgcggccgaaagagacaaatggattgagaatctgcagcgggcagtaaagcccaacaaggtattggggaataaaggggacacaacctgtgcagggcaaaggttgcaccaacacgggaagctgggggcctagggaggaaagtgagttaaaggaggagaggcttggggaaggagaggattgaggtacagtgtatctggacaagcagggggagacccccattattctgagtcccccatttcttttcgctttctgtactgctaccctgccttacgatctctttccctgccatagaagtcatagacttacagagttgatggggctctggaaattctttagtctagcttccctgcaggcaggaatgcttcactaataccccgcaggacacatcaaatacacttagccaagtttctgtaccttggtttcctctctaagacagagggaactggtggcaatgggttggattaaatgatctctaaggtccctttggcacataaattctatgagtctgttcttcccaaaacttcatgcttccagttctgtcagctattctctgtatcccagtttctagcaagcttacagtcctagtcacactcctctggggggaactcttgtgtcttgatgtccttaaagaactcaacccaagatgctgacatgatctgacctatgcagagtacaaacaccatgttccctttcaacacactgcaatatcacctgagccttaccattagcttgtgctcaattgtatccccccaggcccttactgattcttttttttttttttttgagacagagtctcactctgtcacccaggctggagtgcagtggcgtgatcttggctcactgcaacctccgcctcctgggtttaagcaattcctgtgcctcagcctccccagtagctgggattacaggcatgcaccaccacacccgactaatttttgtatttttagtagagacgaggtttcaccatgttggccaggctagtctcaaactcctgacctcaggttgtctgcctgcctcagcctcccaaagtgctaggattacaggcatgagccaccacatctggccaagctttacccattctatatgcaattcttttttcacaatttctgatagtctctgcaggactttccagttcccttcaaattctacattaatatttttggcttgttattccagcttttgaaatctttccaatactgtttgtgttgtctagtgtatgtctacgcttctacagtagcttcctggggctgctataacaaactgctacaaactgggtcacgtaaaacaacagaaatttattctctcacagttcaggaggctggaagtccaaaagcaaggtatcagcagggccacgctctctttgatgctgtagcagggaatccttccttgcctcttcctagcttccggaggttgccagcagtccttggcattcctgggcttataactgcatccgtctaatttctgcctccatcttcatgtagctggcttccttctgtgtgtctctgtatcctgtatctctgtgtctccaaatctccctctccatataaagacaccagtttttataaggtgggttaagggtccactctaattcagtatggcctcttttttttgagatggaatatcgctcttgtttcccaggcttggagtgcaatggcatgagctcagctcactgcaacctccgccttccaggttcaagtgattctcctgcctcagcctcctgagtagctgggattacaggcacgtgccactatgcccagctaatttgtttttgtatttttattagagacggggtttcactatgttggccaggctggctcgaactcctgacctcaggcaatccacccacctcggcctcccaaagtgctgggattacaggaatgagataccatggctggcctcttcttagtttgattacatttgcagggatcctgtttccaaataagatcacattcacaggttcagggtagacatgagttttggtgggatactattcaacccagtacaccgtctcaccttatgattgctgtctgtatcccactttcttactgtctctcctcttttggtgtctctctgtcccttccccctttaacatcatgcccaccccaccatgccaggacaacagccgccgggtagacaatgtgctaaagctgtggatcatagaggcccgggagctgccccccaagaagcggtactactgtgagctctgcctggatgacatgctgtatgcacgcaccacctccaagccccgctctgcctctggggacaccgtcttctggggcgagcacttcgagtttaacaacctgccggctgtccgtgccctgcggctgcatctgtaccgtgactcagacaaaaagcgcaagaaggacaaggcaggctatgtcggcctggtgactgtgccagtggccaccctggctgggcgccacttcacagagcagtggtaccctgtaaccctgccaacaggcagtgggggatctgggggcatgggttcgggagggggagggggctcggggggtggctcagggggcaagggcaaaggaggttgcccggctgtgcggctgaaagcacgttaccagacaatgagcatcttgcccatggagctatataaagagtttgcagagtatgtcaccaaccattatcggatgctgtgtgcagtcttggagcccgccctgaatgtcaaaggcaaggaggaggttgccagtgcactagttcacatcctgcagagtacaggcaaggccaaggtgagtgttgtgccctcagggaaaggtgacttgggaatgggcacttgcttgggggttagtgaggacagggcaaattcacgagattgggttgtgcagaggctgacacttggattttcctgggcctcaggacttcctttcagacatggccatgtctgaggtagaccggttcatggaacgggagcacctcatattccgcgagaacacgcttgccactaaagccatagaagagtatatgagactgattggtcagaaatacctcaaggatgccattggtatggcccacactcaggccctcttcttcccaaacctgccagatgtccaccccagaccccaagtccacccttccacagcttgatacttcctaacccagagtcctaggactccagcctccaacacctgattctgaaatttccccaaccctggccacccccttccctgcccttggaaagtgtgaccacaccctcttgtgcccccaccccccaggagaattcatccgtgctctgtatgaatctgaggaaaactgcgaggtagaccctatcaagtgcacagcatccagtttggcagagcaccaggccaacctgcgaatgtgctgtgagttggccctgtgcaaggtggtcaactcccactggtgagactgggaacgctgggctggggggccagggtcgggggaattatgtgttcatctgttcatctatctgtccatcctcaaagaggactgagcaccatttatgggcaaagcattgttctaggcgctatagagcaaacaggtgaaagaggcctggtccctgccctcagagggcctccaccagaatggggacaaattagaagaaaaaaaaaaaaagccacagagccataatggtgtgtaagtgctgagtaagggtccccccaacctctgtgtgacataaggtcagagagaaggcagagctttgagataagtggggaagaggtgcccccttgggtaggctttgaagactggtttaggttctgatatatggacatagttggcaagaaagacatttcagaagaaggctgtgagaaaggcacatgtgtgatggtgaaaaggcccaggagttttcaggggacattaaagtaggttagtagcaattacatcaggtttagtggagcatgtgcctcataatggggagtggcgggagagatgtctgggcaggaagattagctttagaaactggaaggcctcaaggagtctgaggtcattggtaggccttgggatgccattaaaggtgtcagaaatattgtgatttagaagattaatccataggctgggcacggtggctcacacctgtaataccagcactttgggaggctgaggcgggcagaccacctgaggtcaggagtttgagaccaggctgaccaacatggagaaaccctgtctctactaaaaatacaaaattagccagatgtggtggcacatgcctgtaatcccagctattcgggaggctaaggcaggagaatcacttgaatctgggaggtggaggttgtagtgagccgagatcacacgattgcactctagcctgggcaacaagagtgaaactccatctcaaaaaaaaaaaattaatccataagtagagtgtatgtagtagagtagagggatgtatgttgggggatgactggaatagagccagctgggggctcggaggcatgaaagtcagatcctgaatcagaacagtgacagaagttaggaaggagctactggaaggaccctctcgggaaaggatcagtaggaattgtcagcttattggaaaggagggagcatgtctgtggggagtcacggatgactcaagaggccatgaggctggtggttgggagaccggtggcctcattgacaaccaggaaagtcaggaggaggagccagttggaggtgtgggggcgatggtgagctctgctttacaccagccgagtttaaggtgtcagtgggacattgaagtggaactgggaggtgcggggagtgagctctgagccattccagggactggggatcatgcctggggcacctccatccccatttccctggaatccagaagagttggggggtccgagctccctgtacctcaagtgaccctccatctctctcccatctctgtctctccctggtgtctgtttttcttctcctcctctccttgtctctctcccacacccctccatctctctcccacgtgtctctcccctcaccttctctccccctccatttctctctccctaatctgtctgttccctctgccatggcccccttcttcaagcagcctcccatcttgctcctgcggtccctccttccctgtctctctcacccctgtttccacaccctcacctcctaccacccccctcagcatgttccctggaagctgagggtctctggggctcagtcccggtctctctctttctctctctctctctctgtctccccgacccttccccccagcgtgttcccgagggagctgaaggaggtgtttgcttcgtggcggctgcgctgcgcagagcgaggccgggaggacatcgcagacaggcttatcagcgcctcactcttcctgcgcttcctctgcccagcgattatgtcgcccagtctctttgggcttatgcaggagtacccagatgagcagacctcacgaaccctcaccctcattgccaaggtcatccagaacctggccaacttttccaagtgagggaagcttcaggagtgggcagggcagggagtggcagggcagggagtggcagggctgggggtcggcaagaagggtctcctgagtccccagagatcctgagatggggaggctatgataccttgtgtgtgtgtgtatgtgtgtgtatgtgtgtgtgtgtgtgtgtgtgtgtgtatgtgacctttatcttctgcattcttggctaggtttacctcaaaggaggactttctgggcttcatgaatgagtttctggagctggaatggggttccatgcagcagtttttgtatgagatctccaatctggacacgctaaccaacagcagtagctttgagggttacatcgacttgggccgagagctctccacactgcatgccctactctgggaggtgctgccccagctcagcaaggtcagcagatcccctctttgccctatccccagatggctccagaggttcctggagcctgagaaactaccctttgaagattttttttctccccttgtttctcgaggtgtcaccactactatcccaactcaggccccctccacctgcaccctcagaggccctcttagagctgggcactgagcccccaggtaacagcctcacccttccaggaagccctcctgaagctgggtccactgccccggctcctcaacgacatcagcacagctctgaggaaccccaacatccaaaggcagccaagccgccagagtgagcggccccggcctcagcctgtggtactgcgggggccatcggctgagatgcagggctacatgatgcgggacctcaacaggtgagcaccctgggacagccaggcctgtgccctaggagcccttctcctattctagatactcctcactgggccccacatgcatctctctagggcttgaaagaagggaggaaaaagcaccaagttctcaggggagacgataaggagacaggtacagtcagtggtaggctgagagccctttacagcctgagggagtgagagatttggagctctaggaatagggctgaggctccaccaactcacggcttagttgtaagcctagagcatccctgctgcaagctctgatttgctgtccctctgcctgcccatgctagtccccaggctgaggttcagccagcatgtcatgtcagccatgtgtcaaaatgttcaaacatctcagtaatagctagtgaataagcacttcccccagcccccgaccacaaccccacagacctccccatgatccagcacttagagcagtgacagcagaagcctagcagggcctgcagcctgctccagagtcccagcctccattctgataggtggtgcccgtgcttcatttgctgctcattattttgatgatggccctgctttttcctctgcccgtgtcttcctccatgacaccatacccatcccaccattcccgcctctcctttcatttgtccacatctctctccttctctgtctgtgctcgcccctctttccatctctctccagctccatcgaccttcagtccttcatggctcgaggcctcaacaggtgaggggctctcccctcccccgccctcctctcctctcctgtctgttccctctcccactccactggccttcgccctactcctctcctctcctcctccatggacctcatctcctccatatgtgcccagccctgcccccatcccttctcttgctgcccccatctcccctcctctaggcctcacccccttcccggaggggccctgtcctttccctttactcacctgtcccctcccatcctccctgcctgccctcttcagggctgccaccgctagctctcagcccttccctctgggtcccacttttcaccccaaggcctgtgccagaccacagcaaggttcaattgctaggagccctgaccttaccttctgcttgtgtgcccccttcccttctgacagctctatggacatggctcgcctcccctccccaaccaaggaaaagccacccccaccaccgcctggtggtggtaaagacctgttctatgtaagccgtccacccctggcccgttcctcaccagcatactgcacgagcagctcggacatcacagagccagagcagaagatgctgagtgtcaacaagagtgtgtccatgctggacttacagggtgatgggcctggtggccgcctcaacagcagcagtgtttcgaacctggcggccgtaggggacctgctgcactcaagccaggcctcgctgacagcagccttggggctacggcctgcgcctgccggacgcctctcccaggggagtggctcatccatcacggcggctggcatgcgcctcagccagatgggtgtcaccacagacggtgtccctgcccagcaactgcgaatccccctctccttccagaaccctctcttccacatggctgctgatgggccaggtcccccaggcggccatggagggggcggtggccatggcccaccttcctcccatcaccaccaccaccaccatcaccaccaccgaggtggagagccccctggggacacctttgccccattccatggctatagcaagagtgaggacctctcttccggggtccccaagccccctgctgcctccatccttcatagccacagctacagtgatgagtttggaccctctggcactgacttcacccgtcggcagctttcactccaggacaacctgcagcacatgctgtcccctccccagatcaccattggtccccagaggccagccccctcagggcctggaggtgggagcggtgggggcagcggtgggggtggcgggggccagccgcctccattgcagaggggcaagtctcagcagttgacagtcagcgcagcccagaaaccccggccatccagcgggaatctattgcagtccccagagccaagttatggccccgcccgtccacggcaacagagcctcagcaaggagggcagcattgggggcagcgggggcagcggtggcggagggggtggggggctgaagccctccatcaccaagcaggtaggtgaaggcaggaggaaggcgggctgggtcacaacagggagggaagaaggagatggggggtggggttgaaacagagtctgtggcctgaagttacaatcttcttgcctccttttggccattaaacaaatgtgtatagagggcctgctatgtgccatgtgctatgccaggcactaaggatacggcactgaaccctcttagcactctcattccagaggggattaatccataagtagaatgggggtgactggaatagggccagctgggggcttggaggcatgaagatcagatcctgaatcagaacaacaggagaaattctctggcattcaactcacatctctggcattcagaggtgataaaggctacagcagggcaataggactgggactgtcgtctcctttggctgtgctgttgccctctaaatgtaccctgctttttcccaccttttctttctctgttcgccctcactgtgccttgtcccagcattctcagacaccatccacattgaaccccacaatgccagcctctgagcggacagtggcctgggtctccaacatgcctcacctgtcggctgacatcgagagtgcccacatcgagcgggaagagtacaagctcaaggagtactcaaaatcgatggatgagagccggctggatagggtacgatgggctctaccagctccaagccccagtgtttccttttacccacaggggagatctctagtcacttccaagggaaacctccagggtcagttatggtggtaagaaaaggcaaagacctgatcacctcttgagaagccttccgtccatctggggagaccagatacacacagttgttaaaagtcactttcaactctattttgtaggttatatatatcatagactatctggggatcatctattttaggcttacctccatgttcttccccaggaggaacataagccctggctcctgtgactgcttaggaaaggaaatgttactttactggcagtaggagctaataaattggatggggatggagggggttgcagctatattacaagtgatgtctggcctacccagtcactgtcccatgggcataagtgacagtcagtgggcagggaatgcatgtggtaaagtagtatgtatcagtcatgtgtgaaaagaccatccagaccctcacttggtggaggctgagggcaaagggcaccaagagctctggggctaacacagataacttagcagaaagttacttgaggaagctgttgttcctagggcctgaggaagagggcggctgcaggattaggtcatatataaagaacatatgatcctaacacggggaagtggtctttgagcataggaaatgttgggactgactcagatgggcttgaccagcaaatttaatgagaattaaaccctaatattagccccaaggcagccaccgtaggaagttaattcacatctctcctctgcatgtagaagggttggtgatatatgtatgtatcctcttcagagaatgaggaaatagtcttttgaatcatttttttttctgtgcttcagatgaggaaaccttgttgagaaaggctgtagactagcactatccaatataactttctgcagtgatgaaatgttctgtaatctgtagtgttcacatgtgtctagttgtgattcaggaactgaatttttagatctatttaattttaattttgtaatttttttgagacagggtcttgctctgtcacccaggctggagtgcagtggtgcaaacatggctcgaggcaacctccgcctcctgggctcaagacatcctcccacctcagccccctgagtagctaggactacagggtcgtgctaccatgcctggctagttttttttcttttcttttttttttttttttttttttgtagagacggggttttgccatgttgcccaggctggtctagaacttgtgagctcaagtgatctacctgcctcagccttccaaagtgctgggattacaggtgttgagccactgtgcctggcctaaattttaaatagctacatgtgggctggtggctaccatattagacagcacagctgtagactctagagccaatagggcagataaaatgactcttctgagctcttccagctctggaattcctagattctcagcctagaattcacttgtagacctcctatccttggcaccttgacagagccaaggagagggtcacatggaggacagcacctgcctttcccccgtcaattgccttttcttcctatgtctccagcatgtgttctggggcctggcttagggctgaagagtcaccctatttttaatttctgagcacagttactcaaggtgtgtttgtgagtgtaggtttctgtgtgggtcttcatgaggggccatacatagacctcagggtgtgggagtctgtgaccttgctccattttgagggaacctcagcaccatggcagggtcttctcaaaagcaggtgtgtggatatgatccacaggcagggaaggggttggggaaggcagctggatgtccctcaacatgcagtggctgtgcttggcagacagggatggaggctgggtggtgggcttggggtggggcgcccctcatagtgcggggtcgtgtgcccggcgggcaggtgaaggagtacgaggaggagattcactcactgaaagagcggctgcacatgtccaaccggaagctggaagagtatgagcggaggctgctgtcccaggaagaacaaaccagcaaaatcctgatgcagtatcaggcccgactggagcagagtgagaagaggctaaggcagcagcaggcagagaaggattcccagatcaagagcatcattggcaggtgaggggcggcctggggagggggttgtgagggagagcctgaggctggagagagcaagtgggtgagctactccgctgactcccatccccaaactcaggagcccaccaggagagcccaccactctcctccccaggaagccacccactcactcatcaccagatggagagaaaccccaacctgcttagtgcattaaatatctctttaccaaaccctgacctctcttctgatagagtagcttcggaagcccttggaaaatgtacctgttcctgtccaaccatcactgcatttgcatttaccctaggccagagctccccactagttattctcaacttaacctgtgatgttcactccaaacctaagcagggctccctagccagagtaggccctgcccttcctgggtggaccctccctctctagccttggaaaggtgttctgttagaaagggtcttttagcctgtgtatgttttcagctgctccagcaagtcctgggctccaaagagggtatcctcagcaaagaggtcaattatcttcagagtggtggggtcggggtgggggggaccctgggcgcactccaaccagagccacctccattttgatccattctaaatgtattttatgtaagatttaattagaagaaaagggcttcttgaaatattttttgaaaaccactgctctaattgatatcctttatgataaatcacctcgaggatcttcacagtgaggtgacatgggggatgcagaagccaggtcctcagccatggaaggtctggggaaggggcactgctgtcctgattgggacgatggaggcctggaggtgtctggatggtagaagtctttgaggcacagaaagctgccttagcagggaggtgtaagggttcctgggaggaaggtggagagcatgatcctgaggaacagggagtcttgcatcacggcaatggagggactctgattctaagggataaggatgcctgaggtttttccagagagctatggggttccatgggcaggctctgagcctgtgcccgccactaaccccactgaagcccgtcccttcaggctgatgctggtggaggaggagctgcgccgggaccaccccgccatggctgagccgctgccagaacccaagaagaggctgctcgacgctcaggtggaaattacaatgtcatttatcttctccgtgtcccatccccatccatcccactgtctttcgtgcactcactacaccagccacctagccccatcaccatctgtctctcatagtctgctgtttgtccactggctgctcctggcagccccctagtgaccccatcttcatcccatcgtctgtgcctttgtcactcctggcagcgtcagcccaactcctgtgccttcccatccagtcttcccactcctctctgcatctcaggaccttctctaccagaaccttggtctttctgcccctagaccccacctagttccaagaacccctgccccttctttgctcactcctattcaagccacgttgttcagcttcctctgcgctcttgggccagagggctagaagctgccgttttctggaatagagcacagggcagtatgatctgtagtttctccaggccctggccggtaccctgaaaacttggggacccatcacctctgttctcttggctccctaattttcctgtctccttggcagctcctgcatagcttcctcttcctgactcttcagatcttgaaggccttccatcctgtaacctccctttgccctcagtatttaagtccagcctccctctggcctccctcccactctggccctcagaccttcccagctgcctgctgcccagcctctcttctcacaagccagcttctaggacctcccttctgcacccttaccccttgctttcccaaaattctgctcattttcctacccatactcctctttgctctgactgctaggctccccccgcctgccatccccccaccaaggctcctgaccccatgaccccactctctcccactgcagctcctcatcaggtaattctcctggttccgctttggccacgggcggaggacacagggggaggtgactccggaccactgcaggttggtcgtgaagcccactccctccaacacctccggagcctctcccctctcactgctgccctccacacccagagaacctccacagactccagccctccgacacctgcacagatccatctcccaagacaccacccaaagagagcatttgctgctgcttcccagaactgtccaacaataccttagcaacaccaagagttgggccctagatgggcccagcacattcacaggtcacacccacttccctgcaaaacccaccccctcccagcctcctcctgactctaagccctcctcttcctctacctctccagtgtatgtctgtcaccccccatttcaccagagcgtccttaggggctgggggtgggtttgttaatggggtggaggcaatgatgggttggaggatcttggctataggggctgtgctgactgcagcaggtaggttgggtttccctcttccttccctaatcttggttctctaccctcctttccactcctcacctgattctctctcttcctcctccttatatctgtgaggcagaaggcatctgaagctcatattagcccccattgggtgggaattaggagtgggtagttaactcagggagacttgagataccctggaaaaaatgctattgagatgtcctgacattaggcagggtggatggaacaagaaggagcaagaaaggaacctcaggcagatgttaggacatggacttgatcatgtggcctgggagtttagaaatggggagagacatcctcctagatcagatcgtgggctcagtaggcatgttgattcccagggagaggtgccaggaacagcatggtaaagaatgtactcttcacagctcacatccccaggttgctgatgccactcactccccctctcctgccatcgagtggccttgccggacacatcaccctacctaaaaagccagtaaatgagaacctgtcagctatagccatcatttctgagatgcgattttctttgggattgagctgcagtgggcagtggctccttacactgtaattttaattctctgcctgcccagcctctctgtcaaagtagctggtgatctataaagatgctaaaaggcaccaggggactttgccatttaaaggactcctgcagtgaattcttttgtaaaatgaataatggcaccctaatttatccactttctaaatttgggtccatgggggtgtccagggcatgcttatgtgctgtcaccagcagacaaacagagggaatggaatctgggggttccttccctgctctcccgccatactcaggataccctaccataagtgatttcctctcactgacttgcagaaaatgtgtgagatacccagcaagctaagaaggcagttttgctgggtatctcatacccaaggctggggtttgggtgatctgagaggttagctccttgatcctaggatggaagggagagcttatatagaagcttttacttggaaggttttgtatcctaaggtcagacatagctatattaccaagcctaaatgccatgtggcccaggaaataatttggacatttgttctaaaccacttgtggtaggtattggtctctctgcaactcagccattaattagaaattagttttgagcctgaattttaaaaagccaagtgttgcccccagcccacacacacacacacggacatgtacagtacaaaccccagataattacaacagccaaagagagaaggaagtgaatttcccaaccagaagcgtagggaaattcagatggctttcttttctcccagcagaggaacagaaggcggagctaagggcaggaaccaggagttggtcaaggagctataggaggtgatgagagtagaaccaggggtaggagctggtctggtacccctcaccctctaattgggagcccagggagaaggactgaaaagaagatgggagtggaaaagaataaagccagtttctgcttcccagggatgcagagattggggcatgctgtgtctgcagaagctcctagtcatttccgccataattgtgagagagaggggcagccctcccacaagatttttcccttcccatatcacttccctgaatccccttcctttccccccagtacagttaaacctctctctaatttggaatgttatatttgagaagatggccactgtgaataagtgacaaacggaatgacagtgtctatttaatgaaatgcatgtcttcaaaatatatacagaactcgatgaacaaggctttttccactcctcagggagcatgcattaatgaatagataggattcaaaagtctgttttctggtatgggttaaatatcccctcctacagacatatttccaccactaatttgcttagtacaccttttcttcacagataaaggaaaatgcaagctcagtttttcttcagattatgaagaaattccaaatccacaggggtttggattaatgaggttttgctgtactgcctccccttattcctcaacatgaagttcccacctcggattggggatgggtgggagggggtttcaagaggaggagggtgggatgggcaaggaatatacacaggtgaagccagagaagggttaggttgggggtgcggtgggaacttgctgttttgatctggtttcctggtgtgacactctgggttaaaggcttgaaggcccctgttaggagtctaggggtgagattctcttctctctgatcccagaggacgttaacttctactgcaggtgagaaacaaaataggaggatggtggggactgtcctgggaggagggggtggtccatggcttgtggtgtgggctggctataggggaggccactgctaggggactggcatccaggcccccttgaagcgtctcaataagtccgcgctctcctttttggtgtcttgcaggagaggcagcttccccccttgggtccaacaaacccgcgtgtgacgctggccccaccgtggaatggcctggcccccccagccccaccacccccaccccggctgcagattacggagaacggcgagttccgaaacaccgcagaccactagcccacccagcatcagagaccttctcttcctttcctgtgcaccccaccctgtaacagcaccaaccaccaggattggacatcaccgaggaacagcgggattgcctccccgaatgcctccctgggaggcacactgattgcccacccccaccactgcaccatttccaggagggagagtggggaccctcagccgcccccttttccttcccattggggtgctgccctctctttgacccccagggacccttgccccaggacaccgcctaccccgtacagaccccttcactccggggtgctatccccatcctctgcctcatcgttcccctgagcactgggggacagaccctcacccccaccctgggggtgtggcacctccaaactttcaacttcagggtgatttttttagcagtaaccagagctgacaatctaactcccctccaccgccccattttggcctcccctgccccccttgttatggggaggggaccccgggtgagggggccctattaccccttgatttctcaggagcgtctgggggggctcagcacgcacaaactccttctccttctaccactcttaaatttactccctccccacccagaacccagatggggtggagggggccaccggggcagggagggggcggcaaggggggaatgggagttgtctccccttctccccacacctgatctgctctcggctggtcccagagcggggtgagggggcttatgcccccccctcccccagtgtgttgggtggggtggaattgaggttagggtgaggggtcagggtttaggagggtgtgtatgttgggaggacaggctagttgatctgtcctactctgacacacagtcccctctgccccttccttctctcttcttggtctctactcccagggggaggggggaacttactctaggaaaagccatgtctctctcccccagggtggggggacctgtgttggaggaggggtgttggggggcccccttccatgactctgtcccctgggggaggtaggacagggctgggcttccctctcatcctccccctcccaatctccttccacctccctccctcccgccagctccacgatttttcggtgtttctctgtacatagttttctggcgggataggggaggtaggatggatggggtttggggtgggtaggccatgggaggggagaagcccctccttggcaccccctcttccctgactgctgtcccctacccagccttgcccccttcatccttttgcgtttggtattgagactctcctagactctactcctctttcttttgtatggacagttccccttcagtcccatccccctacacatacacccagccggggccaaatttatacttatataaaagttgtaaatatgtgaaattttatccctgtgccctttccccacctcaggccctacccctggaccctccccaaccttccttctctcttctttggctgttgtaattatctggggtttgtactgtacatatccggggtgtgtgtgtgtgggctgggggcaacccttctgtacagagcttcctggccccctccccccccgcccctctgcttccctccccacccaccacctcaagggtagggagttgctcttcctacctgttttattttgttttctcgttctccctccccaccccactcccagccttatctatcccccctcactgtccccttttctccactcccagccccatttcctttttttctggagtgtgtggtgaaacagaaaaaaacatgtttaataaacggagattgttctttta

SEQ ID NO: 6 - DNA, 33625 bp, Homo sapiens
  source 1..33625: mol_type = other DNA
ctctctctcggctgccgctgctgccgttggctcttattctcctcctcctcctcctctctcctcctctctgcttctctctgctcctctctcctcctctctcctcctcctcctcctccacctcctcctccttctccccctctttctccccctctttctctcttctttctcccccgtccccccgccccctccccccaggcctgatgagcaggtctcgagcctccatccatcgggggagcatccccgcgatgtcctatgcccccttcagaggtacgtggtggggggagggggagggccatatggggggcaacagggggaggggcagggggcgggggagagtcggggccgagggaagggagcggctgaaatggagggagaggggaggaccgggaaggcaggggtgggagcaaccgggaaggagggattggaggccagagggataggggtagataagggatggaggggccaggaaatgggccttgaaagggggctgaagggaaaatgatgaaggggaaagaaagcgccagaaaggggtgggaaggtagaaggattgggttggggaaaaagctgcagaaataatttgggggtgggagctgagtgatgaggaaaacatcaaggaaatagagggacagagtcttgggagaggggctaggaaggagaaatgaaaacataggggtgaaaagagaagccaagaagatgtggggggataatttggtgggggactcagagtatggtaaggggcggaaaaagagaggaggatggagggagagaaggggccagatggagagagagggaggcagtggggggtggggggatggagggtccagatggagggaagagagaagagagagggaacggtgggggaggggagaccagggcagggggagggccaaattatgtgtgcttgggggggtggcagtacaagtaagatgaaatgatgggagtaggggacaggttaggatcctgccccagactctgaccctcttaaaggctcttgaccagtaaaatgtaacctctggggtcttaatgctgtcttcacttcatcacttttacccccttcccccacccctccaatccttatttccattccccctccctcctctgctgtccatcacctatatctttccccccatatggtgtccgttccttatatgagtcccccttctgtccttcccctatatcagtcccctctctggtgttcattgcctctctcttatctcttttctatgtggtcgctcccttatatctctatcacttctttactagtccgtcttgaccccttctcatagcctccgcattaggtctgcctcctttgctctgacacatgtccagtcgagcccttcaggtgtattcccttgcccctgggcatttaccatcatttctgcctccttttctatctactcctattatttgtctcccagaccattccttgtcaccacaagtctgtgcctacccactttcccttaatacctcaactcttattatcatttctcttttcttgtctcctctacttattggggtgttggagatttggggtacaaagacacaggttttgatgattagaacaagggatccccatctcagcccttgagggatttctcatcctagtaaaaatttcccgtttctcagccatgttgctctgcctcttgcatctgcctcccagaatcattcttcaccaagtccagacagtatgtgtgtgtgtgtttctagagttgttcctaagttccctgctgccaggagaaagatgccctctgaagtctaacccgcctcccttggctgcagcctgaaccagccctttccccaaatgcttatgtgtatcaacagatttctcctctgggtataccaagtgtctaagtgcggtgggcatgttgcctgcaatgtctgtagtttcagaggctaggtattggtgatctctgtatctagagaacattagttagtctctgagatgggttgagatagagtttcaaagtatgtggtttctaaatggtggacattctgagggttcatcgctcactgtgatctggaggttgggtaaatttgagagacatatttatagacagaaaagggctgaatttgcaaaactctgctactcagtgcattctgttggaatgaagctcaaagaccagttctcctgcagtatactcatggttatcccataaaggtgacagagtttgatcttttttccatctaaaatctgataccttaagcctaatttctagctctttgctgtcgccaatgtgacatctctcttacttcaatagggcctgtgtggcattggggtttaaaggagaggcagtttgcagggagagcggcatggcaagaacaacagagctggggaaggaggaccctggtctcatctcagccataggcgacacttggtctactgttctctcttctcaaagttttgttcccatctccagctcttgtctttggaggcccctctgtgtcaccaggcctggatattccattctttccccctgacatctttcttcctcaagctcctcactgtgccttgagaaggctcctgacctatggagtggcttccatcctgtccactgagactaggtggggtgaggtggtgggggcagtgttgaatctctgggcactgaatgggagaagagataaatttggtctctacttgtccaaacaagggcatatttcccaccttctgccctggggcctccacagccagatgccgacaaaagccatcttctccttaccaccagaagaaccagaactttccctaaaggatgagcagggttgaactaggggaggagggcacagaggagggaagagttgggaggggaggccctctcatgtctctgactgtgaacaggtaagctccccgacttcttcttttctagatatttagccctcagaatctcatgatcccaatgttcctacacacacaccccatccttctttcccacagccatgtagctttcctcatactcttttatagaggggtggtgccaccttccaagtccttgtcttctgcagctatttggatgccttcctctacccagatatctgacaccaacttctctgtttttctcctgccaattttgcggcacctctaccttattttcctgaagtagatggagagctccttcatctttctctgcccccaaacgtgtaccttctctgtcatataattcccatccctgaacctctgcatttcttcccccaagtctccagagccttctgtgatgtgatcctctcctcttttcccctttgtataaatgcttgtcttgtgttcctttagagttgccaggtgtctcactttcaactgtgatctcctgaaggccggtagggtccccttctcactttgtggactctcttcttgccattttaggcctctgcttccaaatgcatagagcctcccttactgtttctgtgtgtctctgtcctccagatgtacggggaccctctatgcaccgaacccaatacgttcattccccgtatgatcgtcctggttggaaccctcggttctgcatcatctcggggaaccagctgctcatgctggatgaggatgaggtgagtgtggtgagaaggctgggaaaacgcatgtgggagaatggggagagtcctcattagtgaaggggagagagacacaaagaaggcagacaaagaagaaacatttgcaaatggcaggagggtggccattgagacttgggttggagtgtgacaagaggggtagtcattctctcatcttgggcagggagtctttgagggttatgagctgtgttggggaggagatgtggttctgaagatttcgggggtggggaggctcctgttttcacactgtccatattgtaggataggttagaatggggaaggggaactgaagggtttggagaaggggtgaagacatggaaggccctcccaaccaaagcaatcctcaggacccctctctgctttgcccactgagcaagactcccagtcctttctccatcagggggcagagtgcaagaagagaaattgcaggacgcagagaaaaggcaggggatagaggaggttttaggtagctggaggatactgggaaagaagactgaggtcagtggatcttggcatgatggggtgaagactgggagagtgattgagatcaaggtttagagagacactgagagggcttctgcaagggagtggttggacatcaggaaggaatataagggaggggaactgggagtatgtggagttatctaaaggagtacaagtggagtaggggcagggactagccagagtaaaggagatgaaacggacagaggcctcttatgggaggtggtggccaggtatggagatctgagagccagggaagtgtgggttctgatttgggagctgggtcagcaggagtgtccattggaggaaatagaggataaaaactggaatggtgccccacaagtgtgagtcagggaatgggggtgatttgggggacaagctttaaaaaatggtagggtagcctggaaaaccaaacgtggtgagagatggaccctgaaagttattgggaatggtgtgatgaggaagggttagaatttgagtcagggctaactagagtgtcaagggacctatgggggcaggtgagagtgtggagagatggctgggtagacagcttccaggccacagagtaaagtagaggtgttaaggagaggatatggtgactttgaaaggagaggtggcttggggagtctaagttttggacaccattgggagacatttgagtgactgtggtcaaacccctagatcaaggtttggagtctgtaggagaaatgggggaaactggaacccagagtggggactgttatagggatgaagagggaagacaatggattggggtaatgaaggaatgacagagatagggacagactgaagggaccagtggccagcaagttgaggggcaataggaatggccggtctggagcaggaagggcgtgtgtgtgggaggaggagtagatagaattgggaaaatgagcagcctgaacagacgcagggagttggaattggatgggtggtggaactggggctgggggatagtgaggggtctgttcctgtggctctttgtagaggtggaggagactgcaggggtggaggtgtatggaggtagggatggaggtggacagggggagctgggtgtgaagcttagggaaaggtgatggctgaggggaagaagggaactgagagatgctgggtcgggtgatggaaggagatgaaaggtgggggaaggtgaccaggccctctggcgggaggggaggttgggtcccagtctggccgcaagccgggccgtgggagggagacaagctgtggatcctgattataaatgcagcttctgctacccccgtgacaggctcggagggagagaagggggtaagggaggtgggaggttgggaggaaagcagggagaatggaggaactgaaggtcaagggaaaactggattcaggaagtagctaacttctgaggagttgcagaagagctgagtagggtttgggagaattctccggaaggtgtttcaagacaaatggagggagaaaagagggtgggacggcaaagaatgagagagagagactgggggagagaagggagagatccagtcaccaagtgtggaggggccggggcagaaaaagtgggcaggcttagggggaaaaggaggcccgcccttcctgggaggaggcggaggggggaagagggaggggaggggcggcgtgggccagggaggtctgacacacccccacctcccctagatacaccccctactgatccgggaccggaggagcgagtccagtcgcaacaaactgctgagacgcacagtctccgtgccggtggaggggcggccccacggcgagcatggtacggcggccgagcaggctctcatgacccggacaggcgcggggagagggctgaagatggaccgggctggcggggcgggggcgtcggggagcggcgggagctggccgggggcgggagaaggcggagacaaagtcttgcttcccgaggggagatggcgccctcctctggaaggtggagcgggacaggggctgtgggtgagaggcccctgtgggtcctcgctcggcttcgctccctttctcctctccagcagtgaagtttgactattttcctggtgcttgtaaaaatagtctccttagatctttagcttccagtccgtgtgttcctgcccctgcccctcccagcccctggtctgggatcttggaaagaatgacctagttcctgccctccccccagacctcttccccacccctatgctcctgattcttcttccggaccttcagtccttgaggtctccttttattaccccctgttttctcctctcatcagctgcttctattttctgcaattgtatcaccctttctctttcctccctgatttctcttattttctgtacccctcttgattaccccgtccctcccctctgctcccggcgaaagtccatcagactcccatctcatctcctttctgtctgtagagctatctgcgcggtctgtctctccaactctctccctctctccatctgtctcttcccctccctccccctttctgcccccccaaccccctctttagtcatttaaaaaaagatttttgtcttcctgagaagttcgcttaaaaggtttccatgggaacagtgaggggagatgaaggcagagaacagagatctgcagccaaggtgcagagagacgccctcagcccccggaaaccccctcgtcactgcccccttccacttcatttcccttcccggacctagcgatccaggttccccattcctccaacccccaggtgacccctttttcccaccagctccccctcccttcaggaattcaagctaccagtttctctcccttagagagccagtgacctgtccccatcctttggctccttccagccttctgataggtccaggccctactagccttgtgggccggtttggggaccttgggtgggtgggtgggtggtcagggatgtgtgtcattgtcatggaaaccccataccatcggggtgtgtgggtagcacatctctgtgaggcaaagtcaggtggtggtggagtgtatacagagaatgtgtgtgatgtctgtcacatctgtgtgtatatgtatatgtgtgtatgtcatgttcatgtctggggaagatgtgtgtgtttgtgtgcatgtgtgtacacctgcgtttccaggccagaatggtcacatgctcacagagcatgtttatcatgtgtcctgtggttggcagatctgcccacttattccccatctttctccttatcctgagtcctcagggtctaggggtctctggcgggggtctcccgtgtgttttgggtggggacaccgtgtgtgtttggagttgaggtggcatgtggaggttgctgtattggggttcagtaggggctgggaggggttccaggggcatatgtatttggggggagggcttagtgaaagttgatgggggtgaacgtttagggtcttcaggcagacatggaatgtgtaaagggcatggaggatgtgcagtggacataggggtcgttgggaccttggggacttggcaatgccaaggtgtttgctgaggctgtggactctccagcccgggagaggtcgccggacctttgaggggcattggaatcctgggctcctcctctgctgggtggagccgcgaaccctcgttctctccgcggttttgtgtgtgtgggggggtcctgctcaggaggggatggtgggtggcctggtttgttttgggggtccccttgcccccctcccccgtctctctcgcgctgtctccgggcgacagggctcgtgacagaggcggccacggcagcagcggtcgcctagcaacggcggcggggcccgggcaacggcggcagcggcgggagcggcggcggagggagccgttcccgcggccgtcaccgcgcggccgtcccggccgtcccgggccccgccgccgcccccaccgcgccgggcggcgcgagccgggccgtaccgccccctctccctccgcaccccgcccttcccccgcgcaggggcggggcccctcccccaccggggacacccctggcccccccggggggcggtgcgaggtaagggcggggccgggcggactaggagcgcgcgtagaggggggggcgccgcgcgcgtggggcgggggcgcgcgtgtgcgtgggcgcggggaggggggtggggaagccgcggtggcggcggcggcggcggcagctgctccctccgcatgttctcgctgcatcttccgagtggggggagttatggtaaccggagggggacagcggaggacgggagggtcggggaccggcctcgtgcggacagggtcttgagcacgcgggggcggccggtgtgtgtgggatcttggagcccctggtggaaggggcagcccgtgtgtccgcgtgtgtgttcgtgcccatgctcgtgtctcctgtgtgtcggtgccatgtgcacacgggggcccagttcctcgtgcacacatgtgcagccatccttgtgcaagtgtgtcctgtcttatcttgcctctgcctgtttcctgcctagggggctgttgggccagccacgggcttgtggggcagtccgcggccttgctggtccatgtcacctctgaccctgagcatgcatatccctggctggggctcacctcctgtcaggcttctgtgtgtgggaatgtgtggccctatgagcaggattttctgtgtccctggtggcaggtgtgtgtctttgcatgtgtgttccagcactggcctgagtatctgtgtcaatcccagcatgggtgtccagggcgtgtctggatgtgggtggtgtggctggggggagtaagtgtgtgaatgtgttccttggaattctactgtgtatgaaacacctttgggctgggcaggtgtaagccctgcttggtcagtaagtggggacagaaaagcttaaaaagggaagaaatggggtaaagaggaaaagtggggaggggataggagagaaggaatgagagaggcttgaagaagagagcagagagcagcaggccagggagaaagaatagctggagaatgggaatgtgggagaaagccaagccagacttagaagagagaacaggggataagagcttaagatggggaactggatagagatggtgggagaagaaggggtctgggagctggggaggcagtggggaggagagagtcaggccttctggtaaagtgatctctgaaagaagaaagggaagaagatgggagaaggcactggattcttgtaggccttgcaggatcaggcttggttttggatcagggaaggccactcaggcatacgggctggctcacagtgaggcctgtcatagcagggactgtcattagaggttcatatccccagcacagagctctgtctcccatttctctgctctgtgtccaaccctgttcttcgcccttctctggcaacctccctcctctggctccccttgagagctgccagctgggtgtggagaggaagaaaggccaaagggaaggttagggatttggagggcagggggagattctgtgggtgatggcaagattagagatgttggaggggcaggagtccaagcccttgctgtgtgtgtgtgagtgccattgtgtgtgtacacagggtgtgtacatgtgtgggtgtgtgcacaatgcctgtgttacgggtttgtgtaggtggctgttatgtgtgtgcatgcgtgtatacatcgtgcaggcatgggacatggagacccaggatcacagtttgtgcttttcaggtttgctgtgtgtgtgtgtacacgggtgtgtgttgttttctctgtacatgtcactgcatgcagcattaataacaggatgttctgtgtttctgtcactctgccccccctttcctctgtgtttctgctattcttttcctgcggcaacaaaagtgtttaataagcatatgtgtatgtggcctctgtgtacctgtgacttatgagtgagttcatggatcttagttcttggtaatatgtgtaaggttatattacttccaagaaccaagcaagccaattagcaaatttgttcttctgttaatgctttagttccaaagcggaattttaatgcatttaaagttagactttatgcctttattagatgagcttgcacatgaaaataatggctgagctcggttccctgaaatgagagtgaggcagccatacctgagaaatgcagaaaaaatgttactagggaagagatatcctgaaccattggatgcattgcttagtgtgtaccagaaggataattaacacatttgctcctggtttttctttactctggtggcaatctcggatgcctgtgttagagtgagagaagggctgggggaggaaagggggttgtcctgagacagagtggatgtggttgtacttttcacgtgaatgaaaggatgtttgtgcttctgagatatggggatacctttctgtggtcaggatcatggtgtactcaagcatgtatttcaggggttaaactggaattcccctgcatggggatatgtgtgtcttagggttataggtccatttctgtagtgtgctgtccacatgattgcccaagagacttgggccacaaagtccacaccagcagctctcatgtgggtcactgggttcaagtatgtactcccttgttgcagagctcacatatgtcaaagcatgtatcctgtggggtgtgtgggcctcagggtctcagaatgtggatccgtgctatgcccgtgctcacagttgtctgtatgtctcaaaagcacatagatccccagttatttttctgtattgtggttctcatatacacgtacctccatagctcagtatatgtttctgtactatacacctgttcctgagggagtgatagggttctcgtgtcatggggtccacatttttgtatgcaaacctcctaacacctgggttttacaggtaaagggaagctgaggactgattctagggcagtttgcaggcaatttgcaattctggaagcagacagtgaaaatataattgtggtcctcccttgttctgttctggaacctagagggttaacaggcagctcttccggtccacccccctccttccagcctgtagcttcttgttgccgtggagatggtggccccgccccgatgcagcacctgcccccccattggctgcagtggtgactgtggggctgaggggggagcccaggcctgggcagccattctggagctggagccggagcttggcatccccccacttacattctcttccagcccctcggcccctctaaattcccttgcagccgagcctcagcttcctttttcagcgcttgccatccctctaggctcttggcagactgagctgcatccgcattcacctcccctcctcctcctttccctatccttacttgggagtcccgagaacccccactgtccatccttcctgctccctgcccattcccctgcattctttgcttttcccctccccccaccaaccttctgagcccctgccactgcagtgcacgtggaaccggctccttctcccttccccccacctgccccttccttggattttctgtccccagcttctccccctgctactcttcacaggtctcttccaagacctccccctccagtccagggagggggtcatgggaggcagccctggccctccagacagacaggggttccaggaggtggggggcaggtggaagatgtgcccaccagggggaagaggaaagcctgcctatgggcaatgacctttaacccatctttccccccagctagccccttctggggtgggtgggcaccagggacctagcctccctggatctttggtgtccttcattactggggttttactgaccctctcagccatgctcccctgctgactgccagccccagtcatgggcctaaggcctcccaccccatccccctcagggggctcctgctcaggttccttgccccctccttcccgctgccagcctctccgccgtcgctgctcttcctgctgctttccggggggtaggtgaggccggagctgaggggcagagggaggtgggggcatgcactggggtcaggggtgggaacctgggttaacagcttcccttctggccccctccccaacttcccttctggccatttcctcccctccagaataccacttgggtcgctcgaggaggaagagtgtcccaggggggaagcagtacagcatggagggtgcccctgctgcgcccttccggccctcggtgagtggtgcctaccagatgtggctcagttgggccccctcccctccagccccaactggggccctaggagtctgagaaagagggcaggagggggagagagggagtgagagtgagaaaaagagtgtgtgtctgtgtgtgtctccccacctctttggtttcccccttcttggctctgcccccctgcttctgagaccagccctcccaccttctccaagctgtgtgtgtgtgtatatgtgtgtgtgtgtgtgcttgcactgtacccgtgggagcaaggaagacaaactggtattagggttcctatccccctttcctcgcctctggagacttcccttttccccatcccactttcatccaggggctctctaccagctgagggatgagtaggtagaactgaccctgccccaacccaccccatccccatttcccccccagcaaggcttcctgagccgacggctaaaaagctccatcaaacgaacgaagtcacaacccaaacttgaccggaccagcagctttcgccagatcctgcctcgcttccgaagtgctgaccatgaccggtacaggggctggagcatgtgggatgagattgatgtaatgtagggtctcctgtgtgagatgcagagggagggggttatctgtgtgcaaaggttgaaggattcaactcaagttggttgggggatgtcatggcacaggggacagaacagaaaagaactagaatagggatctgtgagcagcaggagaggggtagggtggcagagagaagacagacagacaggctggaaagggaatgaaggtgaagccaaggagggactcctcagggactcctcaggccaagaaggatgggctctagcccaggatcaaaggagctgtacaggaggagagtgaccctggaggaatgtttaaggaatgcagggaaggggttggtaggtgagtgagcaataggctgtaggtggaagggtgtcagggaaggtcaggaaatacaggggcagcaggttggagtggggctgggggtggctgaatgaatggatgatggctagggctcaaggacctcatcagtgagggaagagacagtatagagcatggcagagaaggggaggctgggacaggtgtgcagggtgacagaatgggaagcaacccatggactgaggcatgaagaagcagccagcggagaagtccagaaggcactgtccctgagaccaggctgaaggagacctccactgtttgcctttgttgcctgccatttggggttcctctctgggtttccccctcacccagtcactccccagggagaaccatgccctccctttcccccatgtctggccacccccaggattgggcaggtagggaggttgggataaagtgagtcacacctttccctgcccccctcccatgttgccagagctggatttggggccggcagggggtgagggcatggtattcctggccgcgggggcgggggggggggtccgggggccgggggagcgtcgcgctgacggcagccagagcctgcgatgacggggctgctataaataacttcttggaggctcccacacccaagctcccctcccgctttcccactgctctctactcttcatcccctgcccatctccataccgcttttgtattgctatcctacccctcattattccatgcccctagccccctttatcttctgccctcctgcagtgatttttttgcattccatcccctcttagccctcacctcggttctcccggccatctctccagttggccttcctcctcttctcctgtcctctgtcttgctgcacatacctttgtctccccctttcttcttcttgccctacctcctcttcttccctagtccgtgtattctgtcttttatcctctttgagctcttttctgcccacagctttctcctatttcttatgcttttccctcactctttcccctgcttctgctaaaacttgtcctcttatgctgtgttcattcattctttgaatcattaaatgtttatcaggcactagccgtgtgccaggcccaggctagacatatctcttctctgtgccttcacttctttacttccactttttcctttatactgaggctctggtttctggggttacctggaggtactacctagaagtgccccaggcccactttgttctctccttttttttttttcttttctgccatggtccatttctgggttgagatatttctagatgtccccagtcctcgcaatcccttaggtgtgagatggtgggagtttcttttttttcctttttttttttttaaatagaaatagggtctcactgtgttgcccagactggtcttgaactcctgggctcaagtgaccctcccacctcggcctttgaaatgttgggattacaggtgtgagccaccaggcccaggtggagcaggggagttccttaaaggattctgatttttctcacatccctcacgtccttcctgataggcagggtttctttctgtgtctgtttgggaagggtgttcagggggccttctctccaagtctccatcctggaacagactgatgatgcagggtacctatgtgtctaagaagagtaggggggccgggcgcggtggctcatgcctgtaatcccagcactttgggaggctgaatcacttgaggtcaggagtttgagaccagcctgaccaacagggtgaaaccccgtctcagctaaaaatacaaaaaaaaaaaagaaaaaaaaattagctgggtgtgctgaggcaggagagacgcttgagcccaggaggcagaagttgcagcaagccgagatcacaccactgtactccagcctgggcgacagagcaagactgtctcaaaaaaaaaaaaaaaaaaaaaaaaggaagagtgggaagccctgatcccttcctctcctgaacctcctgcctgccagggcccggctgatgcaaagctttaaggagtcacactctcatgagtccttgctgagtcctagcagtgcagctgaggcattggagctcaacttggatgaagattccattatcaagccagtgcacagctccatcctgggccaggagttctgttttgaggtactgggtctggtgggctggggagggccaaaggacaggggtgatggaaggtggggggcagagaggtctagagaaagtggcacaggtggggacatcaaggaaacagaaacttctgggactggaggaaagggtaggccagggaggaagagaaggtagcagagtctcctcccctctgtagccctttccctcaactccacactcctttctaggtaacaacttcatcaggaacaaaatgctttgcctgtcggtctgcggccgaaagagacaaatggattgagaatctgcagcgggcagtaaagcccaacaaggtattggggaataaaggggacacaacctgtgcagggcaaaggttgcaccaacacgggaagctgggggcctagggaggaaagtgagttaaaggaggagaggcttggggaaggagaggattgaggtacagtgtatctggacaagcagggggagacccccattattctgagtcccccatttcttttcgctttctgtactgctaccctgccttacgatctctttccctgccatagaagtcatagacttacagagttgatggggctctggaaattctttagtctagcttccctgcaggcaggaatgcttcactaataccccgcaggacacatcaaatacacttagccaagtttctgtaccttggtttcctctctaagacagagggaactggtggcaatgggttggattaaatgatctctaaggtccctttggcacataaattctatgagtctgttcttcccaaaacttcatgcttccagttctgtcagctattctctgtatcccagtttctagcaagcttacagtcctagtcacactcctctggggggaactcttgtgtcttgatgtccttaaagaactcaacccaagatgctgacatgatctgacctatgcagagtacaaacaccatgttccctttcaacacactgcaatatcacctgagccttaccattagcttgtgctcaattgtatccccccaggcccttactgattcttttttttttttttttgagacagagtctcactctgtcacccaggctggagtgcagtggcgtgatcttggctcactgcaacctccgcctcctgggtttaagcaattcctgtgcctcagcctccccagtagctgggattacaggcatgcaccaccacacccgactaatttttgtatttttagtagagacgaggtttcaccatgttggccaggctagtctcaaactcctgacctcaggttgtctgcctgcctcagcctcccaaagtgctaggattacaggcatgagccaccacatctggccaagctttacccattctatatgcaattcttttttcacaatttctgatagtctctgcaggactttccagttcccttcaaattctacattaatatttttggcttgttattccagcttttgaaatctttccaatactgtttgtgttgtctagtgtatgtctacgcttctacagtagcttcctggggctgctataacaaactgctacaaactgggtcacgtaaaacaacagaaatttattctctcacagttcaggaggctggaagtccaaaagcaaggtatcagcagggccacgctctctttgatgctgtagcagggaatccttccttgcctcttcctagcttccggaggttgccagcagtccttggcattcctgggcttataactgcatccgtctaatttctgcctccatcttcatgtagctggcttccttctgtgtgtctctgtatcctgtatctctgtgtctccaaatctccctctccatataaagacaccagtttttataaggtgggttaagggtccactctaattcagtatggcctcttttttttgagatggaatatcgctcttgtttcccaggcttggagtgcaatggcatgagctcagctcactgcaacctccgccttccaggttcaagtgattctcctgcctcagcctcctgagtagctgggattacaggcacgtgccactatgcccagctaatttgtttttgtatttttattagagacggggtttcactatgttggccaggctggctcgaactcctgacctcaggcaatccacccacctcggcctcccaaagtgctgggattacaggaatgagataccatggctggcctcttcttagtttgattacatttgcagggatcctgtttccaaataagatcacattcacaggttcagggtagacatgagttttggtgggatactattcaacccagtacaccgtctcaccttatgattgctgtctgtatcccactttcttactgtctctcctcttttggtgtctctctgtcccttccccctttaacatcatgcccaccccaccatgccaggacaacagccgccgggtagacaatgtgctaaagctgtggatcatagaggcccgggagctgccccccaagaagcggtactactgtgagctctgcctggatgacatgctgtatgcacgcaccacctccaagccccgctctgcctctggggacaccgtcttctggggcgagcacttcgagtttaacaacctgccggctgtccgtgccctgcggctgcatctgtaccgtgactcagacaaaaagcgcaagaaggacaaggcaggctatgtcggcctggtgactgtgccagtggccaccctggctgggcgccacttcacagagcagtggtaccctgtaaccctgccaacaggcagtgggggatctgggggcatgggttcgggagggggagggggctcggggggtggctcagggggcaagggcaaaggaggttgcccggctgtgcggctgaaagcacgttaccagacaatgagcatcttgcccatggagctatataaagagtttgcagagtatgtcaccaaccattatcggatgctgtgtgcagtcttggagcccgccctgaatgtcaaaggcaaggaggaggttgccagtgcactagttcacatcctgcagagtacaggcaaggccaaggtgagtgttgtgccctcagggaaaggtgacttgggaatgggcacttgcttgggggttagtgaggacagggcaaattcacgagattgggttgtgcagaggctgacacttggattttcctgggcctcaggacttcctttcagacatggccatgtctgaggtagaccggttcatggaacgggagcacctcatattccgcgagaacacgcttgccactaaagccatagaagagtatatgagactgattggtcagaaatacctcaaggatgccattggtatggcccacactcaggccctcttcttcccaaacctgccagatgtccaccccagaccccaagtccacccttccacagcttgatacttcctaacccagagtcctaggactccagcctccaacacctgattctgaaatttccccaaccctggccacccccttccctgcccttggaaagtgtgaccacaccctcttgtgcccccaccccccaggagaattcatccgtgctctgtatgaatctgaggaaaactgcgaggtagaccctatcaagtgcacagcatccagtttggcagagcaccaggccaacctgcgaatgtgctgtgagttggccctgtgcaaggtggtcaactcccactggtgagactgggaacgctgggctggggggccagggtcgggggaattatgtgttcatctgttcatctatctgtccatcctcaaagaggactgagcaccatttatgggcaaagcattgttctaggcgctatagagcaaacaggtgaaagaggcctggtccctgccctcagagggcctccaccagaatggggacaaattagaagaaaaaaaaaaaaagccacagagccataatggtgtgtaagtgctgagtaagggtccccccaacctctgtgtgacataaggtcagagagaaggcagagctttgagataagtggggaagaggtgcccccttgggtaggctttgaagactggtttaggttctgatatatggacatagttggcaagaaagacatttcagaagaaggctgtgagaaaggcacatgtgtgatggtgaaaaggcccaggagttttcaggggacattaaagtaggttagtagcaattacatcaggtttagtggagcatgtgcctcataatggggagtggcgggagagatgtctgggcaggaagattagctttagaaactggaaggcctcaaggagtctgaggtcattggtaggccttgggatgccattaaaggtgtcagaaatattgtgatttagaagattaatccataggctgggcacggtggctcacacctgtaataccagcactttgggaggctgaggcgggcagaccacctgaggtcaggagtttgagaccaggctgaccaacatggagaaaccctgtctctactaaaaatacaaaattagccagatgtggtggcacatgcctgtaatcccagctattcgggaggctaaggcaggagaatcacttgaatctgggaggtggaggttgtagtgagccgagatcacacgattgcactctagcctgggcaacaagagtgaaactccatctcaaaaaaaaaaaattaatccataagtagagtgtatgtagtagagtagagggatgtatgttgggggatgactggaatagagccagctgggggctcggaggcatgaaagtcagatcctgaatcagaacagtgacagaagttaggaaggagctactggaaggaccctctcgggaaaggatcagtaggaattgtcagcttattggaaaggagggagcatgtctgtggggagtcacggatgactcaagaggccatgaggctggtggttgggagaccggtggcctcattgacaaccaggaaagtcaggaggaggagccagttggaggtgtgggggcgatggtgagctctgctttacaccagccgagtttaaggtgtcagtgggacattgaagtggaactgggaggtgcggggagtgagctctgagccattccagggactggggatcatgcctggggcacctccatccccatttccctggaatccagaagagttggggggtccgagctccctgtacctcaagtgaccctccatctctctcccatctctgtctctccctggtgtctgtttttcttctcctcctctccttgtctctctcccacacccctccatctctctcccacgtgtctctcccctcaccttctctccccctccatttctctctccctaatctgtctgttccctctgccatggcccccttcttcaagcagcctcccatcttgctcctgcggtccctccttccctgtctctctcacccctgtttccacaccctcacctcctaccacccccctcagcatgttccctggaagctgagggtctctggggctcagtcccggtctctctctttctctctctctctctctgtctccccgacccttccccccagcgtgttcccgagggagctgaaggaggtgtttgcttcgtggcggctgcgctgcgcagagcgaggccgggaggacatcgcagacaggcttatcagcgcctcactcttcctgcgcttcctctgcccagcgattatgtcgcccagtctctttgggcttatgcaggagtacccagatgagcagacctcacgaaccctcaccctcattgccaaggtcatccagaacctggccaacttttccaagtgagggaagcttcaggagtgggcagggcagggagtggcagggcagggagtggcagggctgggggtcggcaagaagggtctcctgagtccccagagatcctgagatggggaggctatgataccttgtgtgtgtgtgtatgtgtgtgtatgtgtgtgtgtgtgtgtgtgtgtgtgtatgtgacctttatcttctgcattcttggctaggtttacctcaaaggaggactttctgggcttcatgaatgagtttctggagctggaatggggttccatgcagcagtttttgtatgagatctccaatctggacacgctaaccaacagcagtagctttgagggttacatcgacttgggccgagagctctccacactgcatgccctactctgggaggtgctgccccagctcagcaaggtcagcagatcccctctttgccctatccccagatggctccagaggttcctggagcctgagaaactaccctttgaagattttttttctccccttgtttctcgaggtgtcaccactactatcccaactcaggccccctccacctgcaccctcagaggccctcttagagctgggcactgagcccccaggtaacagcctcacccttccaggaagccctcctgaagctgggtccactgccccggctcctcaacgacatcagcacagctctgaggaaccccaacatccaaaggcagccaagccgccagagtgagcggccccggcctcagcctgtggtactgcgggggccatcggctgagatgcagggctacatgatgcgggacctcaacaggtgagcaccctgggacagccaggcctgtgccctaggagcccttctcctattctagatactcctcactgggccccacatgcatctctctagggcttgaaagaagggaggaaaaagcaccaagttctcaggggagacgataaggagacaggtacagtcagtggtaggctgagagccctttacagcctgagggagtgagagatttggagctctaggaatagggctgaggctccaccaactcacggcttagttgtaagcctagagcatccctgctgcaagctctgatttgctgtccctctgcctgcccatgctagtccccaggctgaggttcagccagcatgtcatgtcagccatgtgtcaaaatgttcaaacatctcagtaatagctagtgaataagcacttcccccagcccccgaccacaaccccacagacctccccatgatccagcacttagagcagtgacagcagaagcctagcagggcctgcagcctgctccagagtcccagcctccattctgataggtggtgcccgtgcttcatttgctgctcattattttgatgatggccctgctttttcctctgcccgtgtcttcctccatgacaccatacccatcccaccattcccgcctctcctttcatttgtccacatctctctccttctctgtctgtgctcgcccctctttccatctctctccagctccatcgaccttcagtccttcatggctcgaggcctcaacaggtgaggggctctcccctcccccgccctcctctcctctcctgtctgttccctctcccactccactggccttcgccctactcctctcctctcctcctccatggacctcatctcctccatatgtgcccagccctgcccccatcccttctcttgctgcccccatctcccctcctctaggcctcacccccttcccggaggggccctgtcctttccctttactcacctgtcccctcccatcctccctgcctgccctcttcagggctgccaccgctagctctcagcccttccctctgggtcccacttttcaccccaaggcctgtgccagaccacagcaaggttcaattgctaggagccctgaccttaccttctgcttgtgtgcccccttcccttctgacagctctatggacatggctcgcctcccctccccaaccaaggaaaagccacccccaccaccgcctggtggtggtaaagacctgttctatgtaagccgtccacccctggcccgttcctcaccagcatactgcacgagcagctcggacatcacagagccagagcagaagatgctgagtgtcaacaagagtgtgtccatgctggacttacagggtgatgggcctggtggccgcctcaacagcagcagtgtttcgaacctggcggccgtaggggacctgctgcactcaagccaggcctcgctgacagcagccttggggctacggcctgcgcctgccggacgcctctcccaggggagtggctcatccatcacggcggctggcatgcgcctcagccagatgggtgtcaccacagacggtgtccctgcccagcaactgcgaatccccctctccttccagaaccctctcttccacatggctgctgatgggccaggtcccccaggcggccatggagggggcggtggccatggcccaccttcctcccatcaccaccaccaccaccatcaccaccaccgaggtggagagccccctggggacacctttgccccattccatggctatagcaagagtgaggacctctcttccggggtccccaagccccctgctgcctccatccttcatagccacagctacagtgatgagtttggaccctctggcactgacttcacccgtcggcagctttcactccaggacaacctgcagcacatgctgtcccctccccagatcaccattggtccccagaggccagccccctcagggcctggaggtgggagcggtgggggcagcggtgggggtggcgggggccagccgcctccattgcagaggggcaagtctcagcagttgacagtcagcgcagcccagaaaccccggccatccagcgggaatctattgcagtccccagagccaagttatggccccgcccgtccacggcaacagagcctcagcaaggagggcagcattgggggcagcgggggcagcggtggcggagggggtggggggctgaagccctccatcaccaagcaggtaggtgaaggcaggaggaaggcgggctgggtcacaacagggagggaagaaggagatggggggtggggttgaaacagagtctgtggcctgaagttacaatcttcttgcctccttttggccattaaacaaatgtgtatagagggcctgctatgtgccatgtgctatgccaggcactaaggatacggcactgaaccctcttagcactctcattccagaggggattaatccataagtagaatgggggtgactggaatagggccagctgggggcttggaggcatgaagatcagatcctgaatcagaacaacaggagaaattctctggcattcaactcacatctctggcattcagaggtgataaaggctacagcagggcaataggactgggactgtcgtctcctttggctgtgctgttgccctctaaatgtaccctgctttttcccaccttttctttctctgttcgccctcactgtgccttgtcccagcattctcagacaccatccacattgaaccccacaatgccagcctctgagcggacagtggcctgggtctccaacatgcctcacctgtcggctgacatcgagagtgcccacatcgagcgggaagagtacaagctcaaggagtactcaaaatcgatggatgagagccggctggatagggtacgatgggctctaccagctccaagccccagtgtttccttttacccacaggggagatctctagtcacttccaagggaaacctccagggtcagttatggtggtaagaaaaggcaaagacctgatcacctcttgagaagccttccgtccatctggggagaccagatacacacagttgttaaaagtcactttcaactctattttgtaggttatatatatcatagactatctggggatcatctattttaggcttacctccatgttcttccccaggaggaacataagccctggctcctgtgactgcttaggaaaggaaatgttactttactggcagtaggagctaataaattggatggggatggagggggttgcagctatattacaagtgatgtctggcctacccagtcactgtcccatgggcataagtgacagtcagtgggcagggaatgcatgtggtaaagtagtatgtatcagtcatgtgtgaaaagaccatccagaccctcacttggtggaggctgagggcaaagggcaccaagagctctggggctaacacagataacttagcagaaagttacttgaggaagctgttgttcctagggcctgaggaagagggcggctgcaggattaggtcatatataaagaacatatgatcctaacacggggaagtggtctttgagcataggaaatgttgggactgactcagatgggcttgaccagcaaatttaatgagaattaaaccctaatattagccccaaggcagccaccgtaggaagttaattcacatctctcctctgcatgtagaagggttggtgatatatgtatgtatcctcttcagagaatgaggaaatagtcttttgaatcatttttttttctgtgcttcagatgaggaaaccttgttgagaaaggctgtagactagcactatccaatataactttctgcagtgatgaaatgttctgtaatctgtagtgttcacatgtgtctagttgtgattcaggaactgaatttttagatctatttaattttaattttgtaatttttttgagacagggtcttgctctgtcacccaggctggagtgcagtggtgcaaacatggctcgaggcaacctccgcctcctgggctcaagacatcctcccacctcagccccctgagtagctaggactacagggtcgtgctaccatgcctggctagttttttttcttttcttttttttttttttttttttttgtagagacggggttttgccatgttgcccaggctggtctagaacttgtgagctcaagtgatctacctgcctcagccttccaaagtgctgggattacaggtgttgagccactgtgcctggcctaaattttaaatagctacatgtgggctggtggctaccatattagacagcacagctgtagactctagagccaatagggcagataaaatgactcttctgagctcttccagctctggaattcctagattctcagcctagaattcacttgtagacctcctatccttggcaccttgacagagccaaggagagggtcacatggaggacagcacctgcctttcccccgtcaattgccttttcttcctatgtctccagcatgtgttctggggcctggcttagggctgaagagtcaccctatttttaatttctgagcacagttactcaaggtgtgtttgtgagtgtaggtttctgtgtgggtcttcatgaggggccatacatagacctcagggtgtgggagtctgtgaccttgctccattttgagggaacctcagcaccatggcagggtcttctcaaaagcaggtgtgtggatatgatccacaggcagggaaggggttggggaaggcagctggatgtccctcaacatgcagtggctgtgcttggcagacagggatggaggctgggtggtgggcttggggtggggcgcccctcatagtgcggggtcgtgtgcccggcgggcaggtgaaggagtacgaggaggagattcactcactgaaagagcggctgcacatgtccaaccggaagctggaagagtatgagcggaggctgctgtcccaggaagaacaaaccagcaaaatcctgatgcagtatcaggcccgactggagcagagtgagaagaggctaaggcagcagcaggcagagaaggattcccagatcaagagcatcattggcaggtgaggggcggcctggggagggggttgtgagggagagcctgaggctggagagagcaagtgggtgagctactccgctgactcccatccccaaactcaggagcccaccaggagagcccaccactctcctccccaggaagccacccactcactcatcaccagatggagagaaaccccaacctgcttagtgcattaaatatctctttaccaaaccctgacctctcttctgatagagtagcttcggaagcccttggaaaatgtacctgttcctgtccaaccatcactgcatttgcatttaccctaggccagagctccccactagttattctcaacttaacctgtgatgttcactccaaacctaagcagggctccctagccagagtaggccctgcccttcctgggtggaccctccctctctagccttggaaaggtgttctgttagaaagggtcttttagcctgtgtatgttttcagctgctccagcaagtcctgggctccaaagagggtatcctcagcaaagaggtcaattatcttcagagtggtggggtcggggtgggggggaccctgggcgcactccaaccagagccacctccattttgatccattctaaatgtattttatgtaagatttaattagaagaaaagggcttcttgaaatattttttgaaaaccactgctctaattgatatcctttatgataaatcacctcgaggatcttcacagtgaggtgacatgggggatgcagaagccaggtcctcagccatggaaggtctggggaaggggcactgctgtcctgattgggacgatggaggcctggaggtgtctggatggtagaagtctttgaggcacagaaagctgccttagcagggaggtgtaagggttcctgggaggaaggtggagagcatgatcctgaggaacagggagtcttgcatcacggcaatggagggactctgattctaagggataaggatgcctgaggtttttccagagagctatggggttccatgggcaggctctgagcctgtgcccgccactaaccccactgaagcccgtcccttcaggctgatgctggtggaggaggagctgcgccgggaccaccccgccatggctgagccgctgccagaacccaagaagaggctgctcgacgctcaggtggaaattacaatgtcatttatcttctccgtgtcccatccccatccatcccactgtctttcgtgcactcactacaccagccacctagccccatcaccatctgtctctcatagtctgctgtttgtccactggctgctcctggcagccccctagtgaccccatcttcatcccatcgtctgtgcctttgtcactcctggcagcgtcagcccaactcctgtgccttcccatccagtcttcccactcctctctgcatctcaggaccttctctaccagaaccttggtctttctgcccctagaccccacctagttccaagaacccctgccccttctttgctcactcctattcaagccacgttgttcagcttcctctgcgctcttgggccagagggctagaagctgccgttttctggaatagagcacagggcagtatgatctgtagtttctccaggccctggccggtaccctgaaaacttggggacccatcacctctgttctcttggctccctaattttcctgtctccttggcagctcctgcatagcttcctcttcctgactcttcagatcttgaaggccttccatcctgtaacctccctttgccctcagtatttaagtccagcctccctctggcctccctcccactctggccctcagaccttcccagctgcctgctgcccagcctctcttctcacaagccagcttctaggacctcccttctgcacccttaccccttgctttcccaaaattctgctcattttcctacccatactcctctttgctctgactgctaggctccccccgcctgccatccccccaccaaggctcctgaccccatgaccccactctctcccactgcagctcctcatcaggtaattctcctggttccgctttggccacgggcggaggacacagggggaggtgactccggaccactgcaggttggtcgtgaagcccactccctccaacacctccggagcctctcccctctcactgctgccctccacacccagagaacctccacagactccagccctccgacacctgcacagatccatctcccaagacaccacccaaagagagcatttgctgctgcttcccagaactgtccaacaataccttagcaacaccaagagttgggccctagatgggcccagcacattcacaggtcacacccacttccctgcaaaacccaccccctcccagcctcctcctgactctaagccctcctcttcctctacctctccagtgtatgtctgtcaccccccatttcaccagagcgtccttaggggctgggggtgggtttgttaatggggtggaggcaatgatgggttggaggatcttggctataggggctgtgctgactgcagcaggtaggttgggtttccctcttccttccctaatcttggttctctaccctcctttccactcctcacctgattctctctcttcctcctccttatatctgtgaggcagaaggcatctgaagctcatattagcccccattgggtgggaattaggagtgggtagttaactcagggagacttgagataccctggaaaaaatgctattgagatgtcctgacattaggcagggtggatggaacaagaaggagcaagaaaggaacctcaggcagatgttaggacatggacttgatcatgtggcctgggagtttagaaatggggagagacatcctcctagatcagatcgtgggctcagtaggcatgttgattcccagggagaggtgccaggaacagcatggtaaagaatgtactcttcacagctcacatccccaggttgctgatgccactcactccccctctcctgccatcgagtggccttgccggacacatcaccctacctaaaaagccagtaaatgagaacctgtcagctatagccatcatttctgagatgcgattttctttgggattgagctgcagtgggcagtggctccttacactgtaattttaattctctgcctgcccagcctctctgtcaaagtagctggtgatctataaagatgctaaaaggcaccaggggactttgccatttaaaggactcctgcagtgaattcttttgtaaaatgaataatggcaccctaatttatccactttctaaatttgggtccatgggggtgtccagggcatgcttatgtgctgtcaccagcagacaaacagagggaatggaatctgggggttccttccctgctctcccgccatactcaggataccctaccataagtgatttcctctcactgacttgcagaaaatgtgtgagatacccagcaagctaagaaggcagttttgctgggtatctcatacccaaggctggggtttgggtgatctgagaggttagctccttgatcctaggatggaagggagagcttatatagaagcttttacttggaaggttttgtatcctaaggtcagacatagctatattaccaagcctaaatgccatgtggcccaggaaataatttggacatttgttctaaaccacttgtggtaggtattggtctctctgcaactcagccattaattagaaattagttttgagcctgaattttaaaaagccaagtgttgcccccagcccacacacacacacacggacatgtacagtacaaaccccagataattacaacagccaaagagagaaggaagtgaatttcccaaccagaagcgtagggaaattcagatggctttcttttctcccagcagaggaacagaaggcggagctaagggcaggaaccaggagttggtcaaggagctataggaggtgatgagagtagaaccaggggtaggagctggtctggtacccctcaccctctaattgggagcccagggagaaggactgaaaagaagatgggagtggaaaagaataaagccagtttctgcttcccagggatgcagagattggggcatgctgtgtctgcagaagctcctagtcatttccgccataattgtgagagagaggggcagccctcccacaagatttttcccttcccatatcacttccctgaatccccttcctttccccccagtacagttaaacctctctctaatttggaatgttatatttgagaagatggccactgtgaataagtgacaaacggaatgacagtgtctatttaatgaaatgcatgtcttcaaaatatatacagaactcgatgaacaaggctttttccactcctcagggagcatgcattaatgaatagataggattcaaaagtctgttttctggtatgggttaaatatcccctcctacagacatatttccaccactaatttgcttagtacaccttttcttcacagataaaggaaaatgcaagctcagtttttcttcagattatgaagaaattccaaatccacaggggtttggattaatgaggttttgctgtactgcctccccttattcctcaacatgaagttcccacctcggattggggatgggtgggagggggtttcaagaggaggagggtgggatgggcaaggaatatacacaggtgaagccagagaagggttaggttgggggtgcggtgggaacttgctgttttgatctggtttcctggtgtgacactctgggttaaaggcttgaaggcccctgttaggagtctaggggtgagattctcttctctctgatcccagaggacgttaacttctactgcaggtgagaaacaaaataggaggatggtggggactgtcctgggaggagggggtggtccatggcttgtggtgtgggctggctataggggaggccactgctaggggactggcatccaggcccccttgaagcgtctcaataagtccgcgctctcctttttggtgtcttgcaggagaggcagcttccccccttgggtccaacaaacccgcgtgtgacgctggccccaccgtggaatggcctggcccccccagccccaccacccccaccccggctgcagattacggagaacggcgagttccgaaacaccgcagaccactagcccacccagcatcagagaccttctcttcctttcctgtgcaccccaccctgtaacagcaccaaccaccaggattggacatcaccgaggaacagcgggattgcctccccgaatgcctccctgggaggcacactgattgcccacccccaccactgcaccatttccaggagggagagtggggaccctcagccgcccccttttccttcccattggggtgctgccctctctttgacccccagggacccttgccccaggacaccgcctaccccgtacagaccccttcactccggggtgctatccccatcctctgcctcatcgttcccctgagcactgggggacagaccctcacccccaccctgggggtgtggcacctccaaactttcaacttcagggtgatttttttagcagtaaccagagctgacaatctaactcccctccaccgccccattttggcctcccctgccccccttgttatggggaggggaccccgggtgagggggccctattaccccttgatttctcaggagcgtctgggggggctcagcacgcacaaactccttctccttctaccactcttaaatttactccctccccacccagaacccagatggggtggagggggccaccggggcagggagggggcggcaaggggggaatgggagttgtctccccttctccccacacctgatctgctctcggctggtcccagagcggggtgagggggcttatgcccccccctcccccagtgtgttgggtggggtggaattgaggttagggtgaggggtcagggtttaggagggtgtgtatgttgggaggacaggctagttgatctgtcctactctgacacacagtcccctctgccccttccttctctcttcttggtctctactcccagggggaggggggaacttactctaggaaaagccatgtctctctcccccagggtggggggacctgtgttggaggaggggtgttggggggcccccttccatgactctgtcccctgggggaggtaggacagggctgggcttccctctcatcctccccctcccaatctccttccacctccctccctcccgccagctccacgatttttcggtgtttctctgtacatagttttctggcgggataggggaggtaggatggatggggtttggggtgggtaggccatgggaggggagaagcccctccttggcaccccctcttccctgactgctgtcccctacccagccttgcccccttcatccttttgcgtttggtattgagactctcctagactctactcctctttcttttgtatggacagttccccttcagtcccatccccctacacatacacccagccggggccaaatttatacttatataaaagttgtaaatatgtgaaattttatccctgtgccctttccccacctcaggccctacccctggaccctccccaaccttccttctctcttctttggctgttgtaattatctggggtttgtactgtacatatccggggtgtgtgtgtgtgggctgggggcaacccttctgtacagagcttcctggccccctccccccccgcccctctgcttccctccccacccaccacctcaagggtagggagttgctcttcctacctgttttattttgttttctcgttctccctccccaccccactcccagccttatctatcccccctcactgtccccttttctccactcccagccccatttcctttttttctggagtgtgtggtgaaacagaaaaaaacatgtttaataaacggagattgttctttta

SEQ ID NO: 7 - DNA, 33623 bp, Homo sapiens
  source 1..33623: mol_type = other DNA
ctctctctcggctgccgctgctgccgttggctcttattctcctcctcctcctcctctctcctcctctctgcttctctctgctcctctctcctcctctctcctcctcctcctcctccacctcctcctccttctccccctctttctccccctctttctctcttctttctcccccgtccccccgccccctccccccaggcctgatgagcaggtctcgagcctccatccatcgggggagcatccccgcgatgtcctatgcccccttcagaggtacgtggtggggggagggggagggccatatggggggcaacagggggaggggcagggggcgggggagagtcggggccgagggaagggagcggctgaaatggagggagaggggaggaccgggaaggcaggggtgggagcaaccgggaaggagggattggaggccagagggataggggtagataagggatggaggggccaggaaatgggccttgaaagggggctgaagggaaaatgatgaaggggaaagaaagcgccagaaaggggtgggaaggtagaaggattgggttggggaaaaagctgcagaaataatttgggggtgggagctgagtgatgaggaaaacatcaaggaaatagagggacagagtcttgggagaggggctaggaaggagaaatgaaaacataggggtgaaaagagaagccaagaagatgtggggggataatttggtgggggactcagagtatggtaaggggcggaaaaagagaggaggatggagggagagaaggggccagatggagagagagggaggcagtggggggtggggggatggagggtccagatggagggaagagagaagagagagggaacggtgggggaggggagaccagggcagggggagggccaaattatgtgtgcttgggggggtggcagtacaagtaagatgaaatgatgggagtaggggacaggttaggatcctgccccagactctgaccctcttaaaggctcttgaccagtaaaatgtaacctctggggtcttaatgctgtcttcacttcatcacttttacccccttcccccacccctccaatccttatttccattccccctccctcctctgctgtccatcacctatatctttccccccatatggtgtccgttccttatatgagtcccccttctgtccttcccctatatcagtcccctctctggtgttcattgcctctctcttatctcttttctatgtggtcgctcccttatatctctatcacttctttactagtccgtcttgaccccttctcatagcctccgcattaggtctgcctcctttgctctgacacatgtccagtcgagcccttcaggtgtattcccttgcccctgggcatttaccatcatttctgcctccttttctatctactcctattatttgtctcccagaccattccttgtcaccacaagtctgtgcctacccactttcccttaatacctcaactcttattatcatttctcttttcttgtctcctctacttattggggtgttggagatttggggtacaaatacacaggttttgatgattagaacaagggatccccatctcagcccttgagggatttctcatcctagtaaaaatttcccgtttctcagccatgttgctctgcctcttgcatctgcctcccagaatcattcttcaccaagtccagacagtatgtgtgtgtgtgtttctagagttgttcctaagttccctgctgccaggagaaagatgccctctgaagtctaacccgcctcccttggctgcagcctgaaccagccctttccccaaatgcttatgtgtatcaacagatttctcctctgggtataccaagtgtctaagtgcggtgggcatgttgcctgcaatgtctgtagtttcagaggctaggtattggtgatctctgtatctagagaacattagttagtctctgagatgggttgagatagagtttcaaagtatgtggtttctaaatggtggacattctgagggttcatcgctcactgtgatctggaggttgggtaaatttgagagacatatttatagacagaaaagggctgaatttgcaaaactctgctactcagtgcattctgttggaatgaagctcaaagaccagttctcctgcagtatactcatggttatcccataaaggtgacagagtttgatcttttttccatctaaaatctgataccttaagcctaatttctagctctttgctgtcgccaatgtgacatctctcttacttcaatagggcctgtgtggcattggggtttaaaggagaggcagtttgcagggagagcggcatggcaagaacaacagagctggggaaggaggaccctggtctcatctcagccataggcgacacttggtctactgttctctcttctcaaagttttgttcccatctccagctcttgtctttggaggcccctctgtgtcaccaggcctggatattccattctttccccctgacatctttcttcctcaagctcctcactgtgccttgagaaggctcctgacctatggagtggcttccatcctgtccactgagactaggtggggtgaggtggtgggggcagtgttgaatctctgggcactgaatgggagaagagataaatttggtctctacttgtccaaacaagggcatatttcccaccttctgccctggggcctccacagccagatgccgacaaaagccatcttctccttaccaccagaagaaccagaactttccctaaaggatgagcagggttgaactaggggaggagggcacagaggagggaagagttgggaggggaggccctctcatgtctctgactgtgaacaggtaagctccccgacttcttcttttctagatatttagccctcagaatctcatgatcccaatgttcctacacacacaccccatccttctttcccacagccatgtagctttcctcatactcttttatagaggggtggtgccaccttccaagtccttgtcttctgcagctatttggatgccttcctctacccagatatctgacaccaacttctctgtttttctcctgccaattttgcggcacctctaccttattttcctgaagtagatggagagctccttcatctttctctgcccccaaacgtgtaccttctctgtcatataattcccatccctgaacctctgcatttcttcccccaagtctccagagccttctgtgatgtgatcctctcctcttttcccctttgtataaatgcttgtcttgtgttcctttagagttgccaggtgtctcactttcaactgtgatctcctgaaggccggtagggtccccttctcactttgtggactctcttcttgccattttaggcctctgcttccaaatgcatagagcctcccttactgtttctgtgtgtctctgtcctccagatgtacggggaccctctatgcaccgaacccaatacgttcattccccgtatgatcgtcctggttggaaccctcggttctgcatcatctcggggaaccagctgctcatgctggatgaggatgaggtgagtgtggtgagaaggctgggaaaacgcatgtgggagaatggggagagtcctcattagtgaaggggagagagacacaaagaaggcagacaaagaagaaacatttgcaaatggcaggagggtggccattgagacttgggttggagtgtgacaagaggggtagtcattctctcatcttgggcagggagtctttgagggttatgagctgtgttggggaggagatgtggttctgaagatttcgggggtggggaggctcctgttttcacactgtccatattgtaggataggttagaatggggaaggggaactgaagggtttggagaaggggtgaagacatggaaggccctcccaaccaaagcaatcctcaggacccctctctgctttgcccactgagcaagactcccagtcctttctccatcagggggcagagtgcaagaagagaaattgcaggacgcagagaaaaggcaggggatagaggaggttttaggtagctggaggatactgggaaagaagactgaggtcagtggatcttggcatgatggggtgaagactgggagagtgattgagatcaaggtttagagagacactgagagggcttctgcaagggagtggttggacatcaggaaggaatataagggaggggaactgggagtatgtggagttatctaaaggagtacaagtggagtaggggcagggactagccagagtaaaggagatgaaacggacagaggcctcttatgggaggtggtggccaggtatggagatctgagagccagggaagtgtgggttctgatttgggagctgggtcagcaggagtgtccattggaggaaatagaggataaaaactggaatggtgccccacaagtgtgagtcagggaatgggggtgatttgggggacaagctttaaaaaatggtagggtagcctggaaaaccaaacgtggtgagagatggaccctgaaagttattgggaatggtgtgatgaggaagggttagaatttgagtcagggctaactagagtgtcaagggacctatgggggcaggtgagagtgtggagagatggctgggtagacagcttccaggccacagagtaaagtagaggtgttaaggagaggatatggtgactttgaaaggagaggtggcttggggagtctaagttttggacaccattgggagacatttgagtgactgtggtcaaacccctagatcaaggtttggagtctgtaggagaaatgggggaaactggaacccagagtggggactgttatagggatgaagagggaagacaatggattggggtaatgaaggaatgacagagatagggacagactgaagggaccagtggccagcaagttgaggggcaataggaatggccggtctggagcaggaagggcgtgtgtgtgggaggaggagtagatagaattgggaaaatgagcagcctgaacagacgcagggagttggaattggatgggtggtggaactggggctgggggatagtgaggggtctgttcctgtggctctttgtagaggtggaggagactgcaggggtggaggtgtatggaggtagggatggaggtggacagggggagctgggtgtgaagcttagggaaaggtgatggctgaggggaagaagggaactgagagatgctgggtcgggtgatggaaggagatgaaaggtgggggaaggtgaccaggccctctggcgggaggggaggttgggtcccagtctggccgcaagccgggccgtgggagggagacaagctgtggatcctgattataaatgcagcttctgctacccccgtgacaggctcggagggagagaagggggtaagggaggtgggaggttgggaggaaagcagggagaatggaggaactgaaggtcaagggaaaactggattcaggaagtagctaacttctgaggagttgcagaagagctgagtagggtttgggagaattctccggaaggtgtttcaagacaaatggagggagaaaagagggtgggacggcaaagaatgagagagagagactgggggagagaagggagagatccagtcaccaagtgtggaggggccggggcagaaaaagtgggcaggcttagggggaaaaggaggcccgcccttcctgggaggaggcggaggggggaagagggaggggaggggcggcgtgggccagggaggtctgacacacccccacctcccctagatacaccccctactgatccgggaccggaggagcgagtccagtcgcaacaaactgctgagacgcacagtctccgtgccggtggaggggcggccccacggcgagcatggtacggcggccgagcaggctctcatgacccggacaggcgcggggagagggctgaagatggaccgggctggcggggcgggggcgtcggggagcggcgggagctggccgggggcgggagaaggcggagacaaagtcttgcttcccgaggggagatggcgccctcctctggaaggtggagcgggacaggggctgtgggtgagaggcccctgtgggtcctcgctcggcttcgctccctttctcctctccagcagtgaagtttgactattttcctggtgcttgtaaaaatagtctccttagatctttagcttccagtccgtgtgttcctgcccctgcccctcccagcccctggtctgggatcttggaaagaatgacctagttcctgccctccccccagatctcttccccacccctatgctcctgattcttcttccggaccttcagtccttgaggtctccttttattaccccctgttttctcctctcatcagctgcttctattttctgcaattgtatcaccctttctctttcctccctgatttctcttattttctgtacccctcttgattaccccgtcgctcccctctgctcccggcgaaagtccatcagactcccatctcatctcctttctgtctgtagagctatctgcgcggtctgtctctccaactctctccctctctccatctgtctcttcccctccctccccctttctgcccccccaaccccctctttagtcatttaaaaaaagatttttgtcttcctgagaagttcgcttaaaaggtttccatgggaacagtgaggggagatgaaggcagagaacagagatctgcagccaaggtgcagagagacgccctcagcccccggaaaccccctcgtcactgcccccttccacttcatttcccttcccggacctagcgatccaggttccccattcctccaacccccaggtgacccctttttcccaccagctccccctcccttcaggaattcaagctaccagtttctctcccttagagagccagtgacctgtccccatcctttggctccttccagccttctgataggtccaggccctactagccttgtgggccggtttggggaccttgggtgggtgggtgggtggtcagggatgtgtgtcattgtcatggaaaccccataccatcggggtgtgtgggtagcacatctctgtgaggcaaagtcaggtggtggtggagtgtatacagagaatgtgtgtgatgtctgtcacatctgtgtgtatatgtatatgtgtgtatgtcatgttcatgtctggggaagatgtgtgtgtttgtgtgcatgtgtgtacacctgcgtttccaggccagaatggtcacatgctcacagagcatgtttatcatgtgtcctgtggttggcagatctgcccacttattccccatctttctccttatcctgagtcctcagggtctaggggtctctggcgggggtctcccgtgtgttttgggtggggacaccgtgtgtgtttggagttgaggtggcatgtggaggttgctgtattggggttcagtaggggctgggaggggttccaggggcatatgtatttggggggagggcttagtgaaagttgatgggggtgaacgtttagggtcttcaggcagacatggaatgtgtaaagggcatggaggatgtgcagtggacataggggtcgttgggaccttggggacttggcaatgccaaggtgtttgctgaggctgtggactctccagcccgggagaggtcgccggacctttgaggggcattggaatcctgggctcctcctctgctgggtggagccgcgaaccctcgttctctccgcggttttgtgtgtgtgggggggtcctgctcaggaggggatggtgggtggcctggtttgttttgggggtccccttgcccccctcccccgtctctctcgcgctgtctccgggcgacagggctcgtgacagaggcggccacggcagcagcggtcgcctagcaacggcggcggggcccgggcaacggcggcagcggcgggagcggcggcggagggagccgttcccgcggccgtcaccgcgcggccgtcccggccgtcccgggccccgccgccgcccccaccgcgccgggcggcgcgagccgggccgtaccgccccctctccctccgcaccccgcccttcccccgcgcaggggcggggcccctcccccaccggggacacccctggcccccccggggggcggtgcgaggtaagggcggggccgggcggactaggagcgcgcgtagaggggggggcgccgcgcgcgtggggcgggggcgcgcgtgtgcgtgggcgcggggaggggggtggggaagccgcggtggcggcggcggcggcggcagctgctccctccgcatgttctcgctgcatcttccgagtggggggagttatggtaaccggagggggacagcggaggacgggagggtcggggaccggcctcgtgcggacagggtcttgagcacgcgggggcggccggtgtgtgtgggatcttggagcccctggtggaaggggcagcccgtgtgtccgcgtgtgtgttcgtgcccatgctcgtgtctcctgtgtgtcggtgccatgtgcacacgggggcccagttcctcgtgcacacatgtgcagccagccttgtgcaagtgtgtcctgtcttatcttgcctctgcctgtttcctgcctagggggctgttgggccagccacgggcttgtggggcagtccgcggccttgctggtccatgtcacctctgaccctgagcatgcatatccctggctggggctcacctcctgtcaggcttctgtgtgtgggaatgtgtggccctatgagcaggattttctgtgtccctggtggcaggtgtgtgtctttgcatgtgtgttccagcactggcctgagtatctgtgtcaatcccagcatgggtgtccagggcgtgtctggatgtgggtggtgtggctggggggagtaagtgtgtgaatgtgttccttggaattctactgtgtatgaaacacctttgggctgggcaggtgtaagccctgcttggtcagtaagtggggacagaaaagcttaaaaagggaagaaatggggtaaagaggaaaagtggggaggggataggagagaaggaatgagagaggcttgaagaagagagcagagagcagcaggccagggagaaagaatagctggagaatgggaatgtgggagaaagccaagccagacttagaagagagaacaggggataagagcttaagatggggaactggatagagatggtgggagaagaaggggtctgggagctggggaggcagtggggaggagagagtcaggccttctggtaaagtgatctctgaaagaagaaagggaagaagatgggagaaggcactggattcttgtaggccttgcaggatcaggcttggttttggatcagggaaggccactcaggcatacgggctggctcacagtgaggcctgtcatagcagggactgtcattagaggttcatatccccagcacagagctctgtctcccatttctctgctctgtgtccaaccctgttcttcgcccttctctggcaacctccctcctctggctccccttgagagctgccagctgggtgtggagaggaagaaaggccaaagggaaggttagggatttggagggcagggggagattctgtgggtgatggcaagattagagatgttggaggggcaggagtccaagcccttgctgtgtgtgtgtgagtgccattgtgtgtgtacacagggtgtgtacatgtgtgggtgtgtgcacaatgcctgtgttacgggtttgtgtaggtggctgttatgtgtgtgcatgcgtgtatacatcgtgcaggcatgggacatggagacccaggatcacagtttgtgcttttcaggtttgctgtgtgtgtgtgtacacgggtgtgtgttgttttctctgtacatgtcactgcatgcagcattaataacaggatgttctgtgtttctgtcactctgccccccctttcctctgtgtttctgctattcttttcctgcggcaacaaaagtgtttaataagcatatgtgtatgtggcctctgtgtacctgtgacttatgagtgagttcatggatcttagttcttggtaatatgtgtaaggttatattacttccaagaaccaagcaagccaattagcaaatttgttcttctgttaatgctttagttccaaagcggaattttaatgcatttaaagttagactttatgcctttattagatgagcttgcacatgaaaataatggctgagctcggttccctgaaatgagagtgaggcagccatacctgagaaatgcagaaaaaatgttactagggaagagatatcctgaaccattggatgcattgcttagtgtgtaccagaaggataattaacacatttgctcctggtttttctttactctggtggcaatctcggatgcctgtgttagagtgagagaagggctgggggaggaaagggggttgtcctgagacagagtggatgtggttgtacttttcacgtgaatgaaaggatgtttgtgcttctgagatatggggatacctttctgtggtcaggatcatggtgtactcaagcatgtatttcaggggttaaactggaattcccctgcatggggatatgtgtgtcttagggttataggtccatttctgtagtgtgctgtccacatgattgcccaagagacttgggccacaaagtccacaccagcagctctcatgtgggtcactgggttcaagtatgtactcccttgttgcagagctcacatatgtcaaagcatgtatcctgtggggtgtgtgggcctcagggtctcagaatgtggatccgtgctatgcccgtgctcacagttgtctgtatgtctcaaaagcacatagatccccagttatttttctgtattgtggttctcatatacacgtacctccacagctcagtatatgtttctgtactatacacctgttcctgagggagtgatagggttctcgtgtcatggggtccacatttttgtatgcaaacctcctaacacctgggttttacaggtaaagggaagctgaggactgattctagggcagtttgcaggcaatttgcaattctggaagcagacagtgaaaatataattgtggtcctcccttgttctgttctggaacctagagggttaacaggcagctcttccggtccacccccctccttccagcctgtagcttcttgttgccgtggagatggtggccccgccccgatgcagcacctgcccccccattggctgcagtggtgactgtggggctgaggggggagcccaggcctgggcagccattctggagctggagccggagcttggcatccccccacttacattctcttccagcccctcggcccctctaaattcccttgcagccgagcctcagcttcctttttcagcgcttgccatccctctaggctcttggcagactgagctgcatccgcattcacctcccctcctcctcctttccctatccttacttgggagtcccgagaacccccactgtccatccttcctgctccctgcccattcccctgcattctttgcttttcccctccccccaccaaccttctgagcccctgccactgcagtgcacgtggaaccggctccttctcccttccccccacctgccccttccttggattttctgtccccagcttctccccctgctactcttcacaggtctcttccaagacctccccctccagtccagggagggggtcatgggaggcagccctggccctccagacagacaggggttccaggaggtggggggcaggtggaagatgtgcccaccagggggaagaggaaagcctgcctatgggcaatgacctttaacccatctttccccccagctagccccttctggggtgggtgggcaccagggacctagcctccctggatctttggtgtccttcattactggggttttactgaccctctcagtcatgctcccctgctgactgccagccccagtcatgggcctaaggcctcccaccccatccccctcagggggctcctgctcaggttccttgccccctccttcccgctgccagcctctccgccgtcgctgctcttcctgctgctttccggggggtaggtgaggccggagctgaggggcagagggaggtgggggcatgcactggggtcaggggtgggaacctgggttaacagcttcccttctggccccctccccaacttcccttctggccatttcctcccctccagaataccacttgggtcgctcgaggaggaagagtgtcccaggggggaagcagtacagcatggagggtgcccctgctgcgcccttccggccctcggtgagtggtgcctaccagatgtggctcagttgggccccctcccctccagccccaactggggccctaggagtctgagaaagagggcaggagggggagagagggagtgagagtgagaaaaagagtgtgtgtctgtgtgtgtctccccacctctttggtttcccccttcttggctctgcccccctgcttctgagaccagccctcccaccttctccaagctgtgtgtgtgtgtatatgtgtgtgtgtgtgtgcttgcactgtacccgtgggagcaaggaagacaaactggtattagggttcctatccccctttcctcgcctctggagacttcccttttccccatcccactttcatccaggggctctctaccagctgagggatgagtaggtagaactgaccctgccccaacccaccccatccccatttcccccccagcaaggcttcctgagccgacggctaaaaagctccatcaaacgaacgaagtcacaacccaaacttgaccggaccagcagctttcgccagatcctgcctcgcttccgaagtgctgaccatgaccggtacaggggctggagcatgtgggatgagattgatgtaatgtagggtctcctgtgtgagatgcagagggagggggttatctgtgtgcaaaggttgaaggattcaactcaagttggttgggggatgtcatggcacaggggacagaacagaaaagaactagaatagggatctgtgagcagcaggagaggggtagggtggcagagagaagacagacagacaggctggaaagggaatgaaggtgaagccaaggagggactcctcagggactcctcaggccaagaaggatgggctctagcccaggatcaaaggagctgtacaggaggagagtgaccctggaggaatgtttaaggaatgcagggaaggggttggtaggtgagtgagcaataggctgtaggtggaagggtgtcagggaaggtcaggaaatacaggggcagcaggttggagtggggctgggggtggctgaatgaatggatgatggctagggctcaaggacctcatcagtgagggaagagacagtatagagcatggcagagaaggggagggtgggacaggtgtgcagggtgacagaatgggaagcaacccatggactgaggcatgaagaagcagccagcggagaagtccagaaggcactgtccctgagaccaggctgaaggagacctccactgtttgcctttgttgcctgccatttggggttcctctctgggtttccccctcacccagtcactccccagggagaaccatgccctccctttcccccatgtctggccacccccaggattgggcaggtagggaggttgggataaagtgagtcacacctttccctgcccccctcccatgttgccagagctggatttggggccggcagggggtgagggcatggtattcctggccgcgggggcgggggggggggtccgggggccgggggagcgtcgcgctgacggcagccagagcctgcgatgacggggctgctataaataacttcttggaggctcccacacccaagctcccctcccgctttcccactgctctctactcttcatcccctgcccatctccataccgcttttgtattgctatcctacccctcattattccatgcccctagccccctttatcttctgccctcctgcagtgatttttttgcattccatcccctcttagccctcacctcggttctcccggccatctctccagttggccttcctcctcttctcctgtcctctgtcttgctgcacatacctttgtctccccctttcttcttcttgccctacctcctcttcttccctagtccctgtattctgtcttttatcctctttgagctcttttctgcccacagctttctcctatttcttatgcttttccctcactctttcccctgcttctgctaaaacttgtcctcttatgctgtgttcattcattctttgaatcattaaatgtttatcaggcactagccgtgtgccaggcccaggctagacatatctcttctctgtgccttcacttctttacttccactttttcctttatactgaggctctggtttctggggttacctggaggtactacctagaagtgccccaggcccactttgttctctccttttttttttttcttttctgccatggtccatttctgggttgagatatttctagatgtccccagtcctcgcaatcccttaggtgtgagatggtgggagtttcttttttttccttttttttttttaaatagaaatagggtctcactgtgttgcccagactggtcttgaactcctgggctcaagtgaccctcccacctcggcctttgaaatgttgggattacaggtgtgagccaccaggcccaggtggagcaggggagttccttaaaggattctgatttttctcacatccctcacgtccttcctgataggcagggtttctttctgtgtctgtttgggaagggtgttcagggggccttctctccaagtctccatcctggaacagactgatgatgcagggtacctatgtgtctaagaagagtaggggggccgggcgcggtggctcatgcctgtaatcccagcactttgggaggctgaatcacttgaggtcaggagtttgagaccagcctgaccaacagggtgaaaccccgtctcagctaaaaatacaaaaaaaaaaaagaaaaaaaaattagctgggtgtgctgaggcaggagagacgcttgagcccaggaggcagaagttgcagcaagccgagatcacaccactgtactccagcctgggcgacagagcaagactgtctcaaaaaaaaaaaaaaaaaaaaaaaaaggaagagtgggaagccctgatcccttcctctcctgaacctcctgcctgccagggcccggctgatgcaaagctttaaggagtcacactctcatgagtccttgctgagtcctagcagtgcagctgaggcattggagctcaacttggatgaagattccattatcaagccagtgcacagctccatcctgggccaggagttctgttttgaggtactgggtctggtgggctggggagggccaaaggacaggggtgatggaaggtggggggcagagaggtctagagaaagtggcacaggtggggacatcaaggaaacagaaacttctgggactggaggaaagggtaggccagggaggaagagaaggtagcagagtctcctcccctctgtagccctttccctcaactccacactcctttctaggtaacaacttcatcaggaacaaaatgctttgcctgtcggtctgcggccgaaagagacaaatggattgagaatctgcagcgggcagtaaagcccaacaaggtattggggaataaaggggacacaacctgtgtagggcaaaggttgcaccaacacgggaagctgggggcctagggaggaaagtgagttaaaggaggagaggcttggggaaggagaggattgaggtacagtgtatctggacaagcagggggagacccccattattctgagtcccccatttcttttcgctttctgtactgctaccctgccttacgatctctttccctgccatagaagtcatagacttacagagttgatggggctctggaaattctttagtctagcttccctgcaggcaggaatgcttcactaataccccgcaggacacatcaaatacacttagccaagtttctgtaccttggtttcctctctaagacagagggaactggtggcaatgggttggattaaatgatctctaaggtccctttggcacataaattctatgagtctgttcttcccaaaacttcatgcttccagttctgtcagctattctctgtatcccagtttctagcaagcttacagtcctagtcacactcctctggggggaactcttgtgtcttgatgtccttaaagaactcaacccaagatgctgacatgatctgacctatgcagagtacaaacaccatgttccctttcaacacactgcaatatcacctgagccttaccattagcttgtgctcaattgtatccccccaggcccttactgattcttttttttttttttttgagacagagtctcactctgtcacccaggctggagtgcagtggcgtgatcttggctcactgcaacctccgcctcctgggtttaagcaattcctgtgcctcagcctccccagtagctgggattacaggcatgcaccaccacacccgactaatttttgtatttttagtagagacgaggtttcaccatgttggccaggctagtctcaaactcctgacctcaggttgtctgcctgcctcagcctcccaaagtgctaggattacaggcatgagccaccacatctggccaagctttacccattctatatgcaattcttttttcacaatttctgatagtctctgcaggactttccagttcccttcaaattctacattaatatttttggcttgttattccagcttttgaaatctttccaatactgtttgtgttgtctagtgtatgtctacgcttctacagtagcttcctggggctgctataacaaactgctacaaactgggtcacgtaaaacaacagaaatttattctctcacagttcaggaggctggaagtccaaaagcaaggtatcagcagggccacgctctctttgatgctgtagcagggaatccttccttgcctcttcctagcttccggaggttgccagcagtccttggcattcctgggcttataactgcatccgtctaatttctgcctccatcttcatgtagctggcttccttctgtgtgtctctgtatcctgtatctctgtgtctccaaatctccctctccatataaagacaccagtttttataaggtgggttaagggtccactctaattcagtatggcctcttttttttgagatggaatatcgctcttgtttcccaggcttggagtgcaatggcatgagctcagctcactgcaacctccgccttccaggttcaagtgattctcctgcctcagcctcctgagtagctgggattacaggcacgtgccactatgcccagctaatttgtttttgtatttttattagagacggggtttcactatgttggccaggctggctcgaactcctgacctcaggcaatccacccacctcggcctcccaaagtgctgggattacaggaatgagataccatggctggcctcttcttagtttgattacatttgcagggatcctgtttccaaataagatcacattcacaggttcagggtagacatgagttttggtgggatactattcaacccagtacaccgtctcaccttatgattgctgtctgtatcccactttcttactgtctctcctcttttggtgtctctctgtcccttccccctttaacatcatgcccaccccaccatgccaggacaacagccgccgggtagacaatgtgctaaagctgtggatcatagaggcccgggagctgccccccaagaagcggtactactgtgagctctgcctggatgacatgctgtatgcacgcaccacctccaagccccgctctgcctctggggacaccgtcttctggggcgagcacttcgagtttaacaacctgccggctgtccgtgccctgcggctgcatctgtaccgtgactcagacaaaaagcgcaagaaggacaaggcaggctatgtcggcctggtgactgtgccagtggccaccctggctgggcgccacttcacagagcagtggtaccctgtaaccctgccaacaggcagtgggggatctgggggcatgggttcgggagggggagggggctcggggggtggctcagggggcaagggcaaaggaggttgcccggctgtgcggctgaaagcacgttaccagacaatgagcatcttgcccatggagctatataaagagtttgcagagtatgtcaccaaccattatcggatgctgtgtgcagtcttggagcccgccctgaatgtcaaaggcaaggaggaggttgccagtgcactagttcacatcctgcagagtacaggcaaggccaaggtgagtgttgtgccctcagggaaaggtgacttgggaatgggcacttgcttgggggttagtgaggacagggcaaattcacgagattgggttgtgcagaggctgacacttggattttcctgggcctcaggacttcctttcagacatggccatgtctgaggtagaccggttcatggaacgggagcacctcatattccgcgagaacacgcttgccactaaagccatagaagagtatatgagactgattggtcagaaatacctcaaggatgccattggtatggcccacactcaggccctcttcttcccaaacctgccagatgtccaccccagaccccaagtccacccttccacagcttgatacttcctaacccagagtcctaggactccagcctccaacacctgattctgaaatttccccaaccctggccacccccttccctgcccttggaaagtgtgaccacaccctcttgtgcccccaccccccaggagaattcatccgtgctctgtatgaatctgaggaaaactgcgaggtagaccctatcaagtgcacagcatccagtttggcagagcaccaggccaacctgcgaatgtgctgtgagttggccctgtgcaaggtggtcaactcccactggtgagactgggaacgctgggctggggggccagggtcgggggaattatgtgttcatctgttcatctatctgtccatcctcaaagaggactgagcaccatttatgggcaaagcattgttctaggcgctatagagcaaacaggtgaaagaggcctggtccctgccctcagagggcctccaccagaatggggacaaattagaagaaaaaaaaaaaaagccacagagccataatggtgtgtaagtgctgagtaagggtccccccaacctctgtgtgacataaggtcagagagaaggcagagctttgagataagtggggaagaggtgcccccttgggtaggctttgaagactggtttaggttctgatatatggacatagttggcaagaaagacatttcagaagaaggctgtgagaaaggcacatgtgtgatggtgaaaaggcccaggagttttcaggggacattaaagtaggttagtagcaattacatcaggtttagtggagcatgtgcctcataatggggagtggcgggagagatgtctgggcaggaagattagctttagaaactggaaggcctcaaggagtctgaggtcattggtaggccttgggatgccattaaaggtgtcagaaatattgtgatttagaagattaatccataggctgggcacggtggctcacacctgtaataccagcactttgggaggctgaggcgggcagaccacctgaggtcaggagtttgagaccaggctgaccaacatggagaaaccctgtctctactaaaaatacaaaattagccagatgtggtggcacatgcctgtaatcccagctattcgggaggctaaggcaggagaatcacttgaatctgggaggtggaggttgtagtgagccgagatcacaccattgcactctagcctgggcaacaagagtgaaactccgtctcaaaaaaaaaaaattaatccataagtagagtgtatgtagtagagtagagggatgtatgttgggggatgactggaatagagccagctgggggctcggaggcatgaaagtcagatcctgaatcagaacagtgacagaagttaggaaggagctactggaaggaccctctcgggaaaggatcagtaggaattgtcagcttattggaaaggagggagcatgtctgtggggagtcacggatgactcaagaggccatgaggctggtggttgggagaccggtggcctcattgacaaccaggaaagtcaggaggaggagccagttggaggtgtgggggcgatggtgagctctgctttacaccagccgagtttaaggtgtcagtgggacattgaagtggaactgggaggtgcggggagtgagctctgagccattccagggactggggatcatgcctggggcacctccatccccatttccctggaatccagaagagttggggggtccgagctccctgtacctcaagtgaccctccatctctctcccatctctgtctctccctggtgtctgtttttcttctcctcctctccttgtctctctcccacacccctccatctctctcccacgtgtctctcccctcaccttctctccccctccatttctctctccctaatctgtctgttccctctgccatggcccccttcttcaagcagcctcccatcttgctcctgcggtccctccttccctgtctctctcacccctgtttccacaccctcacctcctaccacccccctcagcatgttccctggaagctgagggtctctggggctcagtcccggtctctctctttctctctctctctctctgtctccccgacccttccccccagcgtgttcccgagggagctgaaggaggtgtttgcttcatggcggctgcgctgcgcagagcgaggccgggaggacatcgcagacaggcttatcagcgcctcactcttcctgcgcttcctctgcccagcgattatgtcgcccagtctctttgggcttatgcaggagtacccagatgagcagacctcacgaaccctcaccctcattgccaaggtcatccagaacctggccaacttttccaagtgagggaagcttcaggagtgggcagggcagggagtggcagggcagggagtggcagggctgggggtcggcaagaagggtctcctgagtccccagagatcctgagatggggaggctatgataccttgtgtgtgtgtgtatgtgtgtgtatgtgtgtgtgtgtgtgtgtgtgtgtgtatgtgacctttatcttctgcattcttggctaggtttacctcaaaggaggactttctgggcttcatgaatgagtttctggagctggaatggggttccatgcagcagtttttgtatgagatctccaatctggacacgctaaccaacagcagtagctttgagggttacatcgacttgggccgagagctctccacactgcatgccctactctgggaggtgctgccccagctcagcaaggtcagcagatcccctctttgccctatccccagatggctccagaggttcctggagcctgagaaactaccctttgaagattttttttctccccttgtttctcgaggtgtcaccactactatcccaactcaggccccctccacctgcaccctcagaggccctcttagagctgggcactgagcccccaggtaacagcctcacccttccaggaagccctcctgaagctgggtccactgccccggctcctcaacgacatcagcacagctctgaggaaccccaacatccaaaggcagccaagccgccagagtgagcggccccggcctcagcctgtggtactgcgggggccatcggctgagatgcagggctacatgatgcgggacctcaacaggtgagcaccctgggacagccaggcctgtgccctaggagcccttctcctattctagatactcctcactgggccccacatgcatctctctagggcttgaaagaagggaggaaaaagcaccaagttctcaggggagacgataaggagacaggtacagtcagtggtaggctgagagccctttacagcctgagggagtgagagatttggagctctaggaatagggctgaggctccaccaactcacggcttagttgtaagcctagagcatccctgctgcaagctctgatttgctgtccctctgcctgcccatgctagtccccaggctgaggttcagccagcatgtcatgtcagccatgtgtcaaaatgttcaaacatctcagtaatagctagtgaataagcacttcccccagcccccgaccacaaccccacagacctccccatgatccagcacttagagcagtgacagcagaagcctagcagggcctgcagcctgctccagagtcccagcctccattctgataggtggtgcccgtgcttcatttgctgctcattattttgatgatggccctgctttttcctctgcccgtgtcttcctccatgacaccatacccatcccaccattcccgcctctcctttcatttgtccacatctctctccttctctgtctgtgctcgcccctctttccatctctctccagctccatcgaccttcagtccttcatggctcgaggcctcaacaggtgaggggctctcccctcccccgccctcctctcctctcctgtctgttccctctcccactccactggccttcgccctactcctctcctctcctcctccatggacctcatctcctccatatgtgcccagccctgcccccatcccttctcttgctgcccccatctcccctcctctaggcctcacccccttcccggaggggccctgtcctttccctttactcacctgtcccctcccatcctccctgcctgccctcttcagggctgccaccgctagctctcagcccttccctctgggtcccacttttcaccccaaggcctgtgccagaccacagcaaggttcaattgctaggagccctgaccttaccttctgcttgtgtgcccccttcccttctgacagctctatggacatggctcgcctcccctccccaaccaaggaaaagccacccccaccaccgcctggtggtggtaaagacctgttctatgtaagccgtccacccctggcccgttcctcaccagcatactgcacgagcagctcggacatcacagagccagagcagaagatgctgagtgtcaacaagagtgtgtccatgctggacttacagggtgatgggcctggtggccgcctcaacagcagcagtgtttcgaacctggcggccgtaggggacctgctgcactcaagccaggcctcgctgacagcagccttggggctacggcctgcgcctgccggacgcctctcccaggggagtggctcatccatcacggcggctggcatgcgcctcagccagatgggtgtcaccacagacggtgtccctgcccagcaactgcgaatccccctctccttccagaaccctctcttccacatggctgctgatgggccaggtcccccaggcggccatggagggggcggtggccatggcccaccttcctcccatcaccaccaccaccaccatcaccaccaccgaggtggagagccccctggggacacctttgccccattccatggctatagcaagagtgaggacctctcttccggggtccccaagccccctgctgcctccatccttcatagccacagctacagtgatgagtttggaccctctggcactgacttcacccgtcggcagctttcactccaggacaacctgcagcacatgctgtcccctccccagatcaccattggtccccagaggccagccccctcagggcctggaggtgggagcggtgggggcagcggtgggggtggcgggggccagccgcctccattgcagaggggcaagtctcagcagttgacagtcagcgcagcccagaaaccccggccatccagcgggaatctattgcagtccccagagccaagttatggccccgcccgtccacggcaacagagcctcagcaaggagggcagcattgggggcagcgggggcagcggtggcggagggggtggggggctgaagccctccatcaccaagcaggtaggtgaaggcaggaggaaggcgggctgggtcacaacagggagggaagaaggagatggggggtggggttgaaacagagtctgtggcctgaagttacaatcttcttgcctccttttggccattaaacaaatgtgtatagagggcctgctatgtgccatgtgctatgccaggcactaaggatacggcactgaaccctcttagcactctcattccagaggggattaatccataagtagaatgggggtgactggaatagggccagctgggggcttggaggcatgaagatcagatcctgaatcagaacaacaggagaaattctctggcattcaactcacatctctggcattcagaggtgataaaggctacagcagggcaataggactgggactgtcgtctcctttggctgtgctgttgccctctaaatgtaccctgctttttcccaccttttctttctctgttcgccctcactgtgccttgtcccagcattctcagacaccatccacattgaaccccacaatgccagcctctgagcggacagtggcctgggtctccaacatgcctcacctgtcggctgacatcgagagtgcccacatcgagcgggaagagtacaagctcaaggagtactcaaaatcgatggatgagagccggctggatagggtacgatgggctctaccagctccaagccccagtgtttccttttacccacaggggagatctctagtcacttccaagggaaacctccagggtcagttatggtggtaagaaaaggcaaagacctgatcacctcttgagaagccttccgtccatctggggagaccagatacacacagttgttaaaagtcactttcaactctattttgtaggttatatatatcatagactatctggggatcatctattttaggcttacctccatgttcttccccaggaggaacataagccctggctcctgtgactgcttaggaaaggaaatgttactttactggcagtaggagctaataaattggatggggatggagggggttgcagctatattacaagtgatgtctggcctacccagtcactgtcccatgggcataagtgacagtcagtgggcagggaatgcatgtggtaaagtagtatgtatcagtcatgtgtgaaaagaccatccagaccctcacttggtggaggctgagggcaaagggcaccaagagctctggggctaacacagataacttagcagaaagttacttgaggaagctgttgttcctagggcctgaggaagagggcggctgcaggattaggtcatatataaagaacatatgatcctaacacggggaagtggtctttgagcataggaaatgttgggactgactcagatgggcttgaccagcaaatttaatgagaattaaaccctaatattagccccaaggcagccaccgtaggaagttaattcacatctctcctctgcatgtagaagggttggtgatatatgtatgtatcctcttcagagaatgaggaaatagtcttttgaatcattttttttttctgtgcttcagatgaggaaaccttgttgagaaaggctgtagactagcactatccaatataactttctgcagtgatgaaatgttctgtaatctgtagtgttcacatgtgtctagttgtgattcaggaactgaatttttagatctatttaattttaattttgtaatttttttgagacagggtcttgctctgtcacccaggctggagtgcagtggtgcaaacatggctcgaggcaacctccgcctcctgggctcaagacatcctcccacctcagccccctgagtagctaggactacagggtcgtgctaccatgcctggctagttttttttcttttttttttttttttttttttttgtagagacggggttttgccatgttgcccaggctggtctagaacttgtgagctcaagtgatctacctgcctcagccttccaaagtgctgggattacaggtgttgagccactgtgcctggcctaaattttaaatagctacatgtgggctggtggctaccatattagacagcacagctgtagactctagagccaatagggcagataaaatgactcttctgagctcttccagctctggaattcctagattctcagcctagaattcacttgtagacctcctatccttggcaccttgacagagccaaggagagggtcacatggaggacagcacctgcctttcccccgtcaattgccttttcttcctatgtctccagcatgtgttctggggcctggcttagggctgaagagtcaccctatttttaatttctgagcacagttactcaaggtgtgtttgtgagtgtaggtttctgtgtgggtcttcatgaggggccatacatagacctcagggtgtgggagtctgtgaccttgctccattttgagggaacctcagcaccatggcagggtcttctcaaaagcaggtgtgtggatatgatccacaggcagggaaggggttggggaaggcagctggatgtccctcaacatgcagtggctgtgcttggcagacagggatggaggctgggtggtgggcttggggtggggcgcccctcatagtgcggggtcgtgtgcccggcgggcaggtgaaggagtacgaggaggagattcactcactgaaagagcggctgcacatgtccaaccggaagctggaagagtatgagcggaggctgctgtcccaggaagaacaaaccagcaaaatcctgatgcagtatcaggcccgactggagcagagtgagaagaggctaaggcagcagcaggcagagaaggattcccagatcaagagcatcattggcaggtgaggggcggcctggggagggggttgtgagggagagcctgaggctggagagagcaagtgggcgagctactcctctgactcccatccccaaactcaggagcccaccaggagagcccaccactctcctccccaggaagccacccactcactcatcaccagatggagagaaaccccaacctgcttagtgcattaaatatctctttaccaaaccctgacctctcttctgatagagtagcttcggaagcccttggaaaatgtacctgttcctgtccaaccatcactgcatttgcatttaccctaggccagagctccccactagttattctcaacttaacctgtgatgttcactccaaacctaagcagggctccctagccagagtaggccctgcccttcctgggtggaccctccctctctagccttggaaaggtgttctgttagaaagggtcttttagcctgtgtatgttttcagctgctccagcaagtcctgggctccaaagagggtatcctcagcaaagaggtcaattatcttcagagtggtggggtcggggtgggggggaccctgggcgcactccaaccagagccacctccattttgatccattctaaatgtattttatgtaagatttaattagaagaaaagggcttcttgaaatattttttgaaaaccactgctctaattgatatcctttatgataaatcacctcgaggatcttcacagtgaggtgacatgggggatgcagaagccaggtcctcagccatggaaggtctggggaaggggcactgctgtcctgattgggacgatggaggcctggaggtgtctggatggtagaagtctttgaggcacagaaagctgccttagcagggaggtgtaagggttcctgggaggaaggtggagagcatgatcctgaggaacagggagtcttgcatcacggcaatggagggactctgattctaagggataaggatgcctgaggtttttccagagagctatggggttccatgggcaggctctgagcctgtgcccgccactaaccccactgaagcccgtcccttcaggctgatgctggtggaggaggagctgcgccgggaccaccccgccatggctgagccgctgccagaacccaagaagaggctgctcgacgctcaggtggaaattacaatgtcatttatcttctccgtgtcccatccccatccatcccactgtctttcgtgcactcactacaccagccacctagccccatcaccatctgtctctcatagtctgctgtttgtccactggctgctcctggcagccccctagtgaccccatcttcatcccatcgtctgtgcctttgtcactcctggcagcgtcagcccaactcctgtgccttcccatccagtcttcccactcctctctgcatctcaggaccttctctaccagaaccttggtctttctgcccctagaccccacctagttccaagaacccctgccccttctttgctcactcctattcaagccacgttgttcagcttcctctgcgctcttgggccagagggctagaagctgctgttttctggaatagagcacagggcagtatgatctgtagtttctccaggccctggccggtaccctgaaaacttggggacccatcacctctgttctcttggctccctaattttcctgtctccttggcagctcctgcatagcttcctcttcctgactcttcagatcttgaaggccttccatcctgtaacctccctttgccctcagtatttaagtccagcctccctctggcctccctcccactctggccctcagaccttcccagctgcctgctgcccagcctctcttctcacaagccagcttctaggacctcccttctgcacccttaccccttgctttcccaaaattctgctcattttcctacccatactcctctttgctctgactgctaggctccccccgcctgccatccccccaccaaggctcctgaccccatgaccccactctctcccactgcagctcctcatcaggtaattctcctggttccgctttggccacgggcggaggacacagggggaggtgactccggaccactgcaggttggtcgtgaagcccactccctccaacacctccggagcctctcccctctcactgctgccctccacacccagagaacctccacagactccagccctccgacacctgcacagatccatctcccaagacaccacccaaagagagcatttgctgctgcttcccagaactgtccaacaataccttagcaacaccaagagttgggccctagatgggcccagcacattcacaggtcacacccacttccctgcaaaacccaccccctcccagcctcctcctgactctaagccctcctcttcctctacctctccagtgtatgtctgtcaccccccatttcaccagagcgtccttaggggctgggggtgggtttgttaatggggtggaggcaatgatgggttggaggatcttggctataggggctgtgctgactgcagcaggtaggttgggtttccctcttccttccctaatcttggttctctaccctcctttccactcctcacctgattctctctcttcctcctccttatatctgtgaggcagaaggcatctgaagctcatattagcccccattgggtgggaattaggagtgggtagttaactcagggagacttgagataccctggaaaaaatgctattgagatgtcctgacattaggcagggtggatggaacaagaaggagcaagaaaggaacctcaggcagatgttaggacatggacttgatcatgtggcctgggagtttagaaatggggagagacatcctcctagatcagatcgtgggctcagtaggcatgttgattcccagggagaggtgccaggaacagcatggtaaagaatgtactcttcacagctcacatccccaggttgctgatgccactcactccccctctcctgccatcgagtggccttgccggacacatcaccctacctaaaaagccagtaaatgagaacctgtcagctatagccatcatttctgagatgcgattttctttgggattgagctgcagtgggcagtggctccttacactgtaattttaattctctgcctgcccagcctctctgtcaaagtagctggtgatctataaagatgctaaaaggcaccaggggactttgccatttaaaggactcctgcagtgaattcttttgtaaaatgaataatggcaccctaatttatccactttctaaatttgggtccatgggggtgtccagggcatgcttatgtgctgtcaccagcagacaaacagagggaatggaatctgggggttccttccctgctctcccgccatactcaggataccctaccataagtgatttcctctcactgacttgcagaaaatgtgtgagatacccagcaagctaagaaggcagttttgctgggtatctcatacccaaggctggggtttgggtgatctgagaggttagctccttgatcctaggatggaagggagagcttatatagaagcttttacttggaaggttttgtatcctaaggtcagacatagctatattaccaagcctaaatgccatgtggcccaggaaataatttggacatttgttctaaaccacttgtggtaggtattggtctctctgcaactcagccattaattagaaattagttttgagcctgaattttaaaaagccaagtgttgcccccagcccacacacacacacacggacatgtacagtacaaaccccagataattacaacagccaaagagagaaggaagtgaatttcccaaccagaagcgtagggaaattcagatggctttcttttctcccagcagaggaacagaaggcggagctaagggcaggaaccaggagttggtcaaggagctataggaggtgatgagagtagaaccaggggtaggagctggtctggtacccctcaccctctaattgggagcccagggagaaggactgaaaagaagatgggagtggaaaagaataaagccagtttctgcttcccagggatgcagagattggggcatgctgtgtctgcagaagctcctagtcatttccgccataattgtgagagagaggggcagccctcccacaagatttttcccttcccatatcacttccctgaatccccttcctttccccccagtacagttaaacctctctctaatttggaatgttatatttgagaagatggccactgtgaataagtgacaaacggaatgacagtgtctatttaatgaaatgcatgtcttcaaaatatatacagaactcgatgaacaaggctttttccactcctcagggagcatgcattaatgaatagataggattcaaaagtctgttttctggtatgggttaaatatcccctcctacagacatatttccaccactaatttgcttagtacaccttttcttcacagataaaggaaaatgcaagctcagtttttcttcagattatgaagaaattccaaatccacaggggtttggattaatgaggttttgctgtactgcctccccttattcctcaacatgaagttcccacctcggattggggatgggtgggagggggtttcaagaggaggagggtgggatgggcaaggaatatacacaggtgaagccagagaagggttaggttgggggtgcggtgggaacttgctgttttgatctggtttcctggtgtgacactctgggttaaaggcttgaaggcccctgttaggagtctaggggtgagattctcttctctctgatcccagaggacgttaacttctactgcaggtgagaaacaaaataggaggatggtggggactgtcctgggaggagggggtggtccatggcttgtggtgtgggctggctataggggaggccactgctaggggactggcatccaggcccccttgaagcgtctcaataagtccgcgctctcctttttggtgtcttgcaggagaggcagcttccccccttgggtccaacaaacccgcgtgtgacgctggccccaccgtggaatggcctggcccccccagccccaccacccccaccccggctgcagattacggagaacggcgagttccgaaacaccgcagaccactagcccacccagcatcagagaccttctcttcctttcctgtgcaccccaccctgtaacagcaccaaccaccaggattggacatcaccgaggaacagcgggattgcctccccgaatgcctccctgggaggcacactgattgcccacccccaccactgcaccatttccaggagggagagtggggaccctcagccgcccccttttccttcccattggggtgctgccctctctttgacccccagggacccttgccccaggacaccgcctaccccgtacagaccccttcactccggggtgctatccccatcctctgcctcatcgttcccctgagcactgggggacagaccctcacccccaccctgggggtgtggcacctccaaactttcaacttcagggtgatttttttagcagtaaccagagctgacaatctaactcccctccaccgccccattttggcctcccctgccccccttgttatggggaggggaccccgggtgagggggccctattaccccttgatttctcaggagcgtctgggggggctcagcacgcacaaactccttctccttctaccactcttaaatttactccctccccacccagaacccagatggggtggagggggccaccggggcagggagggggcggcaaggggggaatgggagttgtctccccttctccccacacctgatctgctctcggctggtcccagagcggggtgagggggcttatgcccccccctcccccagtgtgttgggtggggtggaattgaggttagggtgaggggtcagggtttaggagggtgtgtatgttgggaggacaggctagttgatctgtcctactctgacacacagtcccctctgccccttccttctctcttcttggtctctactcccagggggaggggggaacttactctaggaaaagccatgtctctctcccccagggtggggggacctgtgttggaggaggggtgttggggggcccccttccatgactctgtcccctgggggaggtaggacagggctgggcttccctctcatcctccccctcccaatctccttccacctccctccctcccgccagctccacgatttttcggtgtttctctgtacatagttttctggcgggataggggaggtaggatggatggggtttggggtgggtaggccatgggaggggagaagcccctccttggcaccccctcttccctgactgctgtcccctacccagccttgcccccttcatccttttgcgtttggtattgagactctcctagactctactcctctttcttttgtatggacagttccccttcagtcccatccccctacacatacacccagccggggccaaatttatacttatataaaagttgtaaatatgtgaaattttatccctgtgccctttccccacctcaggccctacccctggaccctccccaaccttccttctctcttctttggctgttgtaattatctggggtttgtactgtacatatccggggtgtgtgtgtgtgggctgggggcaacccttctgtacagagcttcctggccccctccccccccgcccctctgcttccctccccacccaccacctcaagggtagggagttgctcttcctacctgttttattttgttttctcgttctccctccccaccccactcccagccttatctatcccccctcactgtccccttttctccactcccagccccatttcctttttttctggagtgtgtggtgaaacagaaaaaaacatgtttaataaacggagattgttctttta

SEQ ID NO: 8 - protein, 1343 aa, synthetic construct [sequence omitted]

SEQ ID NO: 9 - protein, 1292 aa, synthetic construct
  REGION 1..1292: note = human SYNGAP1 isoform 2
MSRSRASIHRGSIPAMSYAPFRDVRGPSMHRTQYVHSPYDRPGWNPRFCIISGNQLLMLDEDEIHPLLIRDRRSESSRNKLLRRTVSVPVEGRPHGEHEYHLGRSRRKSVPGGKQYSMEGAPAAPFRPSQGFLSRRLKSSIKRTKSQPKLDRTSSFRQILPRFRSADHDRARLMQSFKESHSHESLLSPSSAAEALELNLDEDSIIKPVHSSILGQEFCFEVTTSSGTKCFACRSAAERDKWIENLQRAVKPNKDNSRRVDNVLKLWIIEARELPPKKRYYCELCLDDMLYARTTSKPRSASGDTVFWGEHFEFNNLPAVRALRLHLYRDSDKKRKKDKAGYVGLVTVPVATLAGRHFTEQWYPVTLPTGSGGSGGMGSGGGGGSGGGSGGKGKGGCPAVRLKARYQTMSILPMELYKEFAEYVTNHYRMLCAVLEPALNVKGKEEVASALVHILQSTGKAKDFLSDMAMSEVDRFMEREHLIFRENTLATKAIEEYMRLIGQKYLKDAIGEFIRALYESEENCEVDPIKCTASSLAEHQANLRMCCELALCKVVNSHCVFPRELKEVFASWRLRCAERGREDIADRLISASLFLRFLCPAIMSPSLFGLMQEYPDEQTSRTLTLIAKVIQNLANFSKFTSKEDFLGFMNEFLELEWGSMQQFLYEISNLDTLTNSSSFEGYIDLGRELSTLHALLWEVLPQLSKEALLKLGPLPRLLNDISTALRNPNIQRQPSRQSERPRPQPVVLRGPSAEMQGYMMRDLNSSMDMARLPSPTKEKPPPPPPGGGKDLFYVSRPPLARSSPAYCTSSSDITEPEQKMLSVNKSVSMLDLQGDGPGGRLNSSSVSNLAAVGDLLHSSQASLTAALGLRPAPAGRLSQGSGSSITAAGMRLSQMGVTTDGVPAQQLRIPLSFQNPLFHMAADGPGPPGGHGGGGGHGPPSSHHHHHHHHHHRGGEPPGDTFAPFHGYSKSEDLSSGVPKPPAASILHSHSYSDEFGPSGTDFTRRQLSLQDNLQHMLSPPQITIGPQRPAPSGPGGGSGGGSGGGGGGQPPPLQRGKSQQLTVSAAQKPRPSSGNLLQSPEPSYGPARPRQQSLSKEGSIGGSGGSGGGGGGGLKPSITKQHSQTPSTLNPTMPASERTVAWVSNMPHLSADIESAHIEREEYKLKEYSKSMDESRLDREYEEEIHSLKERLHMSNRKLEEYERRLLSQEEQTSKILMQYQARLEQSEKRLRQQQAEKDSQIKSIIGRLMLVEEELRRDHPAMAEPLPEPKKRLLDAQRGSFPPWVQQTRV

SEQ ID NO: 10 - DNA, 18 bp, synthetic construct
  misc_feature 1..18: note = ET-015
  source 1..18: mol_type = other DNA
gggagagagatggagggg

SEQ ID NO: 11 - DNA, 18 bp, synthetic construct
  misc_feature 1..18: note = ET-016
  source 1..18: mol_type = other DNA
tgggagagagatggaggg

SEQ ID NO: 12 - DNA, 18 bp, synthetic construct
  misc_feature 1..18: note = ET-017
  source 1..18: mol_type = other DNA
gtgggagagagatggagg

SEQ ID NO: 13 - DNA, 18 bp, synthetic construct
  misc_feature 1..18: note = ET-018
  source 1..18: mol_type = other DNA
cgtgggagagagatggag

SEQ ID NO: 14 - DNA, 18 bp, synthetic construct
  misc_feature 1..18: note = ET-20
  source 1..18: mol_type = other DNA
acacgtgggagagagatg

SEQ ID NO: 15 - DNA, 18 bp, synthetic construct
  misc_feature 1..18: note = ET-21
  source 1..18: mol_type = other DNA
gacacgtgggagagagat

SEQ ID NO: 16 - DNA, 18 bp, synthetic construct
  misc_feature 1..18: note = ET-022
  source 1..18: mol_type = other DNA
agacacgtgggagagaga

SEQ ID NO: 17 - DNA, 18 bp, synthetic construct
  misc_feature 1..18: note = ET-023
  source 1..18: mol_type = other DNA
agagacacgtgggagaga

SEQ ID NO: 18 - DNA, 18 bp, synthetic construct
  misc_feature 1..18: note = ET-024
  source 1..18: mol_type = other DNA
gagagacacgtgggagag

SEQ ID NO: 19 - DNA, 18 bp, synthetic construct
  misc_feature 1..18: note = ET-025
  source 1..18: mol_type = other DNA
ggagagacacgtgggaga

SEQ ID NO: 20 - DNA, 18 bp, synthetic construct
  misc_feature 1..18: note = ET-026
  source 1..18: mol_type = other DNA
gggagagacacgtgggag

SEQ ID NO: 21 - DNA, 18 bp, synthetic construct
  misc_feature 1..18: note = ET-027
  source 1..18: mol_type = other DNA
ggagagagatggaggggt

SEQ ID NO: 22 - DNA, 18 bp, synthetic construct
  misc_feature 1..18: note = ET-028
  source 1..18: mol_type = other DNA
ggggagagacacgtggga

SEQ ID NO: 23 - DNA, 19 bp, synthetic construct
  misc_feature 1..19: note = ET-029
  source 1..19: mol_type = other DNA
cacgtgggagagagatgga

SEQ ID NO: 24 - DNA, 19 bp, synthetic construct
  misc_feature 1..19: note = ET-030
  source 1..19: mol_type = other DNA
acacgtgggagagagatgg

SEQ ID NO: 25 - DNA, 20 bp, synthetic construct
  misc_feature 1..20: note = ET-031
  source 1..20: mol_type = other DNA
gacacgtgggagagagatgg

SEQ ID NO: 26 - DNA, 21 bp, synthetic construct
  misc_feature 1..21: note = ET-032
  source 1..21: mol_type = other DNA
agacacgtgggagagagatgg

SEQ ID NO: 27 - DNA, 22 bp, synthetic construct
  misc_feature 1..22: note = ET-033
  source 1..22: mol_type = other DNA
gagacacgtgggagagagatgg

SEQ ID NO: 28 - DNA, 18 bp, synthetic construct
  misc_feature 1..18: note = ET-062
  source 1..18: mol_type = other DNA
ctcagcttccagggaaca

SEQ ID NO: 29 - DNA, 18 bp, synthetic construct
  misc_feature 1..18: note = ET-063
  source 1..18: mol_type = other DNA
agaccctcagcttccagg

SEQ ID NO: 30 - DNA, 18 bp, synthetic construct
  misc_feature 1..18: note = ET-064
  source 1..18: mol_type = other DNA
cccagagaccctcagctt

SEQ ID NO: 31 - DNA, 18 bp, synthetic construct
  misc_feature 1..18: note = ET-065
  source 1..18: mol_type = other DNA
tgagccccagagaccctc

SEQ ID NO: 32 - DNA, 18 bp, synthetic construct
  misc_feature 1..18: note = ET-066
  source 1..18: mol_type = other DNA
gggactgagccccagaga

SEQ ID NO: 33 - DNA, 18 bp, synthetic construct
  misc_feature 1..18: note = ET-067 ASO
  source 1..18: mol_type = other DNA
agaccgggactgagcccc

SEQ ID NO: 34 - DNA, 18 bp, synthetic construct
  misc_feature 1..18: note = ET-)68
  source 1..18: mol_type = other DNA
gagagagaccgggactga

SEQ ID NO: 35 - DNA, 18 bp, synthetic construct
  misc_feature 1..18: note = ET-069
  source 1..18: mol_type = other DNA
agaaagagagagaccggg

SEQ ID NO: 36 - DNA, 18 bp, synthetic construct
  misc_feature 1..18: note = ET-070
  source 1..18: mol_type = other DNA
gagagagaaagagagaga

SEQ ID NO: 37 - DNA, 18 bp, synthetic construct
  misc_feature 1..18: note = ET-071
  source 1..18: mol_type = other DNA
agagagagagagaaagag

SEQ ID NO: 38 - DNA, 18 bp, synthetic construct
  misc_feature 1..18: note = ET-071
  source 1..18: mol_type = other DNA
cagagagagagagagaga

SEQ ID NO: 39 - DNA, 18 bp, synthetic construct
  misc_feature 1..18: note = ET-073
  source 1..18: mol_type = other DNA
ggagacagagagagagag

SEQ ID NO: 40 - DNA, 18 bp, synthetic construct
  misc_feature 1..18: note = ET-074
  source 1..18: mol_type = other DNA
gtcggggagacagagaga

SEQ ID NO: 41 - DNA, 18 bp, synthetic construct
  misc_feature 1..18: note = ET-075
  source 1..18: mol_type = other DNA
gaagggtcggggagacag

SEQ ID NO: 42 - DNA, 18 bp, synthetic construct
  misc_feature 1..18: note = ET-076
  source 1..18: mol_type = other DNA
ggggggaagggtcgggga

SEQ ID NO: 43 - DNA, 18 bp, synthetic construct
  misc_feature 1..18: note = ET-077
  source 1..18: mol_type = other DNA
aggggggtggtaggaggt

SEQ ID NO: 44 - DNA, 18 bp, synthetic construct
  misc_feature 1..18: note = ET-078
  source 1..18: mol_type = other DNA
tgaggggggtggtaggag

SEQ ID NO: 45 - DNA, 18 bp, synthetic construct
  misc_feature 1..18: note = ET-079
  source 1..18: mol_type = other DNA
gctgaggggggtggtagg

SEQ ID NO: 46 - DNA, 18 bp, synthetic construct
  misc_feature 1..18: note = ET-080
  source 1..18: mol_type = other DNA
atgctgaggggggtggta

SEQ ID NO: 47 - DNA, 18 bp, synthetic construct
  misc_feature 1..18: note = ET-081
  source 1..18: mol_type = other DNA
acatgctgaggggggtgg

SEQ ID NO: 48 - DNA, 18 bp, synthetic construct
  misc_feature 1..18: note = ET-082
  source 1..18: mol_type = other DNA
gaacatgctgaggggggt

SEQ ID NO: 49 - DNA, 18 bp, synthetic construct
  misc_feature 1..18: note = ET-083
  source 1..18: mol_type = other DNA
gggaacatgctgaggggg

SEQ ID NO: 50 - DNA, 18 bp, synthetic construct
  misc_feature 1..18: note = ET-084
  source 1..18: mol_type = other DNA
cagggaacatgctgaggg

SEQ ID NO: 51 - DNA, 18 bp, synthetic construct
  misc_feature 1..18: note = ET-087
  source 1..18: mol_type = other DNA
agcttccagggaacatgc

SEQ ID NO: 52 - DNA, 22 bp, synthetic construct
  misc_feature 1..22: note = ET-X1
  source 1..22: mol_type = other DNA
tggaggggtgtgggagagagac

SEQ ID NO: 53 - DNA, 22 bp, synthetic construct
  misc_feature 1..22: note = ET-X2
  source 1..22: mol_type = other DNA
gatggaggggtgtgggagagag

SEQ ID NO: 54 - DNA, 22 bp, synthetic construct
  misc_feature 1..22: note = ET-X3
  source 1..22: mol_type = other DNA
gagatggaggggtgtgggagag

SEQ ID NO: 55 - DNA, 22 bp, synthetic construct
  misc_feature 1..22: note = ET-X4
  source 1..22: mol_type = other DNA
gagatggaggggtgtgggagag

SEQ ID NO: 56 - DNA, 22 bp, synthetic construct
  misc_feature 1..22: note = ET-X5
  source 1..22: mol_type = other DNA
gagagagatggaggggtgtggg

SEQ ID NO: 57 - DNA, 22 bp, synthetic construct
  misc_feature 1..22: note = ET-X5
  source 1..22: mol_type = other DNA
gggagagagatggaggggtgtg

SEQ ID NO: 58 - DNA, 22 bp, synthetic construct
  misc_feature 1..22: note = ET-X7
  source 1..22: mol_type = other DNA
gtgggagagagatggaggggtg

SEQ ID NO: 59 - DNA, 20 bp, synthetic construct
  misc_feature 1..20: note = Modified Primer
  source 1..20: mol_type = other DNA
ttcagacgtgtgctcttccg

SEQ ID NO: 60 - DNA, 32 bp, synthetic construct
  misc_feature 1..32: note = 5' adapter containing 8-nt UMI
  misc_feature 1..8: note = n is a, c, g, or t
  source 1..32: mol_type = other DNA
nnnnnnnnagatcggaagagcgtcgtgtaggg

SEQ ID NO: 61 - DNA, 22 bp, synthetic construct
  misc_feature 1..22: note = SYNGAP-1 (exon 10-11) forward primer
  source 1..22: mol_type = other DNA
aattcatccgtgctctgtatga

SEQ ID NO: 62 - DNA, 21 bp, synthetic construct
  misc_feature 1..21: note = SYNGAP1 (exon 10-11) reverse primer
  source 1..21: mol_type = other DNA
aagagactgggcgacataatc

SEQ ID NO: 63 - DNA, 20 bp, synthetic construct
  misc_feature 1..20: note = SYNGAP1 (exon 14) forward primer
  source 1..20: mol_type = other DNA
tcctgaagctgggtccactg

SEQ ID NO: 64 - DNA, 20 bp, synthetic construct
  misc_feature 1..20: note = SYNGAP1 (exon 14) reverse primer
  source 1..20: mol_type = other DNA
gggtggcttttccttggttg

SEQ ID NO: 65 - DNA, 21 bp, synthetic construct
  misc_feature 1..21: note = SYNGAP1 (exon 18-19) forward primer
  source 1..21: mol_type = other DNA
aggcagagaaggattcccaga

SEQ ID NO: 66 - DNA, 19 bp, synthetic construct
  misc_feature 1..19: note = SYNGAP (exon 18-19) reverse primer
  source 1..19: mol_type = other DNA
tcacacgcgggtttgttgg

SEQ ID NO: 67 - DNA, 20 bp, synthetic construct
  misc_feature 1..20: note = ms-Syngap1 (ex10-11) forward primer
  source 1..20: mol_type = other DNA
agaccccatcaagtgcacag

SEQ ID NO: 68 - DNA, 20 bp, synthetic construct
  misc_feature 1..20: note = ms-SYNGAP1 (ex10-11)
  source 1..20: mol_type = other DNA
gcctgtcagcaatgtcctct

SEQ ID NO: 69 - DNA, 26 bp, synthetic construct
  misc_feature 1..26: note = ms-Scn1a forward primer
  source 1..26: mol_type = other DNA
cagtttaacagcaaatgccttgggtt

SEQ ID NO: 70 - DNA, 36 bp, synthetic construct
  misc_feature 1..36: note = ms-Scn1a reverse primer
  source 1..36: mol_type = other DNA
aagtacaaatacatgtacaggctttcctcatactta

SEQ ID NO: 71 - DNA, 22 bp, synthetic construct
  misc_feature 1..22: note = GAPDH forward primer
  source 1..22: mol_type = other DNA
tcatccctgagctgaacgggaa

SEQ ID NO: 72 - DNA, 24 bp, synthetic construct
  misc_feature 1..24: note = GAPDH reverse primer
  source 1..24: mol_type = other DNA
gcctgcttcaccaccttcttgatg

SEQ ID NO: 73 - DNA, 24 bp, synthetic construct
  misc_feature 1..24: note = TBR2 (EOMES) forward primer
  source 1..24: mol_type = other DNA
actcaatcccactgcccactacaa

SEQ ID NO: 74 - DNA, 23 bp, synthetic construct
  misc_feature 1..23: note = TBR2 (EOMES) reverse primer
  source 1..23: mol_type = other DNA
gctttgccacaggtcacccattt

SEQ ID NO: 75 - DNA, 26 bp, synthetic construct
  misc_feature 1..26: note = TBR1 forward primer
  source 1..26: mol_type = other DNA
tggagcactgcctttctccttctatc

SEQ ID NO: 76 - DNA, 28 bp, synthetic construct
  misc_feature 1..28: note = TBR1 reverse primer
  source 1..28: mol_type = other DNA
ctccaggttgtcagtggtcgagataatg

SEQ ID NO: 77 - DNA, 24 bp, synthetic construct
  misc_feature 1..24: note = CTIP2 (BCL11B) forward primer
  source 1..24: mol_type = other DNA
agtactgcggcaaggtgttcaaga

SEQ ID NO: 78 - DNA, 24 bp, synthetic construct
  misc_feature 1..24: note = CTIP2 (BCL11B) reverse primer
  source 1..24: mol_type = other DNA
gttgcacagctcgcacttgtaagg

SEQ ID NO: 79 - DNA, 26 bp, synthetic construct
  misc_feature 1..26: note = SATB2 forward primer
  source 1..26: mol_type = other DNA
ccatcatgagccctggtcttctttct

SEQ ID NO: 80 - DNA, 24 bp, synthetic construct
  misc_feature 1..24: note = SATB2 reverse primer
  source 1..24: mol_type = other DNA
cggctaacggcaatctgttggttt

SEQ ID NO: 81 - DNA, 26 bp, synthetic construct
  misc_feature 1..26: note = REELIN (RELN) forward primer
  source 1..26: mol_type = other DNA
cctcgttatgctgagacctgggattt

SEQ ID NO: 82 - DNA, 24 bp, synthetic construct
  misc_feature 1..24: note = REELIN (RELN) reverse primer
  source 1..24: mol_type = other DNA
ctggagctgtacactgtgggagtt

SEQ ID NO: 83 - DNA, 23 bp, synthetic construct
  misc_feature 1..23: note = NGN2 (NEUROG2) forward priimer
  source 1..23: mol_type = other DNA
cgaaaccgcatgcacaacctcaa

SEQ ID NO: 84 - DNA, 24 bp, synthetic construct
  misc_feature 1..24: note = NGN2 (NEUROG2) reverse primer
  source 1..24: mol_type = other DNA
ctcggtgagtgcccagatgtagtt

SEQ ID NO: 85 - DNA, 24 bp, synthetic construct
  misc_feature 1..24: note = VGLUT1 (SLC17A7) forward primer
  source 1..24: mol_type = other DNA
ccatcatgagtggtctgggcttct

SEQ ID NO: 86 - DNA, 21 bp, synthetic construct
  misc_feature 1..21: note = VGLUT1 (SLC17A7) reverse primer
  source 1..21: mol_type = other DNA
gcggtgggtcgtgctgttatt

SEQ ID NO: 87 - DNA, 24 bp, synthetic construct
  misc_feature 1..24: note = VGLUT2 (SLC17A6) forward primer
  source 1..24: mol_type = other DNA
agacccggaggaaacaagtgaaga

SEQ ID NO: 88 - DNA, 23 bp, synthetic construct
  misc_feature 1..23: note = VGLUT2 (SLC17A6) reverse primer
  source 1..23: mol_type = other DNA
aacctccattggcctgtgttgtg

SEQ ID NO: 89 - DNA, 24 bp, synthetic construct
  misc_feature 1..24: note = GAD67 (GAD1) forward primer
  source 1..24: mol_type = other DNA
ggctaagaacggtgaggagcaaac

SEQ ID NO: 90 - DNA, 24 bp, synthetic construct
  misc_feature 1..24: note = GAD67 (GAD1) reverse primer
  source 1..24: mol_type = other DNA
cagcaactggtgtgggtgatgaaa

SEQ ID NO: 91 - DNA, 25 bp, synthetic construct
  misc_feature 1..25: note = GAD65 (GAD2) forward primer
  source 1..25: mol_type = other DNA
accaaatgcatgcctcctacctctt

SEQ ID NO: 92 - DNA, 24 bp, synthetic construct
  misc_feature 1..24: note = GAD65 (GAD2) reverse primer
  source 1..24: mol_type = other DNA
tttgccctccacatcagccatagt